SEQUENCE LISTING (36 sequences)

SEQ ID NO: 1 - DNA, 3 bp, Artificial Sequence
  Synthetic Construct
  misc_feature (1)..(1): n is a, c, g, or t
ngg

SEQ ID NO: 2 - DNA, 3 bp, Artificial Sequence
  Synthetic Construct
  misc_feature (1)..(1): n is a, c, g, or t
nag

SEQ ID NO: 3 - DNA, 4 bp, Artificial Sequence
  Synthetic Construct
  misc_feature (1)..(1): n is a, c, g, or t
ngcg

SEQ ID NO: 4 - DNA, 4 bp, Artificial Sequence
  Synthetic Construct
  misc_feature (1)..(1): n is a, c, g, or t
ngag

SEQ ID NO: 5 - DNA, 4 bp, Artificial Sequence
  Synthetic Construct
  misc_feature (1)..(1): n is a, c, g, or t
  misc_feature (4)..(4): n is a, c, g, or t
ngan

SEQ ID NO: 6 - DNA, 4 bp, Artificial Sequence
  Synthetic Construct
  misc_feature (1)..(1): n is a, c, g, or t
  misc_feature (3)..(3): n is a, c, g, or t
ngng

SEQ ID NO: 7 - DNA, 6 bp, Artificial Sequence
  Synthetic Construct
  misc_feature (1)..(2): n is a, c, g, or t
nngrrt

SEQ ID NO: 8 - DNA, 6 bp, Artificial Sequence
  Synthetic Construct
  misc_feature (1)..(2): n is a, c, g, or t
  misc_feature (6)..(6): n is a, c, g, or t
nngrrn

SEQ ID NO: 9 - DNA, 6 bp, Artificial Sequence
  Synthetic Construct
  misc_feature (1)..(3): n is a, c, g, or t
nnnrrt

SEQ ID NO: 10 - DNA, 4 bp, Artificial Sequence
  Synthetic Construct
tttv

SEQ ID NO: 11 - DNA, 4 bp, Artificial Sequence
  Synthetic Construct
tycv

SEQ ID NO: 12 - DNA, 4 bp, Artificial Sequence
  Synthetic Construct
tatv

SEQ ID NO: 13 - DNA, 8 bp, Artificial Sequence
  Synthetic Construct
  misc_feature (1)..(4): n is a, c, g, or t
nnnngatt

SEQ ID NO: 14 - DNA, 7 bp, Artificial Sequence
  Synthetic Construct
  misc_feature (1)..(2): n is a, c, g, or t
nnagaaw

SEQ ID NO: 15 - DNA, 5 bp, Artificial Sequence
  Synthetic Construct
  misc_feature (1)..(1): n is a, c, g, or t
  misc_feature (4)..(4): n is a, c, g, or t
nggng

SEQ ID NO: 16 - DNA, 6 bp, Artificial Sequence
  Synthetic Construct
  misc_feature (1)..(1): n is a, c, g, or t
naaaac

SEQ ID NO: 17 - DNA, 3 bp, Artificial Sequence
  Synthetic Construct
  misc_feature (1)..(2): n is a, c, g, or t
nng

SEQ ID NO: 18 - DNA, 3 bp, Artificial Sequence
  Synthetic Construct
  misc_feature (1)..(1): n is a, c, g, or t
  misc_feature (3)..(3): n is a, c, g, or t
ngn

SEQ ID NO: 19 - DNA, 4 bp, Artificial Sequence
  Synthetic Construct
  misc_feature (1)..(1): n is a, c, g, or t
naar

SEQ ID NO: 20 - DNA, 23 bp, Artificial Sequence
  Synthetic Construct
tgcggaattgacaagttccgagg

SEQ ID NO: 21 - RNA, 20 nt, Artificial Sequence
  Synthetic Construct
ugcggaauugacaaguuccg

SEQ ID NO: 22 - DNA, 23 bp, Artificial Sequence
  Synthetic Construct
agagtttgtgtgttgcccactgg

SEQ ID NO: 23 - RNA, 20 nt, Artificial Sequence
  Synthetic Construct
agaguuuguguguugcccac

SEQ ID NO: 24 - DNA, 23 bp, Artificial Sequence
  Synthetic Construct
ggctgaagaaagtgacaatgtgg

SEQ ID NO: 25 - RNA, 20 nt, Artificial Sequence
  Synthetic Construct
ggcugaagaaagugacaaug

SEQ ID NO: 26 - DNA, 23 bp, Artificial Sequence
  Synthetic Construct
gaatcagaacttacagtcactgg

SEQ ID NO: 27 - RNA, 20 nt, Artificial Sequence
  Synthetic Construct
gaaucagaacuuacagucac

SEQ ID NO: 28 - DNA, 23 bp, Artificial Sequence
  Synthetic Construct
gttctctgtgtggatgtagcagg

SEQ ID NO: 29 - RNA, 20 nt, Artificial Sequence
  Synthetic Construct
guucucuguguggauguagc

SEQ ID NO: 30 - DNA, 7 bp, Artificial Sequence
  Synthetic Construct
ggcygcg

SEQ ID NO: 31 - DNA, 7 bp, Artificial Sequence
  Synthetic Construct
gtcyggc

SEQ ID NO: 32 - DNA, 23 bp, Artificial Sequence
  Synthetic Construct
caagaacctcgagtggattgcgg

SEQ ID NO: 33 - RNA, 20 nt, Artificial Sequence
  Synthetic Construct
caagaaccucgaguggauug

SEQ ID NO: 34 - DNA, 20 bp, Artificial Sequence
  Synthetic Construct
gacacgtgtgtttgcggtgg

SEQ ID NO: 35 - RNA, 17 nt, Artificial Sequence
  Synthetic Construct
gacacguguguuugcgg

SEQ ID NO: 36 - DNA, 2171 bp, Artificial Sequence
  Synthetic Construct
ttgaattattcatccttcatttatcactagtggatgctctcagaactttatgctaatctagatttgtgcaaatttagcaaatcccagaaaagcaaaaagacattcaaggagaaatctccagtgactgtaagttctgattcttagtggctgcaggggataatttcttctcttcccaggattgttactctgaaagtgaaccaagagagtcagaatttctaggataaggcaggtccatgtgagataaacttccatctgtaagggaggtaggagtgagctggggatctacctggaatgcgcacaatttctggatacacgaattaagccagaaggcattaattctctggtatctgttcaccagtggagactgctacatcttatttcccaattagtttccattccagttgttcgtataaacctctattatataacatcgtggtcttattaaataaataataaacaagaataaaaagagtaccaaagtgtaacccatgctaaaaaaaatcctgttgtaaattcactcaaaaagcaaataatcttttcctttgtgaaattggttcctaatatattgggtctgcatgttgattattttatgtggagttttcttacaatgaaacacatctactctaccactcactgttttctccttacactttgtagacatgcagtgagaagagtaccaacttgcatgactacggcatgttgctgccctgcggaattgacaagttcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcgaggggtagagtttgtgtgttgcccactcgctgaagaaagtgacaatgtcgattctgctgatgcggaggaggatgactcggatgtctggtggggcggagcagacacagactatgcagatgggaggtaaggtggcctttgtgttcagcctcagagatgctgaaacatcttgtatggagtatttgtatcctgtaaattaatctttctgtttatcactgaaaaggtctctgcccactcccatcagagtctgctgttatgcaaaaatctgaactatgaatttttatggcatcctgttgaattaataatatcagtcacccatcacagagttaattttaactatttaatattaaacttgggatcaaaatcccactgataactatcataggttactggtagttctaacagggagttgaaataataatggcgttctgttttggtcattaatttaaaaatatttttaaatgctctctgggattgtcagtcattagtttaaggtgaattttcgttaaaggtttggtttgagattttgggcgttctctgtgtggatgtagcagggtttttttttggccttttatcatttcttttccttcatagtggaagtcaaaggcattaataaatgcttgttaattttttttaactacctttgatttgttgattgtaaagaaacctattttcgcctctttttcttagggagcttagtcccgttacttcttacctgattctcattcttaa

(19) United States
(12) Patent Application Publication
(10) Pub. No.: US 2022/0220468 A1
(43) Pub. Date: Jul. 14, 2022

(54) COMPOSITIONS AND METHODS FOR HOMOLOGY DIRECTED REPAIR

(71) Applicant: Beth Israel Deaconess Medical Center, Inc., Boston, MA (US)

(72) Inventors: Volney SHEEN, Boston, MA (US); Gewei LIAN, Boston, MA (US)

(21) Appl. No.: 17/610,360

(22) PCT Filed: May 15, 2020
(86) PCT No.: PCT/US2020/033256

§ 371 Date: Nov. 10, 2021

Related U.S. Application Data
(60) Provisional application No. 62/849,504, filed on May 17, 2019.

(51) Int. Cl. C12N 15/10; C12N 15/90; C12N 9/22

(52) U.S. Cl. CPC C12N 15/102; C12N 15/907; C12N 9/22; C12N 2310/20; C07K 2319/80; C07K 2319/85; C12Y 301/11003

(57) ABSTRACT

Described are methods of homology directed repair (e.g., for treating a disease or disorder) and fusion proteins, polynucleotides (e.g., guide polynucleotides (e.g., guide RNAs) and polynucleotides encoding the fusion proteins), vectors containing the polynucleotides, viral or non-viral delivery vehicles containing the vectors, and compositions (e.g., pharmaceutical compositions) containing the same for use in methods of homology directed repair.

### STATEMENT REGARDING FEDERALLY SPONSORED RESEARCH

This invention was made with government support under Grant No. NS092062 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on 5.14.20 is named 01948-264WO2_Sequence_Listing_5.14.20_ST25 and is 11,546 bytes in size.

### BACKGROUND

While genome modification through the CRISPR/Cas system, in theory, allows for both introduction and deletion of genetic material, the efficiency by which these processes occur is dependent on the mechanism of repair of the double strand breaks (DSBs). In non-homologous end joining (NHEJ), several nucleotides are frequently lost or added from the ends of the DSBs, and lead to frameshift mutations and subsequent knockout of the targeted alleles at high efficiency. In homology directed repair (HDR), genetic material integrates into the genome by homologous recombination, thereby allowing for knock in of large genetic segments, albeit at low efficiency.

Several approaches have been directed toward increasing the efficiency of HDR for CRISPR/Cas9, each limited by particular caveats. First, suppression of NHEJ molecules to promote the HDR pathway has been reported to improve the efficiency of HDR by 4 to 8 fold. The effects were seen with inhibition of NHEJ molecules (KU70, KU80), application of ligase IV inhibitor SCR70, and co-expression of adenovirus 4 E1B55K and E4orf6 proteins (to promote ligase IV degradation) (Chu et al. Nat Biotechnol, 33(5): 543-548, 2015), although the applicability and reproducibility in other cell systems has not been substantiated over time (Xei et al. Sci Rep. 7(1): 3036, 2017; Quadros et al. Genome Biol. 18(1): 92, 2017). Second, timed delivery of Cas9-guide RNA ribonucleoprotein complexes by cell synchronization with nocodazole in HEK293T, human primary neonatal fibroblast, and human embryonic stem cells increased rates of HDR by up to 38% compared to unsynchronized cells (Lin et al. Elife, 3:e04766, 2014). Nonetheless, perturbation of the DNA and toxicity from microtubule polymerizing agents poses potential issues with this methodology. Third, modification of the HDR donor by double cleavage increased HDR efficiency to 30% when combined with cell synchronization (CCND1 or nocodazole) (Zhang et al. Genome Biol. 18(1): 35, 2017), although this approach is also limited by toxicity. Fourth, optimization of the CRISPR/Cas9 system (electroporation, CRISPR/Cas9 dosage, homologous arm lengths, and dosing of various synchronizing agents) have been shown to reach HDR efficiency of 29.6% at specific “safe harbor” sites such as the Rosa26 locus, which is relatively conserved across species (Xei et al. 2017, supra). Customization of this approach for different systems would prove effort intensive. Fifth, microhomology-mediated end-joining enables efficient integration of exogenous donor DNA, but also is limited by ease of use (Nakade S. Nat Commun. 5: 5560, 2014; and Yoshimi K. Nat Commun. 7: 10431, 2016). Summarily, while these various approaches show low to moderate CRISPR/Cas9 knock in efficiency, they also carry significant inherent limitations. Thus, a significant technological hurdle arises in the use of CRISPR/Cas9 for knock in of genetic material.

### SUMMARY OF THE INVENTION

One aspect of the inventions features homology directed repair, in which the method features delivering to a target cell a gene editing system having: i) a first guide ribonucleic acid (RNA) directed to a first genomic site of an endogenous DNA molecule of the target cell, ii) a second guide RNA directed to a second genomic site of the endogenous DNA molecule of the target cell, iii) a plurality of fusion proteins comprising a first domain comprising an active RNA programmable nuclease and a second domain comprising an exonuclease, and, optionally, and iv) a donor DNA molecule, in which the first guide RNA forms a first complex with a first said fusion protein at the first genomic site and the second guide RNA forms a second complex with a second said fusion protein at the second genomic site, and the first and second complexes promote the homology directed repair by creating a lesion (e.g., double strand break) between the first and second genomic sites and, optionally, where the homology directed repair comprises insertion of the donor DNA molecule at the lesion between the first and second genomic sites.

In some embodiments, the first and second guide RNAs specifically hybridize to the first and second genomic sites, respectively. In another embodiment, the first genomic site and the second genomic site are between 10-100000 nucleotide base pairs apart. In certain embodiments, said first genomic site has a protospacer adjacent motif (PAM) recognition sequence positioned:

a) downstream from said first genomic site, and said second genomic site comprises a PAM recognition sequence downstream of said second genomic site;

b) downstream from said first genomic site, and said second genomic site comprises a PAM recognition sequence upstream of said second genomic site;

c) upstream from said first genomic site, and said second genomic site comprises a PAM recognition sequence upstream of said second genomic site; or

d) upstream from said first genomic site, and said second genomic site comprises a PAM recognition sequence downstream of said second genomic site.

In yet another embodiment, said first and second guide RNAs are two single guide RNAs, where said first guide RNA targets a first strand of the endogenous DNA molecule, and said second guide RNA targets a complementary strand of the endogenous DNA molecule, and said first domain of the fusion protein cleaves each strand of the endogenous DNA molecule, thereby creating a double-stranded break, and said second domain of the fusion protein cleaves the terminal nucleic acids of each strand of the endogenous DNA molecule, thereby creating elongated single stranded nucleic acid overhangs.

In another embodiment, a region between the first and second genomic sites is associated with a disease or disorder. In some embodiments, the disease or disorder is selected from a group consisting of Age-related macular degeneration; a blood or coagulation disease or disorder; a cell dysregulation or oncology disease or disorder; a developmental disorder; drug addiction; an inflammation or immune related disease or disorder; a metabolic, liver, kidney, or protein disease or disorder; a muscular or skeletal disease or disorder; a neurological or neuronal disease or disorder; a neoplasia; an ocular disease or disorder; schizophrenia; epilepsy; Duchenne muscular dystrophy; a viral disease or disorder, such as AIDS (acquired immunodeficiency syndrome); an autoimmune disorder; and an Alpha 1-antitrypsin deficiency.

In particular embodiments, the gene editing system further comprises a third and fourth guide RNA.

In some embodiments, the donor DNA molecule further comprises flanking regions modified to allow for specificity of targeting of one or more guide RNAs. In another embodiment, the one or more guide RNAs are the third and fourth guide RNAs. In certain embodiments, the third guide RNA forms a complex with a first said fusion protein at a first said flanking region on the donor DNA molecule and the fourth guide RNA forms a complex with a second said fusion protein at a second said flanking region on the donor DNA molecule, and said complexes cleave the donor DNA molecule at the flanking regions thereby releasing the donor DNA molecule.

In some embodiments, the first domain is a Cas RNA programmable nuclease. In certain embodiments, the Cas RNA programmable nuclease is a Cas9 RNA programmable nuclease. In particular embodiments, the second domain comprises an exonuclease selected from the group consisting of Lambda exonuclease, RecJf exonuclease, exonuclease III, exonuclease I, thermolabile exonuclease I, exonuclease T, exonuclease V (RecBCD), exonuclease VIII (truncated), exonuclease VII, nuclease BAL-31, T5 exonuclease, and T7 exonuclease. In some embodiments, the exonuclease is Lambda exonuclease.

In another embodiment, the method further comprises delivering an RNA programmable nuclease inhibitor to the target cell. In some embodiments, the RNA programmable nuclease inhibitor is selected from the group consisting of AcrIIA1, AcrIIA2, AcrIIA3, AcrIIA5, AcrIIA5, AcrIIC1, AcrIIC2, or AcrIIC3. In particular embodiments, the RNA programmable nuclease is AcrIIA4.

In particular embodiments, the RNA programmable nuclease inhibitor is delivered as a nucleic acid comprising a sequence encoding the RNA programmable nuclease inhibitor. In certain embodiments, the donor DNA molecule comprises a polynucleotide sequence encoding the RNA programmable nuclease inhibitor. In yet another embodiment, insertion of the donor DNA molecule at the lesion between the first and second genomic sites promotes expression of the RNA programmable nuclease inhibitor in the target cell, thereby inhibiting activity of the RNA programmable nuclease.

In another embodiment, the RNA programmable nuclease inhibitor is delivered as a polypeptide.

In some embodiments, the first or second genomic site comprises a nucleotide polymorphism. In other embodiments, the donor DNA molecule comprises a nucleic acid sequence encoding a gene not associated with a disease or disorder, wherein the homology directed repair comprises insertion of the donor DNA molecule at the lesion between the first and second genomic site, thereby correcting a nucleic acid sequence associated with a disease or disorder.

A second aspect of the invention features a nucleic acid comprising a polynucleotide comprising a nucleic acid sequence encoding a fusion protein comprising an RNA programmable nuclease and an exonuclease. In certain embodiments, the RNA programmable nuclease is a Cas RNA programmable nuclease. In Particular embodiments, the RNA programmable nuclease is a Cas9 RNA programmable nuclease. In some embodiments, the exonuclease is selected from the group consisting of Lambda Exonuclease, RecJf exonuclease, exonuclease III, exonuclease I, thermolabile exonuclease I, exonuclease T, exonuclease V (RecBCD), exonuclease VIII (truncated), exonuclease VII, nuclease BAL-31, T5 exonuclease, and T7 exonuclease. In another embodiment, the exonuclease is Lambda Exonuclease. In certain embodiments, the RNA programmable nuclease and the exonuclease are joined directly or through a linker.

In another embodiment, the nucleic acid further comprising a polynucleotide comprising a nucleic acid sequence encoding a first guide RNA and a second guide RNA. In some embodiments, the first and second guide RNA are directed to first and second genomic sites, respectively, of an endogenous DNA molecule of a cell. In another embodiment, the nucleic acid further comprises a polynucleotide comprising a nucleic acid sequence encoding a donor DNA molecule. In still another embodiment, the nucleic acid further comprising a polynucleotide comprising a nucleic acid sequence encoding a third guide RNA and a fourth guide RNA. In some embodiments, the polynucleotide comprising a nucleic acid sequence encoding a donor DNA molecule further comprises flanking regions of said donor DNA molecule and wherein said flanking regions are modified to allow for specificity of targeting of one or more guide RNAs. In particular embodiments, the donor DNA molecule comprises a nucleic acid sequence encoding a gene not associated with a disease or disorder.

In some embodiments, the donor DNA molecule comprises a polynucleotide sequence encoding the RNA programmable nuclease inhibitor. In another embodiment, RNA programmable nuclease is selected from the group consisting of AcrIIA1, AcrIIA2, AcrIIA3, AcrIIA5, AcrIIA5, AcrIIC1, AcrIIC2, or AcrIIC3. In particular embodiments, the RNA programmable nuclease is AcrIIA4. In certain embodiments, the nucleic acid further comprises a promoter.

A third aspect of the invention features a vector comprising a polynucleotide comprising a nucleic acid sequence encoding a fusion protein comprising an RNA programmable nuclease and an exonuclease, wherein the RNA programmable nuclease and the exonuclease are joined directly or through a linker. In some embodiments, the RNA programmable nuclease is a Cas RNA programmable nuclease (e.g., a Cas9 RNA programmable nuclease). In further embodiments, the exonuclease is selected from the group consisting of Lambda exonuclease, RecJf exonuclease, exonuclease III, exonuclease I, thermolabile exonuclease I, exonuclease T, exonuclease V (RecBCD), exonuclease VIII (truncated), exonuclease VII, nuclease BAL-31, T5 exonuclease, and T7 exonuclease. In particular embodiments, the exonuclease is Lambda exonuclease.

In some embodiments, the vector further comprises a polynucleotide comprising a nucleic acid sequence encoding a first and second guide RNA directed to first and second genomic sites, respectively, of an endogenous DNA molecule of a cell. In another embodiment, the vector further comprises a polynucleotide comprising a nucleic acid sequence encoding a third guide RNA and a fourth guide RNA. In particular embodiments, the vector further comprises a polynucleotide comprising a nucleic acid sequence encoding a donor DNA molecule.

In certain embodiments, the vector comprising a polynucleotide comprising a nucleic acid sequence encoding a donor DNA molecule further comprises flanking regions of said donor DNA molecule wherein the flanking regions of said donor DNA molecule are modified to allow for specificity of targeting of one or more guide RNAs. In a further embodiments, the donor DNA molecule comprises a polynucleotide comprising a nucleic acid sequence encoding an RNA programmable nuclease inhibitor. In certain embodiments, the RNA programmable nuclease inhibitor is selected from the group consisting of AcrIIA1, AcrIIA2, AcrIIA3, AcrIIA5, AcrIIA5, AcrIIC1, AcrIIC2, or AcrIIC3. In particular embodiments, the RNA programmable nuclease is AcrIIA4.

In some embodiments, the vector is an expression vector or a viral vector. In another embodiment, the viral vector is a lentiviral vector.

A fourth aspect of the invention features a composition comprising:

a) a first guide ribonucleic acid (RNA) directed to a first genomic site of an endogenous DNA molecule of a target cell,

b) a second guide RNA directed to a second genomic site of the endogenous DNA molecule of the target cell,

c) a plurality of fusion proteins comprising a first domain comprising an active RNA programmable nuclease and a second domain comprising an exonuclease, and, optionally,

d) a donor DNA molecule.

In some embodiments, the RNA programmable nuclease is a Cas RNA programmable nuclease. In particular embodiments, the Cas RNA programmable nuclease is a Cas9 RNA programmable nuclease. In another embodiment, the exonuclease is selected from the group consisting of Lambda exonuclease, RecJf exonuclease, exonuclease III, exonuclease I, thermolabile exonuclease I, exonuclease T, exonuclease V (RecBCD), exonuclease VIII (truncated), exonuclease VII, nuclease BAL-31, T5 exonuclease, and T7 exonuclease. In certain embodiments, the exonuclease is Lambda exonuclease.

In certain embodiments, the first guide RNA is in a first complex with a first said fusion protein and the second guide RNA is in a second complex with a second said fusion protein, where the first and second complexes are configured to promote homology directed repair of the endogenous DNA molecule, optionally, upon insertion of the donor DNA molecule between the first and second genomic sites. In particular embodiments, the donor DNA molecule comprises a nucleic acid sequence encoding a gene not associated with a disease or disorder.

In some embodiments, the composition further comprises an RNA programmable nuclease inhibitor. In another embodiment, the RNA programmable nuclease inhibitor is selected from the group consisting of AcrIIA1, AcrIIA2, AcrIIA3, AcrIIA5, AcrIIA5, AcrIIC1, AcrIIC2, or AcrIIC3. In certain embodiments, the RNA programmable nuclease is AcrIIA4.

A fifth aspect of the invention features a composition comprising:

a) a first polynucleotide comprising a nucleic acid sequence encoding a first guide ribonucleic acid (RNA) directed to a first genomic site of an endogenous DNA molecule of a target cell;

b) a second polynucleotide comprising a nucleic acid sequence encoding a second guide RNA directed to a second genomic site of the endogenous DNA molecule of the target cell;

c) a third polynucleotide comprising a nucleic acid sequence encoding a fusion protein comprising a first domain comprising an active RNA programmable nuclease and a second domain comprising an exonuclease; and, optionally,

d) a fourth polynucleotide comprising a nucleic acid sequence encoding a donor DNA molecule.

In some embodiments, the active RNA programmable nuclease and the exonuclease are joined directly or through a linker.

In some embodiments, the first guide RNA is configured to form a first complex with a first said fusion protein and the second guide RNA is configured to form a second complex with a second said fusion protein, and wherein the first and second complexes are configured to promote homology directed repair of the endogenous DNA molecule, optionally, upon insertion of the donor DNA molecule between the first and second genomic sites.

In certain embodiments, the composition further comprises a fifth polynucleotide comprising a nucleic acid sequence encoding an RNA programmable nuclease inhibitor or wherein the nucleic acid sequence of the fourth polynucleotide further encodes an RNA programmable nuclease inhibitor. In particular embodiments, the RNA programmable nuclease inhibitor is selected from the group consisting of AcrIIA1, AcrIIA2, AcrIIA3, AcrIIA5, AcrIIA5, AcrIIC1, AcrIIC2, or AcrIIC3. In some embodiments, the RNA programmable nuclease is AcrIIA4. In other embodiments, the donor DNA molecule comprises a nucleic acid sequence encoding a gene not associated with a disease or disorder.

In still another embodiment, the composition further comprises:

i) a sixth polynucleotide comprising a nucleic acid sequence encoding a third guide RNA, and

ii) a seventh polynucleotide comprising a nucleic acid sequence encoding a fourth guide RNA.

In certain embodiments, the polynucleotide comprising a nucleic acid sequence encoding the donor DNA further comprise flanking regions of said donor DNA modified to allow for specificity of targeting of one or more guide RNAs. In another embodiment, the one or more guide RNAs are the third and fourth guide RNAs. In certain embodiments, the third guide RNA is configured to form a complex with a first said fusion protein at a first said flanking region on the donor DNA molecule and the fourth guide RNA is configured to form a complex with a second said fusion protein at a second said flanking region on the donor DNA molecule, and where said complexes cut the donor DNA molecule at the flanking regions thereby releasing the donor DNA molecule.

A sixth aspect of the invention features a pharmaceutical composition comprising the nucleic acid, the vector, or the composition of any one of the previous aspects or embodiments and a pharmaceutically acceptable carrier, excipient, or diluent.

A seventh aspect of the invention features a kit comprising the nucleic acid, the vector, the composition, or the pharmaceutical composition of any one of the previous aspects or embodiments. In some embodiments, the kit comprises the first and second guide RNAs, where the first and second guide RNAs are targeted to a genomic site of an endogenous DNA molecule of a target cell causing a disease. In another embodiment, the first and second guide RNAs target a nucleotide polymorphism at the genomic site of the endogenous DNA molecule of the target cell.

An eighth aspect of the invention features a fusion protein comprising a first domain comprising an active RNA programmable nuclease and a second domain comprising an exonuclease, where the two domains are joined directly or through a linker. In some embodiments, the first domain is a Cas RNA programmable nuclease (e.g., a Cas9 RNA programmable nuclease). In particular embodiments, the second domain comprises an exonuclease selected from the group consisting of Lambda exonuclease, RecJf exonuclease, exonuclease III, exonuclease I, thermolabile exonuclease I, exonuclease T, exonuclease V (RecBCD), exonuclease VIII (truncated), exonuclease VII, nuclease BAL-31, T5 exonuclease, and T7 exonuclease. In certain embodiments, the exonuclease is Lambda exonuclease. In another embodiment, the two domains are joined directly or through a linker.

In some embodiments, the homology directed repair treats a disease or disorder. In certain embodiments, the disease or disorder is selected from a group consisting of age-related macular degeneration; a blood or coagulation disease or disorder; a cell dysregulation or oncology disease or disorder; a developmental disorder; drug addiction; an inflammation or immune related disease or disorder; a metabolic, liver, kidney, or protein disease or disorder; a muscular or skeletal disease or disorder; a neurological or neuronal disease or disorder; a neoplasia; an ocular disease or disorder; schizophrenia; epilepsy; Duchenne muscular dystrophy; a viral disease or disorder, such as AIDS (acquired immunodeficiency syndrome); an autoimmune disorder; and an alpha 1-antitrypsin deficiency.

In some embodiments, the featured compositions are for use in treating a disease or disorder. In certain embodiments, the disease or disorder is selected from a group consisting of age-related macular degeneration; a blood or coagulation disease or disorder; a cell dysregulation or oncology disease or disorder; a developmental disorder; drug addiction; an inflammation or immune related disease or disorder; a metabolic, liver, kidney, or protein disease or disorder; a muscular or skeletal disease or disorder; a neurological or neuronal disease or disorder; a neoplasia; an ocular disease or disorder; schizophrenia; epilepsy; Duchenne muscular dystrophy; a viral disease or disorder, such as AIDS (acquired immunodeficiency syndrome); an autoimmune disorder; and an alpha 1-antitrypsin deficiency.

In certain embodiments, the blood or coagulation disease or disorder is:

a) anemia wherein, preferable, the gene is CDAN1, CDA1, RPS19, DBA, PKLR, PK1, NT5C3, UMPH1, PSN1, RHAG, RH50A, NRAMP2, SPTB, ALAS2, ANH1, ASB, ABCB7, ABC7, and/or ASAT;

b) bare lymphocyte syndrome, wherein, preferably, the gene is TAPBP, TPSN, TAP2, ABCB3, PSF2, RING11, MHC2TA, C2TA, RFX5, RFXAP, or RFX5;

c) a bleeding disorder, wherein, preferably, the gene is TBXA2R, P2RX1, or P2X1:

d) a hemolytic anemia, such as a complement Factor H deficiency disease, e.g., a typical hemolytic anemia syndrome (aHUS), wherein, preferably, the gene is HF1, CFH, or HUS;

e) a factor V or factor VIII deficiency disease, wherein, preferably, the gene is MCFD2;

f) a factor VII deficiency disease, wherein, preferably, the gene is F7;

g) a factor X deficiency disease, wherein, preferably, the gene is F10;

h) a factor XI deficiency disease, wherein, preferably, the gene is F11;

i) a factor XII deficiency disease, wherein, preferably, the gene is F12 or HAF;

j) a factor XIIIA deficiency disease, wherein, preferably, the gene is F13A1 or F13A;

k) a factor XIIIB deficiency disease, wherein, preferably, the gene is F13B;

l) Fanconi anemia, wherein, preferably, the gene is FANCA, FACA, FA1, FA, FAA, FAAP95, FAAP90, FLJ34064, FANCB, FANCC, FACC, BRCA2, FANCD1, FANCD2, FANCD, FACD, FAD, FANCE, FACE, FANCF, XRCC9, FANCG, BRIP1, BACH1, FANCJ, PHF9, FANCL, FANCM, or KIAA1596;

m) a hemophagocytic or lymphohistiocytosis disorder, wherein, preferably, the gene is PRF1, HPLH2, UNC13D, MUNC13-4, HPLH3, HLH3, or FHL3;

n) hemophilia A, wherein, preferably, the gene is F8, F8C, or HEMA;

o) hemophilia B, wherein, preferably, the gene is F9 or HEMB;

p) a hemorrhagic disorder, wherein, preferably, the gene is PI, ATT, F5;

q) a leukocyte deficiency or disorder, wherein, preferably, the gene is ITGB2, CD18, LCAMB, LAD, EIF2B1, EIF2BA, EIF2B2, EIF2B3, EIF2B5, LVWM, CACH, CLE, or EIF2B4;

r) sickle cell anemia, wherein, preferably, the gene is HBB; or

s) thalassemia, wherein, preferably, the gene is HBA2, HBB, HBD, LCRB, or HBA1.

In another embodiment, the cell dysregulation or oncology disease is:

a) B-cell non-Hodgkin lymphoma, wherein, preferably, the gene is BCL7A or BCL7; or

b) a leukemia, wherein, preferably, the gene is TAL1 TCL5, SCL, TAL2, FLT3, NBS1, NBS, ZNFN1A1, IK1, LYF1, HOXD4, HOX4B, BCR, CML, PHL, ALL, ARNT, KRAS2, RASK2, GMPS, AF10, ARHGEF12, LARG, KIAA0382, CALM, CLTH, CEBPA, CEBP, CHIC2, BTL, FLT3, KIT, PBT, LPP, NPM1, NUP214, D9S46E, CAN, CAIN, RUNX1, CBFA2, AML1, WHSC1L1, NSD3, FLT3, AF1Q, NPM1, NUMA1, ZNF145, PLZF, PML, MYL, STAT5B, AF10, CALM, CLTH, ARL11, ARLTS1, P2RX7, P2X7, BCR, CML, PHL, ALL, GRAF, NF1, VRNF, WSS, NFNS, PTPN11, PTP2C, SHP2, NS1, BCL2, CCND1, PRAD1, BCL1, TCRA, GATA1, GF1, ERYF1, NFE1, ABL1, NQO1, DIA4, NMOR1, NUP214, D9S46E, CAN, or CAIN.

In particular embodiments, the developmental disease is:

a) Angelman syndrome, wherein, preferably, the gene is UBE3A or a 15q11-13 deletion;

b) Canavan disease, wherein, preferably, the gene is ASPA;

c) Cri-du-chat syndrome, wherein, preferably, the gene is 5P− (5p minus) or CTNND2;

d) Down syndrome, wherein, preferably, the gene is Trisomy 21;

e) Klinefelter syndrome, wherein, preferably, the gene is XXY or two or more X chromosomes in males;

f) Prader-Willi syndrome, wherein, preferably, the gene is deletion of chromosome 15 segment or a duplication of maternal chromosome 15; or

g) Turner syndrome where the gene is monosomy X or SHOX.

In other embodiments, disease or disorder is a drug addiction, wherein, preferably, the gene is PRKCE, DRD2, DRD4, ABAT (alcohol), GRIA2, GRM5, GRIN1, HTR1B, GRIN2A, DRD3, PDYN, GRIA1 (alcohol).

In certain embodiments, the inflammation or immune related disease is:

a) autoimmune lymphoproliferative syndrome, wherein, preferably, the gene TNFRSF6, APT1, FAS, CD95, or ALPS1A;

b) combined immuno-deficiency, wherein, preferably, the gene is IL2RG, SCIDX1, SCIDX, or IMD4;

c) an immuno-deficiency, wherein, preferably, the gene is CD3E, CD3G, AICDA, AID, HIGM2, TNFRSF5, CD40, UNG, DGU, HIGM4, TNFSF5, CD40LG, HIGM1, IGM, FOXP3, IPEX, AIID, XPID, PIDX, TNFRSF14B, or TACI;

d) inflammation wherein, preferably, the gene is IL-10, IL-1 (IL-1a, IL-1 b), IL-13, IL-17 (IL-17a (CTLA8), IL-17b, IL-17c, IL-17d, IL-17f), 11-23, CX3CR1, PTPN22, TNF-alpha (TNFa), NOD2/CARD15 for IBD, IL-6, IL-12 (IL-12a, IL-12b), CTLA4, or CX3CL1; or

e) severe combined immunodeficiency disease, wherein, preferably, the gene is JAK3, JAKL, DCLRE1C, ARTEMIS, SCIDA, RAG1, RAG2, ADA, PTPRC, CD45, LCA, IL7R, CD3D, T3D, IL2RG, SCIDX1, SCIDX, or IMD4.

In another embodiment, the metabolic, liver, kidney, or protein disease is:

a) amyloid neuropathy, wherein, preferably, the gene is TTR or PALB;

b) amyloidosis, wherein, preferably, the gene is APOA1, APP, AAA, CVAP, AD1, GSN, FGA, LYZ, TTR, or PALB;

c) cirrhosis, wherein, preferably, the gene is KRT18, KRT8, CIRH1A, NAIC, TEX292, or KIAA1988;

d) cystic fibrosis, wherein, preferably, the gene is CFTR, ABCC7, CF, or MRP7;

e) a glycogen storage disease, wherein, preferably, the gene is SLC2A2, GLUT2, G6PC, G6PT, G6PT1, GAA, LAMP2, LAMPB, AGL, GDE, GBE1, GYS2, PYGL, or PFKM;

f) hepatic adenoma, wherein, preferably, the gene is TCF1, HNF1 A, or MODY3;

g) an early onset neurologic disorder, wherein, preferably, the gene is SCOD1 or SCO1;

h) hepatic lipase deficiency, wherein, preferably, the gene is LIPC;

i) hepato-blastoma cancer, wherein, preferably, the gene is CTNNB1, PDGFRL, PDGRL, PRLTS, AXIN1, AXIN, CTNNB1, TP53, P53, LFS1, IGF2R, MPRI, MET, CASP8, or MCH5;

j) medullary cystic kidney disease, wherein, preferably, the gene is UMOD, HNFJ, FJHN, MCKD2, or ADMCKD2;

k) phenylketonuria, wherein, preferably, the gene is PAH, PKU1, QDPR, DHPR, or PTS; or 1) polycystic kidney or hepatic disease, wherein, preferably, the gene is FCYT, PKHD1, ARPKD, PKD1, PKD2, PKD4, PKDTS, PRKCSH, G19P1, PCLD, or SEC63.

In some embodiments, the muscular or skeletal disease is:

a) Becker muscular dystrophy, wherein, preferably, the gene is DMD, BMD, or MYF6;

b) Duchenne muscular dystrophy, wherein, preferably, the gene is DMD or BMD;

c) Emery-Dreifuss muscular dystrophy, wherein, preferably, the gene is LMNA, LMN1, EMD2, FPLD, CMD1 A, HGPS, LGMD1B, LMNA, LMN1, EMD2, FPLD, or CMD1 A;

d) Facio-scapulohumeral muscular dystrophy, wherein, preferably, the gene is FSHMD1A or FSHD1A;

e) muscular dystrophy, wherein, preferably, the gene is FKRP, MDC1C, LGMD2I, LAMA2, LAMM, LARGE, KIAA0609, MDC1 D, FCMD, TTID, MYOT, CAPN3, CANP3, DYSF, LGMD2B, SGCG, LGMD2C, DMDA1, SCG3, SGCA, ADL, DAG2, LGMD2D, DMDA2, SGCB, LGMD2E, SGCD, SGD, LGMD2F, CMD1L, TCAP, LGMD2G, CMD1N, TRIM32, HT2A, LGMD2H, FKRP, MDC1C, LGMD2I, TTN, CMD1G, TMD, LGMD2J, POMT1, CAV3, LGMD1C, SEPN1, SELN, RSMD1, PLEC1, PLTN, or EBS1;

f) osteopetrosis, wherein, preferably, the gene is LRP5, BMND1, LRP7, LR3, OPPG, VBCH2, CLCN7, CLC7, OPTA2, OSTM1, GL, TCIRG1, TIRC7, OC116, or OPTB1;

g) muscular atrophy, wherein, preferably, the gene is VAPB, VAPC, ALS8, SMN1, SMA1, SMA2, SMA3, SMA4, BSCL2, SPG17, GARS, SMAD1, CMT2D, HEXB, IGHMBP2, SMUBP2, CATF1, or SMARD1; or

h) Tay-Sachs disease, wherein, preferably, the gene is HEXA.

In other embodiments, the neurological and neuronal disease is:

a) amyotrophic lateral sclerosis (ALS), wherein, preferably, the gene is SOD1, ALS2, STEX, FUS, TARDBP, or VEGF (VEGF-a, VEGF-b, VEGF-c);

b) Alzheimer's disease, wherein, preferably, the gene is APP, AAA, CVAP, AD1, APOE, AD2, PSEN2, AD4, STM2, APBB2, FE65L1, NOS3, PLAU, URK, ACE, DCP1, ACE1, MPO, PACIP1, PAXIP1L, PTIP, A2M, BLMH, BMH, PSEN1, orAD3;

c) autism, wherein, preferably, the gene is Mecp2, BZRAP1, MDGA2, Sema5A, Neurexin 1, GLO1, MECP2, RTT, PPMX, MRX16, MRX79, NLGN3, NLGN4, KIAA1260, or AUTSX2;

d) Fragile X Syndrome, wherein, preferably, the gene is FMR2, FXR1, FXR2, or mGLUR5;

e) Huntington's disease or a Huntington's disease like disorder, wherein, preferably, the gene is HD, IT15, PRNP, PRIP, JPH3, JP3, HDL2, TBP, or SCA17;

f) Parkinson's disease, wherein, preferably, the gene is NR4A2, NURR1, NOT, TINUR, SNCAIP, TBP, SCA17, SNCA, NACP, PARK1, PARK4, DJ1, PARK7, LRRK2, PARK8, PINK1, PARK6, UCHL1, PARK5, SNCA, NACP, PARK1, PARK4, PRKN, PARK2, PDJ, DBH, or NDUFV2;

g) Rett syndrome, wherein, preferably, the gene is MECP2, RTT, PPMX, MRX16, MRX79, CDKL5, STK9, MECP2, RTT, PPMX, MRX16, MRX79, α-Synuclein, or DJ-1;

h) schizophrenia, wherein, preferably, the gene is NRG1, ERB4, CPLX1), TPH1, TPH2, Neurexin 1, GSK3, GSK3a, GSK3b, 5-HTT (SLC6A4), COMT, DRD (DRD1a), SLC6A3, DAOA, DTNBP1, or DAO (DAO1);

i) secretase related disorders, wherein, preferably, the gene is APH-1 (alpha and beta), presenilin (PSEN1), nicastrin (NCSTN), PEN-2, NOS1, PARP1, NAT1, or NAT2; or

j) trinucleotide repeat disorders, wherein, preferably, the gene is HTT, SBMA/SMAX1/AR, FXN/X25, ATX3, ATXN1, ATXN2, DMPK, Atrophin-1, Atn1, CBP, VLDLR, ATXN7, or ATXN10.

In another embodiment, the disease or disorder is neoplasia, wherein, preferably, the gene is PTEN, ATM, ATR, EGFR, ERBB2, ERBB3, ERBB4, Notch1, Notch2, Notch3, Notch4, AKT, AKT2, AKT3, HIF, HIF1a, HIF3a, MET, HRG, Bcl2, PPAR alpha, PPAR gamma, WT1 (Wilms Tumor), FGF1, FGF2, FGF3, FGF4, FGF5, CDKN2a, APC, RB (retinoblastoma), MEN1, VHL, BRCA1, BRCA2, AR (androgen receptor), TSG101, IGF, IGF receptor, IGF1 (4 variants), IGF2 (3 variants), IGF 1 receptor, IGF 2 receptor, BAX, BCL2, caspase 1, 2, 3, 4, 6, 7, 8, 9, 12, KRAS, or APC.

In particular embodiments, the ocular disease is:

a) age-related macular degeneration, wherein, preferably, the gene is Aber, CCL2, CC2, CP (ceruloplasmin), TIMP3, cathepsin D, VLDLR, or CCR2;

b) cataract, wherein, preferably, the gene is CRYAA, CRYA1, CRYBB2, CRYB2, PITX3, BFSP2, CP49, CP47, CRYAA, CRYA1, PAX6, AN2, MGDA, CRYBA1, CRYB1, CRYGC, CRYG3, CCL, LIM2, MP19, CRYGD, CRYG4, BFSP2, CP49, CP47, HSF4, CTM, HSF4, CTM, MIP, AQPO, CRYAB, CRYA2, CTPP2, CRYBB1, CRYGD, CRYG4, CRYBB2, CRYB2, CRYGC, CRYG3, CCL, CRYAA, CRYA1, GJA8, CX50, CAE1, GJA3, CX46, CZP3, CAE3, CCM1, CAM, or KRIT1;

c) corneal clouding or corneal dystrophy, wherein, preferably, the gene is APOA1, TGFBI, CSD2, CDGG1, CSD, BIGH3, CDG2, TACSTD2, TROP2, M1 S1, VSX1, RINX, PPCD, PPD, KTCN, COL8A2, FECD, PPCD2, PIP5K3, or CFD;

d) cornea plana (congenital), wherein, preferably, the gene is KERA or CNA2;

e) glaucoma, wherein, preferably, the gene is MYOC, TIGR, GLC1A, JOAG, GPOA, OPTN, GLC1E, FIP2, HYPL, NRP, CYP1B1, GLC3A, OPA1, NTG, NPG, CYP1B1, or GLC3A;

f) Leber congenital amaurosis, wherein, preferably, the gene is CRB1, RP12, CRX, CORD2, CRD, RPGRIP1, LCA6, CORD9, RPE65, RP20, AIPL1, LCA4, GUCY2D, GUC2D, LCA1, CORD6, RDH12, or LCA3; or

g) macular dystrophy, wherein, preferably, the gene is ELOVL4, ADMD, STGD2, STGD3, RDS, RP7, PRPH2, PRPH, AVMD, AOFMD, or VMD2.

In certain embodiments, the disease or disorder is schizophrenia, wherein, preferably, the gene is neuregulin1 (NRG1), ERB4, Complexin1 (CPLX1), TPH1, TPH2, NRXN1, GSK3, GSK3a, or GSK3b.

In some embodiments, the disease or disorder is epilepsy, wherein, preferably, the gene is EPM2A, MELF, EPM2, NHLRC1, EPM2A, or EPM2B.

In certain embodiments, the disease is Duchenne muscular dystrophy, wherein, preferably, the gene is DMD or BMD.

In another embodiment, the viral disease or disorder is:

a) AIDS, wherein, preferably, the gene is KIR3DL1, NKAT3, NKB1, AMB11, KIR3DS1, IFNG, CXCL12, or SDF1

b) caused by human immunodeficiency virus (HIV), wherein, preferably, the gene is CCL5, SCYA5, D17S136E, or TCP228; or

c) HIV susceptibility or infection, wherein, preferably, the gene is IL10, CSIF, CMKBR2, CCR2, CMKBR5, or CCCKR5 (CCR5).

In particular embodiments, the disease or disorder is alpha 1-antitrypsin deficiency, wherein, preferably, the gene is SERPINA1 [serpin peptidase inhibitor, clade A (alpha-1 antiproteinase, antitrypsin), member 1], SERPINA2, SERPINA3, SERPINA5, SERPINA6, or SERPINA7.

In other embodiments, the homology directed repair treats a cellular dysfunction.

In particular embodiments, the featured compositions are for use in treating a cellular dysfunction.

In certain embodiments, the cellular dysfunction is associated with PI3K/AKT signaling, ERK/MAPK signaling, glucocorticoid receptor signaling, axonal guidance signaling, ephrin receptor signaling, actin cytoskeleton signaling, Huntington's disease signaling, apoptosis signaling, B cell receptor signaling, leukocyte extravasation signaling, integrin signaling, acute phase response signaling, PTEN signaling, p53 signaling, aryl hydrocarbon receptor signaling, xenobiotic metabolism signaling, SAPK/JNK signaling, PPAr/RXR signaling, NF-KB signaling, neuregulin signaling, Wnt or beta catenin signaling, insulin receptor signaling, IL-6 signaling, hepatic cholestasis, IGF-1 signaling, NRF2-mediated oxidative stress response, hepatic signaling, fibrosis or hepatic stellate cell activation, PPAR signaling, Fc Epsilon RI signaling, G-protein coupled receptor signaling, inositol phosphate metabolism, PDGF signaling, VEGF signaling, natural killer cell signaling, cell cycle G1/S checkpoint regulation, T cell receptor signaling, death receptor signaling, FGF signaling, GM-CSF signaling, amyotrophic lateral sclerosis signaling, JAK/Stat signaling, nicotinate or nicotinamide metabolism, chemokine signaling, IL-2 signaling, synaptic long term depression, estrogen receptor signaling, protein ubiquitination pathway, IL-10 signaling, VDR/RXR activation, TGF-beta signaling, toll-like receptor signaling, p38 MAPK signaling, neurotrophin/TRK signaling, FXR/RXR Activation, synaptic long term potentiation, calcium signaling, EGF signaling, hypoxia signaling in the cardiovascular system, LPS/IL-1 mediated inhibition of RXR function, LXR/RXR activation, amyloid processing, IL-4 signaling, cell cycle G2/M DNA damage checkpoint regulation, nitric oxide signaling in the cardiovascular system, purine metabolism, cAMP-mediated signaling, mitochondrial dysfunction notch signaling, endoplasmic reticulum stress pathway, pyrimidine metabolism, Parkinson's signaling, cardiac or beta adrenergic signaling, glycolysis or gluconeogenesis, interferon signaling, sonic hedgehog signaling, glycerophospholipid metabolism, phospholipid degradation, tryptophan metabolism, lysine degradation, nucleotide excision repair pathway, starch and sucrose metabolism, amino sugars metabolism, arachidonic acid metabolism, circadian rhythm signaling, coagulation system, dopamine receptor signaling, glutathione metabolism, glycerolipid metabolism, linoleic acid metabolism, methionine metabolism, pyruvate metabolism, arginine and proline metabolism, eicosanoid signaling, fructose and mannose metabolism, galactose metabolism, stilbene, coumarine and lignin biosynthesis, antigen presentation, pathway, biosynthesis of steroids, butanoate metabolism, citrate cycle, fatty acid metabolism, histidine metabolism, inositol metabolism, metabolism of xenobiotics by cytochrome p450, methane metabolism, phenylalanine metabolism, propanoate metabolism, selenoamino acid metabolism, sphingolipid metabolism, aminophosphonate metabolism, androgen or estrogen metabolism, ascorbate or aldarate metabolism, bile acid biosynthesis, cysteine metabolism, fatty acid biosynthesis, glutamate receptor signaling, NRF2-mediated oxidative stress response, pentose phosphate pathway, pentose and glucuronate interconversions, retinol metabolism, riboflavin metabolism, tyrosine metabolism, ubiquinone biosynthesis, valine, leucine and isoleucine degradation, glycine, serine and threonine metabolism, lysine degradation, pain/taste, pain, mitochondrial function, or developmental neurology.

In another embodiment, the cellular dysfunction is associated with:

i) PI3K/AKT signaling, wherein, preferably, the gene is PRKCE, ITGAM, ITGA5, IRAK1, PRKAA2, EIF2AK2, PTEN, EIF4E, PRKCZ, GRK6, MAPK1, TSC1, PLK1, AKT2, IKBKB, PIK3CA, CDK8, CDKN1B, NFKB2, BCL2, PIK3CB, PPP2R1A, MAPK8, BCL2L1, MAPK3, TSC2, ITGA1, KRAS, EIF4EBP1, RELA, PRKCD, NOS3, PRKAA1, MAPK9, CDK2, PPP2CA, PIM1, ITGB7, YWHAZ, ILK, TP53, RAF1., IKBKG, RELB, DYRK1A, CDKN1A, ITGB1, MAP2K2, JAK1, AKT1, JAK2, PIK3R1, CHUK, PDPK1, PPP2R5C, CTNNB1., MAP2K1, NFKB1, PAK3, ITGB3, CCND1, GSK3A, FRAP1, SFN, ITGA2, TTK, CSNK1A1, BRAF, GSK3B, AKT3, FOXO1, SGK, HSP90AA1, or RPS6KB1;

ii) ERK/MAPK signaling, wherein, preferably, the gene is PRKCE, ITGAM, ITGA5, HSPB1, IRAK1, PRKAA2, EIF2AK2, RAC1, RAP1A, TLN1, EIF4E, ELK1, GRK6, MAPK1, RAC2, PLK1, AKT2, PIK3CA, CDK8, CREB1, PRKCI, PTK2, FOS, RPS6KA4, PIK3CB, PPP2R1A, PIK3C3, MAPK8, MAPK3, ITGA1, ETS1, KRAS, MYCN, EIF4EBP1, PPARG, PRKCD, PRKAA1, MAPK9, SRC, CDK2, PPP2CA, PIM1, PIK3C2A, ITGB7, YWHAZ, PPP1CC, KSR1, PXN, RAF1, FYN, DYRK1A, ITGB1, MAP2K2, PAK4, PIK3R1, STAT3, PPP2R5C, MAP2K1, PAK3, ITGB3, ESR1, ITGA2, MYC, TTK, CSNK1A1, CRKL, BRAF, ATF4, PRKCA, SRF, STAT1, or SGK;

iii) glucocorticoid receptor signaling, wherein, preferably, the gene is RAC1, TAF4B, EP300, SMAD2, TRAF6, PCAF, ELK1, MAPK1, SMAD3, AKT2, IKBKB, NCOR2, UBE2I, PIK3CA, CREB1, FOS, HSPA5, NFKB2, BCL2, MAP3K14, STAT5B, PIK3CB, PIK3C3, MAPK8, BCL2L1, MAPK3, TSC22D3, MAPK10, NRIP1, KRAS, MAPK13, RELA, STAT5A, MAPK9, NOS2A, PBX1, NR3C1, PIK3C2A, CDKN1C, TRAF2, SERPINE1, NCOA3, MAPK14, TNF, RAF1, IKBKG, MAP3K7, CREBBP, CDKN1A, MAP2K2, JAK1, IL8, NCOA2, AKT1, JAK2, PIK3R1, CHUK, STAT3, MAP2K1, NFKB1, TGFBR1, ESR1, SMAD4, CEBPB, JUN, AR, AKT3, CCL2, MMP1, STAT1, 1L6, or HSP90AA1;

iv) axonal guidance signaling, wherein, preferably, the gene is PRKCE, ITGAM, ROCK1, ITGA5, CXCR4, ADAM12, IGF1, RAC1, RAP1A, E1F4E, PRKCZ, NRP1, NTRK2, ARHGEF7, SMO, ROCK2, MAPK1, PGF, RAC2, PTPN11, GNAS, AKT2, PIK3CA, ERBB2, PRKC1, PTK2, CFL1, GNAQ, PIK3CB, CXCL12, PIK3C3, WNT11, PRKD1, GNB2L1, ABL1, MAPK3, ITGA1, KRAS, RHOA, PRKCD, PIK3C2A, ITGB7, GLI2, PXN, VASP, RAF1, FYN, ITGB1, MAP2K2, PAK4, ADAM17, AKT1, PIK3R1, GLI1, WNT5A, ADAM10, MAP2K1, PAK3, ITGB3, CDC42, VEGFA, ITGA2, EPHA8, CRKL, RND1, GSK3B, AKT3, or PRKCA;

v) ephrin receptor signaling, wherein, preferably, the gene is PRKCE, ITGAM, ROCK1, ITGA5, CXCR4, IRAK1, PRKAA2, EIF2AK2, RAC1, RAP1A, GRK6, ROCK2, MAPK1, PGF, RAC2, PTPN11, GNAS, PLK1, AKT2, DOK1, CDK8, CREB1, PTK2, CFL1, GNAQ, MAP3K14, CXCL12, MAPK8, GNB2L1, ABL1, MAPK3, ITGA1, KRAS, RHOA, PRKCD, PRKAA1, MAPK9, SRC, CDK2, PIM1, ITGB7, PXN, RAF1, FYN, DYRK1A, ITGB1, MAP2K2, PAK4, AKT1, JAK2, STAT3, ADAM10, MAP2K1, PAK3, ITGB3, CDC42, VEGFA, ITGA2, EPHA8, TTK, CSNK1A1, CRKL, BRAF, PTPN13, ATF4, AKT3, or SGK;

vi) actin cytoskeleton signaling, wherein, preferably, the gene is ACTN4, PRKCE, ITGAM, ROCK1, ITGA5, IRAK1, PRKAA2, EIF2AK2, RAC1, INS, ARHGEF7, GRK6, ROCK2, MAPK1, RAC2, PLK1, AKT2, PIK3CA, CDK8, PTK2, CFL1, PIK3CB, MYH9, DIAPH1, PIK3C3, MAPK8, F2R, MAPK3, SLC9A1, ITGA1, KRAS, RHOA, PRKCD, PRKAA1, MAPK9, CDK2, PIM1, PIK3C2A, ITGB7, PPP1CC, PXN, VIL2, RAF1, GSN, DYRK1A, ITGB1, MAP2K2, PAK4, PIP5K1A, PIK3R1, MAP2K1, PAK3, ITGB3, CDC42, APC, ITGA2, TTK, CSNK1A1, CRKL, BRAF, VAV3, or SGK;

vii) Huntington's disease signaling, wherein, preferably, the gene is PRKCE, IGF1, EP300, RCOR1, PRKCZ, HDAC4, TGM2, MAPK1, CAPNS1, AKT2, EGFR, NCOR2, SP1, CAPN2, PIK3CA, HDAC5, CREB1, PRKC1, HSPA5, REST, GNAQ, PIK3CB, PIK3C3, MAPK8, IGF1R, PRKD1, GNB2L1, BCL2L1, CAPN1, MAPK3, CASP8, HDAC2, HDAC7A, PRKCD, HDAC11, MAPK9, HDAC9, PIK3C2A, HDAC3, TP53, CASP9, CREBBP, AKT1, PIK3R1, PDPK1, CASP1, APAF1, FRAP1, CASP2, JUN, BAX, ATF4, AKT3, PRKCA, CLTC, SGK, HDAC6, or CASP3;

viii) apoptosis signaling, wherein, preferably, the gene is PRKCE, ROCK1, BID, IRAK1, PRKAA2, EIF2AK2, BAK1, BIRC4, GRK6, MAPK1, CAPNS1, PLK1, AKT2, IKBKB, CAPN2, CDK8, FAS, NFKB2, BCL2, MAP3K14, MAPK8, BCL2L1, CAPN1, MAPK3, CASP8, KRAS, RELA, PRKCD, PRKAA1, MAPK9, CDK2, PIM1, TP53, TNF, RAF1, IKBKG, RELB, CASP9, DYRK1A, MAP2K2, CHUK, APAF1, MAP2K1, NFKB1, PAK3, LMNA, CASP2, BIRC2, TTK, CSNK1A1, BRAF, BAX, PRKCA, SGK, CASP3, BIRC3, or PARP1;

ix) B cell receptor signaling, wherein, preferably, the gene is RAC1, PTEN, LYN, ELK1, MAPK1, RAC2, PTPN11, AKT2, IKBKB, PIK3CA, CREB1, SYK, NFKB2, CAMK2A, MAP3K14, PIK3CB, PIK3C3, MAPK8, BCL2L1, ABL1, MAPK3, ETS1, KRAS, MAPK13, RELA, PTPN6, MAPK9, EGR1, PIK3C2A, BTK, MAPK14, RAF1, IKBKG, RELB, MAP3K7, MAP2K2, AKT1, PIK3R1, CHUK, MAP2K1, NFKB1, CDC42, GSK3A, FRAP1, BCL6, BCL10, JUN, GSK3B, ATF4, AKT3, VAV3, or RPS6KB1;

x) leukocyte extravasation signaling wherein, preferably, the gene is ACTN4, CD44, PRKCE, ITGAM, ROCK1, CXCR4, CYBA, RAC1, RAP1A, PRKCZ, ROCK2, RAC2, PTPN11, MMP14, PIK3CA, PRKCI, PTK2, PIK3CB, CXCL12, PIK3C3, MAPK8, PRKD1, ABL1, MAPK10, CYBB, MAPK13, RHOA, PRKCD, MAPK9, SRC, PIK3C2A, BTK, MAPK14, NOX1, PXN, VIL2, VASP, ITGB1, MAP2K2, CTNND1, PIK3R1, CTNNB1, CLDN1, CDC42, F11R, ITK, CRKL, VAV3, CTTN, PRKCA, MMP1, or MMP9;

xi) integrin signaling wherein, preferably, the gene is ACTN4, ITGAM, ROCK1, ITGA5, RAC1, PTEN, RAP1A, TLN1, ARHGEF7, MAPK1, RAC2, CAPNS1, AKT2, CAPN2, P1K3CA, PTK2, PIK3CB, PIK3C3, MAPK8, CAV1, CAPN1, ABL1, MAPK3, ITGA1, KRAS, RHOA, SRC, PIK3C2A, ITGB7, PPP1CC, ILK, PXN, VASP, RAF1, FYN, ITGB1, MAP2K2, PAK4, AKT1, PIK3R1, TNK2, MAP2K1, PAK3, ITGB3, CDC42, RND3, ITGA2, CRKL, BRAF, GSK3B, or AKT3;

xii) acute phase response signaling wherein, preferably, the gene is IRAK1, SOD2, MYD88, TRAF6, ELK1, MAPK1, PTPN11, AKT2, IKBKB, PIK3CA, FOS, NFKB2, MAP3K14, PIK3CB, MAPK8, RIPK1, MAPK3, IL6ST, KRAS, MAPK13, IL6R, RELA, SOCS1, MAPK9, FTL, NR3C1, TRAF2, SERPINE1, MAPK14, TNF, RAF1, PDK1, IKBKG, RELB, MAP3K7, MAP2K2, AKT1, JAK2, PIK3R1, CHUK, STAT3, MAP2K1, NFKB1, FRAP1, CEBPB, JUN, AKT3, IL1R1, or IL6;

xiii) PTEN signaling wherein, preferably, the gene is ITGAM, ITGA5, RAC1, PTEN, PRKCZ, BCL2L11, MAPK1, RAC2, AKT2, EGFR, IKBKB, CBL, PIK3CA, CDKN1B, PTK2, NFKB2, BCL2, PIK3CB, BCL2L1, MAPK3, ITGA1, KRAS, ITGB7, ILK, PDGFRB, INSR, RAF1, IKBKG, CASP9, CDKN1A, ITGB1, MAP2K2, AKT1, PIK3R1, CHUK, PDGFRA, PDPK1, MAP2K1, NFKB1, ITGB3, CDC42, CCND1, GSK3A, ITGA2, GSK3B, AKT3, FOXO1, CASP3, or RPS6KB1;

xiv) p53 signaling wherein, preferably, the gene is PTEN, EP300, BBC3, PCAF, FASN, BRCA1, GADD45A, BIRC5, AKT2, PIK3CA, CHEK1, TP53INP1, BCL2, PIK3CB, PIK3C3, MAPK8, THBS1, ATR, BCL2L1, E2F1, PMAIP1, CHEK2, TNFRSF10B, TP73, RB1, HDAC9, CDK2, PIK3C2A, MAPK14, TP53, LRDD, CDKN1A, HIPK2, AKT1, RIK3R1, RRM2B, APAF1, CTNNB1, SIRT1, CCND1, PRKDC, ATM, SFN, CDKN2A, JUN, SNAI2, GSK3B, BAX, or AKT3;

xv) aryl hydrocarbon receptor signaling wherein, preferably, the gene is HSPB1, EP300, FASN, TGM2, RXRA, MAPK1, NQO1, NCOR2, SP1, ARNT, CDKN1B, FOS, CHEK1, SMARCA4, NFKB2, MAPK8, ALDH1A1, ATR, E2F1, MAPK3, NRIP1, CHEK2, RELA, TP73, GSTP1, RB1, SRC, CDK2, AHR, NFE2L2, NCOA3, TP53, TNF, CDKN1A, NCOA2, APAF1, NFKB1, CCND1, ATM, ESR1, CDKN2A, MYC, JUN, ESR2, BAX, IL6, CYP1B1, or HSP90AA1;

xvi) xenobiotic metabolism signaling wherein, preferably, the gene is PRKCE, EP300, PRKCZ, RXRA, MAPK1, NQO1, NCOR2, PIK3CA, ARNT, PRKCI, NFKB2, CAMK2A, PIK3CB, PPP2R1A, PIK3C3, MAPK8, PRKD1, ALDH1A1, MAPK3, NRIP1, KRAS, MAPK13, PRKCD, GSTP1, MAPK9, NOS2A, ABCB1, AHR, PPP2CA, FTL, NFE2L2, PIK3C2A, PPARGC1A, MAPK14, TNF, RAF1, CREBBP, MAP2K2, PIK3R1, PPP2R5C, MAP2K1, NFKB1, KEAP1, PRKCA, EIF2AK3, 1L6, CYP1B1, or HSP90AA1;

xvii) SAPK or JNK signaling wherein, preferably, the gene is PRKCE, IRAK1, PRKAA2, EIF2AK2, RAC1, ELK1, GRK6, MAPK1, GADD45A, RAC2, PLK1, AKT2, PIK3CA, FADD, CDK8, PIK3CB, PIK3C3, MAPK8, RIPK1, GNB2L1, IRS1, MAPK3, MAPK10, DAXX, KRAS, PRKCD, PRKAA1, MAPK9, CDK2, PIM1, PIK3C2A, TRAF2, TP53, LCK, MAP3K7, DYRK1A, MAP2K2, PIK3R1, MAP2K1, PAK3, CDC42, JUN, TTK, CSNK1A1, CRKL, BRAF, or SGK;

xviii) PPAr or RXR signaling wherein, preferably, the gene is PRKAA2, EP300, INS, SMAD2, TRAF6, PPARA, FASN, RXRA, MAPK1, SMAD3, GNAS, IKBKB, NCOR2, ABCA1, GNAQ, NFKB2, MAP3K14, STAT5B, MAPK8, IRS1, MAPK3, KRAS, RELA, PRKAA1, PPARGC1A, NCOA3, MAPK14, INSR, RAF1, IKBKG, RELB, MAP3K7, CREBBP, MAP2K2, JAK2, CHUK, MAP2K1, NFKB1, TGFBR1, SMAD4, JUN, IL1R1, PRKCA, IL6, HSP90AA1, or ADIPOQ;

xix) NF-KB signaling wherein, preferably, the gene is IRAK1, EIF2AK2, EP300, INS, MYD88, PRKCZ: TRAF6, TBK1, AKT2, EGFR, IKBKB, PIK3CA, BTRC, NFKB2, MAP3K14, PIK3CB, PIK3C3, MAPK8, RIPK1, HDAC2, KRAS, RELA, PIK3C2A, TRAF2, TLR4: PDGFRB, TNF, INSR, LCK, IKBKG, RELB, MAP3K7, CREBBP, AKT1, PIK3R1, CHUK, PDGFRA, NFKB1, TLR2, BCL10, GSK3B, AKT3, TNFAIP3, or IL1R1;

xx) neuregulin signaling wherein, preferably, the gene is ERBB4, PRKCE, ITGAM, ITGA5: PTEN, PRKCZ, ELK1, MAPK1, PTPN11, AKT2, EGFR, ERBB2, PRKCI, CDKN1B, STAT5B, PRKD1, MAPK3, ITGA1, KRAS, PRKCD, STAT5A, SRC, ITGB7, RAF1, ITGB1, MAP2K2, ADAM17, AKT1, PIK3R1, PDPK1, MAP2K1, ITGB3, EREG, FRAP1, PSEN1, ITGA2, MYC, NRG1, CRKL, AKT3, PRKCA, HSP90AA1, or RPS6KB1;

xxi) Wnt or beta catenin signaling wherein, preferably, the gene is CD44, EP300, LRP6, DVL3, CSNK1E, GJA1, SMO, AKT2, PIN1, CDH1, BTRC, GNAQ, MARK2, PPP2R1A, WNT11, SRC, DKK1, PPP2CA, SOX6, SFRP2: ILK, LEF1, SOX9, TP53, MAP3K7, CREBBP, TCF7L2, AKT1, PPP2R5C, WNT5A, LRP5, CTNNB1, TGFBR1, CCND1, GSK3A, DVL1, APC, CDKN2A, MYC, CSNK1A1, GSK3B, AKT3, or SOX2;

xxii) insulin receptor signaling wherein, preferably, the gene is PTEN, INS, EIF4E, PTPN1, PRKCZ, MAPK1, TSC1, PTPN11, AKT2, CBL, PIK3CA, PRKCI, PIK3CB, PIK3C3, MAPK8, IRS1, MAPK3, TSC2, KRAS, EIF4EBP1, SLC2A4, PIK3C2A, PPP1CC, INSR, RAF1, FYN, MAP2K2, JAK1, AKT1, JAK2, PIK3R1, PDPK1, MAP2K1, GSK3A, FRAP1, CRKL, GSK3B, AKT3, FOXO1, SGK, or RPS6KB1;

xxiii) IL-6 signaling wherein, preferably, the gene is HSPB1, TRAF6, MAPKAPK2, ELK1, MAPK1, PTPN11, IKBKB, FOS, NFKB2: MAP3K14, MAPK8, MAPK3, MAPK10, IL6ST, KRAS, MAPK13, IL6R, RELA, SOCS1, MAPK9, ABCB1, TRAF2, MAPK14, TNF, RAF1, IKBKG, RELB, MAP3K7, MAP2K2, IL8, JAK2, CHUK, STAT3, MAP2K1, NFKB1, CEBPB, JUN, IL1R1, SRF, or IL6;

xxiv) hepatic cholestasis wherein, preferably, the gene is PRKCE, IRAK1, INS, MYD88, PRKCZ, TRAF6, PPARA, RXRA, IKBKB, PRKCI, NFKB2, MAP3K14, MAPK8, PRKD1, MAPK10, RELA, PRKCD, MAPK9, ABCB1, TRAF2, TLR4, TNF, INSR, IKBKG, RELB, MAP3K7, IL8, CHUK, NR1H2, TJP2, NFKB1, ESR1, SREBF1, FGFR4, JUN, IL1R1, PRKCA, or IL6;

xxv) IGF-1 signaling wherein, preferably, the gene is IGF1, PRKCZ, ELK1, MAPK1, PTPN11, NEDD4, AKT2, PIK3CA, PRKC1, PTK2, FOS, PIK3CB, PIK3C3, MAPK8, 1GF1R, IRS1, MAPK3, IGFBP7, KRAS, PIK3C2A, YWHAZ, PXN, RAF1, CASP9, MAP2K2, AKT1, PIK3R1, PDPK1, MAP2K1, IGFBP2, SFN, JUN, CYR61, AKT3, FOXO1, SRF, CTGF, or RPS6KB1;

xxvi) NRF2-mediated oxidative stress response wherein, preferably, the gene is PRKCE, EP300, SOD2, PRKCZ, MAPK1, SQSTM1, NQO1, PIK3CA, PRKC1, FOS, PIK3CB, P1K3C3, MAPK8, PRKD1, MAPK3, KRAS, PRKCD, GSTP1, MAPK9, FTL, NFE2L2, PIK3C2A, MAPK14, RAF1, MAP3K7, CREBBP, MAP2K2, AKT1, PIK3R1, MAP2K1, PPIB, JUN, KEAP1, GSK3B, ATF4, PRKCA, EIF2AK3, or HSP90AA1;

xxvii) hepatic fibrosis or hepatic stellate cell activation wherein, preferably, the gene is EDN1, IGF1, KDR, FLT1, SMAD2, FGFR1, MET, PGF, SMAD3, EGFR, FAS, CSF1, NFKB2, BCL2, MYH9, IGF1R, IL6R, RELA, TLR4, PDGFRB, TNF, RELB, IL8, PDGFRA, NFKB1, TGFBR1, SMAD4, VEGFA, BAX, IL1R1, CCL2, HGF, MMP1, STAT1, IL6, CTGF, or MMP9;

xxviii) PPAR signaling wherein, preferably, the gene is EP300, INS, TRAF6, PPARA, RXRA, MAPK1, IKBKB, NCOR2, FOS, NFKB2, MAP3K14, STAT5B, MAPK3, NRIP1, KRAS, PPARG, RELA, STAT5A, TRAF2, PPARGC1A, PDGFRB, TNF, INSR, RAF1, IKBKG, RELB, MAP3K7, CREBBP, MAP2K2, CHUK, PDGFRA, MAP2K1, NFKB1, JUN, IL1R1, or HSP90AA1;

xxix) Fc epsilon RI signaling wherein, preferably, the gene is PRKCE, RAC1, PRKCZ, LYN, MAPK1, RAC2, PTPN11, AKT2, PIK3CA, SYK, PRKCI, PIK3CB, PIK3C3, MAPK8, PRKD1, MAPK3, MAPK10, KRAS, MAPK13, PRKCD, MAPK9, PIK3C2A, BTK, MAPK14, TNF, RAF1, FYN, MAP2K2, AKT1, PIK3R1, PDPK1, MAP2K1, AKT3, VAV3, or PRKCA;

xxx) G-protein coupled receptor signaling wherein, preferably, the gene is PRKCE, RAP1A, RGS16, MAPK1, GNAS, AKT2, IKBKB, PIK3CA, CREB1, GNAQ, NFKB2, CAMK2A, PIK3CB, PIK3C3, MAPK3, KRAS, RELA, SRC, PIK3C2A, RAF1, IKBKG, RELB, FYN, MAP2K2, AKT1, PIK3R1, CHUK, PDPK1, STAT3, MAP2K1, NFKB1, BRAF, ATF4, AKT3, or PRKCA;

xxxi) inositol phosphate metabolism wherein, preferably, the gene is PRKCE, IRAK1, PRKAA2, EIF2AK2, PTEN, GRK6, MAPK1, PLK1, AKT2, PIK3CA, CDK8, PIK3CB, PIK3C3, MAPK8, MAPK3, PRKCD, PRKAA1, MAPK9, CDK2, PIM1, PIK3C2A, DYRK1A, MAP2K2, PIP5K1A, PIK3R1, MAP2K1, PAK3, ATM, TTK, CSNK1A1, BRAF, or SGK;

xxxii) PDGF signaling wherein, preferably, the gene is EIF2AK2, ELK1, ABL2, MAPK1, PIK3CA, FOS, PIK3CB, PIK3C3, MAPK8, CAV1, ABL1, MAPK3, KRAS, SRC, PIK3C2A, PDGFRB, RAF1, MAP2K2, JAK1, JAK2, PIK3R1, PDGFRA, STAT3, SPHK1, MAP2K1, MYC, JUN, CRKL, PRKCA, SRF, STAT1, or SPHK2;

xxxiii) VEGF signaling wherein, preferably, the gene is ACTN4, ROCK1, KDR, FLT1, ROCK2, MAPK1, PGF, AKT2, PIK3CA, ARNT, PTK2, BCL2, PIK3CB, PIK3C3, BCL2L1, MAPK3, KRAS, HIF1A, NOS3, PIK3C2A, PXN, RAF1, MAP2K2, ELAVL1, AKT1, PIK3R1, MAP2K1, SFN, VEGFA, AKT3, FOXO1, or PRKCA;

xxxiv) natural killer cell signaling wherein, preferably, the gene is PRKCE, RAC1, PRKCZ, MAPK1, RAC2, PTPN11, KIR2DL3, AKT2, PIK3CA, SYK, PRKCI, PIK3CB, PIK3C3, PRKD1, MAPK3, KRAS, PRKCD, PTPN6, PIK3C2A, LCK, RAF1, FYN, MAP2K2, PAK4, AKT1, PIK3R1, MAP2K1, PAK3, AKT3, VAV3, or PRKCA;

xxxv) cell cycle G1/S checkpoint regulation wherein, preferably, the gene is HDAC4, SMAD3, SUV39H1, HDAC5, CDKN1B, BTRC, ATR, ABL1, E2F1, HDAC2, HDAC7A, RB1, HDAC11, HDAC9, CDK2, E2F2, HDAC3, TP53, CDKN1A, CCND1, E2F4, ATM, RBL2, SMAD4, CDKN2A, MYC, NRG1, GSK3B, RBL1, or HDAC6;

xxxvi) T cell receptor signaling wherein, preferably, the gene is RAC1, ELK1, MAPK1, IKBKB, CBL, PIK3CA, FOS, NFKB2, PIK3CB, PIK3C3, MAPK8, MAPK3, KRAS, RELA, PIK3C2A, BTK, LCK, RAF1, IKBKG, RELB, FYN, MAP2K2, PIK3R1, CHUK, MAP2K1, NFKB1, ITK, BCL10, JUN, or VAV3;

xxxvii) death receptor signaling wherein, preferably, the gene is CRADD, HSPB1, BID, BIRC4, TBK1, IKBKB, FADD, FAS, NFKB2, BCL2, MAP3K14, MAPK8, RIPK1, CASP8, DAXX, TNFRSF10B, RELA, TRAF2, TNF, IKBKG, RELB, CASP9, CHUK, APAF1, NFKB1, CASP2, BIRC2, CASP3, or BIRC3;

xxxviii) FGF signaling wherein, preferably, the gene is RAC1, FGFR1, MET, MAPKAPK2, MAPK1, PTPN11, AKT2, PIK3CA, CREB1, PIK3CB, PIK3C3, MAPK8, MAPK3, MAPK13, PTPN6, PIK3C2A, MAPK14, RAF1, AKT1, PIK3R1, STAT3, MAP2K1, FGFR4, CRKL, ATF4, AKT3, PRKCA, or HGF;

xxxix) GM-CSF signaling wherein, preferably, the gene is LYN, ELK1, MAPK1, PTPN11, AKT2, PIK3CA, CAMK2A, STAT5B, PIK3CB, PIK3C3, GNB2L1, BCL2L1, MAPK3, ETS1, KRAS, RUNX1, PIM1, PIK3C2A, RAF1, MAP2K2, AKT1, JAK2, PIK3R1, STAT3, MAP2K1, CCND1, AKT3, or STAT1;

xl) amyotrophic lateral sclerosis signaling wherein, preferably, the gene is BID, IGF1, RAC1, BIRC4, PGF, CAPNS1, CAPN2, PIK3CA, BCL2, PIK3CB, PIK3C3, BCL2L1, CAPN1, PIK3C2A, TP53, CASP9, PIK3R1, RAB5A, CASP1, APAF1, VEGFA, BIRC2, BAX, AKT3, CASP3, or BIRC3;

xli) JAK-Stat signaling wherein, preferably, the gene is PTPN1, MAPK1, PTPN11, AKT2, PIK3CA, STAT5B, PIK3CB, PIK3C3, MAPK3, KRAS, SOCS1, STAT5A, PTPN6, PIK3C2A, RAF1, CDKN1A, MAP2K2, JAK1, AKT1, JAK2, PIK3R1, STAT3, MAP2K1, FRAP1, AKT3, STAT1;

xlii) nicotinate or nicotinamide metabolism wherein, preferably, the gene is PRKCE, IRAK1, PRKAA2, EIF2AK2, GRK6, MAPK1, PLK1, AKT2, CDK8, MAPK8, MAPK3, PRKCD, PRKAA1, PBEF1, MAPK9, CDK2, PIM1, DYRK1 A, MAP2K2, MAP2K1, PAK3, NT5E, TTK, CSNK1A1, BRAF, or SGK;

xliii) chemokine signaling wherein, preferably, the gene is CXCR4, ROCK2, MAPK1, PTK2, FOS, CFL1, GNAQ, CAMK2A, CXCL12, MAPK8, MAPK3, KRAS, MAPK13, RHOA, CCR3, SRC, PPP1CC, MAPK14, NOX1, RAF1, MAP2K2, MAP2K1, JUN, CCL2, or PRKCA;

xliv) IL-2 signaling wherein, preferably, the gene is ELK1, MAPK1, PTPN11, AKT2, PIK3CA, SYK, FOS, STAT5B, PIK3CB, PIK3C3, MAPK8, MAPK3, KRAS, SOCS1, STAT5A, PIK3C2A: LCK, RAF1, MAP2K2, JAK1, AKT1, PIK3R1, MAP2K1, JUN, or AKT3;

xlv) synaptic long term depression wherein, preferably, the gene is PRKCE, IGF1, PRKCZ, PRDX6, LYN, MAPK1, GNAS, PRKC1, GNAQ, PPP2R1A, IGF1R, PRKID1, MAPK3, KRAS, GRN, PRKCD, NOS3, NOS2A, PPP2CA, YWHAZ, RAF1, MAP2K2, PPP2R5C, MAP2K1, or PRKCA;

xlvi) estrogen receptor signaling wherein, preferably, the gene is TAF4B, EP300, CARM1, PCAF, MAPK1, NCOR2, SMARCA4, MAPK3, NRIP1, KRAS, SRC, NR3C1, HDAC3, PPARGC1A, RBM9, NCOA3, RAF1, CREBBP, MAP2K2, NCOA2, MAP2K1, PRKDC, ESR1, or ESR2;

xlvii) protein ubiquitination pathway wherein, preferably, the gene is TRAF6, SMURF1, BIRC4, BRCA1, UCHL1, NEDD4, CBL, UBE2I, BTRC, HSPA5, USP7, USP10, FBXW7, USP9X, STUB1, USP22, B2M, BIRC2, PARK2, USP8, USP1, VHL, HSP90AA1, or BIRC3;

xlviii) IL-10 signaling wherein, preferably, the gene is TRAF6, CCR1, ELK1, IKBKB, SP1, FOS, NFKB2, MAP3K14, MAPK8, MAPK13, RELA, MAPK14, TNF, IKBKG, RELB, MAP3K7, JAK1, CHUK, STAT3, NFKB1, JUN, IL1R1, or IL6;

xlix) VDR or RXR activation wherein, preferably, the gene is PRKCE, EP300, PRKCZ, RXRA, GADD45A, HES1, NCOR2, SP1, PRKC1, CDKN1B, PRKD1, PRKCD, RUNX2, KLF4, YY1, NCOA3, CDKN1A, NCOA2, SPP1, LRP5, CEBPB, FOXO1, or PRKCA;

I) TGF-beta signaling wherein, preferably, the gene is EP300, SMAD2, SMURF1, MAPK1, SMAD3, SMAD1, FOS, MAPK8, MAPK3, KRAS, MAPK9, RUNX2, SERPINE1, RAF1, MAP3K7, CREBBP, MAP2K2, MAP2K1, TGFBR1, SMAD4, JUN, or SMAD5;

li) toll-like receptor signaling wherein, preferably, the gene is IRAK1, EIF2AK2, MYD88, TRAF6, PPARA, ELK1, IKBKB, FOS, NFKB2, MAP3K14, MAPK8, MAPK13, RELA, TLR4, MAPK14, IKBKG, RELB, MAP3K7, CHUK, NFKB1, TLR2, or JUN;

lii) p38 MAPK signaling wherein, preferably, the gene is HSPB1, IRAK1, TRAF6, MAPKAPK2, ELK1, FADD, FAS, CREB1, DDIT3, RPS6KA4, DAXX, MAPK13, TRAF2, MAPK14, TNF, MAP3K7, TGFBR1, MYC, ATF4, IL1R1, SRF, or STAT1;

liii) neurotrophin or TRK Signaling wherein, preferably, the gene is NTRK2, MAPK1, PTPN11, PIK3CA, CREB1, FOS, PIK3CB, PIK3C3, MAPK8, MAPK3, KRAS, PIK3C2A, RAF1, MAP2K2, AKT1, PIK3R1, PDPK1, MAP2K1, CDC42, JUN, or ATF4;

liv) FXR or RXR activation wherein, preferably, the gene is INS, PPARA, FASN, RXRA, AKT2, SDC1, MAPK8, APOB, MAPK10, PPARG, MTTP, MAPK9, PPARGC1A, TNF, CREBBP, AKT1, SREBF1, FGFR4, AKT3, or FOXO1;

lv) synaptic long term potentiation wherein, preferably, the gene is PRKCE, RAP1A, EP300, PRKCZ, MAPK1, CREB1, PRKC1, GNAQ, CAMK2A, PRKD1, MAPK3, KRAS, PRKCD, PPP1CC, RAF1, CREBBP, MAP2K2, MAP2K1, ATF4, or PRKCA;

lvi) calcium signaling wherein, preferably, the gene is RAP1A, EP300, HDAC4, MAPK1, HDAC5, CREB1, CAMK2A, MYH9, MAPK3, HDAC2, HDAC7A, HDAC11, HDAC9, HDAC3, CREBBP, CALR, CAMKK2, ATF4, or HDAC6;

lvii) EGF signaling wherein, preferably, the gene is ELK1, MAPK1, EGFR, PIK3CA, FOS, PIK3CB, PIK3C3, MAPK8, MAPK3, PIK3C2A, RAF1, JAK1, PIK3R1, STAT3, MAP2K1, JUN, PRKCA, SRF, or STAT1;

lviii) hypoxia signaling in the cardiovascular system wherein, preferably, the gene is EDN1, PTEN, EP300, NQO1, UBE2I, CREB1, ARNT, HIF1A, SLC2A4, NOS3, TP53, LDHA, AKT1, ATM, VEGFA, JUN, ATF4, VHL, or HSP90AA1;

lix) LPS or IL-1 mediated inhibition of RXR function wherein, preferably, the gene is IRAK1, MYD88, TRAF6, PPARA, RXRA, ABCA1, MAPK8, ALDH1A1, GSTP1, MAPK9, ABCB1, TRAF2, TLR4, TNF, MAP3K7, NR1H2, SREBF1, JUN, or IL1R1;

lx) LXR or RXR activation wherein, preferably, the gene is FASN, RXRA, NCOR2, ABCA1, NFKB2, IRF3, RELA, NOS2A, TLR4, TNF, RELB, LDLR, NR1H2, NFKB1, SREBF1, IL1R1, CCL2, 1L6, or MMP9;

lxi) amyloid processing wherein, preferably, the gene is PRKCE, CSNK1E, MAPK1, CAPNS1, AKT2, CAPN2, CAPN1, MAPK3, MAPK13, MAPT, MAPK14, AKT1, PSEN1, CSNK1A1, GSK3B, AKT3, or APP;

lxii) IL-4 signaling wherein, preferably, the gene is AKT2, PIK3CA, PIK3CB, PIK3C3, IRS1, KRAS, SOCS1, PTPN6, NR3C1, PIK3C2A, JAK1, AKT1, JAK2, PIK3R1, FRAP1, AKT3, or RPS6KB1;

lxiii) cell cycle: G2/M DNA damage checkpoint regulation wherein, preferably, the gene is EP300, PCAF, BRCA1, GADD45A, PLK1, BTRC, CHEK1, ATR, CHEK2, YWHAZ, TP53, CDKN1A, PRKDC, ATM, SFN, or CDKN2A;

lxiv) nitric oxide signaling in the cardiovascular system wherein, preferably, the gene is KDR, FLT1, PGF, AKT2, PIK3CA, PIK3CB, PIK3C3, CAV1, PRKCD, NOS3, PIK3C2A, AKT1, PIK3R1, VEGFA, AKT3, or HSP90AA1;

lxv) purine metabolism wherein, preferably, the gene is NME2, SMARCA4, MYH9, RRM2, ADAR, EIF2AK4, PKM2, ENTPD1, RAD51, RRM2B, TJP2, RAD51C, NT5E, POLD1, or NME1;

lxvi) cAMP-mediated Signaling wherein, preferably, the gene is RAP1A, MAPK1, GNAS, CREB1, CAMK2A, MAPK3, SRC, RAF1, MAP2K2, STAT3, MAP2K1, BRAF, or ATF4;

lxvii) mitochondrial dysfunction wherein, preferably, the gene is SOD2, MAPK8, CASP8, MAPK10, MAPK9, CASP9, PARK7, PSEN1, PARK2, APP, or CASP3;

lxviii) notch signaling wherein, preferably, the gene is HES1, JAG1, NUMB, NOTCH4, ADAM17, NOTCH2, PSEN1, NOTCH3, NOTCH1, or DLL4;

lxix) endoplasmic reticulum stress pathway wherein, preferably, the gene is HSPA5, MAPK8, XBP1, TRAF2, ATF6, CASP9, ATF4, EIF2AK3, or CASP3;

lxx) pyrimidine metabolism wherein, preferably, the gene is NME2, AICDA, RRM2, EIF2AK4, ENTPD1, RRM2B, NT5E, POLD1, or NME1;

lxxi) Parkinson's signaling wherein, preferably, the gene is UCHL1, MAPK8, MAPK13, MAPK14, CASP9, PARK7, PARK2, or CASP3;

lxxii) cardiac or beta adrenergic signaling wherein, preferably, the gene is GNAS, GNAQ, PPP2R1A, GNB2L1, PPP2CA, PPP1CC, or PPP2R5C;

lxxiii) glycolysis or gluconeogenesis wherein, preferably, the gene is HK2, GCK, GPI, ALDH1A1, PKM2, LDHA, or HK1;

lxxiv) interferon signaling wherein, preferably, the gene is IRF1, SOCS1, JAK1, JAK2, IFITM1, STAT1, or IFIT3;

lxxv) Sonic Hedgehog signaling wherein, preferably, the gene is ARRB2, SMO, GLI2, DYRK1 A, GLI1, GSK3B, or DYRKIB;

lxxvi) glycerophospholipid metabolism wherein, preferably, the gene is PLD1, GRN, GPAM, YWHAZ, SPHK1, or SPHK2;

lxxvii) phospholipid degradation wherein, preferably, the gene is PRDX6, PLD1, GRN, YWHAZ, SPHK1, or SPHK2;

lxxviii) tryptophan metabolism wherein, preferably, the gene is SIAH2, PRMT5, NEDD4, ALDH1A1, CYP1B1, or SIAH1;

lxxix) lysine degradation wherein, preferably, the gene is SUV39H1, EHMT2, NSD1, SETD7, or PPP2R5C;

lxxx) nucleotide excision repair pathway wherein, preferably, the gene is ERCC5, ERCC4, XPA, XPC, or ERCC1;

lxxxi) starch or sucrose metabolism wherein, preferably, the gene is UCHL1, HK2, GCK, GPI, or HK1;

lxxxii) amino sugars metabolism wherein, preferably, the gene is NQO1, HK2, GCK, or HK1;

lxxxiii) arachidonic acid metabolism wherein, preferably, the gene is PRDX6, GRN, YWHAZ, or CYP1B1;

lxxxiv) circadian rhythm signaling wherein, preferably, the gene is CSNK1E, CREB1, ATF4, or NR1 D1;

lxxxv) coagulation system wherein, preferably, the gene is BDKRB1, F2R, SERPINE1, or F3;

lxxxvi) dopamine receptor signaling wherein, preferably, the gene is PPP2R1 A, PPP2CA, PPP1CC, or PPP2R5C;

lxxxvii) glutathione metabolism wherein, preferably, the gene is IDH2, GSTP1, ANPEP, or IDH1;

lxxxviii) glycerolipid metabolism wherein, preferably, the gene is ALDH1A1, GPAM, SPHK1, or SPHK2;

lxxxix) linoleic acid metabolism wherein, preferably, the gene is PRDX6, GRN, YWHAZ, or CYP1B1;

xc) methionine metabolism wherein, preferably, the gene is DNMT1, DNMT3B, AHCY, or DNMT3A;

xci) pyruvate metabolism wherein, preferably, the gene is GLO1, ALDH1A1, PKM2, or LDHA;

xcii) arginine and proline metabolism wherein, preferably, the gene is ALDH1A1, NOS3, or NOS2A;

xciii) eicosanoid signaling wherein, preferably, the gene is PRDX6, GRN, or YWHAZ;

xciv) fructose and mannose metabolism wherein, preferably, the gene is HK2, GCK, or HK1;

xcv) galactose metabolism wherein, preferably, the gene is HK2, GCK, or HK1;

xcvi) stilbene, coumarine, or lignin biosynthesis wherein, preferably, the gene is PRDX6, PRDX1, or TYR;

xcvii) antigen presentation pathway wherein, preferably, the gene is CALR or B2M;

xcviii) biosynthesis of steroids wherein, preferably, the gene is NQO1 or DHCR7;

xcix) butanoate metabolism wherein, preferably, the gene is ALDH1A1 or NLGN1;

c) citrate cycle wherein, preferably, the gene is IDH2 or IDH1;

ci) fatty acid metabolism wherein, preferably, the gene is ALDH1A1 or CYP1B1;

cii) histidine metabolism wherein, preferably, the gene is PRMT5 or ALDH1A1;

ciii) inositol metabolism wherein, preferably, the gene is ERO1L or APEX1;

civ) metabolism of xenobiotics by Cytochrome p450 wherein, preferably, the gene is GSTP1 or CYP1B1;

cv) methane metabolism wherein, preferably, the gene is PRDX6 or PRDX1;

cvi) phenylalanine metabolism wherein, preferably, the gene is PRDX6 or PRDX1;

cvii) propanoate metabolism wherein, preferably, the gene is ALDH1A1 or LDHA;

ciii) selenoamino acid metabolism wherein, preferably, the gene is PRMT5 or AHCY;

cix) sphingolipid metabolism wherein, preferably, the gene is SPHK1 or SPHK2;

cx) aminophosphonate metabolism wherein, preferably, the gene is PRMT5;

cxi) androgen or estrogen metabolism wherein, preferably, the gene is PRMT5;

cxii) ascorbate and aldarate metabolism wherein, preferably, the gene is ALDH1A1;

cxiii) bile acid biosynthesis wherein, preferably, the gene is ALDH1A1;

cxiv) cysteine metabolism wherein, preferably, the gene is LDHA;

cxv) fatty acid biosynthesis wherein, preferably, the gene is FASN;

cxvi) glutamate receptor signaling wherein, preferably, the gene is GNB2L1;

cxvii) NRF2-mediated oxidative stress response wherein, preferably, the gene is PRDX1;

cxiii) pentose phosphate pathway wherein, preferably, the gene is GPI;

cxix) pentose and glucuronate interconversions wherein, preferably, the gene is UCHL1;

cxx) retinol metabolism wherein, preferably, the gene is ALDH1A1;

cxxi) riboflavin metabolism wherein, preferably, the gene is TYR;

cxxii) tyrosine metabolism wherein, preferably, the gene is PRMT5 or TYR;

cxxiii) ubiquinone biosynthesis wherein, preferably, the gene is PRMT5;

cxxiv) valine, leucine and isoleucine degradation wherein, preferably, the gene is ALDH1A1;

cxxv) glycine, serine and threonine metabolism wherein, preferably, the gene is CHKA;

cxxvi) lysine degradation wherein, preferably, the gene is ALDH1A1;

cxxvii) pain or taste wherein, preferably, the gene is TRPM5 or TRPA1;

cxxiii) pain wherein, preferably, the gene is TRPM7, TRPC5, TRPC6, TRPC1, CNR1, CNR2, GRK2, TRPA1, POMC, CGRP, CRF, PKA, ERA, NR2b, TRPM5, PRKACa, PRKACb, PRKAR1a, or PRKAR2a;

cxxix) mitochondrial function wherein, preferably, the gene is AIF, CYTC, SMAC (Diablo), AIFM-1, or AIFM-2;

cxxx) developmental neurology wherein, preferably, the gene is BMP-4, chordin (CHRD), noggin (Nog), WNT, WNT2, WNT2b, WNT3a, WNT4, WNT5a, WNT6, WNT7b, WNT8b, WNT9a, WNT9b, WNT10a, WNT10b, WNT16, beta-catenin, DKK-1, frizzled related proteins, OTX-2, GBX2, FGF-8, Reelin, DAB1, UNC-86, POU4f1, BRN3a, NUMB, or RELN.

### Definitions

The term “about” means±10% of the stated amount.

As used herein, the term “binds to” or “specifically binds to” refers to measurable and reproducible interactions such as binding between a guide polynucleotide and an RNA programmable nuclease, which is determinative of the presence of the target in the presence of a heterogeneous population of molecules including biological molecules. For example, an RNA programmable nuclease that binds to or specifically binds to a guide polynucleotide (which can be an engineered guide polynucleotide) is an RNA programmable nuclease that binds this guide polynucleotide with greater affinity, avidity, more readily, and/or with greater duration than it binds to other guide polynucleotides. In certain examples, an RNA programmable nuclease that specifically binds to a guide polynucleotide has a dissociation constant (Kd) of ≤1 μM, ≤100 nM, ≤10 nM, ≤1 nM, or ≤0.1 nM. In certain examples, an RNA programmable nuclease binds to a guide polynucleotide (e.g., guide RNA), wherein the RNA programmable nuclease and the guide polynucleotide form a complex at a target site (e.g., a target genomic site) on a target nucleic acid (e.g., a target genome). In another aspect, specific binding can include, but does not require exclusive binding.

The term “Cas” or “Cas nuclease” refers to an RNA-guided nuclease comprising a Cas protein (e.g., a Cas9 protein), or a fragment thereof (e.g., a protein comprising an active cleavage domain of Cas). A Cas nuclease is also referred to alternatively as an RNA-programmable nuclease, and a CRISPR/Cas system. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc) and a Cas protein (e.g., a Cas9 protein). The tracrRNA serves as a guide for ribonuclease 3-aided processing of pre-crRNA. Subsequently, Cas/crRNA/tracrRNA cleaves linear or circular dsDNA target complementary to the spacer. The target strand not complementary to crRNA is first cut by endonuclease activity, then trimmed 3′-5′ by exonuclease activity. In nature, DNA-binding and cleavage typically requires Cas protein, crRNA, and tracrRNA. However, single guide RNAs (“sgRNA”, or simply “gRNA”) can be engineered so as to incorporate aspects of both the crRNA and tracrRNA into a single RNA species. See, e.g., Jinek et al. (Science 337:816-821, 2012), the entire contents of which is hereby incorporated by reference. RNA programmable nucleases (e.g., Cas9) recognize a short motif in the CRISPR repeat sequences (the protospacer adjacent motif (PAM)) to help distinguish self versus non-self. Cas9 nuclease sequences and structures are well known to those of skill in the art (see, e.g., Ferretti et al. (Proc. Natl. Acad. Sci. U.S.A. 98:4658-4663, 2001); Deltcheva et al. (Nature 471:602-607, 2011); and Jinek et al. (2012, supra), the entire contents of each of which are incorporated herein by reference). Cas9 orthologs have been described in various species, including, but not limited to, S. pyogenes and S. thermophilus. Additional suitable RNA programmable nucleases and sequences will be apparent to those of skill in the art based on this disclosure, and such RNA programmable nucleases and sequences include Cas9 sequences from the organisms and loci disclosed in, e.g., Chylinski et al. (RNA Biology 10:5, 726-737, 2013); the entire contents of which are incorporated herein by reference.

As used herein, a “coding region” is a portion of a nucleic acid that contains codons that can be translated into amino acids. Although a “stop codon” (TAG, TGA, TAA) is not translated into an amino acid, it may be considered to be part of a coding region, if present, but any flanking sequences, for example, promoters, ribosome binding sites, transcriptional terminators, introns, 5′ and 3′ untranslated regions, and the like, are not part of the coding region.

As used herein, “codon optimization” refers a process of modifying a nucleic acid sequence in accordance with the principle that the frequency of occurrence of synonymous codons (e.g., codons that code for the same amino acid) in coding DNA is biased in different species. Such codon degeneracy allows an identical polypeptide to be encoded by a variety of nucleotide sequences. Sequences modified in this way are referred to herein as “codon-optimized.” This process may be performed on any of the sequences described in this specification to enhance expression or stability. Codon optimization may be performed in a manner such as that described in, e.g., U.S. Pat. Nos. 7,561,972, 7,561,973, and 7,888,112, the entire contents of each of which is incorporated herein by reference. The sequence surrounding the translational start site can be converted to a consensus Kozak sequence according to known methods. See, e.g., Kozak et al. (Nucleic Acids Res. 15 (20): 8125-8148, 1987), the entire contents of which is hereby incorporated by reference. Multiple stop codons can be incorporated.

The term “complementary,” as used herein in reference to a nucleobase sequence, refers to the nucleobase sequence having a pattern of contiguous nucleobases that permits an oligonucleotide having the nucleobase sequence to hybridize to another oligonucleotide or nucleic acid to form a duplex structure under physiological conditions. Complementary sequences include Watson-Crick base pairs formed from natural and/or modified nucleobases. Complementary sequences can also include non-Watson-Crick base pairs, such as wobble base pairs (guanosine-uracil, hypoxanthine-uracil, hypoxanthine-adenine, and hypoxanthine-cytosine), and Hoogsteen base pairs.

The term “contiguous,” as used herein in the context of an oligonucleotide, refers to nucleosides, nucleobases, sugar moieties, or inter-nucleoside linkages that are immediately adjacent to each other. For example, “contiguous nucleobases” means nucleobases that are immediately adjacent to each other in a sequence.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is, therefore, interpreted to be an open term meaning “at least the following,” and is also interpreted not to exclude additional features, limitations, aspects, etc. Thus, for example, “a process involving steps a, b, and c” means that the process includes at least steps a, b, and c. Wherever the terms “a” or “an” are used, “one or more” is understood, unless such interpretation is nonsensical in context.

The terms “conjugating,” “conjugated,” and “conjugation” refer to an association of two entities, for example, of two molecules such as two proteins, two domains (e.g., a binding domain and a cleavage domain), or a protein and an agent, e.g., a protein binding domain and a small molecule. In some aspects, the association is between a protein (e.g., RNA-programmable nuclease) and a nucleic acid (e.g., a guide RNA). The association can be, for example, via a direct or indirect (e.g., via a linker) covalent linkage. In some embodiments, the association is covalent. In some embodiments, two molecules are conjugated via a linker connecting both molecules. For example, in some embodiments where two proteins are conjugated to each other, e.g., a RNA programmable nuclease and a nuclease (e.g., an exonuclease), to form a protein fusion, the two proteins may be conjugated via a polypeptide linker, e.g., an amino acid sequence connecting the C-terminus of one protein to the N-terminus of the other protein, in either order.

The term “consensus sequence,” as used herein in the context of nucleic acid sequences, refers to a calculated sequence representing the most frequent nucleotide residues found at each position in a plurality of similar sequences. Typically, a consensus sequence is determined by sequence alignment in which similar sequences are compared to each other and similar sequence motifs are calculated. In the context of nuclease target genomic site sequences, a consensus sequence of a nuclease target genomic site may, in some embodiments, be the sequence most frequently bound, or bound with the highest affinity, by a given nuclease.

The term “engineered,” as used herein refers to a protein molecule, a nucleic acid, complex, substance, or entity that has been designed, produced, prepared, synthesized, and/or manufactured by human intervention and an engineered product is a product that does not occur in nature.

The term “effective amount,” as used herein, refers to an amount of a biologically active agent that is sufficient to elicit a desired biological response. For example, in some embodiments, an effective amount of a nuclease may refer to the amount of the nuclease that is sufficient to induce homology directed repair after cleavage of a target genomic site specifically bound and cleaved by the nuclease. As will be appreciated by the skilled artisan, the effective amount of an agent, e.g., a nuclease, a fusion protein, a complex of a protein and a polynucleotide, a polynucleotide, a viral vector, or a non-viral delivery vehicle, may vary depending on various factors as, for example, on the desired biological response, the specific allele, genome, target genomic site, cell, or tissue being targeted, and the agent being used.

The term “delivery vehicle” refers to a construct which is capable of delivering, and, within preferred embodiments expressing, all or a fragment of one or more gene(s) or nucleic acid molecule(s) of interest in a host cell or subject. The term “fragment of,” as used herein, refers to a segment (e.g., segments of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, at least about 98%, at least about 99%, at least about 99.5%, or at least about 99.9%) of the full length gene(s) or nucleic acid molecule(s) of interest. Representative examples of such delivery vehicles include, but are not limited to, vectors (e.g., viral vectors), nucleic acid expression vectors, naked DNA, and cells (e.g., eukaryotic cells).

The term “homologous,” as used herein is an art-understood term that refers to nucleic acids or polypeptides that are highly related at the level of the nucleotide and/or amino acid sequence. Nucleic acids or polypeptides that are homologous to each other are termed “homologues.” Homology between two sequences can be determined by sequence alignment methods known to those of skill in the art, for instance, using publicly available computer software such as BLAST, ALIGN, or Megalign (DNASTAR) software. In accordance with the invention, two sequences are considered to be homologous if they are at least about 50-60% identical (e.g., at least about 70% identical, at least about 80% identical, at least about 90% identical, at least about 95% identical, at least about 98% identical, at least about 99% identical, at least about 99.5% identical, or at least about 99.9% identical), e.g., share identical residues (e.g., amino acid or nucleic acid residues) in at least about 50-60% of all residues comprised in one or the other sequence, for at least one stretch of at least 20, at least 30, at least 40, at least 50, at least 60, at least 70, at least 80, at least 90, at least 100, at least 120, at least 150, at least 200, at least 250, at least 300, at least 350, at least 400, at least 500, at least 600, at least 700, at least 900, at least 1100, at least 1300, at least 1500, at least 2000, at least 2500, at least 3000, at least 4000, at least 5000, at least 7000, at least 9000, at least 10000, or at least 15000 residues (e.g., amino acids or nucleic acids).

As used herein, the term “IRES” refers to an internal ribosomal entry site. In general, an IRES sequence is a feature that allows eukaryotic ribosomes to bind an mRNA transcript and begin translation without binding to a 5′ capped end. An mRNA containing an IRES sequence produces two translation products, one initiating form the 5′ end of the mRNA and the other from an internal translation mechanism mediated by the IRES.

The term “lentiviral vector” refers to a nucleic acid construct derived from a lentivirus which carries, and, within certain embodiments, is capable of directing the expression of, a nucleic acid molecule of interest. Lentiviral vectors can have one or more of the lentiviral wild-type genes deleted in whole or part, but retain functional flanking long-terminal repeat (LTR) sequences (also described below). Functional LTR sequences are necessary for the rescue, replication and packaging of the lentiviral virion. Thus, a lentiviral vector is defined herein to include at least those sequences required in cis for replication and packaging (e.g., functional LTRs) of the virus. The LTRs need not be the wild-type nucleotide sequences, and may be altered, e.g., by the insertion, deletion or substitution of nucleotides, so long as the sequences provide for functional rescue, replication and packaging.

The term “lentiviral vector particle” refers to a recombinant lentivirus which carries at least one gene or nucleotide sequence of interest, which is generally flanked by lentiviral LTRs. The lentivirus may also contain a selectable marker. The recombinant lentivirus is capable of reverse transcribing its genetic material into DNA and incorporating this genetic material into a host cell's DNA upon infection. Lentiviral vector particles may have a lentiviral envelope, a non-lentiviral envelope (e.g., an amphotropic or VSV-G envelope), a chimeric envelope, or a modified envelope (e.g., truncated envelopes or envelopes containing hybrid sequences).

The term “linker” refers to a chemical group or a molecule linking two adjacent molecules or moieties, e.g., a first domain (e.g., an RNA programmable nuclease) and a second domain (e.g., an exonuclease). In some embodiments, a linker joins a nuclear localization signal (NLS) domain to another protein (e.g., an RNA programmable nuclease or a nuclease or a fusion thereof). Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is a peptide linker. In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids. In some embodiments, the peptide linker comprises repeats of the tri-peptide Gly-Gly-Ser, e.g., comprising the sequence (GGS)n, wherein n represents at least 1, 2, 3, 4, 5, 6, 7, 8, 9, 10, or more repeats. In some embodiments, the linker comprises the sequence (GGS)₆. In some embodiments, the peptide linker is the 16 residue “XTEN” linker, or a variant thereof (see, e.g., Schellenberger et al. (Nat. Biotechnol. 27: 1186-1190, 2009).

The term “mutation,” as used herein, refers to a substitution, insertion, or deletion of a residue within a sequence, e.g., a nucleic acid or amino acid sequence, with another residue, or a substitution, insertion, or deletion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are discussed in, for example, Green and Sambrook, Molecular Cloning: A Laboratory Manual (4^{th} ed., Cold Spring Harbor Laboratory Press, Cold Spring Harbor, N.Y. (2012)).

The term “nuclease” refers to an agent, for example, a protein, capable of cleaving a phosphodiester bond connecting two nucleotide residues in a nucleic acid molecule. In some embodiments, a nuclease is a protein, e.g., an enzyme that can bind a nucleic acid molecule and cleave a phosphodiester bond connecting nucleotide residues within the nucleic acid molecule. A nuclease may be an endonuclease, cleaving a phosphodiester bond within a polynucleotide chain, or an exonuclease, cleaving a phosphodiester bond at the end of the polynucleotide chain. In some embodiments, a nuclease is a site-specific nuclease, binding and/or cleaving a specific phosphodiester bond within a specific nucleotide sequence, which is also referred to herein as the “recognition sequence,” the “nuclease target site,” or the “target genomic site.” In some embodiments, a nuclease is a RNA-guided (e.g., RNA-programmable) nuclease, which is associated with (e.g., binds to) an RNA (e.g., a guide RNA (“gRNA”)) having a sequence that complements a target genomic site, thereby providing sequence specificity to the nuclease. In some embodiments, a nuclease recognizes a single stranded target genomic site, while in other embodiments, a nuclease recognizes a double-stranded target genomic site, for example, a double-stranded DNA target genomic site. Some endonucleases cut a double-stranded nucleic acid target site symmetrically, e.g., cutting both strands at the same position so that the ends comprise base-paired nucleotides, also referred to herein as blunt ends. Some nucleases are exonucleases and excise the terminal nucleic acid of a single strand, leaving the complementary strand unpaired. Unpaired nucleotides at the end of a double-stranded DNA molecule are also referred to as “overhangs,” e.g., as “5′-overhang” or as “3′-overhang,” depending on whether the unpaired nucleotide(s) form(s) the 5′ or the 3′ end of the respective DNA strand. Double-stranded DNA molecule ends ending with unpaired nucleotide(s) are also referred to as sticky ends, as they can “stick to” other double-stranded DNA molecule ends comprising complementary unpaired nucleotide(s). A nuclease protein typically comprises a “binding domain” that mediates the interaction of the protein with the nucleic acid substrate, and also, in some cases, specifically binds to a target site, and a “cleavage domain” that catalyzes the cleavage of the phosphodiester bond within the nucleic acid backbone. In some embodiments a nuclease protein can bind and cleave a nucleic acid molecule in a monomeric form. Binding domains and cleavage domains of naturally occurring nucleases, as well as modular binding domains and cleavage domains that can be fused to create nucleases binding specific target sites, are well known to those of skill in the art.

The terms “nucleic acid” and “nucleic acid molecule” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, gRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs, such as analogs having chemically modified bases or sugars and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

As used herein, the term “pharmaceutically acceptable carrier” refers to an excipient or diluent in a pharmaceutical composition. The pharmaceutically acceptable carrier is compatible with the other components of the formulation and not deleterious to the recipient. The pharmaceutically acceptable carrier may impart pharmaceutical stability to the composition (e.g., stability to a Cas-exonuclease fusion protein, a guide polynucleotide (e.g., a gRNA), and/or a donor DNA molecule such as those described herein), or may impart another beneficial characteristic (e.g., sustained release characteristics). The nature of the carrier may differ with the mode of administration. For example, for intravenous administration, an aqueous solution carrier is generally used; for oral administration, a solid carrier may be preferred.

As used herein, the term “pharmaceutical composition” refers to a medicinal or pharmaceutical formulation that contains an active agent at a pharmaceutically acceptable purity, as well as one or more excipients and diluents that are suitable for the method of administration and are generally regarded as safe for the recipient according to recognized regulatory standards. The pharmaceutical composition includes pharmaceutically acceptable components that are compatible with, for example, a Cas-exonuclease fusion protein, or fragment thereof (or a nucleic acid encoding such a fusion protein), a guide polynucleotide (e.g., guide RNA), and/or a donor DNA molecule, as described herein. The pharmaceutical composition may be in aqueous form, for example, for intravenous or subcutaneous administration, in tablet or capsule form, for example, for oral administration, or in cream for, for example, for topical administration.

The terms “protein” and “peptide” and “polypeptide” are used interchangeably and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The terms “RNA-programmable nuclease” and “RNA-guided nuclease” are used interchangeably and refer to a nuclease that forms a complex with (e.g., specifically binds to or associates with) one or more polynucleotide molecules (e.g., RNA molecules), that are not a target for cleavage, but that direct the RNA-programmable nuclease to a target cleavage site complementary to the spacer sequence of a guide polynucleotide. In some embodiments, an RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target site (e.g., a target genomic site) (e.g., to direct binding of a Cas complex (e.g., a Cas9 complex) to the target site); and (2) a domain that binds a Cas nuclease (e.g., a Cas9 protein). In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al. (2012, supra), the entire contents of which are incorporated herein by reference. Still other examples of gRNAs and gRNA structure are provided herein. (see, e.g., the Examples). The gRNA comprises a nucleotide sequence that has a complementary sequence to a target site (e.g., a target genomic site), which mediates binding (e.g., specific binding) of the nuclease/RNA complex to the target site, thereby providing the sequence specificity of the nuclease:RNA complex. In some embodiments, the RNA-programmable nuclease is the (CRISPR-associated system) Cas9 endonuclease, for example Cas9 from Streptococcus pyogenes (see, e.g., Ferretti et al. (2001, supra); Deltcheva et al. (2011, supra); and Jinek et al. (2012, supra)).

Because RNA-programmable nucleases (e.g., Cas9) use RNA:DNA hybridization to determine cleavage sites, these proteins are able to cleave, in principle, any sequence specified by the guide RNA. Methods of using RNA-programmable nucleases, such as Cas9, for site-specific cleavage (e.g., to modify a genome) are known in the art (see e.g., Cong et al. (Science 339: 819-823, 2013); Mali et al. (Science 339: 823-826, 2013; Hwang et al. (Nature biotechnology 31: 227-229, 2013); Jinek et al. (eLife 2, e00471, 2013); Dicarlo et al. (Nucleic acids research 10(7):4336-4343, 2013); and Jiang et al. (Nature biotechnology 31: 233-239, 2013); the entire contents of each of which are incorporated herein by reference).

The term “recombine” or “recombination” in the context of a nucleic acid modification (e.g., a genomic modification), is used to refer to the process by which two or more nucleic acid molecules, or two or more regions of a single nucleic acid molecule, are modified by the action of an RNA programmable nuclease (e.g., a Cas9) fusion protein provided herein. Recombination can result in, inter alia, the insertion, inversion, excision or translocation of nucleic acids, e.g., in or between one or more nucleic acid molecules.

The term “subject” refers to an organism, for example, a vertebrate (e.g., a mammal, bird, reptile, amphibian, and fish). In some embodiments, the subject is a human. In some embodiments, the subject is a non-human mammal (e.g., a non-human primate). In some embodiments, the subject is a sheep, a goat, a cattle, a rodent, a cat, a dog, an insect (e.g., a fly), or a nematode. In some embodiments, the subject is a research animal. In some embodiments, the subject is genetically engineered, e.g., a genetically engineered non-human subject. The subject may be of either sex and at any stage of development.

The terms “target nucleic acid” and “target genome” and “endogenous DNA” as used herein in the context of nucleases, refer to a nucleic acid molecule (e.g., a nucleic acid molecule of a genome, such as a nucleic acid molecule of a chromosome (e.g., a gene)), that comprises at least one target site (e.g., a target genomic site) of an RNA-programmable nuclease. In the context of the featured fusion proteins comprising a Cas endonuclease linked to an exonuclease, a “target nucleic acid” and a “target genome” refers to one or more nucleic acid molecule(s), or a genome, respectively, that comprises at least one target genomic site. In some embodiments, the target nucleic acid(s) comprises at least two, at least three, or at least four target genomic sites. In some embodiments, the target nucleic acid(s) comprise four target genomic sites.

The term “target site” refers to a sequence within a nucleic acid molecule that is bound and cleaved by a nuclease (e.g., Cas fusion proteins described herein). A “target genomic site” refers to a sequence within the genome of a subject (e.g., a site in a chromosome, such as within a gene). A target site or target genomic site may be single-stranded or double-stranded. In the context of RNA-guided (e.g., RNA-programmable) nucleases (e.g., a fusion protein comprising a Cas9 and an exonuclease), a target genomic site typically comprises a nucleotide sequence that is complementary to the gRNA(s) of the RNA-programmable nuclease and a protospacer adjacent motif (PAM) at the 3′ end adjacent to the gRNA-complementary sequence(s) on the non-target strand. In some embodiments, such as those involving Cas fusion proteins, a target site or target genomic site can encompass the particular sequences to which Cas monomers bind and/or the intervening sequence between the bound monomers that are cleaved by the Cas nuclease domain, and the terminal nucleic acids are removed by the exonuclease domains thereby creating 5′ and/or 3′ overhangs mimicking ssDNA. For the RNA-guided nuclease Cas (or gRNA-binding domain thereof) and the featured fusion protein of Cas-exonuclease described herein, the target site or target genomic site may be, in some embodiments, 17-25 base pairs plus a 3 base pair PAM (e.g., NNN, wherein N independently represents any nucleotide). Typically, the first nucleotide of a PAM can be any nucleotide, while the two downstream nucleotides are specified depending on the specific RNA-guided nuclease. Exemplary PAM sites for RNA-guided nucleases, such as Cas9, are known to those of skill in the art and include, without limitation, NGG (SEQ ID NO: 1), NAG (SEQ ID NO: 2), NNG (SEQ ID NO: 17), and NGN (SEQ ID NO: 18), wherein N independently represents any nucleotide. In addition, Cas9 nucleases from different species (e.g., S. thermophilus instead of S. pyogenes) recognize a PAM that comprises the sequence NGGNG (SEQ ID NO: 15). Additional PAM sequences are known, including, but not limited to, NNAGAAW (SEQ ID NO: 14) and NAAR (SEQ ID NO: 19, wherein W independently represents A or T, and wherein R independently represents A or G (see, e.g., Esvelt and Wang (Molecular Systems Biology, 9:641, 2013), the entire contents of which are incorporated herein by reference). In some aspects, the target site or target genomic site of an RNA-guided nuclease, such as, e.g., Cas9, may comprise the structure [Nz]-[PAM], where each N is, independently, any nucleotide, and z is an integer between 1 and 50, inclusive. In some embodiments, z, which is the number of N nucleotides, is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50. In some embodiments, z is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, z is 20.

As used herein, the term “therapeutically effective amount” refers to an amount, e.g., a pharmaceutical dose of a composition described herein (e.g., a composition containing a fusion protein described herein and two or more guide polynucleotides (e.g., gRNA), and, optionally, a donor DNA molecule), effective in inducing a desired biological effect in a subject or in treating a subject with a medical condition or disorder described herein (e.g., disease or disorder in Tables 5 and 6). In some embodiments, the composition further comprises a donor DNA molecule (e.g., a DNA molecule containing a functional version of a gene(s), or a fragment thereof, such as a gene(s) causing a disease or disorder, for example, one of the diseases or disorders listed in Tables 5 and 6) to be inserted at the target site, e.g., to restore the functionality of the gene(s)). It is also to be understood herein that a “therapeutically effective amount” may be interpreted as an amount giving a desired therapeutic effect, either taken in one dose or in any dosage or route, taken alone or in combination with other therapeutic agents.

As used herein, the terms “treatment” or “treating” refer to reducing or ameliorating a medical condition (e.g., a disease or disorder) and/or symptoms associated therewith (e.g., those described herein, see, e.g., Tables 5 and 6). It will be appreciated that, although not precluded, treating a medical condition does not require that the disorder or symptoms associated therewith be completely eliminated. Reducing or decreasing the side effects of a medical condition, such as those described herein, or the risk or progression of the medical condition, may be relative to a subject who did not receive treatment, e.g., a control, a baseline, or a known control level or measurement. The reduction or decrease may be, e.g., by about 1%, 2%, 3%, 4%, 5%, 6%, 7%, 8%, 9%, 10%, 20%, 30%, 40%, 50%, 60%, 70%, 80%, 90%, 95%, 97%, 99%, or about 100% relative to the subject who did not receive treatment or the control, baseline, or known control level or measurement, or may be a reduction in the number of days during which the subject experiences the medical condition or associated symptoms (e.g., a reduction of 1-30 days, 2-12 months, 2-5 years, or 6-12 years). As defined herein, a therapeutically effective amount of a pharmaceutical composition of the present disclosure may be readily determined by one of ordinary skill by routine methods known in the art. Dosage regimen may be adjusted to provide the optimum therapeutic response.

The term “substantially” used herein allows for deviations from the descriptor that do not negatively impact the intended purpose. Descriptive terms may be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

Wherever any of the phrases “such as,” “for example,” “including” and the like are used herein, the phrase “and without limitation” is understood to follow unless explicitly stated otherwise. Similarly “an example,” “exemplary” and the like are understood to be non-limiting.

The term “vector” refers to a polynucleotide comprising one or more recombinant polynucleotides described herein, e.g., those encoding a Cas nuclease (e.g., a Cas9 nuclease), Cas protein or fusion protein thereof, a gRNA, and, optionally, a donor DNA molecule. Vectors include, but are not limited to, plasmids, viral vectors, cosmids, artificial chromosomes, and phagemids. Typically, a vector is able to replicate in a host cell and can be further characterized by one or more endonuclease restriction sites at which the vector may be cut and into which a desired nucleic acid molecule may be inserted. Vectors may contain one or more marker sequences suitable for use in the identification and/or selection of cells which have or have not been transformed or genomically modified with the vector. Markers include, for example, genes encoding proteins which increase or decrease either resistance or sensitivity to antibiotics (e.g., kanamycin, ampicillin) or other compounds, genes which encode enzymes whose activities are detectable by standard assays known in the art (e.g., β-galactosidase, alkaline phosphatase, or luciferase), and genes which visibly affect the phenotype of transformed or transfected cells, hosts, colonies, or plaques. Any vector suitable for the transformation of a host cell (e.g., E. coli, mammalian cells such as CHO cell, insect cells, etc.) as embraced by the present invention, for example, vectors belonging to the pUC series, pGEM series, pET series, pBAD series, pTET series, or pGEX series. In some embodiments, the vector is suitable for transforming a host cell for recombinant protein production. Methods for selecting and engineering vectors and host cells for expressing proteins (e.g., those provided herein), transforming cells, and expressing/purifying recombinant proteins are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

### BRIEF DESCRIPTION OF THE DRAWINGS

FIG. 1 is a cartoon showing the classical CRISPR/Cas9 Model. Shown are the single guide RNA (gRNA) complementary to a target site (e.g., a target genomic site) of the double stranded DNA (dsDNA), the protospacer adjacent motif (PAM) on the non-target DNA strand, and the cleavage by the Cas9 nuclease creating a double strand break (DSB).

FIG. 2A is a schematic showing an example of a modified donor DNA molecule for CRISPR-mediated homologous recombination using eGFP as a donor gene for insertion at a target genomic site (e.g., amyloid precursor protein (APP)). The first part of the modified CRISPR entails the use of two sgRNAs directed toward a site 5′ and 3′ of a target genomic site (shown, as an example, is APP, (Ovals)). In this example, two sgRNAs target two sites approximately 100 bp apart in the APP gene. Without a donor DNA molecule for insertion, the Cas-exonuclease fusion protein could be used to create an approximate 100 bp deletion, efficiently knocking out the APP gene. For efficient knock in of the donor gene (e.g., eGFP) at the target genomic site (e.g., APP gene), the donor DNA molecule is modified to include homology arms (e.g., sequences homologous to the target gene (e.g., APP)) at the 5′ and 3′ arms of the donor gene, in this example eGFP. The donor plasmid is modified to contain PAM sites (or unique gRNA sites) at the 5′ and 3′ arms of the donor DNA molecule (for example at the 5′ and 3′ arms of the APP homology arms) such that two sgRNAs (e.g., sgRNA donor A and sgRNA donor B) can specifically target the Cas-exonuclease fusion protein to the donor plasmid, but not the genomic DNA, subsequently cleaving and releasing the donor DNA molecule for insertion. As the homology arms on the donor DNA molecule are identical to segments of the target gene (e.g., APP), the donor DNA molecule is further modified to remove PAM sites (stars) identical to the PAM sites on the target genomic site. Removing the PAM sites on the donor DNA molecule can promote the targeting of the Cas-exonuclease fusion protein to the target genomic site and not the donor DNA molecule. The dual guide RNAs (e.g., sgRNA1 on the 5′ end and sgRNA 2 or 3 on the 3′ end) targeted against the genomic DNA can be designed to inhibit reannealing and allow time for homologous recombination. The dual guide RNAs (e.g., sgRNA-donor A and sgRNA donor B) are directed to the 3′ and 5′ 600 bp arms, respectively, of the donor DNA molecule (e.g., APP) and allow for directed homologous recombination. In the schematic, homologous recombination of the 5′ and 3′ arms of APP leads to insertion of the eGFP gene.

FIG. 2B is an image showing an example vector (e.g., px 459) containing a Cas9 gene and that can contain all four gRNAs for use in a CRISPR-Cas system.

FIG. 3 is an image showing an example pCAG-GFP vector (donor plasmid) with a SV40 origin of replication modified to include a donor nucleic acid (APP-eGFP-APP). As an example, the Simian Virus large T antigen (SVLT) can be used to induce replication of plasmids bearing the SV40 origin of replication (SV40 ori) within mammalian cells. This donor plasmid vector can be modified by deleting the CAG (CMV) promoter and inserting a donor nucleic acid (e.g., eGFP), which is sandwiched between 3′ and 5′ homology arms, which are substantially identical to a target nucleic acid (e.g., the APP gene) at the target genomic sites, required for the homologous recombination. Co-electroporation of the px459 (expressing the guide RNAs and Cas9; see FIG. 2B) and pCAG-GFP (containing the modified 5′ and 3′ arms flanking the desired inserted genomic material (shown as eGFP for exemplification only)) vectors is performed to initiate modified CRISPR targeting. The SV40 ori promotes replication of the donor plasmid to increase copy number and likelihood of recombination.

FIG. 4 shows the sequence (SEQ ID NO: 36) of an example plasmid (inserted, for example, into the pCAG vector; FIG. 3) with the donor APP-eGFP-APP sequence (eGFP gene in bold). The donor DNA molecule sequence contains mutated sites (designated by boxes) to remove PAM sites from, in this example, the APP arm of the donor DNA molecule that could be targeted by sgRNA (corresponding in this example to sgRNA2 and sgRNA3 targeting the 3′ end of the target genomic site). Removal of the PAM sites from the donor DNA molecule allows the sgRNA(s) to only target the genomic DNA. Of note, in this example, there is no mutated site needed for sgRNA1, as removal of the PAM site is achieved by insertion of the eGFP sequence. The sequences that could be targeted by sgRNA(s) are underlined. Mutation sites can also be introduced into the 5′ and 3′ flanking arms (in this example APP) in order to create PAM sites for targeting of a gene editing system for cleavage. In this example, mutations were also incorporated into the 5′ and 3′ arms of the APP flanking arms to create PAM or unique gRNA sites for sgRNA donor A and sgRNA donor B targeting to the 5′ and 3′ ends of the desired donor DNA molecule, respectively. In this example, two primer sites (highlighted) were incorporated so that homologous recombination could be confirmed. Insertion of the donor DNA molecule results in the detection of a 600 bp band by PCR.

FIG. 5 is an immunoblot demonstrating expression of the unmodified px459 CRISPR/Cas9 vector (Cas9, lane 1) and the vector modified to express the sgRNA2 (Cas9+APP sgRNA2, lane 2) targeting the 3′ arm of APP, a Cas9 fused to exonuclease A (Cas9-Exo, lane 3), the sgRNA2 and a Cas9 fused to an exonuclease (Cas9-Exo+APP sgRNA2, lane 4), a Cas9 fused to modified exonuclease A (codon optimized for eukaryotic cells) (Cas9-mExo, lane 5), and the APP sgRNA2 and a Cas9 fused to a modified exonuclease A (Cas9-mExo+APP sgRNA2, lane 6). Incorporation of APP sgRNA2 does not affect the expression of Cas9 or Cas9-exonuclease fusion proteins. The modified exonuclease, codon optimized for eukaryotic cell expression, shows enhanced expression over non-modified exonuclease. B-actin and APP are proteins used for loading control.

FIG. 6 is an image showing an immunoblot demonstrating knockdown of APP gene expression by CRISPR Cas9. Greatest efficiency of knockdown is achieved by a Cas9-exonuclease fusion protein expressed with sgRNA3 as compared to sgRNA1 or sgRNA2. Lane 5 shows the ability for APP sgRNA3 to knockdown APP gene expression without the Cas9-exonuclease fusion protein, although expression of the Cas9-exonuclease fusion protein with sgRNA3 leads to a slightly more efficient knockdown, as evidenced by a slightly weaker band (Lane 4). B-actin is a housekeeping protein used as a loading control.

FIG. 7 is an image showing an immunoblot demonstrating that the greatest knockdown efficiency of APP gene expression was achieved using the px459 CRISPR/Cas9 vector with Cas9 fused to modified exonuclease (mExo) and the use of two sgRNA (sgRNA1 and sgRNA3; see lane 5). A comparison of lane 5 and lane 2 shows that mExo enhanced the knockdown efficiency. Efficient knockdown is also achieved using another exonuclease, T5 exonuclease (see lanes 7-9), however increased cell death was observed with these constructs.

FIG. 8 is an image of an immunoblot for Amyloid Precursor Protein (APP) showing efficiency of knockdown with the px459-mExo-APPsgRNA1+3 construct expressed in clonal cell lines c1-c6. Clonal lines were expanded and screened for APP knockdown. All six representative clones show APP expression.

FIG. 9 is an image of an immunoblot demonstrating the knock in of eGFP at the APP site by homologous recombination using modified CRISPR Cas9 and APP sgRNA 3 and sgRNA 1 or sgRNA 2 (see lanes 2, 5 and 8, respectively), modified CRISPR Cas9-mExo and APP sgRNA 3 plus sgRNA 1 or sgRNA 2 (see lanes 3, 6 and 9, respectively), and modified CRISPR Cas9-T5 and APP sgRNA 3 plus sgRNA 1 or sgRNA 2 (lanes 4, 7 and 10, respectively). Increased efficiency of integration is achieved with the use of these two sgRNAs (1 and 3) and in combination with a modified exonuclease A fused with Cas9 (see lane 3). The addition of a beta protein from phage lambda did not enhance insertional efficiency (lanes 5-7). The combination of sgRNA 2 and 3 showed lower efficiency (lanes 8-10) compared to those using sgRNA 1 and sgRNA 3 (lanes 2-4). Control cells were transfected with empty Cas9 vectors without the inclusion of sgRNA (see lane 1). The upper blot was obtained with anti-GFP antibody, the middle blot showing APP and APP-GFP bands was obtained with anti-APP antibody, beta-actin (bottom blot) is used as a loading control.

FIG. 10 is an image of a western blot with anti-GFP and anti-APP antibodies performed on clonal cells which have been targeted with the modified CRISPR. The blot shows efficiencies of the APP-GFP gene integration into the genomic DNA by cell cloning analysis (see lanes c5 and c6). The blot is representative of integration efficiency close to 33%. HEK 293 cells were transfected with plasmid px459-mExo-App sgRNA 1+3 and a donor plasmid pCAG carrying APP-EGFP-APP sequence and lacking the pCAG promoter. Single cells were plated in a 96 well plate and cultured over two weeks prior to harvesting and protein isolation. Two of the clones express endogenous APP (c2 and c4), suggesting the APP gene is not knocked out, whereas two other clones (c1 and c3) do not show expression of either endogenous APP or APP-EGFP, suggesting that the endogenous APP gene is knocked out, but that the APP-EGFP-APP sequence has not been integrated into the APP site. Finally, clones c5 and c6 express APP-EGFP but not endogenous APP, confirming that the APP-EGFP-APP sequence has been homogenously integrated into genomic APP site in place of the endogenous APP.

FIG. 11A is a schematic illustrating that Down syndrome (DS) predominantly occurs through meiosis I error. Approximately 80% of DS results from non-disjunction during meiosis I. In this error, one daughter cell inherits the second maternal chromosome. During meiosis II, the sister chromatids separate forming n and n+1 gametes. Following fertilization, the DS cells will adopt 2n+1 configuration with the additional HSA21 chromosome. In this respect the proband will contain three HSA21 copies (one paternal and two maternal) as demonstrated in the D21S1411 microsatellite marker. Each of the three HSA21 copies is distinct, with distinct SNPs, allowing for SNP derived PAM targeting.

FIG. 11B is an image showing a D21S1411 microsatellite marker showing three copies of HSA21 in the progeny (PR): two copies from the mother (Mo) and one copy from the father (Fa).

FIGS. 12A-12D show the knockout of two targeted genes in human cells, AIRE and Col6A2. FIGS. 12A and 12B show the knockout of the AIRE gene locus on Chr21 using modified CRISPR/Cas9 by sequencing in human Down syndrome IPS cells. SNP associated PAM sites (arrowheads) in human DS iPS cells are identified by sequencing. FIG. 12A shows the presence, before CRISPR/Cas9 treatment, of a multiple copies of the AIRE gene (multiple peaks at arrow). FIG. 12B shows that after treatment with modified CRISPR/Cas9 and gRNA targeting the SNP-originated PAM site (single peak at arrow), the nucleotide signal at each position to the right of the arrow appear as multiple peaks, showing that one allele of the AIRE gene locus on Chr 21 was specifically cut by Cas9-gRNA, causing nucleotide Indels (insert/deletion). FIGS. 12C and 12D show a similar effect with the Col6A2 gene that is targeted on HSA21. In this experiment, three alleles are present prior to CRISPR/Cas9 treatment (FIG. 12C, at arrow). Post treatment, two of the three allelic copies of Col6A2, which have the SNP dependent PAM site, are disrupted by the Cas9-gRNA (FIG. 12D, at arrow).

FIG. 13 is a schematic showing an exemplary donor DNA molecule containing homologous arms, a Cas9 inhibitor (AcrII4) gene, a donor gene (shown is the X inactive specific transcript (XIST) gene) operably linked to a tetracycline promoter (Tet/on Pr), that can be incorporated into a vector (e.g., a pUC18 vector) for delivery. The vector containing the donor DNA molecule can co-transfected into DS IPS cells together with a modified vector (e.g., a lentiCRISPRV2 vector) designed to express the Cas9-exonuclease fusion protein and two sgRNAs. The cleavage by the Cas9-exonuclease fusion proteins at the target genomic sites containing SNP can promote the integration of the donor DNA molecule into Chr21 by HDR. The system can be designed to incorporate the donor DNA molecule at a site where an endogenous gene (e.g., App, s100b, or TPTE) promoter can be used to drive AcrIIA4 gene expression, thereby inhibiting further Cas9 enzyme activity. However, in the system shown, XIST gene transcription can be triggered under tetracycline promoter control (tet/on Pr).

FIG. 14 is a schematic showing an exemplary donor DNA molecule containing homologous arms, a Cas9 inhibitor protein gene, a donor gene operably linked to an inducible promoter (Ind. Pr), that can be incorporated into a vector (e.g., a pUC18 vector) for delivery. The vector containing the donor DNA molecule can be co-transfected into a desired cell together with a modified vector (e.g., lentiCRISPRv2) designed to express the Cas-exonuclease fusion protein and two sgRNAs. The cleavage by the Cas-exonuclease fusion proteins at the target genomic sites can cause the integration of the donor DNA molecule into the endogenous genome by HDR. The system can be designed to incorporate the donor DNA molecule at a site where an endogenous gene promoter can be used to drive Cas9 inhibitor gene expression, thereby inhibiting further Cas enzyme activity. However, transcription of the donor gene can be triggered under control of the inducible promoter. In the system shown, the inducible promotor could be omitted, which would result in the expression of the Cas inhibitor under control of an endogenous promoter at the site of integration of the donor gene.

FIGS. 15A-15D show how CRISPR modifications improve the efficiency of HDR in multiple cell types with minimal off target effects. FIG. 15A is an image of a western blot showing an increase in the efficiency of GFP integration when a px459 vector is modified with mExo. The western blot shows the results of GFP integration using a px459 vector carrying a single APP sgRNA (sgRNA1 or sgRNA3; lanes 2 and 3, respectively), dual sgRNAs (sgRNA1 and sgRNA3; lane 4), or dual sgRNAs (sgRNA1 and sgRNA3 and dual donor nucleic acid sgRNAs (sRNA2u and sRNA3u; lane 5) transfected into HEK 293 cells. The empty px459-mExo vector is used as a negative control (lane 1). The upper panel indicates the GFP-APP bands when the blot is incubated with anti-GFP antibody; the middle panel indicate the GFP-APP bands (upper bands) and APP bands (lower bands) when the blot is incubated with anti-APP antibody, and the bottom panel indicates the tubulin bands which represents the loading control for these samples. FIG. 15B is a bar graph depicting the relative efficiency of GFP integration into APP gene after tubulin normalization is statistically analyzed by using Western blot results (GFP-APP by using anti-GFP). The results from multiple assays (n=4) show that adding mExo into px459 and increasing the number of APP sgRNA can enhance the efficiency GFP integration into APP gene, as an exemplary gene and target. FIG. 15C is an image of the results from PCR of clonal HEK 293 cell line and insertion of XIST (3 kb) at the col6a2 site. Efficiency of insertion of XIST in 3 of 7 clones is shown. Similar findings were obtained with DS iPS following SNP-derived PAM targeting. Findings indicate that the modified CRISPR approach has utility in different cell types and can insert larger genomic DNA by HDR. FIG. 15D shows the results from deep sequencing analysis of putative off targeting sites does not reveal any increased mutagenesis using the modified mEXO CRISPR technique. *** indicates p:0.001.

### DETAILED DESCRIPTION

Described herein are methods of homology directed repair (HDR), fusion proteins for HDR, polynucleotides encoding the fusion proteins, vectors (e.g., viral vectors) containing polynucleotides encoding the fusion proteins, methods of delivery of the fusion proteins, and methods of using the fusion proteins for HDR, e.g., for the treatment of diseases and disorders.

Featured gene editing systems include fusion proteins having two domains, a Cas domain (e.g., a Cas9 domain) and an exonuclease domain (Cas-fusion protein), at least two guide RNAs), and, optionally, a donor DNA molecule. The sequences of the guide RNAs are complementary to a target site (e.g., a target genomic site) of a nucleic acid molecule to be edited. The Cas-fusion protein interacts with the guide RNA forming a CRISPR/Cas complex at the target site or a target genomic site. The target site or target genomic site can be upstream or downstream from, or part of, a gene associated with a disease or disorder (e.g., a mutation or a polymorphism). At the target site or target genomic site, the featured Cas-fusion protein of the CRISPR/Cas complex creates double strand breaks (DSBs) and 5′ and 3′ overhangs. The Cas domain (e.g., a Cas9 nuclease) of the Cas-fusion protein creates DSBs at the target site or target genomic site. Following creation of the DSB, the exonuclease creates 5′ and 3′ overhangs that mimic single stranded DNA (ssDNA). The creation of ssDNA overhangs promotes nucleic acid insertion and/or deletion through an HDR pathway as compared to dsDNA. Cas proteins and exonucleases for use in the gene editing system are described herein. The featured compositions can include a donor DNA molecule to be inserted at the target site or target genomic site.

The donor DNA molecule to be inserted into a target nucleic acid (e.g., a genome) can contain a polynucleotide sequence of a gene or a fragment thereof. Upon insertion into the genome, the gene sequence or fragment thereof can restore a function in a host cell (e.g., a beneficial biological activity in the host cell; e.g., by restoring the function of a defective gene). Alternatively, the donor DNA molecule may ablate a function in a host cell (e.g., reducing or inhibiting a detrimental biological activity in the host cell, such as by rendering a pathogenic gene or duplicated gene (e.g., in a trisomy) non-functional), e.g., in cases of pathogenic activity. The donor DNA molecule can further contain a nucleic acid sequence encoding a Cas inhibitor that is expressed upon insertion into the target genomic site by the HDR pathway. The CRISPR/Cas system can be used to treat a myriad of genetic diseases and disorders, target specific chromosomes, and insert a donor DNA molecule into an endogenous chromosome with increased efficiency in HDR, relative to other previously described systems.

In mammalian cells DSBs are generally repaired by non-homologous end-joining (NHEJ), frequently leading to loss of nucleotides from the ends of DSBs. Loss of nucleotides leads to efficient knockout of targeted alleles by introduction of frameshift mutations. By comparison, HDR allows for integration of desired genetic material into the genome by recombination with exogenously introduced targeting vectors. Traditional HDR methods, however, have been problematic given their low efficiency. Described herein are Cas-exonuclease fusion proteins with increased HDR efficiency and gene knock in efficiency when used with a CRISPR gene editing system.

The Cas-exonuclease fusion proteins can use two or more guide polynucleotides (e.g., guide RNAs) to guide fusion proteins to target sites (e.g., target genomic sites) flanking a DNA region of interest. The guide polynucleotides can form a CRISPR/Cas complex with the Cas-exonuclease fusion protein and can promote the creation of DSBs flanking (e.g., upstream and downstream) the target genomic site (e.g., a gene of interest or a mutation site). After creating the DSBs at the target site, the exonuclease domain of the featured Cas fusion protein creates 5′ and 3′ overhangs to promote HDR. The creation of DSBs and 5′ and 3′ overhangs flanking the target genomic site can promote the excision of the nucleic acids between the two target sites (e.g., the sites complementary to the guide polynucleotide sequence) and, preferably but not necessarily, the insertion of a donor DNA molecule. In some embodiments, the DNA region of interest is a deletion mutation. In these instances, the Cas-exonuclease fusion protein creates DSBs flanking the target genomic site promoting the insertion of a donor DNA molecule without the excision of a segment of genomic DNA.

CRISPR/Cas

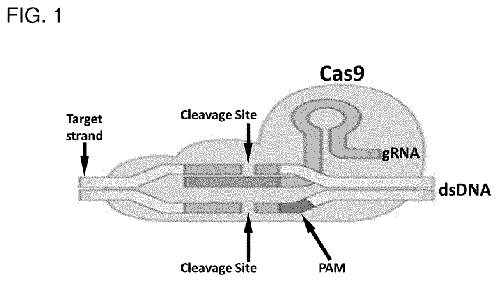

We developed a gene editing system with increased HDR efficiency through modifications to the CRISPR/Cas system. The CRISPR/Cas system derives from a prokaryotic immune system that confers resistance to foreign genetic elements, such as those present within plasmids and phages. CRISPR itself comprises a family of DNA sequences in bacteria, which encode small segments of DNA from viruses that have previously been exposed to the bacterium. These DNA segments are used by the bacterium to detect and destroy DNA from similar viruses during subsequent attacks. In a palindromic repeat, the sequence of nucleotides is the same in both directions. Each repetition is followed by short segments of spacer DNA from previous exposures to foreign DNA (e.g., a virus or plasmid). Small clusters of Cas (CRISPR-associated system) genes are located next to CRISPR sequences. These observations form the basis of the CRISPR/Cas system in eukaryotic cells that allows for genome editing. By delivering an RNA programmable nuclease (e.g., a Cas9 nuclease) with one or more guide polynucleotides (e.g., one or more gRNAs) into a cell, the cell's genome can be edited at desired locations (e.g., coding or non-coding regions of a genome of a host cell), allowing an existing gene(s) to be modified and/or removed and/or new gene(s) to be added (e.g., a functional version of a defective gene). The Cas9-gRNA complex corresponds with the type II CRISPR/Cas RNA complex (FIG. 1).

A number of bacteria express Cas9 protein variants that can be incorporated into the featured fusion protein (see, e.g., Tables 1 and 2). The Cas9 from Streptococcus pyogenes is presently the most commonly used. Several other Cas9 proteins have high levels of sequence identity with the S. pyogenes Cas9 and use the same guide RNAs. Still, others are more diverse, use different gRNAs, and recognize different PAM sequences as well (the 2-5 nucleotide sequence specified by the protein which is adjacent to the sequence specified by the RNA; see, e.g., Table 2). Chylinski et al. (2013, supra) classified Cas9 proteins from a large group of bacteria, and a large number of Cas9 proteins are described herein. Additional Cas9 proteins that can be used in the featured gene editing system are described in, e.g., Esvelt et al. (Nat Methods 10(11): 1116-21, 2013) and Fonfara et al. (Nucleic Acids Res. 42(4): 2577-2590, 2013); incorporated herein by reference.

Cas molecules from a variety of species can be incorporated into the compositions (e.g., the fusion protein), kits, and methods described herein. While the S. pyogenes Cas9 molecule is the subject of much of the disclosure herein, Cas9 molecules of, derived from, or based on the Cas9 proteins of other species listed herein can be used as well. In other words, while much of the description herein refers to S. pyogenes Cas9 molecules, Cas9 molecules from the other species can replace them. Such species include those set forth in the following table:

TABLE 1
Exemplary Cas9 nucleases

| GenBank Acc No. | Bacterium |
|---|---|
| 303229466 | Veillonella atypica ACS-134-V-Col7a |
| 34762592 | Fusobacterium nucleatum subsp. vincentii |
| 374307738 | Filifactor alocis ATCC 35896 |
| 320528778 | Solobacterium moorei F0204 |
| 291520705 | Coprococcus catus GD-7 |
| 42525843 | Treponema denticola ATCC 35405 |
| 304438954 | Peptoniphilus duerdenii ATCC BAA-1640 |
| 224543312 | Catenibacterium mitsuokai DSM 15897 |
| 24379809 | Streptococcus mutans UA159 |
| 15675041 | Streptococcus pyogenes SF370 |
| 16801805 | Listeria innocua Clip11262 |
| 116628213 | Streptococcus thermophilus LMD-9 |
| 323463801 | Staphylococcus pseudintermedius ED99 |
| 352684361 | Acidaminococcus intestini RyC-MR95 |
| 302336020 | Olsenella uli DSM 7084 |
| 366983953 | Oenococcus kitaharae DSM 17330 |
| 310286728 | Bifidobacterium bifidum S17 |
| 258509199 | Lactobacillus rhamnosus GG |
| 300361537 | Lactobacillus gasseri JV-V03 |
| 169823755 | Finegoldia magna ATCC 29328 |
| 47458868 | Mycoplasma mobile 163K |
| 284931710 | Mycoplasma gallisepticum str. F |
| 363542550 | Mycoplasma ovipneumoniae SC01 |
| 384393286 | Mycoplasma canis PG 14 |
| 71894592 | Mycoplasma synoviae 53 |
| 238924075 | Eubacterium rectale ATCC 33656 |
| 116627542 | Streptococcus thermophilus LMD-9 |
| 315149830 | Enterococcus faecalis TX0012 |
| 315659848 | Staphylococcus lugdunensis M23590 |
| 160915782 | Eubacterium dolichum DSM 3991 |
| 336393381 | Lactobacillus coryniformis subsp. torquens |
| 310780384 | Ilyobacter polytropus DSM 2926 |
| 325677756 | Ruminococcus albus 8 |
| 187736489 | Akkermansia muciniphila ATCC BAA-835 |
| 117929158 | Acidothermus cellulolyticus 11B |
| 189440764 | Bifidobacterium longum DJO10A |
| 283456135 | Bifidobacterium dentium Bd1 |
| 38232678 | Corynebacterium diphtheriae NCTC 13129 |
| 187250660 | Elusimicrobium minutum Pei191 |
| 319957206 | Nitratifractor salsuginis DSM 16511 |
| 325972003 | Sphaerochaeta globus str. Buddy |
| 261414553 | Fibrobacter succinogenes subsp. succinogenes |
| 60683389 | Bacteroides fragilis NCTC 9343 |
| 256819408 | Capnocytophaga ochracea DSM 7271 |
| 90425961 | Rhodopseudomonas palustris BisB18 |
| 373501184 | Prevotella micans F0438 |
| 294674019 | Prevotella ruminicola 23 |
| 365959402 | Flavobacterium columnare ATCC 49512 |
| 312879015 | Aminomonas paucivorans DSM 12260 |
| 83591793 | Rhodospirillum rubrum ATCC 11170 |
| 294086111 | Candidatus Puniceispirillum marinum IMCC1322 |
| 121608211 | Verminephrobacter eiseniae EF01-2 |
| 344171927 | Ralstonia syzygii R24 |
| 159042956 | Dinoroseobacter shibae DFL 12 |
| 288957741 | Azospirillum sp- B510 |
| 92109262 | Nitrobacter hamburgensis X14 |
| 148255343 | Bradyrhizobium sp- BTAi1 |
| 34557790 | Wolinella succinogenes DSM 1740 |
| 218563121 | Campylobacter jejuni subsp. jejuni |
| 291276265 | Helicobacter mustelae 12198 |
| 229113166 | Bacillus cereus Rock1-15 |
| 222109285 | Acidovorax ebreus TPSY |
| 189485225 | uncultured Termite group 1 |
| 182624245 | Clostridium perfringens D str. |
| 220930482 | Clostridium cellulolyticum H10 |
| 154250555 | Parvibaculum lavamentivorans DS-1 |
| 257413184 | Roseburia intestinalis L1-82 |
| 218767588 | Neisseria meningitidis Z2491 |
| 15602992 | Pasteurella multocida subsp. multocida |
| 319941583 | Sutterella wadsworthensis 3 1 |
| 254447899 | gamma proteobacterium HTCC5015 |
| 54296138 | Legionella pneumophila str. Paris |
| 331001027 | Parasutterella excrementihominis YIT 11859 |
| 34557932 | Wolinella succinogenes DSM 1740 |
| 118497352 | Francisella novicida U112 |

TABLE 2
Exemplary Cas nucleases and their associated PAM sequence

| | Class | | | SEQ |
|---|---|---|---|---|
| | and | | Target | ID |
| Species/Variant of Cas | Type | PAM Sequence | Length | NO |
| SpCas9 | Class II | 3′ NGG | 20 nt | 1 |
| Streptococcus pyogenes | type II | | | |
| (SP) | | | | |
| SpCas9 | Class II | 3′ NGG | 20 nt | 1 |
| D1135E variant | type II | (3′NAG reduced | | 2 |
| | | binding) | | |
| SpCas9 | Class II | 3′ NGCG | 20 nt | 3 |
| VRER variant | type II | | | |
| SpCas9 | Class II | 3′ NGAG | 20 nt | 4 |
| EQR variant | type II | | | |
| SpCas9 | Class II | 3′ NGAN; or | 20 nt | 5 |
| VQR variant | type II | 3′ NGNG | | 6 |
| SaCas9 | Class II | 3′ NNGRRT or | 20 to 24 nt | 7 |
| Staphylococcus aureus | type II | 3′ NNGRR(N) | | 8 |
| (SA) | | | | |
| SaCas9 | Class II | 3′ NNNRRT | 21 nt | 9 |
| Staphylococcus aureus | type II | | | |
| KKH variant | | | | |
| Cas12a: | Class II | 5′ TTTV | 23, 24 nt | 10 |
| Acidaminococcus sp. | type V | | | |
| (AsCpf1) and | | | | |
| Lachnospiraceae | | | | |
| bacterium (LbCpf1) | | | | |
| Cas12a | Class II | 5′ TYCV | 20 nt | 11 |
| AsCpf1 RR variant | type V | | | |
| Cas12a | Class II | 5′ TYCV | 20 nt | 11 |
| LbCpf1 RR variant | type V | | | |
| Cas12a | Class II | 5′ TATV | 20 nt | 12 |
| AsCpf1 RVR variant | type V | | | |
| NmCas9 | Class II | 3′ NNNNGATT | 23, 24 nt | 13 |
| Neisseria meningitidis | type II | | | |
| (NM) | | | | |
| StCas9 | Class II | 3′ NNAGAAW | 19 to 20 nt | 14 |
| Streptococcus | type II | | | |
| thermophilus1 (ST) | | | | |
| StCas9 | Class II | 3′ NGGNG | 19 nt | 15 |
| Streptococcus | type II | | | |
| thermophilus3 | | | | |
| TdCas9 | Class II | 3′ NAAAAC | 20 nt | 16 |
| Treponema denticola | type II | | | |
| (TD) | | | | |
| Cas13a (C2c2) | Class II | N/A | N/A | |
| Leptotrichia buccalis | type VI | | | |
| Cas13a (C2c2) | Class II | N/A | N/A | |
| Leptotrichia shahii | type VI | | | |

N/A - Cas13a have not been used in mammalian cells. The functional target length and PAM site remains unclear. For PAM sites: N can be any base; R can be A or G; V can be A, C, or G; W can be A or T; and Y can be C or T.

By way of example and not limitation, the constructs and methods described herein can include the use of any of the Cas proteins from Tables 1 and 2 and their corresponding guide polynucleotide(s) (e.g., guide RNA(s)) or other compatible guide RNAs. As an example, and not intended to be limiting in any way, the Cas9 from Streptococcus thermophilus LMD-9 CRISPR1 system has been shown to function in human cells (see, e.g., Cong et al. (2013, supra)). Cas9 orthologs from N. meningitides, which are described, e.g., in Hou et al. (Proc Natl Acad Sci USA. 110(39): 15644-9, 2013) and Esvelt et al. (2013, supra), can also be used in the compositions and methods described herein.

Exonuclease

Creating 3′ and 5′ overhangs at the site of a CRISPR/Cas cleaved DSB increases the efficiency of HDR of the CRISPR/Cas system. We incorporated an enzyme that can create 3′ and 5′ overhangs (e.g., an exonuclease) into the gene editing systems (e.g., the fusion protein), kits, and the methods described herein. Exonucleases are a broad class of enzymes capable of cleaving nucleotides one at a time from the 3′ or 5′ ends of DNA and RNA chains. Biological functions of exonucleases include DNA degradation and turnover, DNA proofreading, and transcriptional regulation. Exonucleases have been used extensively in molecular biology. A list of exonucleases that can be used in the fusion proteins described herein, and their targets, are described in Table 3. Modifying the CRISPR/Cas approach with exonucleases significantly enhances the efficiency of HDR. The exonuclease can be fused to a Cas nuclease to promote 3′ and 5′ overhangs for the insertion of donor DNA molecule. Non-limiting examples of exonucleases that can be incorporated into the compositions, (e.g., the fusion protein), kits, and methods described herein include lambda exonuclease, RecJf, exonuclease III (E. coli), exonuclease I (E. coli), thermolabile exonuclease I, exonuclease T, exonuclease V (RecBCD), exonuclease VIII, truncated, exonuclease VII, nuclease BAL-31, T5 exonuclease, T7 exonuclease.

TABLE 3
List of exonucleases

| | | | | Activity | | | | | | |
|---|---|---|---|---|---|---|---|---|---|---|
| | | DNA | | without | Able to Initiate | | | | Partial | |
| | | substrate | | 5′ | on DNA with | | | | Digestion of | Products |
| Enzyme | Polarity | ss | ds | phosphate | 5′ ext | 3′ ext | blunt | nick | ss Extension | Produced |
| Lambda | 5′ → 3′ | +/− | + | +/− | +/− | Yes | Yes | No | 3′ | dNMP, ssDNA |
| exonuclease | | | | | | | | | | |
| RecJf | 5′ → 3′ | + | − | Yes | +/− | No | +/− | No | NA | dNMP |
| exonuclease III | 3′ → 5′ | +/− | + | Yes | Yes | +/− | Yes | Yes | 5′ | ssDNA, dNMP |
| (E. coli) | | | | | | | | | | |
| exonuclease I | 3′ → 5′ | + | − | Yes | No | +/− | +/− | NR | NA | dNMP, |
| (E. coli) | | | | | | | | | | dinucleotide |
| thermolabile | 3′ → 5′ | + | − | Yes | No | +/− | +/− | NA | | dNMP, |
| exonuclease I | | | | | | | | | | dinucleotide |
| exonuclease T | 3′ → 5′ | + | − | Yes | No | Yes | +/− | NR | NA | dNMP |
| exonuclease V | both | + | + | Yes | Yes | Yes | Yes | No | Yes | short oligos |
| (RecBCD) | | | | | | | | | | |
| exonuclease VIII, | 5′ → 3′ | +/− | + | Yes | Yes | Yes | Yes | No | 3′ | dNMP, ssDNA |
| truncated | | | | | | | | | | |
| exonuclease VII | both | + | − | + | +/− | +/− | No | No | 5′ | short oligos |
| nuclease BAL-31 | 3′ → 5′ | + | + | Yes | Yes | Yes | Yes | Yes | NA | ssDNA, dNMP |
| | and Endo- | | | | | | | | | |
| | nuclease | | | | | | | | | |
| T5 exonuclease | 5′ → 3′ | + | + | Yes | Yes | Yes | Yes | Yes | NA | dNMP to 6 mer |
| T7 exonuclease | 5′ → 3′ | +/− | + | Yes | +/− | Yes | Yes | Yes | 3′ | ssDNA, dNMP, |
| | | | | | | | | | | dinucleotide |

Inhibitors

The incorporation of a Cas inhibitor into the gene editing system can limit the off-target effects of the CRISPR/Cas system described herein and further improve the efficiency of HDR. There are currently limited means to exert control over CRISPR/Cas system activity once the components have been delivered, leading to practical safety concerns. For example, off target effects are exacerbated by excessive or prolonged Cas activity.

To address these issues, featured herein are donor DNA molecules for knock in of exogenous genetic material through HDR that contain a nucleic acid sequence encoding a Cas inhibitor. Upon insertion of the donor DNA molecule, expression of the anti-CRISPR protein can inhibit any further CRISPR/Cas system activity, thereby limiting the possibility of offsite targeting and over activation (see, e.g., Example 5). In some instances, the inhibitor can be provided as a nucleic acid molecule with a delayed expression as compared to the CRISPR/Cas system. For example, the expression of the inhibitor can be operably linked to a promoter that is less robust than a promoter operably linked to the CRISPR/Cas system (e.g., when the inhibitor is delivered to the host cell with the CRISPR/Cas complex), delaying the expression and/or slowing the accumulation of the inhibitor (e.g., until a primary or desired editing event has been completed). Alternatively, the CRISPR/Cas inhibitor can be provided to a cell after HDR to prevent off target effects. For example, the CRISPR/Cas inhibitor can be provided to a target cell as a protein molecule after HDR to inhibit further activity of the CIRSPR/Cas fusion protein.

Non-limiting examples of anti-CRISPR proteins that can be encoded by a nucleotide sequence (e.g., for delivery to a cell in a vector, which may also encode the CRISPR/Cas complex components), or delivered to a target cell as a protein molecule, can be seen in Table 4 below (reproduced from Zhu et al. BMC Biology 16:32, 2018). Featured nucleic acid sequences that express anti-CRISPR proteins are those having at least 85% or more (e.g., 90%, 95%, 97%, 98%, 99%, or 100%) sequence identity to one or more of the anti-CRISPR proteins listed in Table 4 or any fragment thereof (e.g., fragments of at least about 10, at least about 20, at least about 30, at least about 40, at least about 50, at least about 60, at least about 70, or more consecutive amino acids in length), and that are capable of reducing (e.g., by at least 50% or more (e.g., 60%, 70%, 80%, 90%, 95%, or 100%) cleavage of genomic DNA by the featured CRISPR/Cas systems following an initial gene editing event. In some embodiments, the expressed anti-CRISPR protein is a Type II anti-CRISPR protein.

TABLE 4
Exemplary anti-CRISPR proteins

| | Size | | | Structure |
|---|---|---|---|---|
| Anti-CRISPR | (amino | CRISPR | Inhibition | (PDB |
| (source) | acids) | inhibited | mechanism | code) |
| AcrIIA1 (L. | 149 | Type | — | 5Y6A |
| monocytogenes) | | II-A | | |
| AcrIIA2 (L. | 123 | Type | Inhibits DNA | — |
| monocytogenes) | | II-A | binding | |
| AcrIIA3 (L. | 125 | Type | — | — |
| monocytogenes) | | II-A | | |
| AcrIIA4 (L. | 87 | Type | PAM mimic, inhibits | 5XBL, |
| monocytogenes) | | II-A | DNA binding; | 5VW1, |
| | | | interacts with active | 5VZL |
| | | | site within the RuvC | |
| | | | domain; hinders the | |
| | | | conformation change | |
| | | | of the HNH domain | |
| AcrIIA5 (S. | 140 | Type | — | — |
| thermophilus) | | II-A | | |
| AcrIIC1 (N. | 85 | Type | Binds the HNH | 5VGB |
| meningitidis) | | II-C | domain; shields the | |
| | | | catalytic center | |
| AcrIIC2 (N. | 123 | Type | — | — |
| meningitidis) | | II-C | | |
| AcrIIC3 (N. | 116 | Type | Induces Cas9 | — |
| meningitidis) | | II-C | dimerization; inhibits | |
| | | | DNA binding | |

Guide Polynucleotides

The featured fusion proteins can be guided to a target site (e.g., a target genomic site) using a guide polynucleotide (e.g., gRNA). Generally speaking, gRNAs come in two different systems: System 1, which uses separate crRNA and tracrRNAs that function together to guide cleavage by a Cas nuclease (e.g., Cas9), and System 2, which uses a chimeric crRNA-tracrRNA hybrid that combines the two separate guide RNAs in a single system (referred to as a single guide RNA or sgRNA: see also, e.g., Jinek et al. (2012, supra)). While the disclosure focuses on designing System 2 sgRNA specific for a target site, e.g., designing a guide polynucleotide (e.g., a guide RNA) having a sequence complementary to the target site (e.g., target genomic site), any of the methods described herein can be used to design separate System 1 crRNA and tracrRNA guide polynucleotides for use with the featured CRISPR/Cas system. When manipulation of gene expression is desired, for System 2, in some instances, gRNAs can be complementary to a target site region that is within about 100-800 base pairs (bp) upstream of a transcription start site of a gene, (e.g., within about 500 bp, about 400 bp, about 300 bp, about 200 bp, about 150 bp, about 100 bp, or about 50 bp upstream of the transcription start site), includes the transcription start site, or is within about 100-800 bp downstream of a transcription start site (e.g., within about 500 bp, about 400 bp, about 300 bp, about 200 bp, about 150 bp, about 100 bp, or about 50 bp downstream of the transcription start site). In some embodiments, the gRNA can be complementary to any desired site within an endogenous DNA molecule (e.g., a target gene, a region within a target gene, a regulatory element (e.g., a start site for transcription, a promoter region, a transcription factor (e.g., an enhancer or silencer)), or any target site for the featured fusion proteins to form a complex. In some embodiments, vectors (e.g., viral vectors (e.g., lentiviral vectors)) encoding more than one gRNA can be used, e.g., vectors encoding, 2, 3, 4, 5, or more gRNAs directed to different target sites or target genomic sites in the same region of the target nucleic acid molecule (e.g., a gene or other site on a chromosome).

Featured fusion proteins can be guided to specific 17-25 nucleotide (nt) target sites (e.g., genomic target sites) bearing an additional PAM (e.g., sequence NGG for Cas9), using a guide RNA (e.g., a single gRNA or a tracrRNA/crRNA) bearing 17-25 nts at its 5′ end that are complementary to the complementary strand of a target nucleic acid molecule (e.g., genomic DNA at a target genomic site). Thus, the gene editing system can include the use of a single guide RNA comprising a crRNA fused to a normally trans-encoded tracrRNA, e.g., a single Cas guide RNA (such as those described in Mali et al. (2013, supra)), with a sequence at the 5′ end that is complementary to the target sequence, e.g., of 17-25 nts, optionally 20 or fewer nts, e.g., 20, 19, 18, or 17 nts, preferably 17 or 18 nts, of the complementary strand to a target sequence immediately 5′ of a PAM.

In some embodiments, it will be desired to further limit off target effects (e.g., CRISPR/Cas complex formation at a site other than the target site) or to target a single chromosome. In certain embodiments, a single nucleotide polymorphism (SNP) associated PAM (e.g., a unique PAM site created by a SNP) can be used to direct the CRISPR/Cas complex to a single desired target site (e.g., a target genomic site). Next generation gene sequencing can be used to identify the location of unique PAM sites created by SNPs. Certain diseases can be correlated to the presence of a SNP associated PAM site on a single chromosome. The gRNA of the CRISPR/Cas complex can be selected to target the SNP associated PAM on the single chromosome. Non-limiting examples of diseases in which it may be desired to target a single chromosome are trisomy diseases (e.g., Down syndrome, Edwards syndrome, Patau syndrome, and Klinefelter syndrome). Targeting a single chromosome using SNP PAM sites is further discussed in Example 4.

Existing Cas-based nucleases use gRNA-DNA heteroduplex formation to guide targeting to genomic sites of interest. However, RNA-DNA heteroduplexes can form a more promiscuous range of structures than their DNA-DNA counterparts. In effect, DNA-DNA duplexes are more sensitive to mismatches, suggesting that a DNA-guided nuclease may not bind as readily to off-target sequences, making them comparatively more specific than RNA-guided nucleases. Thus, the guide RNAs featured in the methods described herein can be hybrids, e.g., wherein one or more deoxyribonucleotides, e.g., a short DNA oligonucleotide, replaces all or part of the gRNA, e.g., all or part of the complementarity region of a gRNA. This DNA-based molecule could replace either all or part of the gRNA in a single gRNA system (e.g., system 2) or alternatively might replace all of part of the crRNA and/or tracrRNA in a dual crRNA/tracrRNA system (e.g., system 1). Such a system that incorporates DNA into the complementarity region can be used to target, e.g., an intended genomic DNA site due to the general intolerance of DNA-DNA duplexes to mismatching as compared to RNA-DNA duplexes. Methods for making such duplexes are known in the art (see, e.g., Barker et al. (BMC Genomics 6:57, 2005) and Sugimoto et al. (Biochemistry 39(37):11270-81, 2000)).

In general, a guide polynucleotide (e.g., a gRNA) can be any polynucleotide having a nucleic acid sequence with sufficient complementarity with the sequence of a target polynucleotide to promote specific hybridization with the target polynucleotide and direct sequence-specific binding of a featured CRISPR/Cas fusion protein to the target site. In some embodiments, the degree of complementarity between the sequence of a guide polynucleotide and corresponding sequence of the target site, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAST, Novoalign (Novocraft Technologies, ELAND (Illumina, San Diego, Calif.), SOAP (available at soap.genomics.org.cn), and Maq (available at maq.sourceforge.net). In some embodiments, a guide polynucleotide (e.g., a gRNA) has about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide polynucleotide (e.g., a gRNA) has fewer than about 75, 50, 45, 40, 35, 30, 25, 20, 15, or 12 nucleotides. The ability of a guide polynucleotide to direct sequence-specific binding of a CRISPR complex to a target site may be assessed by any suitable assay. For example, the components of a CRISPR system sufficient to form a CRISPR/Cas complex, including the guide polynucleotide to be tested, may be provided to a host cell having the corresponding target site sequence, such as by transfection with vectors encoding the components of the CRISPR/Cas complex, followed by an assessment of preferential cleavage within the sequence of the target site, such as by the incorporation of a reporter gene (e.g., a nucleic acid encoding enhanced green fluorescent protein (eGFP)), which is further described in the examples. Similarly, cleavage of a target site polynucleotide may be evaluated in a test tube by providing the target site, components of the featured CRISPR/Cas complex, including the guide polynucleotide to be tested and a control guide polynucleotide different from the test guide polynucleotide, and comparing binding or rate of cleavage at the target site between the test and control guide polynucleotide reactions. Other assay methods known to those skilled in the art can also be used.

In some instances, the one or more guide polynucleotides (e.g., sgRNAs) are a first guide polynucleotide (e.g., a first sgRNA) directed to a first genomic site and a second guide polynucleotide (e.g., a second sgRNA) directed to a second genomic site (e.g., two different target genomic sites). In some instances, the first genomic site and the second genomic site are between about 10 and about 15000 bps apart (e.g., between about 10 and about 500 bps (e.g., about 50 bp, about 75 bp, about 100 bp, about 150 bp, about 200 bp, about 250 bp, about 300 bp, about 350 bp, about 400 bp, about 450 bp, or about 500 bp apart), between about 400 and about 1500 bps apart (e.g., about 450 bp, about 500 bp, about 600 bp, about 700 bp, about 800 bp, about 900 bp, about 1000 bp, about 1100 bp, about 1200 bp, about 1300 bp, about 1400 bp, or about 1450 bp apart), between about 1400 and about 3000 bps apart (e.g., about 1450 bp, about 1500 bp, about 1600 bp, about 1700 bp, about 1800 bp, about 1900 bp, about 2000 bp, about 2100 bp, about 2200 bp, about 2300 bp, about 2400 bp, about 2500 bp, about 2600 bp, about 2700 bp, about 2800 bp, about 2900 bp, or about 2950 bp apart), between about 2900 and about 5000 bps apart (e.g., about 2950 bp, about 3000 bp, about 3100 bp, about 3200 bp, about 3300 bp, about 3400 bp, about 3500 bp, about 3600 bp, about 3700 bp, about 3800 bp, about 3900 bp, about 4000 bp, about 4100 bp, about 4200 bp, about 4300 bp, about 4400 bp, about 4500 bp, about 4600 bp, about 4700 bp, about 4800 bp, about 4900 bp, about 4950 bp apart, or about 5000 bp apart), between about 4800 and about 10,000 bp apart (e.g., about 4850 bp, about 4900 bp, about 5000 bp, about 5050 bp, about 5100 bp, about 5300 bp, about 5500 bp, about 5800 bp, about 6000 bp, about 6200 bp, about 6500 bp, about 6800 bp, about 7000 bp, about 8200 bp, about 8500 bp, about 8800 bp, about 9000 bp, about 9200 bp, about 9500 bp, about 9800 bp, about 9900 bp, about 9950 bp apart), or between about 9900 and about 15000 bp apart (e.g., about 9550 bp, about 10000 bp, about 10200 bp, about 10500 bp, about 10800 bp, about 1100 bp, about 11200 bp, about 11500 bp, about 11800 bp, about 12000 bp, about 12200 bp, about 12500 bp, about 12800 bp, about 13000 bp, about 13200 bp, about 13500 bp, about 13800 bp, about 14000 bp, about 14200 bp, about 14500 bp, about 14800 bp, about 14900 bp, or about 14950 bp apart). In particular embodiments, the first target genomic site and the second target genomic site are between about 50 and about 200 bps apart (e.g., about 60 bp, about 70 bp, about 80 bp, about 90 bp, about 100 bp, about 110 bp, about 120 bp, about 130 bp, about 140 bp, about 150 bp, about 160 bp, about 170 bp, about 180 bp, or about 190 bp apart).

Donor DNA Molecule

Featured compositions, kits, and methods described herein may also include one or more donor DNA molecules. In general, a donor DNA molecule is a polynucleotide to be inserted at a target site (e.g., a target genomic site). In one embodiment, the donor DNA molecule can include a sequence which results in an alteration in the coding sequence of a translated sequence (e.g., one which results in the substitution of one or more amino acids for another in a protein product (e.g., transforming a mutant allele into a wild type allele, transforming a wild type allele into a mutant allele, and/or introducing a stop codon, insertion of an amino acid residue(s), or deletion of an amino acid residue(s)). In another embodiment, the donor DNA molecule can include a sequence which results in the inactivation of a gene or chromosome (e.g., in the case of a duplication event that creates one or more extra copies of a gene or chromosome (e.g., a trisomy, such as trisomy 21, in a cell). In other embodiments, the donor DNA molecule can include a sequence which results in an alteration in a non-coding sequence, e.g., an alteration in an exon or in a 5′ or 3′ non-translated or non-transcribed region. Alterations may also include a change in a control element of a gene (e.g., inclusion or alteration of a promoter or enhancer or an alteration in a cis-acting or trans-acting regulatory element) or a change in an extra-coding or non-coding region of DNA (e.g., a region encoding a microRNA or long non-coding RNA). In some instance, the sequence alteration may be introduced to affect its ability to be identified by select gRNAs (e.g., inclusion or introduction or a PAM sequence). In certain embodiments, the donor DNA molecule contains a 5′ homology arm. In other embodiments, the donor DNA molecule contains a 3′ homology arm. In certain embodiments, the donor DNA molecule contains both a 3′ and a 5′ homology arm. In some embodiments, the 3′ and 5′ homology arms are substantially the same length. In other embodiments, the 3′ and 5′ homology arms are of different length.

In some embodiments, the donor DNA molecule is linear double stranded DNA. The length may be about 10-15000 bps. The length may be, e.g., about 20-15000 bps, about 30 bps, about 40 bps, about 50 bps, about 60 bps, about 70 bps, about 80 bps, about 90 bps, about 100 bps, about 150 bps, about 200 bps, about 250 bps, about 300 bps, about 350 bps, about 400 bps, about 450 bps, about 500 bps, about 550 bps, about 600 bps, about 650 bps, about 700 bps, about 750 bps, about 800 bps, about 850 bps, about 900 bps, about 950 bps, about 1000 bps, about 1050 bps, about 1100 bps, about 1150 bps, about 1200 bps, about 1250 bps, about 1300 bps, about 1350 bps, about 1400 bps, about 1450 bps, about 1500 bps, about 1550 bps, about 1600 bps, about 1650 bps, about 1700 bps, about 1750 bps, about 1800 bps, about 1850 bps, about 1900 bps, about 1950 bps, about 2000 bps, about 2050 bp, about 2100 bp, about 2150 bp, about 2200 bp, about 2250 bp, about 2300 bp, about 2350 bp, about 2400 bp, about 2450 bp, about 2500 bp, about 2550 bp, about 2600 bp, about 2650 bp, about 2700 bp, about 2750 bp, about 2800 bp, about 2850 bp, about 2900 bp, about 2950 bp, about 3000 bp, about 3050 bp, about 3100 bp, about 3150 bp, about 3200 bp, about 3250 bp, about 3300 bp, about 3350 bp, about 3400 bp, about 3450 bp, about 3500 bp, about 3550 bp, about 3600 bp, about 3650 bp, about 3700 bp, about 3750 bp, about 3800 bp, about 3850 bp, about 3900 bp, about 3950 bp, about 4000 bp, about 4050 bp, about 4100 bp, about 4150 bp, about 4200 bp, about 4250 bp, about 4300 bp, about 4350 bp, about 4400 bp, about 4450 bp, about 4500 bp, about 4550 bp, about 4600 bp, about 4650 bp, about 4700 bp, about 4750 bp, about 4800 bp, about 4850 bp, about 4900 bp, about 4950 bp, about 5000 bp, about 5200 bp, about 5500 bp, about 5800 bp, about 6000 bp, about 6200 bp, about 6500 bp, about 6800 bp, about 7000 bp, about 7200 bp, about 7500 bp, about 7800 bp, about 8000 bp, about 8200 bp, about 8500 bp, about 8800 bp, about 9000 bp, about 9200 bp, about 9500 bp, about 9800 bp, about 10000 bp, about 10200 bp, about 10500 bp, about 10800 bp, about 11000 bp, about 11200 bp, about 11500 bp, about 11800 bp, about 12000 bp, about 12200 bp, about 12500 bp, about 12800 bp, about 13000 bp, about 13200 bp, about 13500 bp, about 13800 bp, about 14000 bp, about 14200 bp, about 14500 bp, about 14800 bp, about 14900 bp, or about 1495 bp. In some embodiments, the length may be, e.g., about 20-2000 bps, about 30 bps, about 40 bps, about 50 bps, about 60 bps, about 70 bps, about 80 bps, about 90 bps, about 100 bps, about 150 bps, about 200 bps, about 250 bps, about 300 bps, about 350 bps, about 400 bps, about 450 bps, about 500 bps, about 550 bps, about 600 bps, about 650 bps, about 700 bps, about 750 bps, about 800 bps, about 850 bps, about 900 bps, about 950 bps, about 1000 bps, about 1050 bps, about 1100 bps, about 1150 bps, about 1200 bps, about 1250 bps, about 1300 bps, about 1350 bps, about 1400 bps, about 1450 bps, about 1500 bps, about 1550 bps, about 1600 bps, about 1650 bps, about 1700 bps, about 1750 bps, about 1800 bps, about 1850 bps, about 1900 bps, about 1950 bps, or about 2000 bps.

In certain embodiments, the donor DNA molecule also contains the nucleic acid sequence of a CRISPR/Cas inhibitor (see, e.g., Table 4). In other embodiments, an endogenous gene promoter will drive expression of the CRISPR/Cas inhibitor to inhibit Cas enzyme activity (e.g., after an initial editing event inserting the donor DNA has been completed). In some embodiments, the donor DNA molecule contains a promoter operably linked to the CRISPR/Cas inhibitor nucleic acid sequence. The donor DNA molecule may further contain a second promoter operably linked to the donor DNA sequence.

### Delivery Methods

Vectors

In addition to achieving high rates of transcription and translation, stable expression of an exogenous polynucleotide sequence (e.g., a polynucleotide sequence encoding the modified CRISPR/Cas system described herein) in a mammalian cell can be achieved by integration of the polynucleotide containing the sequence into the nuclear genome of the mammalian cell. A variety of vectors for the delivery and integration of polynucleotides encoding exogenous proteins into the nuclear DNA of a mammalian cell have been developed. Expression vectors are well known in the art and include, but are not limited to, viral vectors and plasmids.

Vectors for use in the compositions and methods described herein contain at least one polynucleotide encoding a featured fusion protein or fragment thereof (e.g., a fragment that retains the ability to form a complex with a guide polynucleotide (e.g., a gRNA) at a target site or target genomic site and create a double strand break and 5′ and/or 3′ overhangs), at least one guide polynucleotide (e.g., a gRNA), and, optionally, a donor DNA molecule. The vectors may also provide additional sequence elements (e.g., regulatory elements) used for the expression of these agents and/or the integration of these polynucleotide sequences into the genome of a mammalian cell. Certain vectors that can be used for the expression of the gene editing system components include plasmids that contain regulatory elements, such as promoter and enhancer regions, which direct transcription of the nucleic acid molecules encoding the featured components. Other useful vectors for expression of the gene editing system components contain polynucleotide sequences that enhance the rate of translation of these genes or improve the stability or nuclear export of the mRNA that results from gene transcription. These sequence elements include, e.g., 5′ and 3′ untranslated regions, and/or a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, and nourseothricin.

The vector may further include a polynucleotide with a linker sequence positioned in the vector between a first domain (e.g., a domain encoding a Cas protein) and a second domain (e.g., a domain encoding an exonuclease) so as to produce a fusion protein containing the two domains joined by the linker. Linking sequences can encode random amino acids or can contain functional sites (e.g., a cleavage site).

In some embodiments, a vector encoding a Cas fusion protein, guide polynucleotide(s) (e.g., gRNA(s)), and/or a donor DNA molecule is codon optimized for expression in particular cells, such as eukaryotic cells. The eukaryotic cells may be those of, or derived from, a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura et al. (Nucl. Acids Res. 28:292, 2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR fusion protein, a gRNA, and/or a donor DNA molecule correspond to the most frequently used codon for a particular amino acid.

Viral Delivery Vehicles

Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Viral-based vectors for delivery of a desired polynucleotide and expression in a desired cell are well known in the art. Exemplary viral-based vehicles include, but are not limited to, recombinant retroviruses (e.g., a lentiviral vector, see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127), adenovirus vectors, alphavirus-based vectors (e.g., Sindbis virus vectors, Semliki forest virus), Ross River virus, adeno-associated virus (AAV) vectors (see, e.g., PCT Publication Nos. WO 94/12649, WO 93/03769; WO 93/19191; WO 94/28938; WO 95/11984 and WO 95/00655), vaccinia virus (e.g., Modified Vaccinia virus Ankara (MVA) or fowlpox), Baculovirus recombinant system, and herpes virus. Further examples of viral vectors for delivery of the featured CRISPR/Cas system include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double-stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the entire contents of which is hereby incorporated by reference.

Exemplary viral vectors include lentiviral vectors, AAVs, and retroviral vectors. Lentiviral vectors and AAVs can integrate into the genome without cell divisions, and both types have been tested in pre-clinical animal studies.

Methods for preparation of AAVs are described in the art, e.g., in U.S. Pat. Nos. 5,677,158, 6,309,634, and 6,683,058, the entire contents of each of which is incorporated herein by reference.

Methods for preparation and in vivo administration of lentiviruses are described in US 20020037281, the entire contents of which is hereby incorporated by reference. Lentiviral vectors (LVs) transduce a wide range of dividing and non-dividing cell types with high efficiency, conferring stable, long-term expression of the transgene. An overview of optimization strategies for packaging and transducing LVs is provided in Delenda (J. Gen Med 6: S125, 2004), the entire contents of which are incorporated herein by reference.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, incapsidation, and expression, in which the sequences to be expressed are inserted.

Enhancer elements can be used to increase expression of modified DNA molecules or increase the lentiviral integration efficiency. The LV for use with the featured gene editing system described herein may include a nef sequence. The LV for use with the featured gene editing system described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV for use with the featured gene editing system described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to an LV results in a substantial improvement in the level of transgene expression from several different promoters, both in vitro and in vivo. The LV for use with the featured gene editing system described herein may include both a cPPT sequence and Woodchuck Hepatitis Virus (WHP) Posttranscriptional Regulatory Element (WPRE) sequence. The LV may also include an IRES sequence that permits the expression of multiple polypeptides from a single promoter.

The vector for use with the featured gene editing system described herein may include multiple promoters that permit expression of more than one polynucleotide and/or polypeptide. The vector for use with the featured gene editing system described herein may include a protein cleavage site that allows expression of more than one polypeptide. Examples of protein cleavage sites that allow expression of more than one polypeptide are described in, e.g., Klump et al. (Gene Ther 8:811 2001), Osborn et al. (Molecular Therapy 12:569, 2005), Szymczak and Vignali (Expert Opin Biol Ther. 5:627, 2005), and Szymczak et al. (Nat Biotechnol. 22:589, 2004), the disclosures of which are incorporated herein by reference. It will be readily apparent to one skilled in the art that other elements that permit expression of multiple polypeptides identified in the future are useful and may be utilized in the vectors suitable for use with the compositions and methods described herein.

The vector used in the methods and compositions described herein may be a clinical grade vector.

The viral vector may also include viral regulatory elements, which are components of delivery vehicles used to introduce nucleic acid molecules into a host cell. The viral regulatory elements are optionally retroviral regulatory elements. For example, the viral regulatory elements may be the LTR and gag sequences from HSC1 or MSCV. The retroviral regulatory elements may be from lentiviruses or they may be heterologous sequences identified from other genomic regions. One skilled in the art would also appreciate that as other viral regulatory elements are identified, these may be used with the viral vectors described herein.

Non-Viral Delivery Vehicles

Several non-viral vehicles can be used for delivery of the featured CRISPR/Cas system, polynucleotides encoding the CRISPR/Cas system, the guide polynucleotides (e.g., gRNAs), and the donor DNA molecules. These include, non-viral vectors, such as plasmids, that include but are not limited to prokaryotic and eukaryotic vectors (e.g., yeast- and bacteria-based plasmids), as well as plasmids for expression in mammalian cells. Methods of introducing the vectors into a host cell and isolating and purifying the expressed protein are also well known in the art (e.g., Molecular Cloning: A Laboratory Manual, second edition, Sambrook, et al. 1989, Cold Spring Harbor Press). Examples of host cells include, but are not limited to, mammalian cells, such as NSO, CHO cells, HEK and COS, and bacterial cells, such as E. coli.

Other non-viral delivery vehicles include polymeric, biodegradable microparticle, or microcapsule delivery devices known in the art. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of an aqueous buffer containing large macromolecules.

The composition of the liposome is usually a combination of phospholipids, usually in combination with steroids, in particular cholesterol. Other phospholipids or other lipids may also be used. The physical characteristics of liposomes depend on pH, ionic strength, and the presence of divalent cations.

Lipids useful in liposome production include phosphatidyl compounds, such as phosphatidylglycerol, phosphatidylcholine, phosphatidylserine, phosphatidyl-ethanolamine, sphingolipids, cerebrosides, and gangliosides. Exemplary phospholipids include egg phosphatidylcholine, dipalmitoylphosphatidylcholine, and distearoyl-phosphatidylcholine. The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255.

Pharmaceutical Compositions

The polynucleotides, vectors comprising the polynucleotides, gene delivery vectors, fusion proteins, and CRISPR/Cas complexes described herein can be prepared as compositions that contain a pharmaceutically acceptable carrier, excipient, or stabilizer known in the art (Remington: The Science and Practice of Pharmacy 20th Ed., 2000, Lippincott Williams and Wilkins, Ed. K. E. Hoover). The compositions may also be provided in the form of a lyophilized formulation, as an aqueous solution, or as a pharmaceutical product suitable for direct administration. Acceptable carriers, excipients, or stabilizers are non-toxic to recipients at the employed dosages and concentrations, and may include buffers such as phosphate, citrate, and other organic acids; antioxidants including ascorbic acid and methionine; preservatives (e.g., octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol); low molecular weight (less than about 10 residues) polypeptides; proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, marmose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Pharmaceutically acceptable excipients are further described herein.

The compositions (e.g., when used in the methods described herein) generally include, by way of example and not limitation, an effective amount (e.g., an amount sufficient to mitigate disease, alleviate a symptom of disease and/or prevent or reduce the progression of disease) of polynucleotides, vectors comprising the polynucleotides (e.g., viral vectors), fusion proteins, and or CRISPR/Cas complexes described herein.

The composition may be formulated to include between about 1 μg/mL and about 1 g/mL of the fusion protein, the guide polynucleotides (e.g., gRNAs), and/or donor DNA molecule, or any combination thereof (e.g., between 10 μg/mL and 300 μg/mL, 20 μg/mL and 120 μg/mL, 40 μg/mL and 200 μg/mL, 30 μg/mL and 150 μg/mL, 40 μg/mL and 100 μg/mL, 50 μg/mL and 80 μg/mL, or 60 μg/mL and 70 μg/mL, or 10 mg/mL and 300 mg/mL, 20 mg/mL and 120 mg/mL, 40 mg/mL and 200 mg/mL, 30 mg/mL and 150 mg/mL, 40 mg/mL and 100 mg/mL, 50 mg/mL and 80 mg/mL, 60 mg/mL and 70 mg/mL, or 100 mg/ml and 1 g/ml (e.g., 150 mg/ml, 200 mg/ml, 250 mg/ml, 300 mg/ml, 350 mg/ml, 400 mg/ml, 450 mg/ml, 500 mg/ml, 550 mg/ml, 600 mg/ml, 650 mg/ml, 700 mg/ml, 750 mg/ml, 800 mg/ml, 850 mg/ml, 900 mg/ml, or 950 mg/ml of the fusion protein, the guide polynucleotides (e.g., gRNAs), and/or the donor DNA molecule, or any combination thereof).

The compositions containing any of the non-viral vectors of the invention may contain a unit dose containing a quantity of polynucleotides from 10 μg to 10 mg (e.g., from 25 μg to 5.0 mg, from 50 μg to 2.0 mg, or from 100 μg to 1.0 mg of polynucleotides, e.g., from 10 μg to 20 μg, from 20 μg to 30 μg, from 30 μg to 40 μg, from 40 μg to 50 μg, from 50 μg to 75 μg, from 75 μg to 100 μg, from 100 μg to 200 μg, from 200 μg to 300 μg, from 300 μg to 400 μg, from 400 μg to 500 μg, from 500 μg to 1.0 mg, from 1.0 mg to 5.0 mg, or from 5.0 mg to 10 mg of polynucleotides, e.g., about 10 μg, about 20 μg, about 30 μg, about 40 μg, about 50 μg, about 60 μg, about 70 μg, about 80 μg, about 90 μg, about 100 μg, about 150 μg, about 200 μg, about 250 μg, about 300 μg, about 350 μg, about 400 μg, about 450 μg, about 500 μg, about 600 μg, about 700 μg, about 750 μg, about 1.0 mg, about 2.0 mg, about 2.5 mg, about 5.0 mg, about 7.5 mg, or about 10 mg of polynucleotides). The polynucleotides may be formulated in the unit dose above in a volume of 0.1 ml to 10 ml (e.g., 0.2 ml, 0.5 ml, 0.75 ml, 1 ml, 1.5 ml, 2 ml, 3 ml, 4 ml, 5 ml, 6 ml, 7 ml, 8 ml, 9 ml, or 10 ml).

The compositions may also include the featured viral vector containing a nucleic acid sequence encoding a fusion protein (e.g., a Cas-exonuclease fusion protein), one or more guide polynucleotides (e.g., gRNAs), and/or a donor DNA molecule or a composition containing a fusion protein (e.g., a Cas-exonuclease fusion protein), one or more guide polynucleotides (e.g., gRNAs), and/or a donor DNA molecule. The compositions containing viral particles can be prepared in 1 ml to 10 ml (e.g., 1 ml, 2 ml, 3 ml, 4 ml, 5 ml, 6 ml, 7 ml, 8 ml, 9 ml, or 10 ml) aliquots, having a viral titer of at least about 1×10⁶ pfu/ml (plaque-forming unit/milliliter), and, in general, not exceeding 1×10¹¹ pfu/ml. Thus, the composition may contain, for example, about 1×10⁶ pfu/ml, about 2×10⁶ pfu/ml, about 4×10⁶ pfu/ml, about 1×10⁷ pfu/ml, about 2×10⁷ pfu/ml, about 4×10⁷ pfu/ml, about 1×10⁸ pfu/ml, about 2×10⁸ pfu/ml, about 4×10⁸ pfu/ml, about 1×10⁹ pfu/ml, about 2×10⁹ pfu/ml, about 4×10⁹ pfu/ml, about 1×10¹⁰ pfu/ml, about 2×10¹⁰ pfu/ml, about 4×10¹⁰ pfu/ml, and about 1×10¹¹ pfu/ml. The composition can include a pharmaceutically acceptable carrier described herein. The pharmaceutically acceptable carrier can be, for example, a liquid carrier such as a saline solution, protamine sulfate (Elkins-Sinn, Inc., Cherry Hill, N.J.) or Polybrene (Sigma) as well as others described herein.

Methods of Use

The featured gene editing system can be used to insert a polynucleotide (e.g., a donor DNA molecule) into a target site (e.g., a target genomic site) using HDR. Next generation gene sequencing can be used to identify a site having a genetic mutation (e.g., a missense mutation, a nonsense mutation, an insertion, a deletion, a duplication, a frameshift mutation, or a repeat expansion) or a gene of interest. Using gene sequence data, suitable target sites or target genomic sites upstream and downstream of the site of interest can be identified for development of the guide polynucleotides (e.g., gRNAs). Each target site (e.g., target genomic site) can be selected to correspond to a sequence of 17-25 nts (and is preferably a unique sequence) that can be used to direct the gRNA to that site. The selected target site may also be chosen based on its proximity to a 3-5 nucleic acid PAM site, which may be selected based on the characteristics of the selected Cas nuclease of the fusion protein. The 17-25 nt sequence of each target site or target genomic site can be selected to limit any off-targeting sites. Upon determination of suitable target sites or target genomic sites, guide polynucleotides (e.g., gRNAs) can be designed with a complementary sequence to the target site or target genomic site sequence.

The featured gene editing system can be used to create 5′ and 3′ overhangs for knock in of a donor DNA molecule. Sequencing data can be used to identify the nucleic acid sequence of the 5′ and 3′ overhangs created by the exonuclease domain of the featured fusion protein. The 5′ and 3′ overhangs are achieved by fusion of Cas protein to an exonuclease. Fusion of the exonuclease to the Cas protein localizes the exonuclease to the cleavage site and facilitates nuclease activity at those sites.

The featured gene editing system can use two or more guide polynucleotides (e.g., guide RNAs) to target the donor DNA. Homology arms can be incorporated into the donor DNA molecule to increase the efficiency of HDR. The featured guide polynucleotides (e.g., guide RNAs) can be targeted, individually, to a target site within these homology arms. In these instances, the guide polynucleotides are targeted to sites in the endogenous DNA flanking a region of interest to be edited. The sequence of the homology arms can be modified such that the donor DNA arms can be cut by the gene editing system whereas the endogenous DNA is not. Furthermore, the sequence of the homology arms can be modified to remove possible PAM sites so as to limit the targeting of the donor DNA by the gene editing system compared to the target genomic DNA. The donor DNA molecule can contain a gene or a fragment thereof desired to be inserted in place of an existing nucleic acid molecule in a host cell, as well as one or more of homology arms, a CRISPR/Cas inhibitor, and one or more promoters. The vector containing the donor DNA molecule may also contain, e.g., an SV40 on to enhance plasmid expression.

The featured gene editing system can use two or more guide polynucleotides (e.g., guide RNAs) to target the endogenous genomic DNA. The featured guide polynucleotides (e.g., guide RNAs) can be targeted, individually, to a target site upstream from and a target site downstream from a desired genomic site (e.g., a gene of interest or a mutation site) in the endogenous genomic DNA. In these instances, the guide polynucleotides are targeted to sites in the DNA flanking a region of interested to be edited. The guide polynucleotides can form a CRISPR/Cas complex with the Cas fusion protein and can promote the creation of double strand breaks (DSBs) both upstream and downstream from the target genomic site (e.g., a gene of interest or a mutation site). The dual DSBs at the target site can reduce the likelihood of spontaneous reannealing at the cleavage site (e.g., without incorporation of the donor nucleic acid, if desired). After creating the DSBs, the exonuclease domain of the featured Cas fusion protein creates 5′ and 3′ overhangs to promote HDR. The creation of DSBs and 5′ and 3′ overhangs flanking the target genomic site promote the excision of the nucleic acids between the two target sites (e.g., the sites complementary to the guide polynucleotide sequence) and, preferably but not necessarily, the insertion of a donor DNA molecule. In addition, guide polynucleotides unique to the donor plasmid will cleave the donor plasmid (e.g., at an upstream site and a downstream site), thereby releasing the DNA region of interest with, e.g., flanking 5′ and 3′ arms, for incorporation into the DSBs created in the target genomic site by HDR. The guide polynucleotide (e.g., guide RNA) target sites (e.g., target genomic sites) flanking (e.g., upstream and downstream from) the endogenous DNA region of interest can be selected to promote the insertion of a donor DNA molecule (e.g., a donor DNA molecule containing a functional gene sequence of interest) without the excision of genomic DNA, if desired. In some embodiments, the DNA region of interest (the target site) contains a deletion mutation, and the inserted donor DNA molecule contains the DNA region of interest without the mutation.

The featured gene editing system can be incorporated into a suitable delivery vehicle, e.g., a viral delivery system, described herein. The delivery system can be used to introduce the gene editing system to a target cell for delivery of a gene or other nucleic acid modification to the target genome of the cell. A non-limiting example of a delivery system is a lentiviral vector with a nucleic acid sequence encoding the featured fusion protein, a nucleic acid sequence encoding the guide polynucleotides (e.g., RNAs), and, optionally, a nucleic acid sequence encoding the donor DNA, and one or more promoter sequences.

In some embodiments, the gene editing system can be incorporated into a nanoparticle for delivery of the components of the gene editing system (including the CRISPR/Cas complex). The nanoparticle can be formulated to deliver the gene editing system to the target genome for insertion. In certain embodiments, each of the fusion protein, the guide polynucleotide(s) (e.g., guide RNA(s), and the donor DNA molecule can be encapsulated in a single nanoparticle for delivery to the target genome or the different components can be encapsulated separately in multiple nanoparticles.

In some embodiments, the gene editing system can be used to introduce a genetic mutation (e.g., a missense mutation, a nonsense mutation, an insertion, a deletion, a duplication, a frameshift mutation, or a repeat expansion) or a gene of interest into a genome of a target cell. In these instances, the mutation may be inserted to treat (e.g., in a human) a disease or disorder or to replicate a known disease or disorder in the subject (e.g., in a non-human subject used to research treatments for the disease of disorder). In some embodiments, a mutation is introduced into a genome or a target cell at a target site to understand the function of a gene(s) of a subject.

In some embodiments, the gene editing system can be used to target one or more copies of a given allele on a chromosome using a SNP derived PAM targeting site. Differences in SNP sequences between the two allelic copies (or three in a trisomic state) allow for selection of PAM sites present on one (or more) of the alleles. In these instances, only the PAM site with the Cas-gRNA will be cut, thereby promoting insertion or deletion of genomic material in the allelic copy (copies) with the SNP derived PAM site.

Examples of target genome sites (e.g., target polynucleotides) include a polynucleotide sequence associated with a signaling biochemical pathway, e.g., a signaling biochemical pathway-associated gene or polynucleotide. Further examples of target genome sites (e.g., target polynucleotides) include a disease associated gene or polynucleotide. A “disease-associated” gene or polynucleotide refers to any gene or polynucleotide that yields transcription or translation products at an abnormal level or in an abnormal form in cells derived from a disease-affected tissue as compared with tissues or cells of a non-disease control. It may be a gene that results in a disease or disorder owing to expression at an abnormally high level. It may be a gene that results in a disease or disorder owing to expression at an abnormally low level, where the altered expression correlates with the occurrence or and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

In some embodiments, the gene editing system can be targeted to a site outside of the disease-causing gene (e.g., a site that is upstream from the disease-causing gene or a site that is downstream from the disease-causing gene). In these instances, the donor DNA molecule can be integrated at the site outside of the disease-causing gene. In some embodiments, the gene editing system can be targeted to a site in a gene so as to not interfere with the expression of the gene. In some embodiments, the gene editing system can be targeted to a mutation that causes a gene to be non-functional. In some embodiments, the gene editing system can be used to excise an entire gene. In these instances, the disease or disorder can be caused by a functional gene, e.g., a disease or disorder that results from a duplication of the gene (e.g., a trisomy, such as trisomy 21).

In some embodiments, the CRISPR/Cas inhibitor can be provided to a cell in a way that delays the inhibition of the CRISPR/Cas fusion protein until after HDR has been performed. In some embodiments, the CRISPR/Cas inhibitor can be provided to the cell as a polynucleotide, in which the expression of the inhibitor can be operably linked to a promoter, and in which the promoter is a less robust promoter than a promoter operably linked to the CRISPR/Cas system. In another embodiment, a polynucleotide sequence encoding the CRISPR/Cas inhibitor is incorporated into the donor DNA molecule, such that expression of the inhibitor can occur after insertion of the donor DNA molecule into the target nucleic acid (e.g., a nucleic acid molecule of a genome, such as a nucleic acid molecule of a chromosome (e.g., a gene)). In certain embodiments, the CRISPR/Cas inhibitor can be provided to a cell after HDR to prevent off target effects. In some embodiments, the CRISPR/Cas inhibitor is provided to a target cell as a protein molecule after HDR to inhibit further activity of the CIRSPR/Cas fusion protein.

Methods of Treatment

Generally, a composition containing the featured gene editing system can be administered (e.g., intravenously) to a subject (e.g., a subject in need thereof, such as a human) as a medicament (e.g., for treating a disease or disorder). The modified gene editing system described herein can be used to efficiently target any of a number of genomic sites associated with a disease or disorder. Gene sequencing methods (e.g., next-generation gene sequencing methods, e.g., high-throughput sequencing, including but not limited to, Illumina sequencing, Roche 454 sequencing, Ion torrent: Proton/PGM sequencing, and SOLiD sequencing) can be used to identify a mutation (e.g., a missense mutation, a nonsense mutation, an insertion, a deletion, a duplication, a frameshift mutation, or a repeat expansion) associated with the disease or disorder in a subject (e.g., a subject suspected of having the disease or disorder), which can identify the subject as one in need of treatment. The gene sequencing data can also be used to identify a suitable target site(s) or target genomic site(s) to be targeted by a guide polynucleotide(s) (e.g., a guide RNA(s)) so as to limit any effect at off-target sites. Target sites and target genomic sites will, preferably, but not necessarily, be unique to the disease or disorder, and to the Cas nuclease of the featured fusion protein (e.g., owing to the selection of sites having a PAM sequence associated with the Cas nuclease).

The nucleic acid sequence of the donor DNA molecule can be determined by the location of the target site(s) or target genomic site(s), the disease or disorder being treated, and the fusion protein of the gene editing system. The donor DNA molecule can contain a nucleic acid sequence that, when inserted into the genomic DNA, corrects the cause of the disease or disorder (e.g., a genetic mutation). The donor DNA molecule can also contain a nucleic acid sequence encoding a Cas nuclease inhibitor. In some embodiments, the disease or disorder to be treated is one caused by a deletion mutation in a gene, which can be corrected using the gene editing system.

The fusion protein, guide polynucleotide (e.g., gRNA), and donor DNA molecule can be administered to a subject in need thereof (e.g., a human) to insert the donor DNA molecule at or between the identified target sites or target genomic sites. Compositions and methods for delivering the CRISPR/Cas system components includes, e.g., a vector (e.g., a viral vector, such as a lentiviral vector particle), and non-vector delivery vehicles (e.g., nanoparticles), as discussed above. For example, the featured CRISPR/Cas system described herein may be formulated for and/or administered to a subject (e.g., a human) in need thereof (e.g., a subject who has been diagnosed with a disease or disorder) by a variety of routes, such as local administration at or near the site affected by the disease or disorder (e.g., injection near a cancer, injection to a joint for treating rheumatoid arthritis, injection into the subretinal space for treating wet age-related macular degeneration, direct administration to the central nervous system (CNS) (e.g., intracerebral, intraventricular, intrathecal, intracisternal, or stereotactic administration) for treating a neurological medical condition, such as Parkinson's disease, or direct injection into the cardiac muscle for treating cardiac infarction)), intravenous, parenteral, intradermal, transdermal, intramuscular, intranasal, subcutaneous, percutaneous, intratracheal, intraperitoneal, intraarterial, intravascular, inhalation, perfusion, lavage, topical, and/or oral administration. The most suitable route for administration in any given case may depend on the particular subject, pharmaceutical formulation methods, administration methods (e.g., administration time and administration route), the subject's age, body weight, sex, severity of the disease being treated, the subject's diet, and the subject's excretion rate. Compositions may be administered once, or more than once (e.g., once annually, twice annually, three times annually, bi-monthly, monthly). For local administration, the featured CRISPR/Cas system and featured viral vectors containing polynucleotides encoding the featured CRISPR/Cas system may be administered by any means that places the CRISPR/Cas system in a desired location, including catheter, syringe, shunt, stent, or microcatheter, pump. The subject can be monitored for incorporation of the donor DNA molecule into the target genome. Methods of monitoring the incorporation of the donor DNA molecule into the target genome are discussed further below. The dosing regimen may be adjusted based on the monitoring results to ensure a therapeutic response. One of ordinary skill in the art will understand how to adjust the dosing regimen based on the monitoring results.

Non-limiting examples of diseases and disorders and their associated genes and polynucleotides are provided in Table 5. Furthermore, the modified exonuclease CRISPR/Cas system can be targeted to genomic sites associated with cellular function. Non-limiting examples of cellular functions and their associated genes is provided Table 6. Mutations in these genes and pathways can result in production of improper proteins or proteins in improper amounts which affect function. Such genes, proteins, and pathways may be the target polynucleotide sequence of a CRISPR/Cas complex.

In some embodiments, the methods described herein relate to treating a subject having or diagnosed as having a disease or disorder, e.g., a disease or disorders listed in Table 5. In another embodiment, the methods described herein relate to treating a subject having or diagnosed as having a dysfunctional cellular pathway, e.g., a cellular pathway listed in Table 6.

Generally, a composition containing the gene editing system, either incorporated as a nucleic acid molecule (e.g., in a vector, such as a viral vector) encoding the components of the gene editing system (e.g., fusion Cas-exonuclease protein, guide polynucleotides (e.g., guide RNA), and, optionally, donor DNA) or in protein form (e.g., a composition containing a fusion Cas-exonuclease fusion protein in combination with one or more guide polynucleotide(s) (e.g., gRNA(s), and/or a donor DNA molecule), can be administered (e.g., intravenously) to a subject (e.g., a subject in need thereof) as a medicament (e.g., for treating a medical condition).

TABLE 5
Exemplary diseases and disorders and their associated genes that
may be targeted for treatment using the gene editing system

| DISEASE/ | |
|---|---|
| DISOR- | |
| DER(S) | DISORDER(S)/GENE(S) |
| Age-related | Aber, Ccl2, Cc2, cp (ceruloplasmin), Timp3; |
| Macular | Cathepsin D; VLDLR, Ccr2 |
| Degeneration | |
| Blood and | Anemia (CDAN1, CDA1, RPS19, DBA, PKLR, PK1, |
| coagulation | NT5C3, UMPH1, PSN1, RHAG, RH50A, NRAMP2, |
| diseases | SPTB, ALAS2, ANH1, ASB, ABCB7, ABC7, ASAT); |
| and | Bare lymphocyte syndrome (TAPBP, TPSN, TAP2, |
| disorders | ABCB3, PSF2, RING11, MHC2TA, C2TA, RFX5, |
| | RFXAP, RFX5), |
| | Bleeding disorders (TBXA2R, P2RX1, P2X1); |
| | Factor H and factor H-like 1 (HF1, CFH, HUS); |
| | Factor V and factor VIII (MCFD2); |
| | Factor VII deficiency (F7); |
| | Factor X deficiency (F10); |
| | Factor XI deficiency (F11); |
| | Factor XII deficiency (F12, HAF); |
| | Factor XIIIA deficiency (F13A1, F13A); |
| | Factor XIIIB deficiency (F13B); |
| | Fanconi anemia (FANCA, FACA, FA1, FA, FAA, |
| | FAAP95, FAAP90, FLJ34064, FANCB, FANCC, FACC, |
| | BRCA2, FANCD1, FANCD2, FANCD, FACD, FAD, |
| | FANCE, FACE, FANCF, XRCC9, FANCG, BRIP1, |
| | BACH1, FANCJ, PHF9, FANCL, FANCM, KIAA1596); |
| | Hemophagocytic lymphohistiocytosis disorders (PRF1, |
| | HPLH2, UNC13D, MUNC13-4, HPLH3, HLH3, FHL3); |
| | Hemophilia A (F8, F8C, HEMA); |
| | Hemophilia B (F9, HEMB); |
| | Hemorrhagic disorders (PI, ATT, F5); |
| | Leukocyte deficiencies and disorders (ITGB2, CD18, |
| | LCAMB, LAD, E1F2B1, ElF2BA, ElF2B2, ElF2B3, |
| | ElF2B5, LVWM, CACH, CLE, ElF2B4); |
| | Sickle cell anemia (HBB); |
| | Thalassemia (HBA2, HBB, HBD, LCRB, HBA1) |
| Cell | B-cell non-Hodgkin lymphoma (BCL7A, BCL7); |
| dysregulation | Leukemia (TAL1 TCL5, SCL, TAL2, FLT3, NBS1, NBS, |
| and | ZNFN1A1, IK1, LYF1, HOXD4, HOX4B, BCR, CML, |
| oncology | PHL, ALL, ARNT, KRAS2, RASK2, GMPS, AF10, |
| diseases | ARHGEF12, LARG, KIAA0382, CALM, CLTH, |
| and | CEBPA, CEBP, CHIC2, BTL, FLT3, KIT, PBT, LPP, |
| disorders | NPM1, NUP214, D9S46E, CAN, CAIN, RUNX1, |
| | CBFA2, AML1, WHSC1L1, NSD3, FLT3, AF1Q, NPM1, |
| | NUMA1, ZNF145, PLZF, PML, MYL, STAT5B, AF10, |
| | CALM, CLTH, ARL11, ARLTS1, P2RX7, P2X7, BCR, |
| | CML, PHL, ALL, GRAF, NF1, VRNF, WSS, NFNS, |
| | PTPN11, PTP2C, SHP2, NS1, BCL2, CCND1, PRAD1, |
| | BCL1, TCRA, GATA1, GF1, ERYF1, NFE1, ABL1, |
| | NQO1, DIA4, NMOR1, NUP214, D9S46E, CAN, CAIN). |
| Devel- | Angelman Syndrome (UBE3A, 15q11-13 deletion); |
| opmental | Canavan disease (ASPA); |
| | Cri du chat (5P-, CTNND2); |
| | Down syndrome (Trisomy 21); |
| | Klinefelter syndrome (XXY, two or more X |
| | chromosomes in males); |
| | Prader-Willi syndrome (deletion of chromosome 15 |
| | segment); |
| | Turner syndrome (monosomy X, SHOX). |
| Drug | Prkce (alcohol); Drd2; Drd4; ABAT (alcohol); |
| addiction | GRIA2; Grm5; Grin1; Htr1b; Grin2a; Drd3; Pdyn; |
| | Gria1 (alcohol) |
| Inflam- | AIDS (KIR3DL1, NKAT3, NKB1, AMB11, KIR3DS1, |
| mation | IFNG, CXCL12, SDF1); |
| and | Autoimmune lymphoproliferative syndrome (TNFRSF6, |
| immune | APT1, FAS, CD95, ALPS1A); |
| related | Combined immuno-deficiency, (IL2RG, SCIDX1, |
| diseases | SCIDX, IMD4); |
| and | HIV-1 (CCL5, SCYA5, D175136E, TCP228); |
| disorders | HIV susceptibility or infection (IL10, CSIF, CMKBR2, |
| | CCR2, CMKBR5, CCCKR5 (CCR5)); |
| | Immuno-deficiencies (CD3E, CD3G, AICDA, AID, |
| | HIGM2, TNFRSF5, CD40, UNG, DGU, HIGM4, |
| | TNFSF5, CD4OLG, HIGM1, IGM, FOXP3, IPEX, AHD, |
| | XPID, PIDX, TNFRSF14B, TACI); |
| | Inflammation (IL-10, IL-1 (IL-1a, IL-1b), IL-13, IL-17 |
| | (IL-17a (CTLA8), IL-17b, IL-17c, IL-17d, IL-17f, 11-23, |
| | Cx3cr1, ptpn22, TNFa, NOD2/CARD15 for IBD, IL-6, |
| | IL-12 (IL-12a, IL-12b), CTLA4, Cx3c11); |
| | Severe combined immunodeficiencies (SCIDs) (JAK3, |
| | JAKL, DCLRE1C, ARTEMIS, SCIDA, RAG1, RAG2, |
| | ADA, PTPRC, CD45, LCA, IL7R, CD3D, T3D, IL2RG, |
| | SCIDX1, SCIDX, IMD4). |
| Metabolic, | Amyloid neuropathy (TTR, PALB); |
| liver, | Amyloidosis (APOA1, APP, AAA, CVAP, AD1, GSN, |
| kidney, | FGA, LYZ, TTR, PALB); |
| and protein | Cirrhosis (KRT18, KRT8, CIRH1A, NAIC, TEX292, |
| diseases | KIAA1988); |
| and | Cystic fibrosis (CFTR, ABCC7, CF, MRP7); |
| disorders | Glycogen storage diseases (SLC2A2, GLUT2, G6PC, |
| | G6PT, G6PT1, GAA, LAMP2, LAMPB, AGL, GDE, |
| | GBE1, GYS2, PYGL, PFKM); |
| | Hepatic adenoma, 142330 (TCF1, HNF1A, MODY3), |
| | Hepatic failure, early onset, and neurologic disorder |
| | (SCOD1, SCO1), |
| | Hepatic lipase deficiency (LIPC), |
| | Hepato-blastoma, cancer and carcinomas (CTNNB1, |
| | PDGFRL, PDGRL, PRLTS, AXIN1, AXIN, CTNNB1, |
| | TP53, P53, LFS1, IGF2R, MPRI, MET, CASP8, MCH5; |
| | Medullary cystic kidney disease (UMOD, HNFJ, FJHN, |
| | MCKD2, ADMCKD2); |
| | Phenylketonuria (PAH, PKU1, QDPR, DHPR, |
| | PTS); |
| | Polycystic kidney and hepatic disease (FCYT, PKHD1, |
| | ARPKD, PKD1, PKD2, PKD4, PKDTS, PRKCSH, |
| | G19P1, PCLD, SEC63). |
| Muscular/ | Becker muscular dystrophy (DMD, BMD, MYF6), |
| Skeletal | Duchenne Muscular Dystrophy (DMD, BMD); |
| diseases | Emery-Dreifuss muscular dystrophy (LMNA, LMN1, |
| and | EMD2, FPLD, CMD1A, HGPS, LGMD1B, LMNA, |
| disorders | LMN1, EMD2, FPLD, CMD1A); |
| | Facio-scapulohumeral muscular dystrophy (FSHMD1A, |
| | FSHD1A); |
| | Muscular dystrophy (FKRP, MDC1C, LGMD2I, LAMA2, |
| | LAMM, LARGE, KIAA0609, MDC1D, FCMD, TTID, |
| | MYOT, CAPN3, CANP3, DYSF, LGMD2B, SGCG, |
| | LGMD2C, DMDA1, SCG3, SGCA, ADL, DAG2, |
| | LGMD2D, DMDA2, SGCB, LGMD2E, SGCD, SGD, |
| | LGMD2F, CMD1L, TCAP, LGMD2G, CMD1N, |
| | TRIM32, HT2A, LGMD2H, FKRP, MDC1C, LGMD2I, |
| | TTN, CMD1G, TMD, LGMD2J, POMT1, CAV3, |
| | LGMD1C, SEPN1, SELN, RSMD1, PLEC1, PLTN, |
| | EBS1); |
| | Osteopetrosis (LRP5, BMND1, LRP7, LR3, OPPG, |
| | VBCH2, CLCN7, CLC7, OPTA2, OSTM1, GL, TCIRG1, |
| | TIRC7, OC116, 0PTB1); |
| | Muscular atrophy (VAPB, VAPC, ALS8, SMN1, SMA1, |
| | SMA2, SMA3, SMA4, BSCL2, SPG17, GARS, SMAD1, |
| | CMT2D, HEXB, IGHMBP2, SMUBP2, CATF1, |
| | SMARD1); |
| | Tay-Sachs disease (HEXA). |
| Neurological | ALS (SOD1, ALS2, STEX, FUS, TARDBP, VEGF |
| and | (VEGF-a, VEGF-b, VEGF-c); |
| neuronal | Alzheimer disease (APP, AAA, CVAP, AD1, APOE, |
| diseases | AD2, PSEN2, AD4, STM2, APBB2, FE65L1, NOS3, |
| and | PLAU, URK, ACE, DCP1, ACE1, MPO, PACIP1, |
| disorders | PAXIP1L, PTIP, A2M, BLMH, BMH, PSEN1, AD3); |
| | Autism (Mecp2, BZRAP1, MDGA2, Sema5A, Neurexin |
| | 1, GLO1, MECP2, RTT, PPMX, MRX16, MRX79, |
| | NLGN3, NLGN4, KIAA1260, AUTSX2); |
| | Fragile X Syndrome (FMR2, FXR1, FXR2, mGLUR5); |
| | Huntington's disease and disease like disorders (HD, |
| | IT15, PRNP, PRIP, JPH3, JP3, HDL2, TBP, SCA17); |
| | Parkinson disease (NR4A2, NURR1, NOT, TINUR, |
| | SNCAIP, TBP, SCA17, SNCA, NACP, PARK1, PARK4, |
| | DJ1, PARK7, LRRK2, PARK8, PINK1, PARK6, UCHL1, |
| | PARK5, SNCA, NACP, PARK1, PARK4, PRKN, |
| | PARK2, PDJ, DBH, NDUFV2); |
| | Rett syndrome (MECP2, RTT, PPMX, MRX16, MRX79, |
| | CDKL5, STK9, MECP2, RTT, PPMX, MRX16, MRX79, |
| | x-Synuclein, DJ-1); |
| | Schizophrenia (Neuregulin1 (Nrg1), Erb4 (receptor for |
| | Neuregulin), Complexin1 (Cp1x1), Tph1 Tryptophan |
| | hydroxylase, Tph2, Tryptophan hydroxylase 2, |
| | Neurexin 1, GSK3, GSK3a, GSK3b, 5-HTT (51c6a4), |
| | COMT, DRD (Drd1a), SLC6A3, DAOA, DTNBP1, Dao |
| | (Dao1)); |
| | Secretase Related Disorders (APH-1 (alpha and beta), |
| | Presenilin (Psen1), nicastrin, (Ncstn), PEN-2, Nos1, |
| | Parp1, Nat1, Nat2); |
| | Trinucleotide Repeat Disorders (HTT (Huntington's Dx), |
| | SBMA/SMAX1/AR (Kennedy's Dx), FXN/X25 |
| | (Friedrich's Ataxia), ATX3 (Machado- Joseph's Dx), |
| | ATXN1 and ATXN2 (spinocerebellar ataxias), DMPK |
| | (myotonic dystrophy), Atrophin-1 and Atn1 (DRPLA |
| | Dx),CBP (Creb-BP - global instability), VLDLR |
| | (Alzheimer's), Atxn7, Atxn10). |
| Neoplasia | PTEN; ATM; ATR; EGFR; ERBB2; ERBB3; ERBB4; |
| | Notch1; Notch2; Notch3; Notch4; AKT; AKT2; AKT3; |
| | HIF; HIFI a; HIF3a; Met; HRG; Bc12; PPAR alpha; |
| | PPAR gamma; WT1 (Wilms Tumor); |
| | FGF Receptor Family members (5 members: 1, 2, 3, 4, |
| | 5); CDKN2a; APC; RB (retinoblastoma); MEN1; VHL; |
| | BRCA1; BRCA2; AR (Androgen Receptor); TSG101; |
| | IGF; IGF Receptor; Igf1 (4 variants); Igf2 (3 variants); |
| | Igf 1 Receptor; Igf 2 Receptor; Bax; Bc12; caspases |
| | family (9 members: 1, 2, 3, 4, 6, 7, 8, 9, 12); Kras; Apc |
| Ocular | Age-related macular degeneration (Aber, Ccl2, Cc2, cp |
| diseases | (ceruloplasmin), Timp3, cathepsinD, Vldlr, Ccr2); |
| and | Cataract (CRYAA, CRYA1, CRYBB2, CRYB2, PITX3, |
| disorders | BFSP2, CP49, CP47, CRYAA, CRYA1, PAX6, AN2, |
| | MGDA, CRYBA1, CRYB1, CRYGC, CRYG3, CCL, |
| | LIM2, MP19, CRYGD, CRYG4, BFSP2, CP49, CP47, |
| | HSF4, CTM, HSF4, CTM, MIP, AQP0, CRYAB, |
| | CRYA2, CTPP2, CRYBB1, CRYGD, CRYG4, |
| | CRYBB2, CRYB2, CRYGC, CRYG3, CCL, CRYAA, |
| | CRYA1, GJA8, CX50, CA El, GJA3, CX46, CZP3, |
| | CAE3, CCM1, CAM, KRIT1); |
| | Corneal clouding and dystrophy (APOA1, TGFBI, |
| | CSD2, CDGG1, CSD, BIGH3, CDG2, TACSTD2, |
| | TROP2, Ml Si, VSX1, RINX, PPCD, PPD, KTCN, |
| | COL8A2, FECD, PPCD2, PIP5K3, CFD); |
| | Cornea plana congenital (KERA, CNA2); |
| | Glaucoma (MYOC, TIGR, GLC1A, JOAG, GPOA, |
| | OPTN, GLC1E, FIP2, HYPL, NRP, CYP1B1, GLC3A, |
| | OPA1, NTG, NPG, CYP1B1, GLC3A); |
| | Leber congenital amaurosis (CRB1, RP12, CRX, |
| | CORD2, CRD, RPGRIP1, LCA6, CORD9, RPE65, |
| | RP20, AIPL1, LCA4, GUCY2D, GUC2D, LCA1, |
| | CORD6, RDH12, LCA3); |
| | Macular dystrophy (ELOVL4, ADMD, STGD2, |
| | STGD3, RDS, RP7, PRPH2, PRPH, AVMD, AOFMD, |
| | VMD2). |
| Schizo- | Neuregulin1 (Nrg1); Erb4 (receptor for Neuregulin); |
| phrenia | Complexin1 (Cplx1); Tph1 Tryptophan hydroxylase; |
| | Tph2 Tryptophan hydroxylase 2; Neurexin 1; GSK3; |
| | GSK3a; GSK3b |
| Epilepsy | EPM2A, MELF, EPM2, NHLRC1, EPM2A, EPM2B |
| myoclonic | |
| Lafora | |
| type | |
| 254780 | |
| Duchenne | DMD, BMD |
| muscular | |
| dystrophy | |
| type | |
| 310200 | |
| (3) | |
| AIDS | KIR3DL1, NKAT3, NKB1, AMB11, KIR3DS1 |
| (delayed/ | |
| rapid | |
| progression | |
| to (3)) | |
| AIDS (rapid | IFNG |
| progression | |
| to | |
| 609423 (3)) | |
| AIDS | CXCL12, SDF1 |
| (resistance | |
| to (3)) | |
| Alpha 1- | SERPINA1 [serpin peptidase inhibitor, clade A |
| Antitrypsin | (alpha-1 antiproteinase, antitrypsin), member 1]; |
| Deficiency | SERPINA2 [serpin peptidase inhibitor, clade A |
| | (alpha-1 antiproteinase, antitrypsin), member 2]; |
| | SERPINA3 [serpin peptidase inhibitor, clade A |
| | (alpha-1 antiproteinase, antitrypsin), member 3]; |
| | SERPINA5 [serpin peptidase inhibitor, clade A |
| | (alpha-1 antiproteinase, antitrypsin), member 5]; |
| | SERPINA6 [serpin peptidase inhibitor, clade A |
| | (alpha-1 antiproteinase, antitrypsin), member 6]; |
| | SERPINA7 [serpin peptidase inhibitor, clade A |
| | (alpha-1 antiproteinase, antitrypsin), member 7];” |
| | AND “SERPLNA6 (serpin peptidase inhibitor, |
| | clade A (alpha-1 antiproteinase, antitrypsin), |
| | member 6) |

TABLE 6
Exemplary cellular functions and their genes that may be targeted for
treatment using the gene editing system

| CELLULAR FUNCTION | GENES |
|---|---|
| PI3K/AKT signaling | PRKCE; ITGAM; ITGA5; IRAK1; |
| | PRKAA2; EIF2AK2; PTEN; EIF4E; |
| | PRKCZ; GRK6; MAPK1; TSC1; PLK1; |
| | AKT2; IKBKB; PIK3CA; CDK8; |
| | CDKN1B; NFKB2; BCL2; PIK3CB; |
| | PPP2R1A; MAPK8; BCL2L1; MAPK3; |
| | TSC2; ITGA1; KRAS; EIF4EBP1; RELA; |
| | PRKCD; NOS3; PRKAA1; MAPK9; |
| | CDK2; PPP2CA; PIM1; ITGB7; YWHAZ; |
| | ILK; TP53; RAF1.; IKBKG; RELB; |
| | DYRK1A; CDKN1A; ITGB1; MAP2K2; |
| | JAK1; AKT1; JAK2; PIK3R1; CHUK; |
| | PDPK1; PPP2R5C; CTNNB1.; MAP2K1; |
| | NFKB1; PAK3; ITGB3; CCND1; GSK3A; |
| | FRAP1; SFN; ITGA2; TTK; CSNK1A1; |
| | BRAF; GSK3B; AKT3; FOXO1; SGK; |
| | HSP90AA1; RPS6KB1 |
| ERK/MAPK signaling | PRKCE; ITGAM; ITGA5; HSPB1; IRAK1; |
| | PRKAA2; EIF2AK2; RAC1; RAP1A; |
| | TLN1; EIF4E; ELK1; GRK6; MAPK1; |
| | RAC2; PLK1; AKT2; PIK3CA; CDK8; |
| | CREB1; PRKCI; PTK2; FOS; RPS6KA4; |
| | PIK3CB; PPP2R1A; PIK3C3; MAPK8; |
| | MAPK3; ITGA1; ETS1; KRAS; MYCN; |
| | EIF4EBP1; PPARG; PRKCD; PRKAA1; |
| | MAPK9; SRC; CDK2; PPP2CA; PIM1; |
| | PIK3C2A; ITGB7; YWHAZ; PPP1CC; |
| | KSR1; PXN; RAF1; FYN; DYRK1A; |
| | ITGB1; MAP2K2; PAK4; PIK3R1; STAT3; |
| | PPP2R5C; MAP2K1; PAK3; ITGB3; ESR1; |
| | ITGA2; MYC; TTK; CSNK1A1; CRKL; |
| | BRAF; ATF4; PRKCA; SRF; STAT1; SGK |
| Glucocorticoid Receptor | RAC1; TAF4B; EP300; SMAD2; TRAF6; |
| signaling | PCAF; ELK1; MAPK1; SMAD3; AKT2; |
| | IKBKB; NCOR2; UBE2I; PIK3CA; |
| | CREB1; FOS; HSPA5; NFKB2; BCL2; |
| | MAP3K14; STAT5B; PIK3CB; PIK3C3; |
| | MAPK8; BCL2L1; MAPK3; TSC22D3; |
| | MAPK10; NRIP1; KRAS; MAPK13; |
| | RELA; STAT5A; MAPK9; NOS2A; PBX1; |
| | NR3C1; PIK3C2A; CDKN1C; TRAF2; |
| | SERPINE1; NCOA3; MAPK14; TNF; |
| | RAF1; IKBKG; MAP3K7; CREBBP; |
| | CDKN1A; MAP2K2; JAK1; IL8; NCOA2; |
| | AKT1; JAK2; PIK3R1; CHUK; STAT3; |
| | MAP2K1; NFKB1; TGFBR1; ESR1; |
| | SMAD4; CEBPB; JUN; AR; AKT3; CCL2; |
| | MMP1; STAT1; IL6; HSP90AA1 |
| Axonal Guidance signaling | PRKCE; ITGAM; ROCK1; ITGA5; |
| | CXCR4; ADAM12; IGF1; RAC1; RAP1A; |
| | E1F4E; PRKCZ; NRP1; NTRK2; |
| | ARHGEF7; SMO; ROCK2; MAPK1; PGF; |
| | RAC2; PTPN11; GNAS; AKT2; PIK3CA; |
| | ERBB2; PRKC1; PTK2; CFL1; GNAQ; |
| | PIK3CB; CXCL12; PIK3C3; WNT11; |
| | PRKD1; GNB2L1; ABL1; MAPK3; ITGA1; |
| | KRAS; RHOA; PRKCD; PIK3C2A; ITGB7; |
| | GLI2; PXN; VASP; RAF1; FYN; ITGB1; |
| | MAP2K2; PAK4; ADAM17; AKT1; |
| | PIK3R1; GLI1; WNT5A; ADAM10; |
| | MAP2K1; PAK3; ITGB3; CDC42; VEGFA; |
| | ITGA2; EPHA8; CRKL; RND1; GSK3B; |
| | AKT3; PRKCA |
| Ephrin Receptor signaling | PRKCE; ITGAM; ROCK1; ITGA5; |
| | CXCR4; IRAK1; PRKAA2; EIF2AK2; |
| | RAC1; RAP1A; GRK6; ROCK2; MAPK1; |
| | PGF; RAC2; PTPN11; GNAS; PLK1; |
| | AKT2; DOK1; CDK8; CREB1; PTK2; |
| | CFL1; GNAQ; MAP3K14; CXCL12; |
| | MAPK8; GNB2L1; ABL1; MAPK3; |
| | ITGA1; KRAS; RHOA; PRKCD; PRKAA1; |
| | MAPK9; SRC; CDK2; PIM1; ITGB7; PXN; |
| | RAF1; FYN; DYRK1A; ITGB1; MAP2K2; |
| | PAK4, AKT1; JAK2; STAT3; ADAM10; |
| | MAP2K1; PAK3; ITGB3; CDC42; VEGFA; |
| | ITGA2; EPHA8; TTK; CSNK1A1; CRKL; |
| | BRAF; PTPN13; ATF4; AKT3; SGK |
| Actin Cytoskeleton | ACTN4; PRKCE; ITGAM; ROCK1; |
| signaling | ITGA5; IRAK1; PRKAA2; EIF2AK2; |
| | RAC1; INS; ARHGEF7; GRK6; ROCK2; |
| | MAPK1; RAC2; PLK1; AKT2; PIK3CA; |
| | CDK8; PTK2; CFL1; PIK3CB; MYH9; |
| | DIAPH1;PIK3C3; MAPK8; F2R; MAPK3; |
| | SLC9A1; ITGA1; KRAS; RHOA; PRKCD; |
| | PRKAA1; MAPK9; CDK2; PIM1; |
| | PIK3C2A; ITGB7; PPP1CC; PXN; VIL2; |
| | RAF1; GSN; DYRK1A; ITGB1; MAP2K2; |
| | PAK4; PIP5K1A; PIK3R1; MAP2K1; |
| | PAK3; ITGB3; CDC42; APC; ITGA2; |
| | TTK; CSNK1A1; CRKL; BRAF; VAV3; |
| | SGK |
| Huntington's Disease | PRKCE; IGF1; EP300; RCOR1.; PRKCZ; |
| signaling | HDAC4; TGM2; MAPK1; CAPNS1; |
| | AKT2; EGFR; NCOR2; SP1; CAPN2; |
| | PIK3CA; HDAC5; CREB1; PRKC1; |
| | HSPA5; REST; GNAQ; PIK3CB; PIK3C3; |
| | MAPK8; IGF1R; PRKD1; GNB2L1; |
| | BCL2L1; CAPN1; MAPK3; CASP8; |
| | HDAC2; HDAC7A; PRKCD; HDAC11; |
| | MAPK9; HDAC9; PIK3C2A; HDAC3; |
| | TP53; CASP9; CREBBP; AKT1; PIK3R1; |
| | PDPK1; CASP1; APAF1; FRAP1; CASP2; |
| | JUN; BAX; ATF4; AKT3; PRKCA; CLTC; |
| | SGK; HDAC6; CASP3 |
| Apoptosis signaling | PRKCE; ROCK1; BID; IRAK1; PRKAA2; |
| | EIF2AK2; BAK1; BIRC4; GRK6; MAPK1; |
| | CAPNS1; PLK1; AKT2; IKBKB; CAPN2; |
| | CDK8; FAS; NFKB2; BCL2; MAP3K14; |
| | MAPK8; BCL2L1; CAPN1; MAPK3; |
| | CASP8; KRAS; RELA; PRKCD; PRKAA1; |
| | MAPK9; CDK2; PIM1; TP53; TNF; RAF1; |
| | IKBKG; RELB; CASP9; DYRK1A; |
| | MAP2K2; CHUK; APAF1; MAP2K1; |
| | NFKB1; PAK3; LMNA; CASP2; BIRC2; |
| | TTK; CSNK1A1; BRAF; BAX; PRKCA; |
| | SGK; CASP3; BIRC3; PARP1 |
| B Cell Receptor signaling | RAC1; PTEN; LYN; ELK1; MAPK1; |
| | RAC2; PTPN11; AKT2; IKBKB; PIK3CA; |
| | CREB1; SYK; NFKB2; CAMK2A; |
| | MAP3K14; PIK3CB; PIK3C3; MAPK8; |
| | BCL2L1; ABL1; MAPK3; ETS1; KRAS; |
| | MAPK13; RELA; PTPN6; MAPK9; EGR1; |
| | PIK3C2A; BTK; MAPK14; RAF1; IKBKG; |
| | RELB; MAP3K7; MAP2K2; AKT1; |
| | PIK3R1; CHUK; MAP2K1; NFKB1; |
| | CDC42; GSK3A; FRAP1; BCL6; BCL10; |
| | JUN; GSK3B; ATF4; AKT3; VAV3; |
| | RPS6KB1 |
| Leukocyte Extravasation | ACTN4; CD44; PRKCE; ITGAM; ROCK1; |
| signaling | CXCR4; CYBA; RAC1; RAP1A; PRKCZ; |
| | ROCK2; RAC2; PTPN11; MMP14; |
| | PIK3CA; PRKCI; PTK2; PIK3CB; |
| | CXCL12; PIK3C3; MAPK8; PRKD1; |
| | ABL1; MAPK10; CYBB; MAPK13; |
| | RHOA; PRKCD; MAPK9; SRC; PIK3C2A; |
| | BTK; MAPK14; NOX1; PXN; VIL2; |
| | VASP; ITGB1; MAP2K2; CTNND1; |
| | PIK3R1; CTNNB1; CLDN1; CDC42; F11R; |
| | ITK; CRKL; VAV3; CTTN; PRKCA; |
| | MMP1; MMP9 |
| Integrin signaling | ACTN4; ITGAM; ROCK1; ITGA5; RAC1; |
| | PTEN; RAP1A; TLN1; ARHGEF7; |
| | MAPK1; RAC2; CAPNS1; AKT2; CAPN2; |
| | P1K3CA; PTK2; PIK3CB; PIK3C3; |
| | MAPK8; CAV1; CAPN1; ABL1; MAPK3; |
| | ITGA1; KRAS; RHOA; SRC; PIK3C2A; |
| | ITGB7; PPP1CC; ILK; PXN; VASP; RAF1; |
| | FYN; ITGB1; MAP2K2; PAK4; AKT1; |
| | PIK3R1; TNK2; MAP2K1; PAK3; ITGB3; |
| | CDC42; RND3; ITGA2; CRKL; BRAF; |
| | GSK3B; AKT3 |
| Acute Phase Response | IRAK1; SOD2; MYD88; TRAF6; ELK1; |
| signaling | MAPK1; PTPN11; AKT2; IKBKB; |
| | PIK3CA; FOS; NFKB2; MAP3K14; |
| | PIK3CB; MAPK8; RIPK1; MAPK3; IL6ST; |
| | KRAS; MAPK13; IL6R; RELA; SOCS1; |
| | MAPK9; FTL; NR3C1; TRAF2; |
| | SERPINE1; MAPK14; TNF; RAF1; PDK1; |
| | IKBKG; RELB; MAP3K7; MAP2K2; |
| | AKT1; JAK2; PIK3R1; CHUK; STAT3; |
| | MAP2K1; NFKB1; FRAP1; CEBPB; JUN; |
| | AKT3; IL1R1; IL6 |
| PTEN signaling | ITGAM; ITGA5; RAC1; PTEN; PRKCZ; |
| | BCL2L11; MAPK1; RAC2; AKT2; EGFR; |
| | IKBKB; CBL; PIK3CA; CDKN1B; PTK2; |
| | NFKB2; BCL2; PIK3CB; BCL2L1; |
| | MAPK3; ITGA1; KRAS; ITGB7; ILK; |
| | PDGFRB; INSR; RAF1; IKBKG; CASP9; |
| | CDKN1A; ITGB1; MAP2K2; AKT1; |
| | PIK3R1; CHUK; PDGFRA; PDPK1; |
| | MAP2K1; NFKB1; ITGB3; CDC42; |
| | CCND1; GSK3A; ITGA2; GSK3B; AKT3; |
| | FOXO1; CASP3; RPS6KB1 |
| p53 signaling | PTEN; EP300; BBC3; PCAF; FASN; |
| | BRCA1; GADD45A; BIRC5; AKT2; |
| | PIK3CA; CHEK1; TP53INP1; BCL2; |
| | PIK3CB; PIK3C3; MAPK8; THBS1; ATR; |
| | BCL2L1; E2F1; PMAIP1; CHEK2; |
| | TNFRSF10B; TP73; RB1; HDAC9; CDK2; |
| | PIK3C2A; MAPK14; TP53; LRDD; |
| | CDKN1A; HIPK2; AKT1; RIK3R1; |
| | RRM2B; APAF1; CTNNB1; SIRT1; |
| | CCND1; PRKDC; ATM; SFN; CDKN2A; |
| | JUN; SNAI2; GSK3B; BAX; AKT3 |
| Aryl Hydrocarbon Receptor | HSPB1; EP300; FASN; TGM2; RXRA; |
| signaling | MAPK1; NQO1; NCOR2; SP1; ARNT; |
| | CDKN1B; FOS; CHEK1; SMARCA4; |
| | NFKB2; MAPK8; ALDH1A1; ATR; E2F1; |
| | MAPK3; NRIP1; CHEK2; RELA; TP73; |
| | GSTP1; RI31; SRC; CDK2; AHR; NFE2L2; |
| | NCOA3; TP53; TNF; CDKN1A; NCOA2; |
| | APAF1; NFKB1; CCND1; ATM; ESR1; |
| | CDKN2A; MYC; JUN; ESR2; BAX; IL6; |
| | CYP1B1; HSP90AA1 |
| Xenobiotic Metabolism | PRKCE; EP300; PRKCZ; RXRA; MAPK1; |
| signaling | NQO1; NCOR2; PIK3CA; ARNT; PRKCI; |
| | NFKB2; CAMK2A; PIK3CB; PPP2R1A; |
| | PIK3C3; MAPK8; PRKD1; ALDH1A1; |
| | MAPK3; NRIP1; KRAS; MAPK13; |
| | PRKCD; GSTP1; MAPK9; NOS2A; |
| | ABCB1; AHR; PPP2CA; FTL; NFE2L2; |
| | PIK3C2A; PPARGC1A; MAPK14; TNF; |
| | RAF1; CREBBP; MAP2K2; PIK3R1; |
| | PPP2R5C; MAP2K1; NFKB1; KEAP1; |
| | PRKCA; EIF2AK3; IL6; CYP1B1; |
| | HSP90AA1 |
| SAPK/JNK signaling | PRKCE; IRAK1; PRKAA2; EIF2AK2; |
| | RAC1; ELK1; GRK6; MAPK1; GADD45A; |
| | RAC2; PLK1; AKT2; PIK3CA; FADD; |
| | CDK8; PIK3CB; PIK3C3; MAPK8; RIPK1; |
| | GNB2L1; IRS1; MAPK3; MAPK10; |
| | DAXX; KRAS; PRKCD; PRKAA1; |
| | MAPK9; CDK2; PIM1; PIK3C2A; TRAF2; |
| | TP53; LCK; MAP3K7; DYRK1A; |
| | MAP2K2; PIK3R1; MAP2K1; PAK3; |
| | CDC42; JUN; TTK; CSNK1A1; CRKL; |
| | BRAF; SGK |
| PPAr/RXR signaling | PRKAA2; EP300; INS; SMAD2; TRAF6; |
| | PPARA; FASN; RXRA; MAPK1; SMAD3; |
| | GNAS; IKBKB; NCOR2; ABCA1; GNAQ; |
| | NFKB2; MAP3K14; STAT5B; MAPK8; |
| | IRS1; MAPK3; KRAS; RELA; PRKAA1; |
| | PPARGC1A; NCOA3; MAPK14; INSR; |
| | RAF1; IKBKG; RELB; MAP3K7; |
| | CREBBP; MAP2K2; JAK2; CHUK; |
| | MAP2K1; NFKB1; TGFBR1; SMAD4; |
| | JUN; IL1R1; PRKCA; IL6; HSP90AA1; |
| | ADIPOQ |
| NF-KB signaling | IRAK1; EIF2AK2; EP300; INS; MYD88; |
| | PRKCZ: TRAF6; TBK1; AKT2; EGFR; |
| | IKBKB; PIK3CA; BTRC; NFKB2; |
| | MAP3K14; PIK3CB; PIK3C3; MAPK8; |
| | RIPK1; HDAC2; KRAS; RELA; PIK3C2A; |
| | TRAF2; TLR4: PDGFRB; TNF; INSR; |
| | LCK; IKBKG; RELB; MAP3K7; CREBBP; |
| | AKT1; PIK3R1; CHUK; PDGFRA; |
| | NFKB1; TLR2; BCL10; GSK3B; AKT3; |
| | TNFAIP3; IL1R1 |
| Neuregulin signaling | ERBB4; PRKCE; ITGAM; ITGA5: PTEN; |
| | PRKCZ; ELK1; MAPK1; PTPN11; AKT2; |
| | EGFR; ERBB2; PRKCI; CDKN1B; |
| | STAT5B; PRKD1; MAPK3; ITGA1; |
| | KRAS; PRKCD; STAT5A; SRC; ITGB7; |
| | RAF1; ITGB1; MAP2K2; ADAM17; |
| | AKT1; PIK3R1; PDPK1; MAP2K1; ITGB3; |
| | EREG; FRAP1; PSEN1; ITGA2; MYC; |
| | NRG1; CRKL; AKT3; PRKCA; |
| | HSP90AA1; RPS6KB1 |
| Wnt & Beta catenin | CD44; EP300; LRP6; DVL3; CSNK1E; |
| signaling | GJA1; SMO; AKT2; PIN1; CDH1; BTRC; |
| | GNAQ; MARK2; PPP2R1A; WNT11; SRC; |
| | DKK1; PPP2CA; SOX6; SFRP2: ILK; |
| | LEF1; SOX9; TP53; MAP3K7; CREBBP; |
| | TCF7L2; AKT1; PPP2R5C; WNT5A; |
| | LRP5; CTNNB1; TGFBR1; CCND1; |
| | GSK3A; DVL1; APC; CDKN2A; MYC; |
| | CSNK1A1; GSK3B; AKT3; SOX2 |
| Insulin Receptor signaling | PTEN; INS; EIF4E; PTPN1; PRKCZ; |
| | MAPK1; TSC1; PTPN11; AKT2; CBL; |
| | PIK3CA; PRKCI; PIK3CB; PIK3C3; |
| | MAPK8; IRS1; MAPK3; TSC2; KRAS; |
| | EIF4EBP1; SLC2A4; PIK3C2A; PPP1CC; |
| | INSR; RAF1; FYN; MAP2K2; JAK1; |
| | AKT1; JAK2; PIK3R1; PDPK1; MAP2K1; |
| | GSK3A; FRAP1; CRKL; GSK3B; AKT3; |
| | FOXO1; SGK; RPS6KB1 |
| IL-6 signaling | HSPB1; TRAF6; MAPKAPK2; ELK1; |
| | MAPK1; PTPN11; IKBKB; FOS; NFKB2: |
| | MAP3K14; MAPK8; MAPK3; MAPK10; |
| | IL6ST; KRAS; MAPK13; IL6R; RELA; |
| | SOCS1; MAPK9; ABCB1; TRAF2; |
| | MAPK14; TNF; RAF1; IKBKG; RELB; |
| | MAP3K7; MAP2K2; IL8; JAK2; CHUK; |
| | STAT3; MAP2K1; NFKB1; CEBPB; JUN; |
| | IL1R1; SRF; IL6 |
| Hepatic Cholestasis | PRKCE; IRAK1; INS; MYD88; PRKCZ; |
| | TRAF6; PPARA; RXRA; IKBKB; PRKCI; |
| | NFKB2; MAP3K14; MAPK8; PRKD1; |
| | MAPK10; RELA; PRKCD; MAPK9; |
| | ABCB1; TRAF2; TLR4; TNF; INSR; |
| | IKBKG; RELB; MAP3K7; IL8; CHUK; |
| | NR1H2; TJP2; NFKB1; ESR1; SREBF1; |
| | FGFR4; JUN; IL1R1; PRKCA; IL6 |
| IGF-1 signaling | IGF1; PRKCZ; ELK1; MAPK1; PTPN11; |
| | NEDD4; AKT2; PIK30A; PRKC1; PTK2; |
| | FOS; PIK3CB; PIK3C3; MAPK8; 1GF1R; |
| | IRS1; MAPK3; IGFBP7; KRAS; PIK3C2A; |
| | YWHAZ; PXN; RAF1; CASP9; MAP2K2; |
| | AKT1; PIK3R1; PDPK1; MAP2K1; |
| | IGFBP2; SFN; JUN; CYR61; AKT3; |
| | FOXO1; SRF; CTGF; RPS6KB1 |
| NRF2-mediated Oxidative | PRKCE; EP300; SOD2; PRKCZ; MAPK1; |
| Stress Response | SQSTM1; NQO1; PIK3CA; PRKC1; FOS; |
| | PIK3CB; P1K3C3; MAPK8; PRKD1; |
| | MAPK3; KRAS; PRKCD; GSTP1; |
| | MAPK9; FTL; NFE2L2; PIK3C2A; |
| | MAPK14; RAF1; MAP3K7; CREBBP; |
| | MAP2K2; AKT1; PIK3R1; MAP2K1; |
| | PPIB; JUN; KEAP1; GSK3B; ATF4; |
| | PRKCA; EIF2AK3; HSP90AA1 |
| Hepatic, Fibrosis/Hepatic | EDN1; IGF1; KDR; FLT1; SMAD2; |
| Stellate Cell Activation | FGFR1; MET; PGF; SMAD3; EGFR; FAS; |
| | CSF1; NFKB2; BCL2; MYH9; IGF1R; |
| | IL6R; RELA; TLR4; PDGFRB; TNF; |
| | RELB; IL8; PDGFRA; NFKB1; TGFBR1; |
| | SMAD4; VEGFA; BAX; IL1R1; CCL2; |
| | HGF; MMP1; STAT1; IL6; CTGF; MMP9 |
| PPAR signaling | EP300; INS; TRAF6; PPARA; RXRA; |
| | MAPK1; IKBKB; NCOR2; FOS; NFKB2; |
| | MAP3K14; STAT5B; MAPK3; NRIP1; |
| | KRAS; PPARG; RELA; STAT5A; TRAF2; |
| | PPARGC1A; PDGFRB; TNF; INSR; RAF1; |
| | IKBKG; RELB; MAP3K7; CREBBP; |
| | MAP2K2; CHUK; PDGFRA; MAP2K1; |
| | NFKB1; JUN; IL1R1; HSP90AA1 |
| Fc Epsilon RI signaling | PRKCE; RAC1; PRKCZ; LYN; MAPK1; |
| | RAC2; PTPN11; AKT2; PIK3CA; SYK; |
| | PRKCI; PIK3CB; PIK3C3; MAPK8; |
| | PRKD1; MAPK3; MAPK10; KRAS; |
| | MAPK13; PRKCD; MAPK9; PIK3C2A; |
| | BTK; MAPK14; TNF; RAF1; FYN; |
| | MAP2K2; AKT1; PIK3R1; PDPK1; |
| | MAP2K1; AKT3; VAV3; PRKCA |
| G-Protein Coupled | PRKCE; RAP1A; RGS16; MAPK1; GNAS; |
| Receptor signaling | AKT2; IKBKB; PIK3CA; CREB1; GNAQ; |
| | NFKB2; CAMK2A; PIK3CB; PIK3C3; |
| | MAPK3; KRAS; RELA; SRC; PIK3C2A; |
| | RAF1; IKBKG; RELB; FYN; MAP2K2; |
| | AKT1; PIK3R1; CHUK; PDPK1; STAT3; |
| | MAP2K1; NFKB1; BRAF; ATF4; AKT3; |
| | PRKCA |
| Inositol Phosphate | PRKCE; IRAK1; PRKAA2; EIF2AK2; |
| Metabolism | PTEN; GRK6; MAPK1; PLK1; AKT2; |
| | PIK3CA; CDK8; PIK3CB; PIK3C3; |
| | MAPK8; MAPK3; PRKCD; PRKAA1; |
| | MAPK9; CDK2; PIM1; PIK3C2A; |
| | DYRK1A; MAP2K2; PIP5K1A; PIK3R1; |
| | MAP2K1; PAK3; ATM; TTK; CSNK1A1; |
| | BRAF; SGK |
| PDGF signaling | EIF2AK2; ELK1; ABL2; MAPK1; |
| | PIK3CA; FOS; PIK3CB; PIK3C3; MAPK8; |
| | CAV1; ABL1; MAPK3; KRAS; SRC; |
| | PIK3C2A; PDGFRB; RAF1; MAP2K2; |
| | JAK1; JAK2; PIK3R1; PDGFRA; STAT3; |
| | SPHK1; MAP2K1; MYC; JUN; CRKL; |
| | PRKCA; SRF; STAT1; SPHK2 |
| VEGF signaling | ACTN4; ROCK1; KDR; FLT1; ROCK2; |
| | MAPK1; PGF; AKT2; PIK3CA; ARNT; |
| | PTK2; BCL2; PIK3CB; PIK3C3; BCL2L1; |
| | MAPK3; KRAS; HIF1A; NOS3; PIK3C2A; |
| | PXN; RAF1; MAP2K2; ELAVL1; AKT1; |
| | PIK3R1; MAP2K1; SFN; VEGFA; AKT3; |
| | FOXO1; PRKCA |
| Natural Killer Cell | PRKCE; RAC1; PRKCZ; MAPK1; RAC2; |
| signaling | PTPN11; KIR2DL3; AKT2; PIK3CA; SYK; |
| | PRKCI; PIK3CB; PIK3C3; PRKD1; |
| | MAPK3; KRAS; PRKCD; PTPN6; |
| | PIK3C2A; LCK; RAF1; FYN; MAP2K2; |
| | PAK4; AKT1; PIK3R1; MAP2K1; PAK3; |
| | AKT3; VAV3; PRKCA |
| Cell Cycle: G1/S | HDAC4; SMAD3; SUV39H1; HDAC5; |
| Checkpoint Regulation | CDKN1B; BTRC; ATR; ABL1; E2F1; |
| | HDAC2; HDAC7A; RB1; HDAC11; |
| | HDAC9; CDK2; E2F2; HDAC3; TP53; |
| | CDKN1A; CCND1; E2F4; ATM; RBL2; |
| | SMAD4; CDKN2A; MYC; NRG1; GSK3B; |
| | RBL1; HDAC6 |
| T Cell Receptor signaling | RAC1; ELK1; MAPKI; IKBKB; CBL; |
| | PIK3CA; FOS; NFKB2; PIK3CB; PIK3C3; |
| | MAPK8; MAPK3; KRAS; RELA, |
| | PIK3C2A; BTK; LCK; RAF1; IKBKG; |
| | RELB, FYN; MAP2K2; PIK3R1; CHUK; |
| | MAP2K1; NFKB1; ITK; BCL10; JUN; |
| | VAV3 |
| Death Receptor signaling | CRADD; HSPB1; BID; BIRC4; TBK1; |
| | IKBKB; FADD; FAS; NFKB2; BCL2; |
| | MAP3K14; MAPK8; RIPK1; CASP8; |
| | DAXX; TNFRSF10B; RELA; TRAF2; |
| | TNF; IKBKG; RELB; CASP9; CHUK; |
| | APAF1; NFKB1; CASP2; BIRC2; CASP3; |
| | BIRC3 |
| FGF signaling | RAC1; FGFR1; MET; MAPKAPK2; |
| | MAPK1; PTPN11; AKT2; PIK3CA; |
| | CREB1; PIK3CB; PIK3C3; MAPK8; |
| | MAPK3; MAPK13; PTPN6; PIK3C2A; |
| | MAPK14; RAF1; AKT1; PIK3R1; STAT3; |
| | MAP2K1; FGFR4; CRKL; ATF4; AKT3; |
| | PRKCA; HGF |
| GM-CSF signaling | LYN; ELK1; MAPK1; PTPN11; AKT2; |
| | PIK3CA; CAMK2A; STAT5B; PIK3CB; |
| | PIK3C3; GNB2L1; BCL2L1; MAPK3; |
| | ETS1; KRAS; RUNX1; PIM1; PIK3C2A; |
| | RAF1; MAP2K2; AKT1; JAK2; PIK3R1; |
| | STAT3; MAP2K1; CCND1; AKT3; STAT1 |
| Amyotrophic Lateral | BID; IGF1; RAC1; BIRC4; PGF; CAPNS1; |
| Sclerosis signaling | CAPN2; PIK3CA; BCL2; PIK3CB; |
| | PIK3C3; BCL2L1; CAPN1; PIK3C2A; |
| | TP53; CASP9; PIK3R1; RAB5A; CASP1; |
| | APAF1; VEGFA; BIRC2; BAX; AKT3; |
| | CASP3; BIRC3 |
| JAK/Stat signaling | PTPN1; MAPK1; PTPN11; AKT2; |
| | PIK3CA; STAT5B; PIK3CB; PIK3C3; |
| | MAPK3; KRAS; SOCS1; STAT5A; |
| | PTPN6; PIK3C2A; RAF1; CDKN1A; |
| | MAP2K2; JAK1; AKT1; JAK2; PIK3R1; |
| | STAT3; MAP2K1; FRAP1; AKT3; STAT1 |
| Nicotinate and | PRKCE; IRAK1; PRKAA2; EIF2AK2; |
| Nicotinamide Metabolism | GRK6; MAPK1; PLK1; AKT2; CDK8; |
| | MAPK8; MAPK3; PRKCD; PRKAA1; |
| | PBEF1; MAPK9; CDK2; PIM1; DYRK1A; |
| | MAP2K2; MAP2K1; PAK3; NT5E; TTK; |
| | CSNK1A1; BRAF; SGK |
| Chemokine signaling | CXCR4; ROCK2; MAPK1; PTK2; FOS; |
| | CFL1; GNAQ; CAMK2A; CXCL12; |
| | MAPK8; MAPK3; KRAS; MAPK13; |
| | RHOA; CCR3; SRC; PPP1CC; MAPK14; |
| | NOX1; RAF1; MAP2K2; MAP2K1; JUN; |
| | CCL2; PRKCA |
| IL-2 signaling | ELK1; MAPK1; PTPN11; AKT2; PIK3CA; |
| | SYK; FOS; STAT5B; PIK3CB; PIK3C3; |
| | MAPK8; MAPK3; KRAS; SOCS1; |
| | STAT5A; PIK3C2A: LCK; RAF1; |
| | MAP2K2; JAK1; AKT1; PIK3R1; |
| | MAP2K1; JUN; AKT3 |
| Synaptic Long Term | PRKCE; IGF1; PRKCZ; PRDX6; LYN; |
| Depression | MAPK1; GNAS; PRKC1; GNAQ; |
| | PPP2R1A; IGF1R; PRKID1; MAPK3; |
| | KRAS; GRN; PRKCD; NOS3; NOS2A; |
| | PPP2CA; YWHAZ; RAF1; MAP2K2; |
| | PPP2R5C; MAP2K1; PRKCA |
| Estrogen Receptor signaling | TAF4B; EP300; CARM1; PCAF; MAPK1; |
| | NCOR2; SMARCA4; MAPK3; NRIP1; |
| | KRAS; SRC; NR3C1; HDAC3; |
| | PPARGC1A; RBM9; NCOA3; RAF1; |
| | CREBBP; MAP2K2; NCOA2; MAP2K1; |
| | PRKDC; ESR1; ESR2 |
| Protein Ubiquitination | TRAF6; SMURF1; BIRC4; BRCA1; |
| Pathway | UCHL1; NEDD4; CBL; UBE2I; BTRC; |
| | HSPA5; USP7; USP10; FBXW7; USP9X; |
| | STUB1; USP22; B2M; BIRC2; PARK2; |
| | USP8; USP1; VHL; HSP90AA1; BIRC3 |
| IL-10 signaling | TRAF6; CCR1; ELK1; IKBKB; SP1; FOS; |
| | NFKB2; MAP3K14; MAPK8; MAPK13; |
| | RELA; MAPK14; TNF; IKBKG; RELB; |
| | MAP3K7; JAK1; CHUK; STAT3; NFKB1; |
| | JUN; IL1R1; IL6 |
| VDR/RXR Activation | PRKCE; EP300; PRKCZ; RXRA; |
| | GADD45A; HES1; NCOR2; SP1; PRKC1; |
| | CDKN1B; PRKD1; PRKCD; RUNX2; |
| | KLF4; YY1; NCOA3; CDKN1A; NCOA2; |
| | SPP1; LRP5; CEBPB; FOXO1; PRKCA |
| TGF-beta signaling | EP300; SMAD2; SMURF1; MAPK1; |
| | SMAD3; SMAD1; FOS; MAPK8; MAPK3; |
| | KRAS; MAPK9; RUNX2; SERPINE1; |
| | RAF1; MAP3K7; CREBBP; MAP2K2; |
| | MAP2K1; TGFBR1; SMAD4; JUN; |
| | SMAD5 |
| Toll-like Receptor signaling | IRAK1; EIF2AK2; MYD88; TRAF6; |
| | PPARA; ELK1; IKBKB; FOS; NFKB2; |
| | MAP3K14; MAPK8; MAPK13; RELA; |
| | TLR4; MAPK14; IKBKG; RELB; |
| | MAP3K7; CHUK; NFKB1; TLR2; JUN |
| p38 MAPK signaling | HSPB1; IRAK1; TRAF6; MAPKAPK2; |
| | ELK1; FADD; FAS; CREB1; DDIT3; |
| | RPS6KA4; DAXX; MAPK13; TRAF2; |
| | MAPK14; TNF; MAP3K7; TGFBR1; MYC; |
| | ATF4; IL1R1; SRF; STAT1 |
| Neurotrophin/TRK | NTRK2; MAPK1; PTPN11; PIK3CA; |
| signaling | CREB1; FOS; PIK3CB; PIK3C3; MAPK8; |
| | MAPK3; KRAS; PIK3C2A; RAF1; |
| | MAP2K2; AKT1; PIK3R1; PDPK1; |
| | MAP2K1; CDC42; JUN; ATF4 |
| FXR/RXR Activation | INS; PPARA; FASN; RXRA; AKT2; |
| | SDC1; MAPK8; APOB; MAPK10; PPARG; |
| | MTTP; MAPK9; PPARGC1A; TNF; |
| | CREBBP; AKT1; SREBF1; FGFR4; AKT3; |
| | FOXO1 |
| Synaptic Long Term | PRKCE; RAP1A; EP300; PRKCZ; MAPK1; |
| Potentiation | CREB1; PRKC1; GNAQ; CAMK2A; |
| | PRKD1; MAPK3; KRAS; PRKCD; |
| | PPP1CC; RAF1; CREBBP; MAP2K2; |
| | MAP2K1; ATF4; PRKCA |
| Calcium signaling | RAP1A; EP300; HDAC4; MAPK1; |
| | HDAC5; CREB1; CAMK2A; MYH9; |
| | MAPK3; HDAC2; HDAC7A; HDAC11; |
| | HDAC9; HDAC3; CREBBP; CALR; |
| | CAMKK2; ATF4; HDAC6 |
| EGF signaling | ELK1; MAPK1; EGFR; PIK3CA; FOS; |
| | PIK3CB; PIK3C3; MAPK8; MAPK3; |
| | PIK3C2A; RAF1; JAK1; PIK3R1; STAT3; |
| | MAP2K1; JUN; PRKCA; SRF; STAT1 |
| Hypoxia signaling in the | EDN1; PTEN; EP300; NQO1; UBE21; |
| Cardiovascular System | CREB1; ARNT; HIF1A; SLC2A4; NOS3; |
| | TP53; LDHA; AKT1; ATM; VEGFA; JUN; |
| | ATF4; VHL; HSP90AA1 |
| LPS/IL-1 Mediated | IRAK1; MYD88; TRAF6; PPARA; RXRA; |
| Inhibition of RXR Function | ABCA1, MAPK8; ALDH1A1; GSTP1; |
| | MAPK9; ABCB1; TRAF2; TLR4; TNF; |
| | MAP3K7; NR1H2; SREBF1; JUN; IL1R1 |
| LXR/RXR Activation | FASN; RXRA; NCOR2; ABCA1; NFKB2; |
| | IRF3; RELA; NOS2A; TLR4; TNF; RELB; |
| | LDLR; NR1H2; NFKB1; SREBF1; IL1R1; |
| | CCL2; IL6; MMP9 |
| Amyloid Processing | PRKCE; CSNK1E; MAPK1; CAPNS1; |
| | AKT2; CAPN2;CAPN1; MAPK3; |
| | MAPK13; MAP T; MAPK14; AKT1; |
| | PSEN1; CSNK1A1; GSK3B; AKT3; APP |
| IL-4 signaling | AKT2; PIK3CA; PIK3CB; PIK3C3; IRS1; |
| | KRAS; SOCS1; PTPN6; NR3C1; PIK3C2A; |
| | JAK1; AKT1; JAK2; PIK3R1; FRAP1; |
| | AKT3; RPS6KB1 |
| Cell Cycle: G2/M DNA | EP300; PCAF; BRCA1; GADD45A; PLK1; |
| Damage Checkpoint | BTRC; CHEK1; ATR; CHEK2; YWHAZ; |
| Regulation | TP53; CDKN1A; PRKDC; ATM; SFN; |
| | CDKN2A |
| Nitric Oxide signaling in | KDR; FLT1; PGF; AKT2; PIK3CA; |
| the Cardiovascular System | PIK3CB; PIK3C3; CAV1; PRKCD; NOS3; |
| | PIK3C2A; AKT1; PIK3R1; VEGFA; |
| | AKT3; HSP90AA1 |
| Purine Metabolism | NME2; SMARCA4; MYH9; RRM2; |
| | ADAR; EIF2AK4; PKM2; ENTPD1; |
| | RAD51; RRM2B; TJP2; RAD51C; NT5E; |
| | POLD1; NME1 |
| cAMP-mediated signaling | RAP1A; MAPK1; GNAS; CREB1; |
| | CAMK2A; MAPK3; SRC; RAF1; |
| | MAP2K2; STAT3; MAP2K1; BRAF; ATF4 |
| Mitochondrial Dysfunction | SOD2; MAPK8; CASP8; MAPK10; |
| | MAPK9; CASP9; PARK7; PSEN1; PARK2; |
| | APP; CASP3 |
| Notch signaling | HES1; JAG1; NUMB; NOTCH4; |
| | ADAM17; NOTCH2; PSEN1; NOTCH3; |
| | NOTCH1; DLL4 |
| Endoplasmic Reticulum | HSPA5; MAPK8; XBP1; TRAF2; ATF6; |
| Stress Pathway | CASP9; ATF4; EIF2AK3; CASP3 |
| Pyrimidine Metabolism | NME2; AICDA; RRM2; EIF2AK4; |
| | ENTPD1; RRM2B; NT5E; POLD1; NME1 |
| Parkinson's signaling | UCHL1; MAPK8; MAPK13; MAPK14; |
| | CASP9; PARK7; PARK2; CASP3 |
| Cardiac & Beta Adrenergic | GNAS; GNAQ; PPP2R1A; GNB2L1; |
| signaling | PPP2CA; PPP1CC; PPP2R5C |
| Glycolysis/Gluco- | HK2; GCK; GPI; ALDH1A1; PKM2; |
| neogenesis | LDHA; HK1 |
| Interferon signaling | IRF1; SOCS1; JAK1; JAK2; IFITM1; |
| | STAT1; IFIT3 |
| Sonic Hedgehog signaling | ARRB2; SMO; GLI2; DYRK1A; GLI1; |
| | GSK3B; DYRKIB |
| Glycerophospholipid | PLD1; GRN; GPAM; YWHAZ; SPHK1; |
| Metabolism | SPHK2; PRDX6; CHKA |
| Phospholipid Degradation | PRDX6; PLD1; GRN; YWHAZ; SPHK1; |
| | SPHK2 |
| Tryptophan Metabolism | SIAH2; PRMT5; NEDD4; ALDH1A1; |
| | CYP1B1; SIAH1 |
| Lysine Degradation | SUV39H1; EHMT2; NSD1; SETD7; |
| | PPP2R5C |
| Nucleotide Excision Repair | ERCC5; ERCC4; XPA; XPC; ERCC1 |
| Pathway | |
| Starch and Sucrose | UCHL1; HK2; GCK; GPI; HK1 |
| Metabolism | |
| Amino sugars Metabolism | NQO1; HK2; GCK; HK1 |
| Arachidonic Acid | PRDX6; GRN; YWHAZ; CYP1B1 |
| Metabolism | |
| Circadian Rhythm signaling | CSNK1E; CREB1; ATF4; NR1D1 |
| Coagulation System | BDKRB1; F2R; SERPINE1; F3 |
| Dopamine Receptor | PPP2R1A; PPP2CA; PPP1CC; PPP2R5C |
| signaling | |
| Glutathione Metabolism | IDH2; GSTP1; ANPEP; IDH1 |
| Glycerolipid Metabolism | ALDH1A1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Histidine Metabolism | PRMT5; ALDH1A1 |
| Inositol Metabolism | ERO1L; APEX1 |
| Metabolism of Xenobiotics | GSTP1; CYP1B1 |
| by Cytochrome p450 | |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| signaling | |
| NRF2-mediated Oxidative | PRDX1 |
| Stress Response | |
| Pentose Phosphate Pathway | GPI |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; |
| | cnr2; Grk2; Trpa1; Pomc; Cgrp; Crf; Pka; |
| | Era; Nr2b; TRPM5; Prkaca; Prkacb; |
| | Prkar1a; Prkar2a |
| Mitochondrial Function | AIF; CytC; SMAC (Diablo); Aifm-1; |
| | Aifm-2 |
| Developmental neurology | BMP-4; Chordin (Chrd); Noggin (Nog); |
| | WNT, Wnt2; Wnt2b; Wnt3a; Wnt4; Wnt5a; |
| | Wnt6; Wnt7b; Wnt8b; Wnt9a; Wnt9b; |
| | Wnt10a; Wnt10b; Wnt16; beta-catenin; |
| | Dkk-1; Frizzled related proteins; Otx-2; |
| | Gbx2; FGF-8; Reelin; Dab1; unc-86 (Pou4f1 |
| | or Brn3a); Numb; Rein |

### Dosage and Administration

The pharmaceutical compositions described herein can be administered to a subject (e.g., a human) in a variety of ways. For example, the pharmaceutical compositions may be formulated for and/or administered orally, buccally, sublingually, parenterally, intravenously, subcutaneously, intramedullary, intranasally, as a suppository, using a flash formulation, topically, intradermally, subcutaneously, via pulmonary delivery, via intra-arterial injection, ophthalmically, optically, intrathecally, or via a mucosal route.

A viral vector, such as a lentiviral vector, can be administered in an amount effective to produce a therapeutic effect in a subject. The exact dosage of viral particles to be administered is dependent on a variety of factors, including the age, weight, and sex of the subject to be treated, and the nature and extent of the disease or disorder to be treated. The viral particles can be administered as part of a preparation having a titer of viral vectors of at least 1×10⁶ pfu/ml (plaque-forming unit/milliliter), and in general not exceeding 1×10¹¹ pfu/ml, in a volume between about 0.5 ml to about 10 ml (e.g., 1 ml, about 2 ml, about 3 ml, about 4 ml, about 5 ml, about 6 ml, about 7 ml, about 8 ml, about 9 ml, or about 10 ml). Thus, the administered composition may contain, for example, about 1×10⁶ pfu/ml, about 2×10⁶ pfu/ml, about 4×10⁶ pfu/ml, about 1×10⁷ pfu/ml, about 2×10⁷ pfu/ml, about 4×10⁷ pfu/ml, about 1×10⁸ pfu/ml, about 2×10⁸ pfu/ml, about 4×10⁸ pfu/ml, about 1×10⁹ pfu/ml, about 2×10⁹ pfu/ml, about 4×10⁹ pfu/ml, about 1×10¹⁰ pfu/ml, about 2×10¹⁰ pfu/ml, about 4×10¹⁰ pfu/ml, and about 1×10¹¹ pfu/ml. The dosage may be adjusted to balance the therapeutic benefit against any side effects.

Any of the non-viral vectors of the present invention can be administered to a subject in a dosage from about 10 μg to about 10 mg of polynucleotides (e.g., from 25 μg to 5.0 mg, from 50 μg to 2.0 mg, or from 100 μg to 1.0 mg of polynucleotides, e.g., from 10 μg to 20 μg, from 20 μg to 30 μg, from 30 μg to 40 μg, from 40 μg to 50 μg, from 50 μg to 75 μg, from 75 μg to 100 μg, from 100 μg to 200 μg, from 200 μg to 300 μg, from 300 μg to 400 μg, from 400 μg to 500 μg, from 500 μg to 1.0 mg, from 1.0 mg to 5.0 mg, or from 5.0 mg to 10 mg of polynucleotides, e.g., about 10 μg, about 20 μg, about 30 μg, about 40 μg, about 50 μg, about 60 μg, about 70 μg, about 80 μg, about 90 μg, about 100 μg, about 150 μg, about 200 μg, about 250 μg, about 300 μg, about 350 μg, about 400 μg, about 450 μg, about 500 μg, about 600 μg, about 700 μg, about 750 μg, about 1.0 mg, about 2.0 mg, about 2.5 mg, about 5.0 mg, about 7.5 mg, or about 10 mg of polynucleotides) in a volume of a pharmaceutically acceptable carrier between about 0.1 ml to about 10 ml (e.g., about 0.2 ml, about 0.5 ml, about 1 ml, about 1.5 ml, about 2 ml, about 3 ml, about 4 ml, about 5 ml, about 6 ml, about 7 ml, about 8 ml, about 9 ml, or about 10 ml).

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, e.g., a pH in the physiologically acceptable range. Examples of buffer solutions include saline, phosphate buffered saline, Tris buffered saline, Hank's buffered saline, and the like.

In some embodiments, the method may also include a step of assessing the subject for successful targeting by the gene editing system. In some embodiments, the subject in need of a treatment (e.g., a human subject having a disease or disorder) is monitored for alleviation of the symptoms of the disease or disorder. In these instances, the subject will be monitored for a reduction or decrease in the side effects of a disease or disorder, such as those described herein, or the risk or progression of the disease or disorder, may be relative to a subject who did not receive treatment, e.g., a control, a baseline, or a known control level or measurement. The reduction or decrease may be, e.g., by about 1%, 2%, 3%, 4%, 5%, 6%, 7%, 8%, 9%, 10%, 20%, 30%, 40%, 50%, 60%, 70%, 80%, 90%, 95%, 97%, 99%, or about 100% relative to a subject who did not receive treatment or a control, baseline, or known control level or measurement, or may be a reduction in the number of days during which the subject experiences the disease or disorder or associated symptoms (e.g., a reduction of 1-30 days, 2-12 months, 2-5 years, or 6-12 years). The results of monitoring a subject's response to a treatment can be used to adjust the treatment regimen.

In certain embodiments, the gene editing system can be used to introduce a genetic mutation (e.g., a missense mutation, a nonsense mutation, an insertion, a deletion, a duplication, a frameshift mutation, or a repeat expansion) or a gene of interest into a genome of a target cell. In these instances, the mutation may be inserted to treat (e.g., in a human) a disease or disorder or to replicate a known disease or disorder in the subject (e.g., in a non-human subject used to research treatments for the disease or disorder) In these instances, the subject (e.g., a human subject or a research animal) can be monitored for a change in the disease or disorder (e.g., a change in the progression of the disease or disorder or in a lessening of etiologies of the disease or disorder in a subject that has been treated, or, alternatively, in the production or increase in the etiologies of a disease or disorder in a subject (e.g., a research animal) that has had one or more cells edited to replicate the disease or disorder). The changes can be monitored relative to a subject who did not receive the treatment or editing modification, e.g., a control, a baseline, or a known control level or measurement. The change may be, e.g., by about 1%, 2%, 3%, 4%, 5%, 6%, 7%, 8%, 9%, 10%, 20%, 30%, 40%, 50%, 60%, 70%, 80%, 90%, 95%, 97%, 99%, or about 100% relative to a subject who did not receive treatment or editing modification or a control, baseline, or known control level or measurement, or may be a change in the number of days during which the subject experiences the disease or disorder or associated symptoms (e.g., a reduction of 1-30 days, 2-12 months, 2-5 years, or 6-12 years in a treated subject).

In certain embodiments, the treatment is monitored at the protein level. Successful expression of the featured fusion protein in a cell or tissue can be assessed by standard immunological assays, for example the ELISA (see, Ausubel et al. Current Protocols in Molecular Biology, Greene Publishing Associates, New York, V. 1-3, 2000; Harlow and Lane, Antibodies, A Laboratory Manual, Cold Spring Harbor Laboratory, 1988, the entire contents of which is hereby incorporated by reference).

Alternatively, the biological activity of the gene product of interest can be measured directly by the appropriate assay, for example, the assays provided herein. The skilled artisan would be able to select and successfully carry out the appropriate assay to assess the biological activity of the gene product of interest in a particular sample. Such assays (e.g., real time PCR (qPCR)) might require removing a sample (e.g., cells or tissue) from the individual to use in the assay. Expression of the featured fusion protein or gene product of the donor DNA molecule may be monitored by any of a variety of immune detection methods available in the art. For example, the gene product of the donor DNA molecule may be detected directly using an antibody directed to the receptor itself or an antibody directed to an epitope tag (e.g., a FLAG tag) that has been included on the receptor for facile detection.

Gene sequencing methods can be used to identify the successful insertion of the polynucleotide encoding the CRISPR/Cas fusion protein into the endogenous DNA molecule, and/or the successful insertion of the donor DNA molecule by the CRISPR/Cas system. The subsequent expression of the donor DNA molecule can be monitored, for example, by measuring the expression of the Cas inhibitor. In some embodiments, the insertion of the donor DNA molecule can be monitored by a change (e.g., an increase or decrease) in the expression level (e.g., protein level or mRNA level) from the polynucleotide sequence of the donor DNA molecule.

### Kits

Also featured are kits containing any one or more of the CRISPR/Cas system elements disclosed in the above methods and compositions. Kits of the invention include one or more containers comprising, for example, one or more of fusion proteins, or fragments thereof, one or more guide polynucleotide(s) (e.g., gRNAs), and, optionally, one or more donor DNA molecules, and/or one or more containers with nucleic acids encoding a fusion protein(s), or fragment(s) thereof, one or more gRNA(s), and, optionally, one or more donor DNA molecule(s) (e.g., vectors containing the nucleic acid molecules (e.g., a viral vector, such as a lentiviral vector)) and, optionally, instructions for use in accordance with any of the methods described herein.

Generally, these instructions comprise a description of administration or instructions for performance of an assay. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also envisioned.

The kits may be provided in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (e.g., the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). Kits may optionally provide additional components such as buffers and interpretive information. Normally, the kit comprises a container and a label or package insert(s) on or associated with the container.

### EXAMPLES

The following examples are put forth to provide those of ordinary skill in the art with a description of how the compositions and methods described herein may be used, made, and evaluated, and are intended to be purely exemplary of the invention and are not intended to limit the scope of what the inventors regard as their invention.

The following examples discuss uses of the modified CRISPR/Cas gene editing system described herein.

### Example 1

We show that several modifications to the CRISPR/Cas9 approach significantly enhance the efficiency of HDR: 1) use of two sgRNA directed toward the targeted genomic DNA site, 2) use of two sgRNA directed toward the 5′ and 3′ flanking arms of the donor DNA, which have been modified to include a PAM or unique sgRNA sequence and allow for Cas9-sgRNA cleavage and to prevent cleavage by the sgRNAs used to target the genomic DNA, 3) insertion of the donor plasmid into a vector (e.g., an SV40 ori-containing vector) capable of plasmid reproduction in order to increase copy number of the donor DNA to be inserted into the genomic DNA in the subject to be treated, 4) use of an exonuclease fused to Cas9 to promote 3′ and 5′ overhang creation at the site of the DSB for the donor DNA molecule, and 5) sequence conversion of the bacterial exonuclease to a eukaryotic exonuclease to enhance expression and promote knock in of a donor DNA (e.g., as exemplified using enhanced Green Fluorescent Protein (eGFP) reporter) at an efficiency of approximately 35%. We have also observed that SNP (single nucleotide polymorphism) associated PAM targeting can be used to direct insertion into a single desired chromosome. Finally, the use of a Cas nuclease inhibitor (e.g., a Cas9 inhibitor) provides additional improvements to HDR by limiting off target effects of the CRISPR/Cas system.

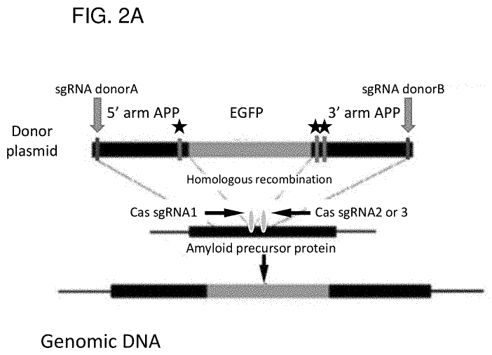

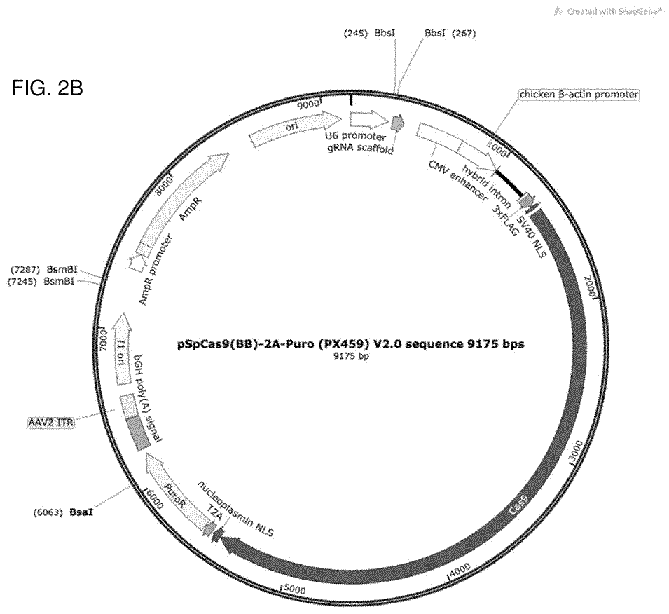

We used two sgRNAs (sgRNA1 on the 5′ end and sgRNA 2 or 3 on the 3′ end) to target exon 2 of the amyloid precursor protein (APP) and create an approximate 100 bp deletion (see FIGS. 2A and 2B). Sequence modifications were made in the donor DNA (see FIG. 2A, stars) in the 5′ and 3′ flanking regions such that they will not be cleaved by the sgRNA-Cas9. Cleavage using two sgRNAs reduces the likelihood of spontaneous re-annealing and allows for greater time to promote HDR.

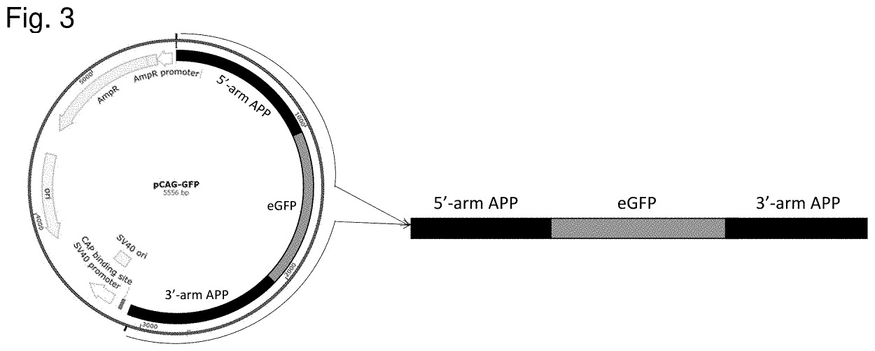

We also used two additional sgRNAs (labeled sgRNA-donor A and sgRNA donor B in FIG. 2A) which specifically target the 5′ and 3′ APP arms of the donor DNA (see also FIG. 3). The donor DNA sequence has been modified (see FIG. 2A 5′ arm and 3′ arm APP arrows) to allow for cleavage by the sgRNA-donorA/B-Cas9 complex in the APP sequence while sparing the endogenous DNA from targeting and cleavage by the CRISPR/Cas system. The donor plasmid construct can be inserted into a modified pCAG-GFP vector lacking the CAG promoter and containing a SV40 origin of replication which promotes plasmid replication following insertion into the cell. Increasing plasmid replication following insertion into the cell increases the concentration of donor DNA molecule. An increase in the number of the donor plasmids promotes an increase in the number of donor DNA molecules available for successful knock in, thereby promoting an increased efficiency of HDR. Upon insertion into the cell with the px459 gene editing system construct (including the sgRNA donor A/B), the donor plasmid can be cleaved and be made available for HDR.

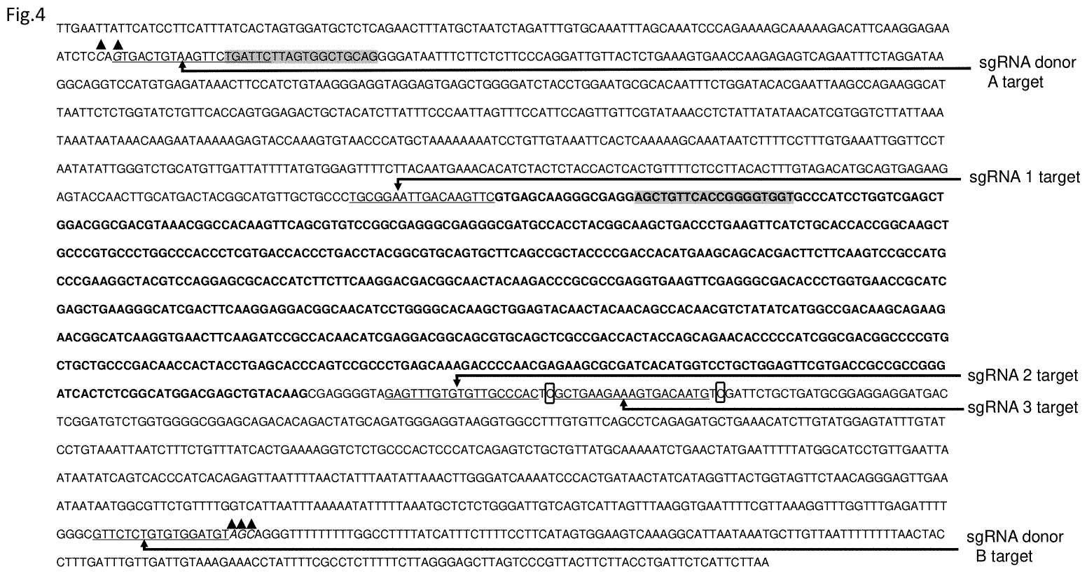

We can use the CRISPR/Cas9 targeting strategy described herein, exemplified by the construct shown in FIG. 4, to knock in donor genomic material of interest into the genome of a target cell. We confirmed the knock in efficiencies of this system using an eGFP gene sequence as the donor genomic material. The eGFP gene sequence includes 500 to 600 bp 3′- and 5′-homologous arms of the APP gene sequence (see FIG. 4). The APP-eGFP-APP sequence can be ligated to a modified pCAG-eGFP vector lacking the CAG promoter by overlapping PCR. To prevent undesired cutting of the APP-eGFP-APP sequence from the donor plasmid by Cas9 sgRNAs 1-3, we can modify the PAM sites for sgRNA2 and sgRNA3 in the original APP sequence by introducing G to C nucleotide point-mutations while maintaining the original amino acid codes (see boxed letter “C”). The PAM site for sgRNA1 in the donor plasmid is already disrupted by insertion of the eGFP. To allow the sgRNA-donor A/B sequences to specifically cut donor plasmid, but not genomic DNA, we can mutate the nucleotides of APP-eGFP-APP sequence into CAG in intron 2 and AGC in intron 3 (marked with triangles above). Three genomic target sequences for the guide RNA to target the genomic DNA (APP gene) are identified and are notated in the emphasized portions of the APP-eGFP-APP sequence (sgRNA1 genomic target sequence: TGCGGAATTGACAAGTTCCGAGG (SEQ ID NO: 20) (RefSeq: NM_000484.4) (e.g., sgRNA1 has the target sequence: UGCGGAAUUGACAAGUUCCG (SEQ ID NO: 21), sgRNA2 genomic target sequence: AGAGTTTGTGTGTTGCCCACTGG (SEQ ID NO: 22) (RefSeq: NM_000484.4) (e.g., sgRNA2 has the target sequence: AGAGUUUGUGUGUUGCCCAC (SEQ ID NO: 23), and sgRNA3 genomic target sequence: GGCTGAAGAAAGTGACAATGTGG (SEQ ID NO: 24) (RefSeq: NM_000484.4) (e.g., sgRNA3 has the target sequence: GGCUAAGAAAGUGACAAUG (SEQ ID NO: 25)). The sgRNA target sequences for cutting donor plasmid included APPintron2mu-sgRNA target sequence: GAATCAGAACTTACAGTCACTGG (SEQ ID NO: 26) (RefSeq: NM_000484.4) (e.g., the APPintro2mu-sgRNA has the target sequence: GAAUCAGAACUUACAGUCAC (SEQ ID NO: 27) and APPintron3mu-sgRNA target sequence: GTTCTCTGT GTGGATGTAGCAGG (SEQ ID NO: 28) (RefSeq: NM_000484.4) (e.g., the APPintron3mu-sgRNA has the target sequence: GUUCUCUGUGUGGAUGUAGC (SEQ ID NO: 29). These sgRNAs' sense and anti-sense DNA sequences were synthesized (IDT Company), annealed and ligated into BbsI-restriction enzyme-cut sites of px459, px459-mExo and px459-T5.

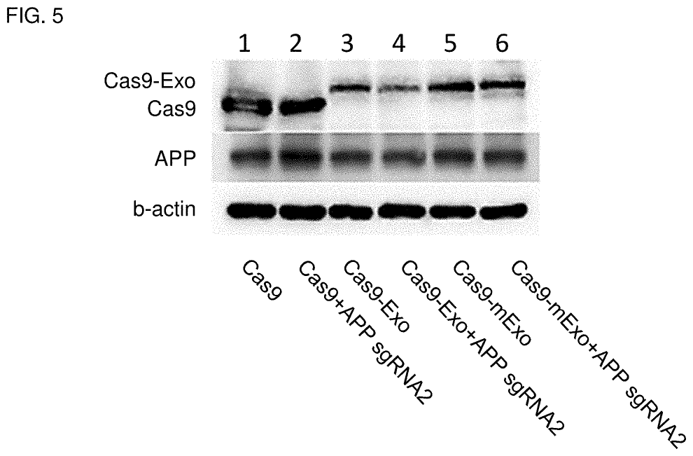

The pSpCas9(BB)-2A-Puro (PX459) V2.0 (plus a puromycin resistance marker and human codon-optimized Cas9, Addgene #62988) was modified to incorporate a single sgRNA2 targeting APP (App SgRNA2), and either a Cas9 fused to exonuclease lambda (Exo, prokaryotic) or Cas9 fused to a modified exonuclease lambda (mExo, eukaryotic). The plasmid was transfected into HEK293 cells and expression of various modifications of the PX459 plasmid are noted in the Western blot. Comparison of lanes 3 and 4 (Exo) and lanes 5 and 6 (mExo) show enhanced expression of the mExo construct (FIG. 5). Enhanced expression of the modified exonuclease promotes exonuclease efficiency.

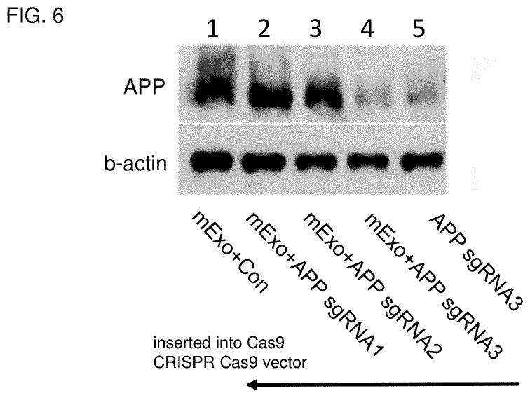

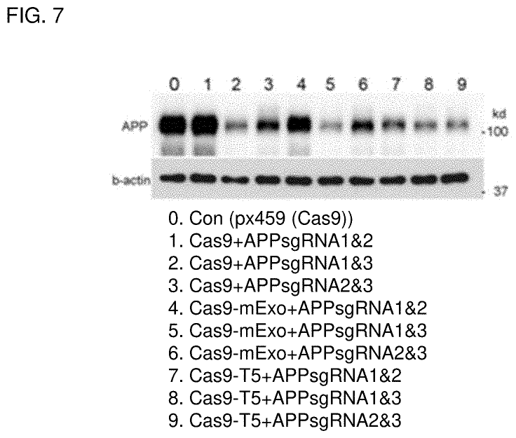

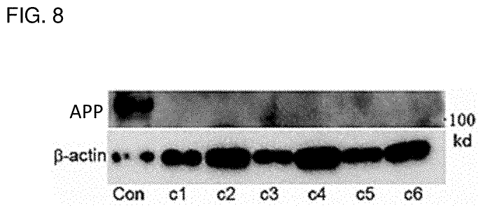

We designed three sgRNAs (APP sgRNA1, sgRNA2, sgRNA3) to target the APP gene at exon 2. Western blot analyses show that the greatest efficiency of APP knockdown is seen with sgRNA3 (FIG. 6, lane 4). Co-expression with mExo slightly enhances the knockdown efficiency. As previously reported, dual sgRNAs facilitate knockdown of the genomic target. We observe a similar effect on APP expression with the greatest decrease seen in the APP sgRNA1 and sgRNA3 (FIG. 7, lane 2). Expression levels of the APP gene are further inhibited with the addition of mExo (FIG. 7, lane 5). Efficient knockdown is also seen with the dual sgRNA and a T5 exonuclease (FIG. 7, lanes 7-9), but increased cell death was observed with these constructs. The efficiency of APP knockdown using the px459-mEXo, AppsgRNA1 and sgRNA3 is high (approaching 80%) as randomly selected, representative clonal lines derived following transfection show no APP expression (FIG. 8). These studies suggest that dual sgRNA appropriately cause DSBs and effectively target the intended APP site.

### Example 2

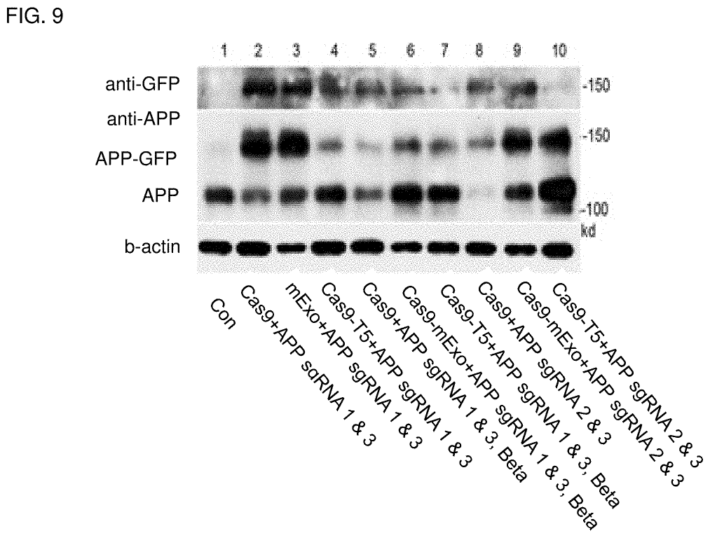

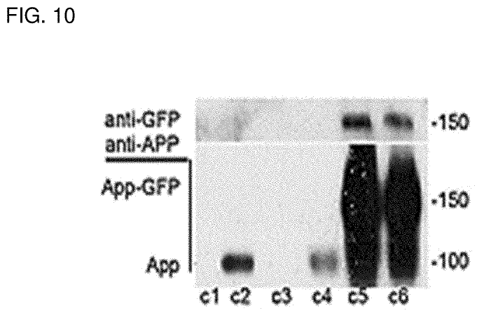

While enhanced knockdown efficiency is expected from the dual sgRNAs, we hypothesized that this same approach would inhibit reannealing of the DSB, prolong exonuclease activity, and enhance insertion of genomic material (e.g., eGFP). We observed greater efficiency of eGFP insertion with dual APP sgRNA1 and sgRNA3, and to a greater degree with APP sgRNA1 and sgRNA3 with mExo (FIG. 9, note the lower loading levels on b-actin, lanes 2 and 3). The addition of a beta protein from phage lambda did not enhance insertional efficiency (FIG. 9, lanes 5-7). Bacteriophage lambda encodes a 28 kDa protein (beta) that binds to single-stranded DNA and promotes the renaturation of complementary single strands. The knock in efficiency using dual sgRNAs and mExo approached 33%, as demonstrated by amplification of clonal cell lines and examination for APP-GFP expression (FIG. 10, clones c5 and c6 show appropriate insertion). Clones c1 and c3 show knockout of APP but no insertion of GFP whereas clones c2 and c4 show no effective knockout or GFP insertion.

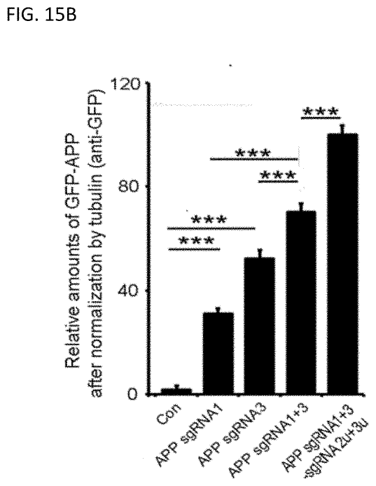

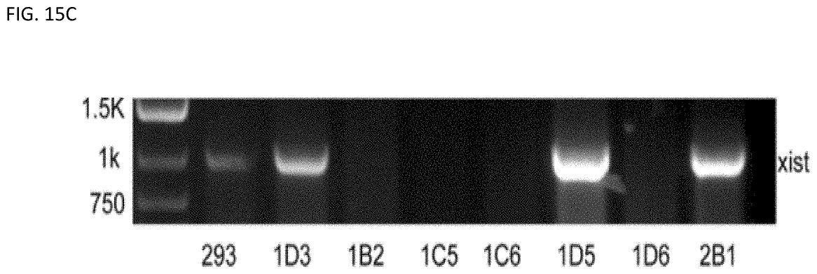

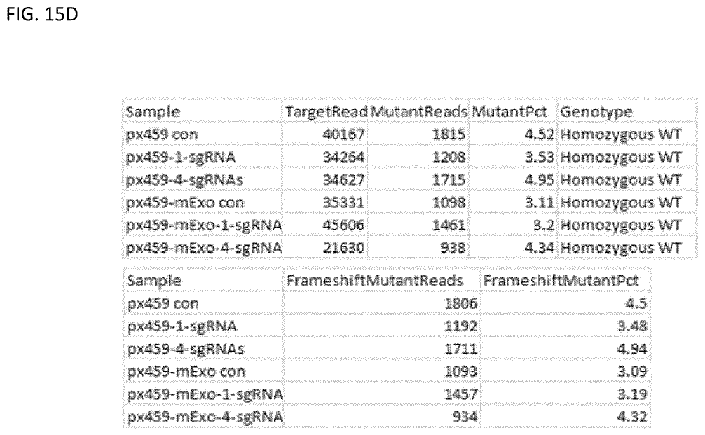

The increased efficiency of the gene editing system is further seen in a representative western blot (FIG. 15A) showing the integration of GFP within the APP gene in transfected HEK 293 cells using a px459-mExo vector containing a single APP sgRNA (sgRNA 1 or sgRNA 3; lanes 2 and 3, respectively), a px459-mExo vector containing dual sgRNAs (sgRNA1 and sgRNA3; lane 4), and a px459-mExo vector containing dual sgRNAs (sgRNA1 and sgRNA3) and donor sgRNAs (sRNA2u and sRNA3u; lane 5) that target the donor nucleic acid material in the vector. An empty px459-mExo vector is used as a control (lane 1). The upper panel of FIG. 15A shows the GFP-APP bands when the blot is incubated with anti-GFP antibody, the middle panel shows the GFP-APP bands (upper bands) and APP bands (lower bands) when the blot is incubated with anti-APP antibody, and the bottom panel shows the tubulin bands which represents the protein amounts of these samples. Statistical analysis using the western blot results (GFP-APP detection using an anti-GFP antibody) show the relative efficiency of GFP integration into the APP gene site (FIG. 15B; results presented after tubulin normalization). These results from multiple assays (n=4) show that the efficiency of target nucleic acid insertion (e.g., a donor DNA) increases with the use of a mExo in a px459 vector, the use of multiple APP sgRNAs, and the use of donor sgRNAs that produce a donor nucleic acid molecule with 5′ and 3′ overhangs. The use of all three components (mEXO, dual target sgRNAs, and dual donor sgRNAs) exhibits the greatest enhancement of HDR efficiency (observed as GFP integration into the APP gene, which is a non-limiting example of the gene targeting and donor nucleic acid insertion efficiency of the system and method of the present disclosure).

### Example 3

The efficiency of the CRISPR/Cas system described herein can be tested using an eGFP construct and sgRNAs in human DS iPS cells, primary Tc1 mouse neural progenitor cells, and glial cells. In this manner, different cell types and cells at different stages of development can be evaluated to ensure reproducibility and robustness of the integration.

We have established 2 DS iPS cells and their isogenic controls through the Harvard Human Pluripotent Stem Cell Core (CHB, Dr. Schlaeger). Genetic testing of one of the DS iPS lines shows three distinct microsatellite marker repeats at the D21S11 locus, implying that the three HSA21 copies are distinct and therefore each HSA21 chromosome would have different SNP variants. Primary neural, neuronal, and glial cultures are available for testing, and the Tc1 mouse is readily available from Jackson laboratories.

### Example 4

Treatment of certain genetic disorders can occur by targeting a mutation in a chromosome (e.g., in a gene of the chromosome) or of a chromosome (e.g., a mutation to duplicates a chromosome, such as a trisomy). Down Syndrome is a prototypical model system given the trisomy of chromosome 21 (HSA21). Appropriate insertion of the X-inactivation gene (XIST) onto HSA21 has been shown to rescue the DS neurological phenotype through inactivation of one of the three HSA21 copies.

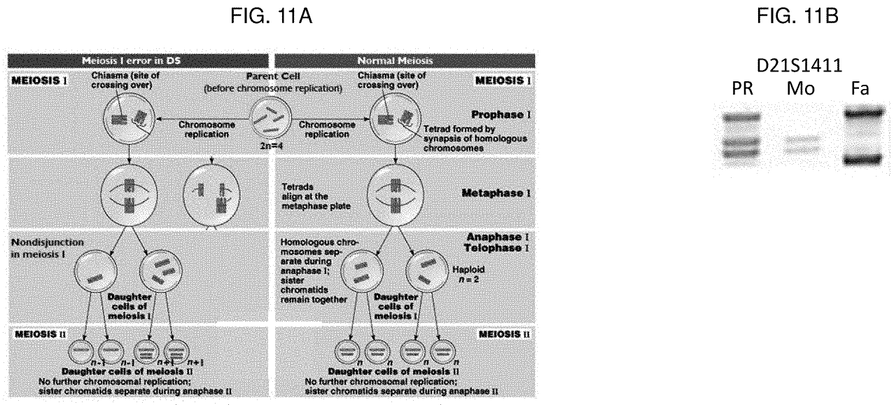

For clinical applications, treatment could be pursued using XIST targeting involving integration into only one of the three HSA21 chromosomes. This treatment approach has not been previously considered a viable option, in part, because the efficiency of genomic integration is so low that the likelihood of having two or more HSA21 copies within the same cell incorporate at the desired genomic material would be very low. However, the origin of nondisjunction in DS leading to the trisomic HSA21 predominantly (80-95%) occurs during meiosis I within cells of maternal origin. Non-disjunction leads to failure of homologous chromosome to separate during anaphase such that one of the gametes will have an extra HSA21 chromosome while the other will be missing an HSA21 chromosome (FIG. 11A). Importantly, each of the HSA21 chromosomes from the mother will be distinct and this uniqueness will allow for specificity of targeting using the CRISPR/Cas system of this disclosure. For example, the microsatellite marker D21S1411 on HSA21 (FIG. 11B) shows the proband (Pr) with DS (trisomic HSA21 with three bands). One of the bands is of paternal origin, whereas the other two are of maternal origin (consistent with maternal non-disjunction). Each of the three HSA21 chromosomes is distinct.

Leveraging the chromosome differences by identifying HSA21 SNPs on one of the maternal alleles can be used to create a unique PAM (protospacer adjacent motif, “NGG”, where N refers to any nucleobase followed by two guanine “G” nucleobases), thereby allowing targeting of a single chromosome. PAMs with the guide RNA can be used to promote formation of the DNA-RNA hybrid. In its absence, Cas9 would not efficiently, if at all, base-pair with genomic DNA, and would be ineffective at cutting the genomic DNA. Thus, unique PAM sites on one of the sequenced HSA21 alleles can be used to promote targeting of the particular chromosome.

Using the NCBI dbSNP site, we have identified 421 candidate SNP sites with Global MAF (global minor allele frequency 0.25-0.5) and screening of functional classes with synonymous codons (in exonic regions) on HSA21. A GMAF score of 0.25-0.5 indicates that the SNP variation frequency falls between 25-50%. Of this total, 178 exhibit either a potential CCN or GGN motif necessary to be a PAM site.

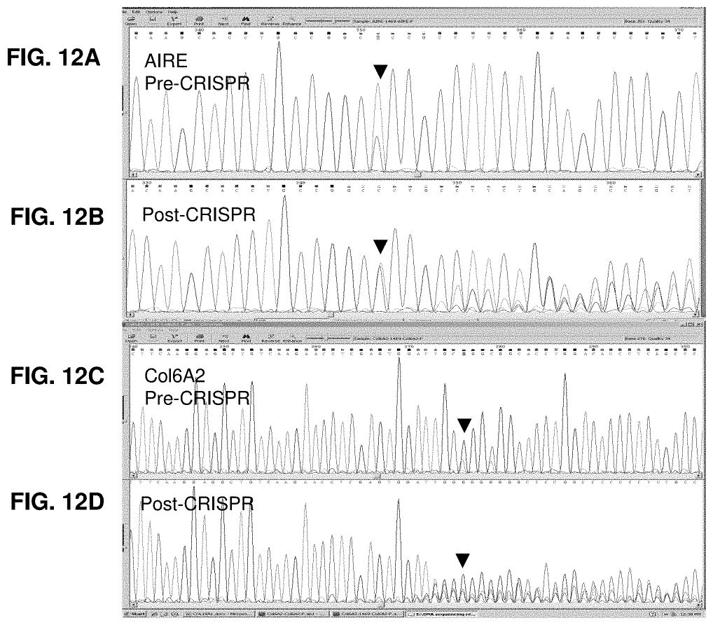

We first selected 6 potential sites near the 21q22.3 end terminus of HSA21, and identified 3 SNP sites that allow for targeting one of the three HSA21 copies. Non pathological SNP sites, identified using the NCBI database (www.ncbi.nlm.nih.gov/snp), are located at genes encoding autoimmune regulator (AIRE) (GGCYGCG) (SEQ ID NO: 30)), cystathionine-beta-synthase (CBS) (GGCYGCG (SEQ ID NO: 30)), and collagen type VI alpha 1 (COL6A1) (GTCYGGC (SEQ ID NO: 31)), in which Y is either C or T [C/T]. PCR sequencing results showed that the nucleotide signal at each position of AIRE gene sequence in non-transfected DS IPS cells appear as a single peak, except for the SNP site [T/C] (FIG. 12A, AIRE pre-CRISPR). After treatment with Cas9 and gRNA following the SNP-derived PAM, the nucleotide signal at each position appear as multiple peaks starting on AIRE gRNA sequence (FIG. 12B, AIRE post-CRISPR), suggesting that one allele of AIRE gene locus on HSA21 was specifically cut by Cas9-gRNA, causing nucleotide Indels (insert/deletion). The allele of AIRE gene locus without the SNP-derived PAM was not cut by Cas9-gRNA, therefore causing the appearance of hybridized signal peaks in the sequencing results. In addition, the DS iPS line shows that two of the three HSA21 Col6A2 alleles have a suitable SNP-derived PAM site [G/A] (FIG. 12C, Col6A2 pre-CRISPR). Introduction of the Cas9-gRNA to the allele of Col6A2 gene results in two of the three alleles being cut (FIG. 12D, Col6A2 post-CRISPR). These data prove feasibility of SNP-derived PAM sites in CRISPR mediated knockdown of particular genes of interest. Using Col6A2 gene as an example, the target genomic site sequences to which the sgRNAs are targeted have SNPs for targeted cleavage and correspond to CAAGAACCTCGAGTGGATTGCGG (SEQ ID NO: 32) (e.g., the corresponding sgRNA has the target sequence: CAAGAACCUCGAGUGGAUUG (SEQ ID NO: 33) and GACACGTGTGTTTGCGGTGG (SEQ ID NO: 34) (e.g., the corresponding sgRNA has the target sequence: GACACGUGUGUUUGCGG (SEQ ID NO: 35).

Several other assessments of phenotype reversal in the human DS iPSC lines can also be used. Non-limiting examples of assessments include, e.g., Barr body formation, Allele specific silencing, and genome wide silencing.

Barr body formation can be tested using previously established methods to assess XIST activation. HSA21 Barr body formation (DAPI) as well as enrichment for heterochromatin marks (H3K27Me3, UbH2A, H4k20Me antibodies) with XIST can be assessed in targeted iPS cells at days 0, 5 and 20 following XIST induction.

Allele specific silencing can be tested by measuring transcription of HSA21 genes localized at varying differences from XIST, such as by multi-color RNA FISH.

Genome wide silencing can be assessed by transcriptional mRNA microarray and methylation profiling. Platforms known in the art can be used, for example: Affymetrix HU 133 plus 2.0 chip for transcriptional RNA (Lu et al. (PLoS One 6(7): e22126, 2011)) and HumanMethylation450 BeadChips for methylation profiling (Lu et al. (Hum Mol Genet 25(9): 1714-1727)). Profiling can be performed on targeted XIST DS IPS lines prior to XIST induction, and, e.g., 20 days after XIST induction, as well as the corresponding isogenic lines (three clones per variable performed in triplicate). For mRNA microarray analyses, statistical significance of gene expression differences between sample variables can be determined by pairwise comparisons at each age using Significance Analysis of Microarrays. Differential methylation analysis can be performed using the R software, with comparisons first made by student's t-test with a cut-off P≤0.05, then further filtered with p3-value difference of >10%.

### Example 5

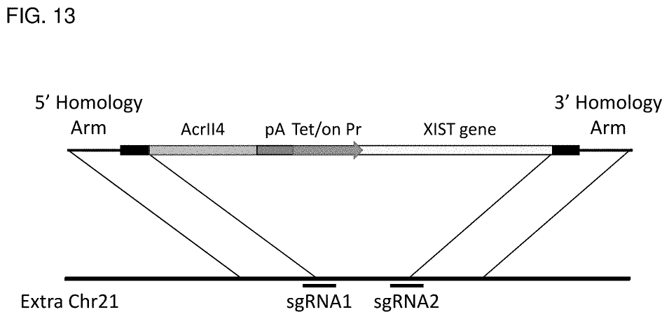

CRISPR technology brings concerns for the potential of off targeting effects. This possibility is minimized by two separate approaches. First, for each site-specific cleavage, the CRISPR/Cas9 system can be assessed for potential off-target loci and for faithfulness of on-target activity (computed as 100% minus a weighted sum of off target hit-scores in the target genome) using, e.g., standard nucleotide BLAST through NCBI. Second, a modified donor DNA molecule can be used in the system that contains a Cas9 inhibitor (see, e.g., FIG. 13; e.g., AcrIIA4 encodes the Cas9 inhibitor). With integration of the donor DNA molecule at the desired HSA21 site, the endogenous gene promoter can drive AcrIIA4 expression to inhibit Cas9 enzyme activity. XIST gene transcription can be directed using, e.g., a regulator system (e.g., a tetracycline system that results in transcription at the target site using a tetracycline promoter).

To assess off targeting effects, the selected potential off-target genomic sites can be PCR amplified using genomic DNA as templates. The PCR products can be subjected to the T7EN1 cleavage assay. Potential off-target genomic sites that yield typical cleavage bands would be considered as candidates, and then PCR products of the candidates can be cloned and sequenced to confirm the off-target effects. Additionally, sgRNA off targeting sites can be evaluated by CHIP-Seq.

### Example 6

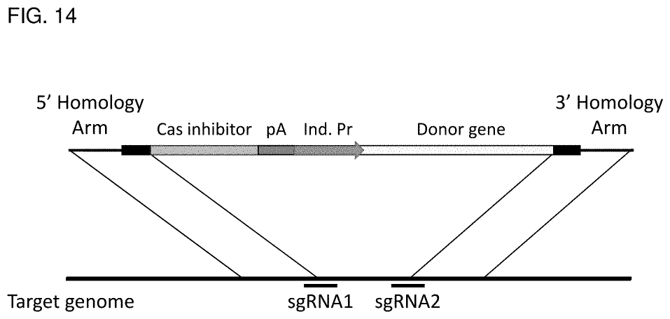

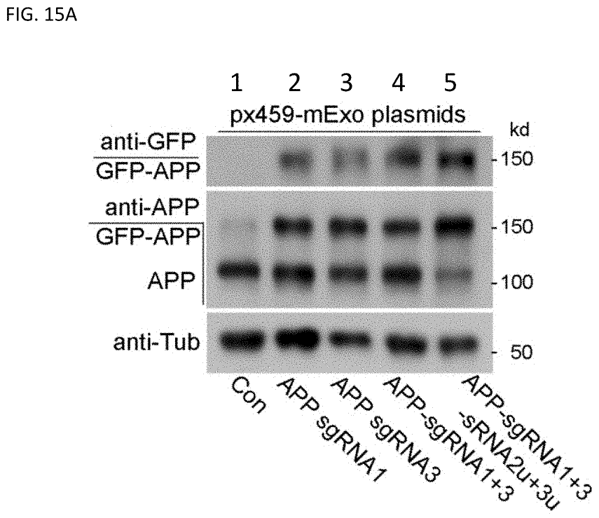

The Examples above show how the gene editing system can be used to incorporate an eGFP signal protein or a XIST gene into an endogenous genome. The gene editing system can also be used for the incorporation of a donor DNA molecule at the site of other genes. After identifying a genomic site of interest, (e.g., a genomic site causing a disease or disorder), the gene sequence can be analyzed to identify PAM sites near the genomic site of interest. Analysis of the gene sequence for PAM sites can be performed using any of a number of methods known in the art. Once PAM sites are identified in the endogenous genome, two sgRNA can be designed to target the Cas-exonuclease fusion protein to the endogenous genome at sites 5′ and 3′ to the genomic site of interest. Methods of designing sgRNAs while limiting off target effects are described herein. The donor DNA molecule (FIG. 14) can be designed after the identification of PAM sites and the design of the sgRNAs. The donor DNA molecule can be designed to have 5′ and 3′ homology arms that are homologous to the target genomic site for HDR. The homology arms can be designed with modifications at sites homologous to the endogenous target genomic sites, so as to not include a PAM site for the targeting of the Cas-exonuclease fusion protein, which avoids cleavage by the sgRNAs designed to cleave the target DNA molecule. A polynucleotide having an amino acid sequence encoding a Cas inhibitor can also be included in the donor DNA molecule. For knock in of a target gene of interest, the donor DNA molecule may also include a gene sequence encoding the target gene of interest, a mutation of a target gene of interest, or a fragment thereof. If desired, the gene editing system can be designed to insert the donor DNA molecule into the endogenous genome at a site where an endogenous gene promoter induces the expression of the donor DNA molecule. If an endogenous gene promoter cannot induce expression, one or more promoters can be incorporated into the donor DNA molecule and operably linked to the Cas inhibitor or target gene of interest to drive expression thereof. Examples of different promoters are well known in the art. A plasmid can be developed that includes the donor DNA molecule with target sites on the 5′ end and 3′ end of the donor DNA molecule, corresponding to target site A and target site B, respectively. Two sgRNAs, sgRNA donor A and sgRNA donor B, can be used to direct a Cas-exonuclease fusion protein to the plasmid for cleavage and subsequent release the donor DNA molecule, making it available for insertion into the endogenous genome. For delivery to a cell, a viral vector can be designed with polynucleotides having nucleic acid sequences encoding the four sgRNAs: two directed to the endogenous genome and two directed to release the donor DNA molecule, the Cas-exonuclease fusion protein, and the donor DNA molecule. Incorporation of the donor DNA molecule and the subsequent expression of the Cas inhibitor can be used to inhibit the activity of the Cas-exonuclease fusion protein, thereby limiting off target effects.

### Example 7

The gene editing system described herein can also be used to knock out a gene, or remove endogenous genomic material. For gene knock out, the gene editing system can be designed as described in Example 6, but with minor modifications. In this use, the donor DNA molecule can be prepared without a nucleic acid sequence encoding a target gene of interest. The donor DNA molecule can also contain a nucleic acid encoding the Cas inhibitor that, upon expression, would inhibit further activity of the Cas-exonuclease fusion protein.

### Example 8

The gene editing system described herein can be used to introduce a mutation into the genome of a subject (e.g., a non-human subject) to replicate a disease or disorder, such as Cystic Fibrosis, in the subject (e.g., for use in preparing an animal model of human disease). As an example, the gene editing system can be designed to replace the cystic fibrosis transmembrane conductance regulator (CFTR) gene of a subject (e.g., a pig) with a gene having a mutation that causes Cystic Fibrosis, such as the most common mutation, AF508. Possible PAM sites 5′ and 3′ to the CFTR gene can be identified using the methods described herein. After identifying PAM sites in the endogenous genome, two sgRNAs can be designed to direct the Cas-exonuclease fusion protein to the target genomic sites. Once the two target genomic sites are identified, a donor DNA molecule can be developed. The donor gene within the donor DNA molecule would be a CFTR gene having the three nucleotide deletion causing the AF508 mutation. The 5′ and 3′ homology arms would be homologous to the target genomic site for HDR. The homology arms can be designed with a modification to remove PAM sites, thereby avoiding targeting and cleavage of the donor DNA molecule by the sgRNA.

The donor DNA molecule can be incorporated into a plasmid for delivery. Two different sgRNA, sgRNA donor A and sgRNA donor B, ban be designed to direct the Cas-exonuclease fusion protein to the plasmid to cleave and release the donor DNA for insertion into the endogenous genome. One or more viral vectors can be designed with polynucleotides having nucleic acid sequences encoding the four sgRNAs: two directed to the endogenous genome and two directed to release the donor DNA molecule, the Cas-exonuclease fusion protein, and the donor DNA molecule. The one or more viral vectors can be delivered to the subject to be genetically modified, thereby allowing the gene editing system to perform HDR and to replicate Cystic Fibrosis in the subject.

### Example 9

A similar method as described in Example 8 can be used to remove a mutation causing Cystic Fibrosis from a subject (e.g., a human) suffering from the disease. For the treatment of Cystic Fibrosis, the donor gene can be designed to contain the wild-type sequence of the CFTR gene for replacement of the mutated CFTR gene. Upon insertion by HDR, the subject would no longer have a CFTR mutation, thereby treating the disease.

### Example 10

The previous examples show that the gene editing system of the present disclosure, which utilizes HDR, achieves improved donor nucleic acid insertion efficiency relative to prior systems for both gene knockin and gene knockdown. We have also demonstrated that the efficiency of the gene editing system is not cell-type dependent. As is shown in FIG. 15C, insertion of XIST (3 kb) at the col6a2 site is observed in 3 of 7 clones of an HEK 293 cell line. Similar findings were obtained with DS iPS following SNP-derived PAM targeting. Our data show that the modified CRISPR approach has utility in different cell types and can be used to remove and/or insert relatively large nucleic acid materials by HDR (on the order of several kb).

We also performed deep sequencing analysis of putative off-targeting sites to determine whether our gene editing system causes any significant off-target changes. Our data do not reveal any increased mutagenesis resulting from use of the modified mEXO CRISPR technique (FIG. 15D). *** indicates p≤0.001.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. All publications, patents, and patent applications mentioned in the above specification are hereby incorporated by reference to the same extent as if each individual publication, patent or patent application was specifically and individually indicated to be incorporated by reference in its entirety.

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

Other embodiments are within the claims.

1. A method of homology directed repair, wherein the method comprises:
a) delivering to a target cell a gene editing system comprising:
i) a first guide ribonucleic acid (RNA) directed to a first genomic site of an endogenous DNA molecule of the target cell,
ii) a second guide RNA directed to a second genomic site of the endogenous DNA molecule of the target cell,
iii) a plurality of fusion proteins comprising a first domain comprising an active RNA programmable nuclease and a second domain comprising an exonuclease, and, optionally, and
iv) a donor DNA molecule,
wherein the first guide RNA forms a first complex with a first said fusion protein at the first genomic site and the second guide RNA forms a second complex with a second said fusion protein at the second genomic site, and wherein the first and second complexes promote the homology directed repair by creating a lesion between the first and second genomic sites and, optionally, wherein the homology directed repair comprises insertion of the donor DNA molecule at the lesion between the first and second genomic sites.

2. The method of claim 1, wherein the first and second guide RNAs specifically hybridize to the first and second genomic sites, respectively.

3. The method of claim 1 or 2, wherein the first genomic site and the second genomic site are between 10-100000 nucleotide base pairs apart.

4. The method of any one of claims 1-3, wherein said first genomic site comprises a protospacer adjacent motif (PAM) recognition sequence positioned:
a) downstream from said first genomic site, and said second genomic site comprises a PAM recognition sequence downstream of said second genomic site;
b) downstream from said first genomic site, and said second genomic site comprises a PAM recognition sequence upstream of said second genomic site;
c) upstream from said first genomic site, and said second genomic site comprises a PAM recognition sequence upstream of said second genomic site; or
d) upstream from said first genomic site, and said second genomic site comprises a PAM recognition sequence downstream of said second genomic site.

5. The method of any one of claims 1-4, wherein said first and second guide RNAs are two single guide RNAs, wherein said first guide RNA targets a first strand of the endogenous DNA molecule, and said second guide RNA targets a complementary strand of the endogenous DNA molecule, and said first domain of the fusion protein cleaves each strand of the endogenous DNA molecule, thereby creating a double-stranded break, and said second domain of the fusion protein cleaves the terminal nucleic acids of each strand of the endogenous DNA molecule, thereby creating elongated single stranded nucleic acid overhangs.

6. The method of any one of claims 1-5, wherein a region between the first and second genomic sites is associated with a disease.

7. The method of any one of claims 1-6, wherein the gene editing system further comprises a third and fourth guide RNA.

8. The method of any one of claims 1-7, wherein the donor DNA molecule further comprises flanking regions modified to allow for specificity of targeting of one or more guide RNAs.

9. The method of claim 8, wherein the one or more guide RNAs are the third and fourth guide RNAs.

10. The method of claim 9, wherein the third guide RNA forms a complex with a first said fusion protein at a first said flanking region on the donor DNA molecule and the fourth guide RNA forms a complex with a second said fusion protein at a second said flanking region on the donor DNA molecule, and wherein said complexes cleave the donor DNA molecule at the flanking regions thereby releasing the donor DNA molecule.

11. The method of any one of claims 1-10, wherein the first domain is a Cas RNA programmable nuclease.

12. The method of claim 11, wherein the Cas RNA programmable nuclease is a Cas9 RNA programmable nuclease.

13. The method of any one of claims 1-12, wherein the second domain comprises an exonuclease selected from the group consisting of Lambda exonuclease, RecJf exonuclease, exonuclease III, exonuclease I, thermolabile exonuclease I, exonuclease T, exonuclease V (RecBCD), exonuclease VIII (truncated), exonuclease VII, nuclease BAL-31, T5 exonuclease, and T7 exonuclease.

14. The method of claim 13, wherein the exonuclease is Lambda exonuclease.

15. The method of any one of claims 1-14, wherein the method further comprises delivering an RNA programmable nuclease inhibitor to the target cell.

16. The method of claim 15, wherein the RNA programmable nuclease inhibitor is delivered as a nucleic acid comprising a sequence encoding the RNA programmable nuclease inhibitor.

17. The method of claim 15 or 16, wherein the donor DNA molecule comprises a polynucleotide sequence encoding the RNA programmable nuclease inhibitor.

18. The method of any one of claims 15-17, wherein insertion of the donor DNA molecule at the lesion between the first and second genomic sites promotes expression of the RNA programmable nuclease inhibitor in the target cell, thereby inhibiting activity of the RNA programmable nuclease.

19. The method of claim 15, wherein the RNA programmable nuclease inhibitor is delivered as a polypeptide.

20. The method of any one of claims 15-19, wherein the RNA programmable nuclease inhibitor is selected from the group consisting of AcrIIA1, AcrIIA2, AcrIIA3, AcrIIA5, AcrIIA5, AcrIIC1, AcrIIC2, or AcrIIC3.

21. The method of claim 20, wherein the RNA programmable nuclease is AcrIIA4.

22. The method of any one of claims 1-21, wherein the first or second genomic site comprises a nucleotide polymorphism.

23. The method of any one of claims 1-22, wherein the donor DNA molecule comprises a nucleic acid sequence encoding a gene not associated with a disease or disorder, wherein the homology directed repair comprises insertion of the donor DNA molecule at the lesion between the first and second genomic site, thereby correcting a nucleic acid sequence associated with a disease or disorder.

24. A nucleic acid comprising a polynucleotide comprising a nucleic acid sequence encoding a fusion protein comprising an RNA programmable nuclease and an exonuclease.

25. The nucleic acid of claim 24, further comprising a polynucleotide comprising a nucleic acid sequence encoding a first guide RNA and a second guide RNA.

26. The nucleic acid of claim 25, wherein the first and second guide RNA are directed to first and second genomic sites, respectively, of an endogenous DNA molecule of a cell.

27. The nucleic acid of any one of claims 24-26, further comprising a polynucleotide comprising a nucleic acid sequence encoding a donor DNA molecule.

28. The nucleic acid of any one of claims 24-27, further comprising a polynucleotide comprising a nucleic acid sequence encoding a third guide RNA and a fourth guide RNA.

29. The nucleic acid of claim 27 or 28, where the polynucleotide comprising a nucleic acid sequence encoding a donor DNA molecule further comprises flanking regions of said donor DNA molecule and wherein said flanking regions are modified to allow for specificity of targeting of one or more guide RNAs.

30. The nucleic acid of any one of claims 27-29, wherein the donor DNA molecule comprises a nucleic acid sequence encoding a region of a gene, wherein preferably the region lacks a mutation or polymorphism associated with a disease or disorder.

31. The nucleic acid of any one of claims 24-30, further comprising a promoter.

32. The nucleic acid of any one of claims 24-31, wherein the RNA programmable nuclease is a Cas RNA programmable nuclease.

33. The nucleic acid of claim 32, wherein the Cas RNA programmable nuclease is a Cas9 RNA programmable nuclease.

34. The nucleic acid of any one of claims 24-33, wherein the exonuclease is selected from the group consisting of Lambda exonuclease, RecJf exonuclease, exonuclease III, exonuclease I, thermolabile exonuclease I, exonuclease T, exonuclease V (RecBCD), exonuclease VIII (truncated), exonuclease VII, nuclease BAL-31, T5 exonuclease, and T7 exonuclease.

35. The nucleic acid of claim 34, wherein the exonuclease is Lambda exonuclease.

36. The nucleic acid of any one of claims 24-35, wherein the nucleic acid comprises a nucleic acid encoding a fusion protein comprising an RNA programmable nuclease and an exonuclease, wherein the RNA programmable nuclease and the exonuclease are joined directly or through a linker.

37. The nucleic acid of any one of claims 27-36, wherein the donor DNA molecule comprises a polynucleotide sequence encoding the RNA programmable nuclease inhibitor.

38. The nucleic acid of claim 37, wherein the RNA programmable nuclease is selected from the group consisting of AcrIIA1, AcrIIA2, AcrIIA3, AcrIIA5, AcrIIA5, AcrIIC1, AcrIIC2, or AcrIIC3.

39. The nucleic acid of claim 38, wherein the RNA programmable nuclease is AcrIIA4.

40. A vector comprising a polynucleotide comprising a nucleic acid sequence encoding a fusion protein comprising an RNA programmable nuclease and an exonuclease.

41. The vector of claim 40, wherein the vector further comprises a polynucleotide comprising a nucleic acid sequence encoding a first and second guide RNA directed to first and second genomic sites, respectively, of an endogenous DNA molecule of a cell.

42. The vector of claim 40 or 41, wherein the vector further comprises a polynucleotide comprising a nucleic acid sequence encoding a third guide RNA and a fourth guide RNA.

43. The vector of any one of claims 40-42, wherein the vector further comprises a polynucleotide comprising a nucleic acid sequence encoding a donor DNA molecule.

44. The vector of claim 43, wherein flanking regions of said donor DNA molecule are modified to allow for specificity of targeting of one or more guide RNAs.

45. The vector of claim 43 or 44, wherein the donor DNA molecule further comprises a polynucleotide comprising a nucleic acid sequence encoding an RNA programmable nuclease inhibitor.

46. The vector of any one of claims 40-45, wherein the RNA programmable nuclease is a Cas RNA programmable nuclease.

47. The vector of claim 46, wherein the Cas RNA programmable nuclease is a Cas9 RNA programmable nuclease.

48. The vector of any one of claims 40-47, wherein the exonuclease is selected from the group consisting of Lambda exonuclease, RecJf exonuclease, exonuclease III, exonuclease I, thermolabile exonuclease I, exonuclease T, exonuclease V (RecBCD), exonuclease VIII (truncated), exonuclease VII, nuclease BAL-31, T5 exonuclease, and T7 exonuclease.

49. The vector of claim 48, wherein the exonuclease is Lambda exonuclease.

50. The vector of anyone of claims 45-49, wherein the RNA programmable nuclease inhibitor is selected from the group consisting of AcrIIA1, AcrIIA2, AcrIIA3, AcrIIA5, AcrIIA5, AcrIIC1, AcrIIC2, or AcrIIC3.

51. The vector of claim 50, wherein the RNA programmable nuclease is AcrIIA4.

52. The vector of claims 43-51, wherein the donor DNA molecule comprises a polynucleotide comprising a nucleic acid sequence encoding a region of a gene, wherein preferably the region lacks a mutation or polymorphism associated with a disease or disorder.

53. The vector of any one of claims 40-52, wherein the RNA programmable nuclease and the exonuclease are joined directly or through a linker.

54. A vector comprising the nucleic acid of any one of claims 24-39.

55. The vector of any one of claims 40-54, wherein the vector is an expression vector or a viral vector.

56. The vector of claim 55, wherein the viral vector is a lentiviral vector.

57. A composition comprising:
a) a first guide ribonucleic acid (RNA) directed to a first genomic site of an endogenous DNA molecule of a target cell,
b) a second guide RNA directed to a second genomic site of the endogenous DNA molecule of the target cell,
c) a plurality of fusion proteins, wherein each fusion protein comprises a first domain comprising an active RNA programmable nuclease and a second domain comprising an exonuclease, and, optionally,
d) a donor DNA molecule.

58. The composition of claim 57, wherein the first guide RNA is in a first complex with a first said fusion protein and the second guide RNA is in a second complex with a second said fusion protein,
wherein the first and second complexes are configured to promote homology directed repair of the endogenous DNA molecule, optionally, upon insertion of the donor DNA molecule between the first and second genomic sites.

59. The composition of claim 57 or 58, wherein the donor DNA molecule comprises a nucleic acid sequence encoding a region of a gene, wherein preferably the region lacks a mutation or polymorphism associated with a disease or disorder.

60. The composition of any one of claims 57-59, further comprising an RNA programmable nuclease inhibitor.

61. The composition of claim 60, wherein the RNA programmable nuclease inhibitor is selected from the group consisting of AcrIIA1, AcrIIA2, AcrIIA3, AcrIIA5, AcrIIA5, AcrIIC1, AcrIIC2, or AcrIIC3.

62. The composition of claim 61, wherein the RNA programmable nuclease is AcrIIA4.

63. A composition comprising:
a) a first polynucleotide comprising a nucleic acid sequence encoding a first guide ribonucleic acid (RNA) directed to a first genomic site of an endogenous DNA molecule of a target cell;
b) a second polynucleotide comprising a nucleic acid sequence encoding a second guide RNA directed to a second genomic site of the endogenous DNA molecule of the target cell;
c) a third polynucleotide comprising a nucleic acid sequence encoding a fusion protein comprising a first domain comprising an active RNA programmable nuclease and a second domain comprising an exonuclease; and, optionally,
d) a fourth polynucleotide comprising a nucleic acid sequence encoding a donor DNA molecule.

64. The composition of claim 63, wherein the first guide RNA is configured to form a first complex with a first said fusion protein and the second guide RNA is configured to form a second complex with a second said fusion protein, and wherein the first and second complexes are configured to promote homology directed repair of the endogenous DNA molecule, optionally, upon insertion of the donor DNA molecule between the first and second genomic sites.

65. The composition of claim 63 or 64, wherein the active RNA programmable nuclease and the exonuclease are joined directly or through a linker.

66. The composition of any one of claims 63-65, further comprising a fifth polynucleotide comprising a nucleic acid sequence encoding an RNA programmable nuclease inhibitor or wherein the nucleic acid sequence of the fourth polynucleotide further encodes an RNA programmable nuclease inhibitor

67. The composition of any one of claims 63-66, further comprising:
i) a sixth polynucleotide comprising a nucleic acid sequence encoding a third guide RNA, and
ii) a seventh polynucleotide comprising a nucleic acid sequence encoding a fourth guide RNA.

68. The composition of any one of claims 63-67, wherein the polynucleotide comprising a nucleic acid sequence encoding the donor DNA molecule further comprises flanking regions modified to allow for specificity of targeting of one or more guide RNAs.

69. The composition of claim 68, wherein the one or more guide RNAs are the third and fourth guide RNAs.

70. The composition of claim 69, wherein the third guide RNA is configured to form a complex with a first said fusion protein at a first said flanking region on the donor DNA molecule and the fourth guide RNA is configured to form a complex with a second said fusion protein at a second said flanking region on the donor DNA molecule, and wherein said complexes cut the donor DNA molecule at the flanking regions, thereby releasing the donor DNA molecule.

71. The composition of any one of claims 63-70, wherein the donor DNA molecule comprises a nucleic acid sequence encoding a region of a gene, wherein preferably the region lacks a mutation or polymorphism associated with a disease or disorder.

72. The composition of any one of claims 63-71, wherein the RNA programmable nuclease is a Cas RNA programmable nuclease.

73. The composition of claim 72, wherein the Cas RNA programmable nuclease is a Cas9 RNA programmable nuclease.

74. The composition of any one of claims 63-73, wherein the exonuclease is selected from the group consisting of Lambda exonuclease, RecJf exonuclease, exonuclease III, exonuclease I, thermolabile exonuclease I, exonuclease T, exonuclease V (RecBCD), exonuclease VIII (truncated), exonuclease VII, nuclease BAL-31, T5 exonuclease, and T7 exonuclease.

75. The composition of claim 74, wherein the exonuclease is Lambda exonuclease.

76. The composition of any one of claims 66-75, wherein the RNA programmable nuclease inhibitor is selected from the group consisting of AcrIIA1, AcrIIA2, AcrIIA3, AcrIIA5, AcrIIA5, AcrIIC1, AcrIIC2, or AcrIIC3.

77. The composition of claim 76, wherein the RNA programmable nuclease is AcrIIA4.

78. A pharmaceutical composition comprising the nucleic acid of any one of claims 24-39, the vector of any one of claims 40-56, or the composition of any one of claims 57-77 and a pharmaceutically acceptable carrier, excipient, or diluent.

79. A kit comprising the nucleic acid of any one of claims 24-39, the vector of any one of claims 40-56, the composition of any one of claims 57-77, or the pharmaceutical composition of claim 78.

80. The kit of claim 79, wherein the kit comprises the first and second guide RNAs, wherein the first and second guide RNAs are targeted to a genomic site of an endogenous DNA molecule of a target cell causing a disease.

81. The kit of claim 80, wherein the first and second guide RNAs target a nucleotide polymorphism at the genomic site of the endogenous DNA molecule of the target cell.

82. A fusion protein comprising a first domain comprising an active RNA programmable nuclease and a second domain comprising an exonuclease.

83. The fusion protein of claim 82, wherein the first domain is a Cas RNA programmable nuclease.

84. The fusion protein of claim 83, wherein the Cas RNA programmable nuclease is a Cas9 RNA programmable nuclease.

85. The fusion protein of any one of claims 82-84, wherein the second domain comprises an exonuclease selected from the group consisting of Lambda exonuclease, RecJf exonuclease, exonuclease III, exonuclease I, thermolabile exonuclease I, exonuclease T, exonuclease V (RecBCD), exonuclease VIII (truncated), exonuclease VII, nuclease BAL-31, T5 exonuclease, and T7 exonuclease.

86. The fusion protein of claim 85, wherein the exonuclease is Lambda exonuclease.

87. The fusion protein of any one of claims 82-86, wherein the two domains are joined directly or through a linker.

88. The method of claims 1-23, wherein the homology directed repair treats a disease or disorder.

89. The method of claim 88, wherein the disease or disorder is selected from a group consisting of age-related macular degeneration; a blood or coagulation disease or disorder; a cell dysregulation or oncology disease or disorder; a developmental disorder; drug addiction; an inflammation or immune related disease or disorder; a metabolic, liver, kidney, or protein disease or disorder; a muscular or skeletal disease or disorder; a neurological or neuronal disease or disorder; a neoplasia; an ocular disease or disorder; schizophrenia; epilepsy; Duchenne muscular dystrophy; a viral disease or disorder, such as AIDS (acquired immunodeficiency syndrome); an autoimmune disorder; and an alpha 1-antitrypsin deficiency.

90. The method of claim 89, wherein the blood or coagulation disease or disorder is:
a) anemia wherein, preferable, the gene is CDAN1, CDA1, RPS19, DBA, PKLR, PK1, NT5C3, UMPH1, PSN1, RHAG, RH50A, NRAMP2, SPTB, ALAS2, ANH1, ASB, ABCB7, ABC7, and/or ASAT;
b) bare lymphocyte syndrome, wherein, preferably, the gene is TAPBP, TPSN, TAP2, ABCB3, PSF2, RING11, MHC2TA, C2TA, RFX5, RFXAP, or RFX5;
c) a bleeding disorder, wherein, preferably, the gene is TBXA2R, P2RX1, or P2X1:
d) a hemolytic anemia, such as a complement Factor H deficiency disease, e.g., a typical hemolytic anemia syndrome (aHUS), wherein, preferably, the gene is HF1, CFH, or HUS;
e) a factor V or factor VIII deficiency disease, wherein, preferably, the gene is MCFD2;
f) a factor VII deficiency disease, wherein, preferably, the gene is F7;
g) a factor X deficiency disease, wherein, preferably, the gene is F10;
h) a factor XI deficiency disease, wherein, preferably, the gene is F11;
i) a factor XII deficiency disease, wherein, preferably, the gene is F12 or HAF;
j) a factor XIIIA deficiency disease, wherein, preferably, the gene is F13A1 or F13A;
k) a factor XIIIB deficiency disease, wherein, preferably, the gene is F13B;
l) Fanconi anemia, wherein, preferably, the gene is FANCA, FACA, FA1, FA, FAA, FAAP95, FAAP90, FLJ34064, FANCB, FANCC, FACC, BRCA2, FANCD1, FANCD2, FANCD, FACD, FAD, FANCE, FACE, FANCF, XRCC9, FANCG, BRIP1, BACH1, FANCJ, PHF9, FANCL, FANCM, or KIAA1596;
m) a hemophagocytic or lymphohistiocytosis disorder, wherein, preferably, the gene is PRF1, HPLH2, UNC13D, MUNC13-4, HPLH3, HLH3, or FHL3;
n) hemophilia A, wherein, preferably, the gene is F8, F8C, or HEMA;
o) hemophilia B, wherein, preferably, the gene is F9 or HEMB;
p) a hemorrhagic disorder, wherein, preferably, the gene is PI, ATT, F5;
q) a leukocyte deficiency or disorder, wherein, preferably, the gene is ITGB2, CD18, LCAMB, LAD, EIF2B1, EIF2BA, EIF2B2, EIF2B3, EIF2B5, LVWM, CACH, CLE, or EIF2B4;
r) sickle cell anemia, wherein, preferably, the gene is HBB; or
s) thalassemia, wherein, preferably, the gene is HBA2, HBB, HBD, LCRB, or HBA1.

91. The method of claim 89, wherein the cell dysregulation or oncology disease is:
a) B-cell non-Hodgkin lymphoma, wherein, preferably, the gene is BCL7A or BCL7; or
b) a leukemia, wherein, preferably, the gene is TAL1 TCL5, SCL, TAL2, FLT3, NBS1, NBS, ZNFN1A1, IK1, LYF1, HOXD4, HOX4B, BCR, CML, PHL, ALL, ARNT, KRAS2, RASK2, GMPS, AF10, ARHGEF12, LARG, KIAA0382, CALM, CLTH, CEBPA, CEBP, CHIC2, BTL, FLT3, KIT, PBT, LPP, NPM1, NUP214, D9S46E, CAN, CAIN, RUNX1, CBFA2, AML1, WHSC1L1, NSD3, FLT3, AF1Q, NPM1, NUMA1, ZNF145, PLZF, PML, MYL, STAT5B, AF10, CALM, CLTH, ARL11, ARLTS1, P2RX7, P2X7, BCR, CML, PHL, ALL, GRAF, NF1, VRNF, WSS, NFNS, PTPN11, PTP2C, SHP2, NS1, BCL2, CCND1, PRAD1, BCL1, TCRA, GATA1, GF1, ERYF1, NFE1, ABL1, NQO1, DIA4, NMOR1, NUP214, D9S46E, CAN, or CAIN.

92. The method of claim 89, wherein the developmental disease is:
a) Angelman syndrome, wherein, preferably, the gene is UBE3A or a 15q11-13 deletion;
b) Canavan disease, wherein, preferably, the gene is ASPA;
c) Cri-du-chat syndrome, wherein, preferably, the gene is 5P− (5p minus) or CTNND2;
d) Down syndrome, wherein, preferably, the gene is Trisomy 21;
e) Klinefelter syndrome, wherein, preferably, the gene is XXY or two or more X chromosomes in males;
f) Prader-Willi syndrome, wherein, preferably, the gene is deletion of chromosome 15 segment or a duplication of maternal chromosome 15; or
g) Turner syndrome where the gene is monosomy X or SHOX.

93. The method of claim 89, wherein the disease or disorder is a drug addiction, wherein, preferably, the gene is PRKCE, DRD2, DRD4, ABAT (alcohol), GRIA2, GRM5, GRIN1, HTR1B, GRIN2A, DRD3, PDYN, GRIA1 (alcohol).

94. The method of claim 89, wherein the inflammation or immune related disease is:
a) autoimmune lymphoproliferative syndrome, wherein, preferably, the gene TNFRSF6, APT1, FAS, CD95, or ALPS1A;
b) combined immuno-deficiency, wherein, preferably, the gene is IL2RG, SCIDX1, SCIDX, or IMD4;
c) an immuno-deficiency, wherein, preferably, the gene is CD3E, CD3G, AICDA, AID, HIGM2, TNFRSF5, CD40, UNG, DGU, HIGM4, TNFSF5, CD40LG, HIGM1, IGM, FOXP3, IPEX, AIID, XPID, PIDX, TNFRSF14B, or TACI;
d) inflammation wherein, preferably, the gene is IL-10, IL-1 (IL-1a, IL-1 b), IL-13, IL-17 (IL-17a (CTLA8), IL-17b, IL-17c, IL-17d, IL-17f), Il-23, CX3CR1, PTPN22, TNFa, NOD2/CARD15 for IBD, IL-6, IL-12 (IL-12a, IL-12b), CTLA4, or CX3CL1; or
e) severe combined immunodeficiency disease, wherein, preferably, the gene is JAK3, JAKL, DCLRE1C, ARTEMIS, SCIDA, RAG1, RAG2, ADA, PTPRC, CD45, LCA, IL7R, CD3D, T3D, IL2RG, SCIDX1, SCIDX, or IMD4.

95. The method of claim 89, wherein the metabolic, liver, kidney, or protein disease is:
a) amyloid neuropathy, wherein, preferably, the gene is TTR or PALB;
b) amyloidosis, wherein, preferably, the gene is APOA1, APP, AAA, CVAP, AD1, GSN, FGA, LYZ, TTR, or PALB;
c) cirrhosis, wherein, preferably, the gene is KRT18, KRT8, CIRH1A, NAIC, TEX292, or KIAA1988;
d) cystic fibrosis, wherein, preferably, the gene is CFTR, ABCC7, CF, or MRP7;
e) a glycogen storage disease, wherein, preferably, the gene is SLC2A2, GLUT2, G6PC, G6PT, G6PT1, GAA, LAMP2, LAMPB, AGL, GDE, GBE1, GYS2, PYGL, or PFKM;
f) hepatic adenoma, wherein, preferably, the gene is TCF1, HNF1A, or MODY3;
g) an early onset neurologic disorder, wherein, preferably, the gene is SCOD1 or SCO1;
h) hepatic lipase deficiency, wherein, preferably, the gene is LIPC;
i) hepato-blastoma cancer, wherein, preferably, the gene is CTNNB1, PDGFRL, PDGRL, PRLTS, AXIN1, AXIN, CTNNB1, TP53, P53, LFS1, IGF2R, MPRI, MET, CASP8, or MCH5;
j) medullary cystic kidney disease, wherein, preferably, the gene is UMOD, HNFJ, FJHN, MCKD2, or ADMCKD2;
k) phenylketonuria, wherein, preferably, the gene is PAH, PKU1, QDPR, DHPR, or PTS; or
l) polycystic kidney or hepatic disease, wherein, preferably, the gene is FCYT, PKHD1, ARPKD, PKD1, PKD2, PKD4, PKDTS, PRKCSH, G19P1, PCLD, or SEC63.

96. The method of claim 89, wherein the muscular or skeletal disease is:
a) Becker muscular dystrophy, wherein, preferably, the gene is DMD, BMD, or MYF6;
b) Duchenne muscular dystrophy, wherein, preferably, the gene is DMD or BMD;
c) Emery-Dreifuss muscular dystrophy, wherein, preferably, the gene is LMNA, LMN1, EMD2, FPLD, CMD1A, HGPS, LGMD1B, LMNA, LMN1, EMD2, FPLD, or CMD1A;
d) Facio-scapulohumeral muscular dystrophy, wherein, preferably, the gene is FSHMD1A or FSHD1A;
e) muscular dystrophy, wherein, preferably, the gene is FKRP, MDC1C, LGMD2I, LAMA2, LAMM, LARGE, KIAA0609, MDC1D, FCMD, TTID, MYOT, CAPN3, CANP3, DYSF, LGMD2B, SGCG, LGMD2C, DMDA1, SCG3, SGCA, ADL, DAG2, LGMD2D, DMDA2, SGCB, LGMD2E, SGCD, SGD, LGMD2F, CMD1L, TCAP, LGMD2G, CMD1N, TRIM32, HT2A, LGMD2H, FKRP, MDC1C, LGMD2I, TTN, CMD1G, TMD, LGMD2J, POMT1, CAV3, LGMD1C, SEPN1, SELN, RSMD1, PLEC1, PLTN, or EBS1;
f) osteopetrosis, wherein, preferably, the gene is LRP5, BMND1, LRP7, LR3, OPPG, VBCH2, CLCN7, CLC7, OPTA2, OSTM1, GL, TCIRG1, TIRC7, OC116, or OPTB1;
g) muscular atrophy, wherein, preferably, the gene is VAPB, VAPC, ALS8, SMN1, SMA1, SMA2, SMA3, SMA4, BSCL2, SPG17, GARS, SMAD1, CMT2D, HEXB, IGHMBP2, SMUBP2, CATF1, or SMARD1; or
h) Tay-Sachs disease, wherein, preferably, the gene is HEXA.

97. The method of claim 89, wherein the neurological and neuronal disease is:
a) amyotrophic lateral sclerosis (ALS), wherein, preferably, the gene is SOD1, ALS2, STEX, FUS, TARDBP, or VEGF (VEGF-a, VEGF-b, VEGF-c);
b) Alzheimer's disease, wherein, preferably, the gene is APP, AAA, CVAP, AD1, APOE, AD2, PSEN2, AD4, STM2, APBB2, FE65L1, NOS3, PLAU, URK, ACE, DCP1, ACE1, MPO, PACIP1, PAXIP1L, PTIP, A2M, BLMH, BMH, PSEN1, orAD3;
c) autism, wherein, preferably, the gene is Mecp2, BZRAP1, MDGA2, Sema5A, Neurexin 1, GLO1, MECP2, RTT, PPMX, MRX16, MRX79, NLGN3, NLGN4, KIAA1260, or AUTSX2;
d) Fragile X Syndrome, wherein, preferably, the gene is FMR2, FXR1, FXR2, or mGLUR5;
e) Huntington's disease or a Huntington's disease like disorder, wherein, preferably, the gene is HD, IT15, PRNP, PRIP, JPH3, JP3, HDL2, TBP, or SCA17;
f) Parkinson's disease, wherein, preferably, the gene is NR4A2, NURR1, NOT, TINUR, SNCAIP, TBP, SCA17, SNCA, NACP, PARK1, PARK4, DJ1, PARK7, LRRK2, PARK8, PINK1, PARK6, UCHL1, PARK5, SNCA, NACP, PARK1, PARK4, PRKN, PARK2, PDJ, DBH, or NDUFV2;
g) Rett syndrome, wherein, preferably, the gene is MECP2, RTT, PPMX, MRX16, MRX79, CDKL5, STK9, MECP2, RTT, PPMX, MRX16, MRX79, α-Synuclein, or DJ-1;
h) schizophrenia, wherein, preferably, the gene is NRG1, ERB4, CPLX1), TPH1, TPH2, Neurexin 1, GSK3, GSK3a, GSK3b, 5-HTT (SLC6A4), COMT, DRD (DRD1a), SLC6A3, DAOA, DTNBP1, or DAO (DAO1);
i) secretase related disorders, wherein, preferably, the gene is APH-1 (alpha and beta), presenilin (PSEN1), nicastrin (NCSTN), PEN-2, NOS1, PARP1, NAT1, or NAT2; or
j) trinucleotide repeat disorders, wherein, preferably, the gene is HTT, SBMA/SMAX1/AR, FXN/X25, ATX3, ATXN1, ATXN2, DMPK, Atrophin-1, Atn1, CBP, VLDLR, ATXN7, or ATXN10.

98. The method of claim 89, wherein the disease or disorder is neoplasia, wherein, preferably, the gene is PTEN, ATM, ATR, EGFR, ERBB2, ERBB3, ERBB4, Notch1, Notch2, Notch3, Notch4, AKT, AKT2, AKT3, HIF, HIF1a, HIF3a, MET, HRG, Bcl2, PPAR alpha, PPAR gamma, WT1 (Wilms Tumor), FGF1, FGF2, FGF3, FGF4, FGF5, CDKN2a, APC, RB (retinoblastoma), MEN1, VHL, BRCA1, BRCA2, AR (androgen receptor), TSG101, IGF, IGF receptor, IGF1 (4 variants), IGF2 (3 variants), IGF 1 receptor, IGF 2 receptor, BAX, BCL2, caspase 1, 2, 3, 4, 6, 7, 8, 9, 12, KRAS, or APC.

99. The method of claim 89, wherein the ocular disease is:
a) age-related macular degeneration, wherein, preferably, the gene is Aber, CCL2, CC2, CP (ceruloplasmin), TIMP3, cathepsinD, VLDLR, or CCR2;
b) cataract, wherein, preferably, the gene is CRYAA, CRYA1, CRYBB2, CRYB2, PITX3, BFSP2, CP49, CP47, CRYAA, CRYA1, PAX6, AN2, MGDA, CRYBA1, CRYB1, CRYGC, CRYG3, CCL, LIM2, MP19, CRYGD, CRYG4, BFSP2, CP49, CP47, HSF4, CTM, HSF4, CTM, MIP, AQPO, CRYAB, CRYA2, CTPP2, CRYBB1, CRYGD, CRYG4, CRYBB2, CRYB2, CRYGC, CRYG3, CCL, CRYAA, CRYA1, GJA8, CX50, CAE1, GJA3, CX46, CZP3, CAE3, CCM1, CAM, or KRIT1;
c) corneal clouding or corneal dystrophy, wherein, preferably, the gene is APOA1, TGFBI, CSD2, CDGG1, CSD, BIGH3, CDG2, TACSTD2, TROP2, M1 S1, VSX1, RINX, PPCD, PPD, KTCN, COL8A2, FECD, PPCD2, PIP5K3, or CFD;
d) cornea plana (congenital), wherein, preferably, the gene is KERA or CNA2;
e) glaucoma, wherein, preferably, the gene is MYOC, TIGR, GLC1A, JOAG, GPOA, OPTN, GLC1E, FIP2, HYPL, NRP, CYP1B1, GLC3A, OPA1, NTG, NPG, CYP1B1, or GLC3A;
f) Leber congenital amaurosis, wherein, preferably, the gene is CRB1, RP12, CRX, CORD2, CRD, RPGRIP1, LCA6, CORD9, RPE65, RP20, AIPL1, LCA4, GUCY2D, GUC2D, LCA1, CORD6, RDH12, or LCA3; or
g) macular dystrophy, wherein, preferably, the gene is ELOVL4, ADMD, STGD2, STGD3, RDS, RP7, PRPH2, PRPH, AVMD, AOFMD, or VMD2.

100. The method of claim 89, wherein the disease or disorder is schizophrenia, wherein, preferably, the gene is neuregulin1 (NRG1), ERB4, Complexin1 (CPLX1), TPH1, TPH2, NRXN1, GSK3, GSK3a, or GSK3b.

101. The method of claim 89, wherein the disease or disorder is epilepsy, wherein, preferably, the gene is EPM2A, MELF, EPM2, NHLRC1, EPM2A, or EPM2B.

102. The method of claim 89, wherein the disease is Duchenne muscular dystrophy, wherein, preferably, the gene is DMD or BMD.

103. The method of claim 89, wherein the viral disease or disorder is:
a) AIDS, wherein, preferably, the gene is KIR3DL1, NKAT3, NKB1, AMB11, KIR3DS1, IFNG, CXCL12, or SDF1
b) caused by human immunodeficiency virus (HIV), wherein, preferably, the gene is CCL5, SCYA5, D17S136E, or TCP228;
c) HIV susceptibility or infection, wherein, preferably, the gene is IL10, CSIF, CMKBR2, CCR2, CMKBR5, or CCCKR5 (CCR5).

104. The method of claim 89, wherein the disease or disorder is alpha 1-antitrypsin deficiency, wherein, preferably, the gene is SERPINA1 [serpin peptidase inhibitor, clade A (alpha-1 antiproteinase, antitrypsin), member 1], SERPINA2, SERPINA3, SERPINA5, SERPINA6, or SERPINA7.

105. The method of any one of claims 1-23, wherein the homology directed repair treats a cellular dysfunction.

106. The method of claim 105, wherein the cellular dysfunction is associated with PI3K/AKT signaling, ERK/MAPK signaling, glucocorticoid receptor signaling, axonal guidance signaling, ephrin receptor signaling, actin cytoskeleton signaling, Huntington's disease signaling, apoptosis signaling, B cell receptor signaling, leukocyte extravasation signaling, integrin signaling, acute phase response signaling, PTEN signaling, p53 signaling, aryl hydrocarbon receptor signaling, xenobiotic metabolism signaling, SAPK/JNK signaling, PPAr/RXR signaling, NF-KB signaling, neuregulin signaling, Wnt or beta catenin signaling, insulin receptor signaling, IL-6 signaling, hepatic cholestasis, IGF-1 signaling, NRF2-mediated oxidative stress response, hepatic signaling, fibrosis or hepatic stellate cell activation, PPAR signaling, Fc Epsilon RI signaling, G-protein coupled receptor signaling, inositol phosphate metabolism, PDGF signaling, VEGF signaling, natural killer cell signaling, cell cycle G1/S checkpoint regulation, T cell receptor signaling, death receptor signaling, FGF signaling, GM-CSF signaling, amyotrophic lateral sclerosis signaling, JAK/Stat signaling, nicotinate or nicotinamide metabolism, chemokine signaling, IL-2 signaling, synaptic long term depression, estrogen receptor signaling, protein ubiquitination pathway, IL-10 signaling, VDR/RXR activation, TGF-beta signaling, toll-like receptor signaling, p38 MAPK signaling, neurotrophin/TRK signaling, FXR/RXR Activation, synaptic long term potentiation, calcium signaling, EGF signaling, hypoxia signaling in the cardiovascular system, LPS/IL-1 mediated inhibition of RXR function, LXR/RXR activation, amyloid processing, IL-4 signaling, cell cycle G2/M DNA damage checkpoint regulation, nitric oxide signaling in the cardiovascular system, purine metabolism, cAMP-mediated signaling, mitochondrial dysfunction notch signaling, endoplasmic reticulum stress pathway, pyrimidine metabolism, Parkinson's signaling, cardiac or beta adrenergic signaling, glycolysis or gluconeogenesis, interferon signaling, sonic hedgehog signaling, glycerophospholipid metabolism, phospholipid degradation, tryptophan metabolism, lysine degradation, nucleotide excision repair pathway, starch and sucrose metabolism, amino sugars metabolism, arachidonic acid metabolism, circadian rhythm signaling, coagulation system, dopamine receptor signaling, glutathione metabolism, glycerolipid metabolism, linoleic acid metabolism, methionine metabolism, pyruvate metabolism, arginine and proline metabolism, eicosanoid signaling, fructose and mannose metabolism, galactose metabolism, stilbene, coumarine and lignin biosynthesis, antigen presentation, pathway, biosynthesis of steroids, butanoate metabolism, citrate cycle, fatty acid metabolism, histidine metabolism, inositol metabolism, metabolism of xenobiotics by cytochrome p450, methane metabolism, phenylalanine metabolism, propanoate metabolism, selenoamino acid metabolism, sphingolipid metabolism, aminophosphonate metabolism, androgen or estrogen metabolism, ascorbate or aldarate metabolism, bile acid biosynthesis, cysteine metabolism, fatty acid biosynthesis, glutamate receptor signaling, NRF2-mediated oxidative stress response, pentose phosphate pathway, pentose and glucuronate interconversions, retinol metabolism, riboflavin metabolism, tyrosine metabolism, ubiquinone biosynthesis, valine, leucine and isoleucine degradation, glycine, serine and threonine metabolism, lysine degradation, pain/taste, pain, mitochondrial function, or developmental neurology.

107. The method of claim 105, wherein the cellular dysfunction is associated with:
i) PI3K/AKT signaling, wherein, preferably, the gene is PRKCE, ITGAM, ITGA5, IRAK1, PRKAA2, EIF2AK2, PTEN, EIF4E, PRKCZ, GRK6, MAPK1, TSC1, PLK1, AKT2, IKBKB, PIK3CA, CDK8, CDKN1B, NFKB2, BCL2, PIK3CB, PPP2R1A, MAPK8, BCL2L1, MAPK3, TSC2, ITGA1, KRAS, EIF4EBP1, RELA, PRKCD, NOS3, PRKAA1, MAPK9, CDK2, PPP2CA, PIM1, ITGB7, YWHAZ, ILK, TP53, RAF1., IKBKG, RELB, DYRK1A, CDKN1A, ITGB1, MAP2K2, JAK1, AKT1, JAK2, PIK3R1, CHUK, PDPK1, PPP2R5C, CTNNB1., MAP2K1, NFKB1, PAK3, ITGB3, CCND1, GSK3A, FRAP1, SFN, ITGA2, TTK, CSNK1A1, BRAF, GSK3B, AKT3, FOXO1, SGK, HSP90AA1, or RPS6KB1;
ii) ERK/MAPK signaling, wherein, preferably, the gene is PRKCE, ITGAM, ITGA5, HSPB1, IRAK1, PRKAA2, EIF2AK2, RAC1, RAP1A, TLN1, EIF4E, ELK1, GRK6, MAPK1, RAC2, PLK1, AKT2, PIK3CA, CDK8, CREB1, PRKCI, PTK2, FOS, RPS6KA4, PIK3CB, PPP2R1A, PIK3C3, MAPK8, MAPK3, ITGA1, ETS1, KRAS, MYCN, EIF4EBP1, PPARG, PRKCD, PRKAA1, MAPK9, SRC, CDK2, PPP2CA, PIM1, PIK3C2A, ITGB7, YWHAZ, PPP1CC, KSR1, PXN, RAF1, FYN, DYRK1A, ITGB1, MAP2K2, PAK4, PIK3R1, STAT3, PPP2R5C, MAP2K1, PAK3, ITGB3, ESR1, ITGA2, MYC, TTK, CSNK1A1, CRKL, BRAF, ATF4, PRKCA, SRF, STAT1, or SGK;
iii) glucocorticoid receptor signaling, wherein, preferably, the gene is RAC1, TAF4B, EP300, SMAD2, TRAF6, PCAF, ELK1, MAPK1, SMAD3, AKT2, IKBKB, NCOR2, UBE2I, PIK3CA, CREB1, FOS, HSPA5, NFKB2, BCL2, MAP3K14, STAT5B, PIK3CB, PIK3C3, MAPK8, BCL2L1, MAPK3, TSC22D3, MAPK10, NRIP1, KRAS, MAPK13, RELA, STAT5A, MAPK9, NOS2A, PBX1, NR3C1, PIK3C2A, CDKN1C, TRAF2, SERPINE1, NCOA3, MAPK14, TNF, RAF1, IKBKG, MAP3K7, CREBBP, CDKN1A, MAP2K2, JAK1, IL8, NCOA2, AKT1, JAK2, PIK3R1, CHUK, STAT3, MAP2K1, NFKB1, TGFBR1, ESR1, SMAD4, CEBPB, JUN, AR, AKT3, CCL2, MMP1, STAT1, 1L6, or HSP90AA1;
iv) axonal guidance signaling, wherein, preferably, the gene is PRKCE, ITGAM, ROCK1, ITGA5, CXCR4, ADAM12, IGF1, RAC1, RAP1A, E1F4E, PRKCZ, NRP1, NTRK2, ARHGEF7, SMO, ROCK2, MAPK1, PGF, RAC2, PTPN11, GNAS, AKT2, PIK3CA, ERBB2, PRKC1, PTK2, CFL1, GNAQ, PIK3CB, CXCL12, PIK3C3, WNT11, PRKD1, GNB2L1, ABL1, MAPK3, ITGA1, KRAS, RHOA, PRKCD, PIK3C2A, ITGB7, GLI2, PXN, VASP, RAF1, FYN, ITGB1, MAP2K2, PAK4, ADAM17, AKT1, PIK3R1, GLI1, WNT5A, ADAM10, MAP2K1, PAK3, ITGB3, CDC42, VEGFA, ITGA2, EPHA8, CRKL, RND1, GSK3B, AKT3, or PRKCA;
v) ephrin receptor signaling, wherein, preferably, the gene is PRKCE, ITGAM, ROCK1, ITGA5, CXCR4, IRAK1, PRKAA2, EIF2AK2, RAC1, RAP1A, GRK6, ROCK2, MAPK1, PGF, RAC2, PTPN11, GNAS, PLK1, AKT2, DOK1, CDK8, CREB1, PTK2, CFL1, GNAQ, MAP3K14, CXCL12, MAPK8, GNB2L1, ABL1, MAPK3, ITGA1, KRAS, RHOA, PRKCD, PRKAA1, MAPK9, SRC, CDK2, PIM1, ITGB7, PXN, RAF1, FYN, DYRK1A, ITGB1, MAP2K2, PAK4, AKT1, JAK2, STAT3, ADAM10, MAP2K1, PAK3, ITGB3, CDC42, VEGFA, ITGA2, EPHA8, TTK, CSNK1A1, CRKL, BRAF, PTPN13, ATF4, AKT3, or SGK;
vi) actin cytoskeleton signaling, wherein, preferably, the gene is ACTN4, PRKCE, ITGAM, ROCK1, ITGA5, IRAK1, PRKAA2, EIF2AK2, RAC1, INS, ARHGEF7, GRK6, ROCK2, MAPK1, RAC2, PLK1, AKT2, PIK3CA, CDK8, PTK2, CFL1, PIK3CB, MYH9, DIAPH1, PIK3C3, MAPK8, F2R, MAPK3, SLC9A1, ITGA1, KRAS, RHOA, PRKCD, PRKAA1, MAPK9, CDK2, PIM1, PIK3C2A, ITGB7, PPP1CC, PXN, VIL2, RAF1, GSN, DYRK1A, ITGB1, MAP2K2, PAK4, PIP5K1A, PIK3R1, MAP2K1, PAK3, ITGB3, CDC42, APC, ITGA2, TTK, CSNK1A1, CRKL, BRAF, VAV3, or SGK;
vii) Huntington's disease signaling, wherein, preferably, the gene is PRKCE, IGF1, EP300, RCOR1., PRKCZ, HDAC4, TGM2, MAPK1, CAPNS1, AKT2, EGFR, NCOR2, SP1, CAPN2, PIK3CA, HDAC5, CREB1, PRKC1, HSPA5, REST, GNAQ, PIK3CB, PIK3C3, MAPK8, IGF1R, PRKD1, GNB2L1, BCL2L1, CAPN1, MAPK3, CASP8, HDAC2, HDAC7A, PRKCD, HDAC11, MAPK9, HDAC9, PIK3C2A, HDAC3, TP53, CASP9, CREBBP, AKT1, PIK3R1, PDPK1, CASP1, APAF1, FRAP1, CASP2, JUN, BAX, ATF4, AKT3, PRKCA, CLTC, SGK, HDAC6, or CASP3;
viii) apoptosis signaling, wherein, preferably, the gene is PRKCE, ROCK1, BID, IRAK1, PRKAA2, EIF2AK2, BAK1, BIRC4, GRK6, MAPK1, CAPNS1, PLK1, AKT2, IKBKB, CAPN2, CDK8, FAS, NFKB2, BCL2, MAP3K14, MAPK8, BCL2L1, CAPN1, MAPK3, CASP8, KRAS, RELA, PRKCD, PRKAA1, MAPK9, CDK2, PIM1, TP53, TNF, RAF1, IKBKG, RELB, CASP9, DYRK1A, MAP2K2, CHUK, APAF1, MAP2K1, NFKB1, PAK3, LMNA, CASP2, BIRC2, TTK, CSNK1A1, BRAF, BAX, PRKCA, SGK, CASP3, BIRC3, or PARP1;
ix) B cell receptor signaling, wherein, preferably, the gene is RAC1, PTEN, LYN, ELK1, MAPK1, RAC2, PTPN11, AKT2, IKBKB, PIK3CA, CREB1, SYK, NFKB2, CAMK2A, MAP3K14, PIK3CB, PIK3C3, MAPK8, BCL2L1, ABL1, MAPK3, ETS1, KRAS, MAPK13, RELA, PTPN6, MAPK9, EGR1, PIK3C2A, BTK, MAPK14, RAF1, IKBKG, RELB, MAP3K7, MAP2K2, AKT1, PIK3R1, CHUK, MAP2K1, NFKB1, CDC42, GSK3A, FRAP1, BCL6, BCL10, JUN, GSK3B, ATF4, AKT3, VAV3, or RPS6KB1;
x) leukocyte extravasation signaling wherein, preferably, the gene is ACTN4, CD44, PRKCE, ITGAM, ROCK1, CXCR4, CYBA, RAC1, RAP1A, PRKCZ, ROCK2, RAC2, PTPN11, MMP14, PIK3CA, PRKCI, PTK2, PIK3CB, CXCL12, PIK3C3, MAPK8, PRKD1, ABL1, MAPK10, CYBB, MAPK13, RHOA, PRKCD, MAPK9, SRC, PIK3C2A, BTK, MAPK14, NOX1, PXN, VIL2, VASP, ITGB1, MAP2K2, CTNND1, PIK3R1, CTNNB1, CLDN1, CDC42, F11R, ITK, CRKL, VAV3, CTTN, PRKCA, MMP1, or MMP9;
xi) integrin signaling wherein, preferably, the gene is ACTN4, ITGAM, ROCK1, ITGA5, RAC1, PTEN, RAP1A, TLN1, ARHGEF7, MAPK1, RAC2, CAPNS1, AKT2, CAPN2, P1K3CA, PTK2, PIK3CB, PIK3C3, MAPK8, CAV1, CAPN1, ABL1, MAPK3, ITGA1, KRAS, RHOA, SRC, PIK3C2A, ITGB7, PPP1CC, ILK, PXN, VASP, RAF1, FYN, ITGB1, MAP2K2, PAK4, AKT1, PIK3R1, TNK2, MAP2K1, PAK3, ITGB3, CDC42, RND3, ITGA2, CRKL, BRAF, GSK3B, or AKT3;
xii) acute phase response signaling wherein, preferably, the gene is IRAK1, SOD2, MYD88, TRAF6, ELK1, MAPK1, PTPN11, AKT2, IKBKB, PIK3CA, FOS, NFKB2, MAP3K14, PIK3CB, MAPK8, RIPK1, MAPK3, IL6ST, KRAS, MAPK13, IL6R, RELA, SOCS1, MAPK9, FTL, NR3C1, TRAF2, SERPINE1, MAPK14, TNF, RAF1, PDK1, IKBKG, RELB, MAP3K7, MAP2K2, AKT1, JAK2, PIK3R1, CHUK, STAT3, MAP2K1, NFKB1, FRAP1, CEBPB, JUN, AKT3, IL1R1, or IL6;
xiii) PTEN signaling wherein, preferably, the gene is ITGAM, ITGA5, RAC1, PTEN, PRKCZ, BCL2L11, MAPK1, RAC2, AKT2, EGFR, IKBKB, CBL, PIK3CA, CDKN1B, PTK2, NFKB2, BCL2, PIK3CB, BCL2L1, MAPK3, ITGA1, KRAS, ITGB7, ILK, PDGFRB, INSR, RAF1, IKBKG, CASP9, CDKN1A, ITGB1, MAP2K2, AKT1, PIK3R1, CHUK, PDGFRA, PDPK1, MAP2K1, NFKB1, ITGB3, CDC42, CCND1, GSK3A, ITGA2, GSK3B, AKT3, FOXO1, CASP3, or RPS6KB1;
xiv) p53 signaling wherein, preferably, the gene is PTEN, EP300, BBC3, PCAF, FASN, BRCA1, GADD45A, BIRC5, AKT2, PIK3CA, CHEK1, TP53INP1, BCL2, PIK3CB, PIK3C3, MAPK8, THBS1, ATR, BCL2L1, E2F1, PMAIP1, CHEK2, TNFRSF10B, TP73, RB1, HDAC9, CDK2, PIK3C2A, MAPK14, TP53, LRDD, CDKN1A, HIPK2, AKT1, RIK3R1, RRM2B, APAF1, CTNNB1, SIRT1, CCND1, PRKDC, ATM, SFN, CDKN2A, JUN, SNAI2, GSK3B, BAX, or AKT3;
xv) aryl hydrocarbon receptor signaling wherein, preferably, the gene is HSPB1, EP300, FASN, TGM2, RXRA, MAPK1, NQO1, NCOR2, SP1, ARNT, CDKN1B, FOS, CHEK1, SMARCA4, NFKB2, MAPK8, ALDH1A1, ATR, E2F1, MAPK3, NRIP1, CHEK2, RELA, TP73, GSTP1, RB1, SRC, CDK2, AHR, NFE2L2, NCOA3, TP53, TNF, CDKN1A, NCOA2, APAF1, NFKB1, CCND1, ATM, ESR1, CDKN2A, MYC, JUN, ESR2, BAX, IL6, CYP1B1, or HSP90AA1;
xvi) xenobiotic metabolism signaling wherein, preferably, the gene is PRKCE, EP300, PRKCZ, RXRA, MAPK1, NQO1, NCOR2, PIK3CA, ARNT, PRKCI, NFKB2, CAMK2A, PIK3CB, PPP2R1A, PIK3C3, MAPK8, PRKD1, ALDH1A1, MAPK3, NRIP1, KRAS, MAPK13, PRKCD, GSTP1, MAPK9, NOS2A, ABCB1, AHR, PPP2CA, FTL, NFE2L2, PIK3C2A, PPARGC1A, MAPK14, TNF, RAF1, CREBBP, MAP2K2, PIK3R1, PPP2R5C, MAP2K1, NFKB1, KEAP1, PRKCA, EIF2AK3, 1L6, CYP1B1, or HSP90AA1;
xvii) SAPK or JNK signaling wherein, preferably, the gene is PRKCE, IRAK1, PRKAA2, EIF2AK2, RAC1, ELK1, GRK6, MAPK1, GADD45A, RAC2, PLK1, AKT2, PIK3CA, FADD, CDK8, PIK3CB, PIK3C3, MAPK8, RIPK1, GNB2L1, IRS1, MAPK3, MAPK10, DAXX, KRAS, PRKCD, PRKAA1, MAPK9, CDK2, PIM1, PIK3C2A, TRAF2, TP53, LCK, MAP3K7, DYRK1A, MAP2K2, PIK3R1, MAP2K1, PAK3, CDC42, JUN, TTK, CSNK1A1, CRKL, BRAF, or SGK;
xviii) PPAr or RXR signaling wherein, preferably, the gene is PRKAA2, EP300, INS, SMAD2, TRAF6, PPARA, FASN, RXRA, MAPK1, SMAD3, GNAS, IKBKB, NCOR2, ABCA1, GNAQ, NFKB2, MAP3K14, STAT5B, MAPK8, IRS1, MAPK3, KRAS, RELA, PRKAA1, PPARGC1A, NCOA3, MAPK14, INSR, RAF1, IKBKG, RELB, MAP3K7, CREBBP, MAP2K2, JAK2, CHUK, MAP2K1, NFKB1, TGFBR1, SMAD4, JUN, IL1R1, PRKCA, IL6, HSP90AA1, or ADIPOQ;
xix) NF-KB signaling wherein, preferably, the gene is IRAK1, EIF2AK2, EP300, INS, MYD88, PRKCZ: TRAF6, TBK1, AKT2, EGFR, IKBKB, PIK3CA, BTRC, NFKB2, MAP3K14, PIK3CB, PIK3C3, MAPK8, RIPK1, HDAC2, KRAS, RELA, PIK3C2A, TRAF2, TLR4: PDGFRB, TNF, INSR, LCK, IKBKG, RELB, MAP3K7, CREBBP, AKT1, PIK3R1, CHUK, PDGFRA, NFKB1, TLR2, BCL10, GSK3B, AKT3, TNFAIP3, or IL1R1;
xx) neuregulin signaling wherein, preferably, the gene is ERBB4, PRKCE, ITGAM, ITGA5: PTEN, PRKCZ, ELK1, MAPK1, PTPN11, AKT2, EGFR, ERBB2, PRKCI, CDKN1B, STAT5B, PRKD1, MAPK3, ITGA1, KRAS, PRKCD, STAT5A, SRC, ITGB7, RAF1, ITGB1, MAP2K2, ADAM17, AKT1, PIK3R1, PDPK1, MAP2K1, ITGB3, EREG, FRAP1, PSEN1, ITGA2, MYC, NRG1, CRKL, AKT3, PRKCA, HSP90AA1, or RPS6KB1;
xxi) Wnt or beta catenin signaling wherein, preferably, the gene is CD44, EP300, LRP6, DVL3, CSNK1E, GJA1, SMO, AKT2, PIN1, CDH1, BTRC, GNAQ, MARK2, PPP2R1A, WNT11, SRC, DKK1, PPP2CA, SOX6, SFRP2: ILK, LEF1, SOX9, TP53, MAP3K7, CREBBP, TCF7L2, AKT1, PPP2R5C, WNT5A, LRP5, CTNNB1, TGFBR1, CCND1, GSK3A, DVL1, APC, CDKN2A, MYC, CSNK1A1, GSK3B, AKT3, or SOX2;
xxii) insulin receptor signaling wherein, preferably, the gene is PTEN, INS, EIF4E, PTPN1, PRKCZ, MAPK1, TSC1, PTPN11, AKT2, CBL, PIK3CA, PRKCI, PIK3CB, PIK3C3, MAPK8, IRS1, MAPK3, TSC2, KRAS, EIF4EBP1, SLC2A4, PIK3C2A, PPP1CC, INSR, RAF1, FYN, MAP2K2, JAK1, AKT1, JAK2, PIK3R1, PDPK1, MAP2K1, GSK3A, FRAP1, CRKL, GSK3B, AKT3, FOXO1, SGK, or RPS6KB1;
xxiii) IL-6 signaling wherein, preferably, the gene is HSPB1, TRAF6, MAPKAPK2, ELK1, MAPK1, PTPN11, IKBKB, FOS, NFKB2: MAP3K14, MAPK8, MAPK3, MAPK10, IL6ST, KRAS, MAPK13, IL6R, RELA, SOCS1, MAPK9, ABCB1, TRAF2, MAPK14, TNF, RAF1, IKBKG, RELB, MAP3K7, MAP2K2, IL8, JAK2, CHUK, STAT3, MAP2K1, NFKB1, CEBPB, JUN, IL1R1, SRF, or IL6;
xxiv) hepatic cholestasis wherein, preferably, the gene is PRKCE, IRAK1, INS, MYD88, PRKCZ, TRAF6, PPARA, RXRA, IKBKB, PRKCI, NFKB2, MAP3K14, MAPK8, PRKD1, MAPK10, RELA, PRKCD, MAPK9, ABCB1, TRAF2, TLR4, TNF, INSR, IKBKG, RELB, MAP3K7, IL8, CHUK, NR1H2, TJP2, NFKB1, ESR1, SREBF1, FGFR4, JUN, IL1R1, PRKCA, or IL6;
xxv) IGF-1 signaling wherein, preferably, the gene is IGF1, PRKCZ, ELK1, MAPK1, PTPN11, NEDD4, AKT2, PIK3CA, PRKC1, PTK2, FOS, PIK3CB, PIK3C3, MAPK8, 1GF1R, IRS1, MAPK3, IGFBP7, KRAS, PIK3C2A, YWHAZ, PXN, RAF1, CASP9, MAP2K2, AKT1, PIK3R1, PDPK1, MAP2K1, IGFBP2, SFN, JUN, CYR61, AKT3, FOXO1, SRF, CTGF, or RPS6KB1;
xxvi) NRF2-mediated oxidative stress response wherein, preferably, the gene is PRKCE, EP300, SOD2, PRKCZ, MAPK1, SQSTM1, NQO1, PIK3CA, PRKC1, FOS, PIK3CB, P1K3C3, MAPK8, PRKD1, MAPK3, KRAS, PRKCD, GSTP1, MAPK9, FTL, NFE2L2, PIK3C2A, MAPK14, RAF1, MAP3K7, CREBBP, MAP2K2, AKT1, PIK3R1, MAP2K1, PPIB, JUN, KEAP1, GSK3B, ATF4, PRKCA, EIF2AK3, or HSP90AA1;
xxvii) hepatic fibrosis or hepatic stellate cell activation wherein, preferably, the gene is EDN1, IGF1, KDR, FLT1, SMAD2, FGFR1, MET, PGF, SMAD3, EGFR, FAS, CSF1, NFKB2, BCL2, MYH9, IGF1R, IL6R, RELA, TLR4, PDGFRB, TNF, RELB, IL8, PDGFRA, NFKB1, TGFBR1, SMAD4, VEGFA, BAX, IL1R1, CCL2, HGF, MMP1, STAT1, IL6, CTGF, or MMP9;
xxviii) PPAR signaling wherein, preferably, the gene is EP300, INS, TRAF6, PPARA, RXRA, MAPK1, IKBKB, NCOR2, FOS, NFKB2, MAP3K14, STAT5B, MAPK3, NRIP1, KRAS, PPARG, RELA, STAT5A, TRAF2, PPARGC1A, PDGFRB, TNF, INSR, RAF1, IKBKG, RELB, MAP3K7, CREBBP, MAP2K2, CHUK, PDGFRA, MAP2K1, NFKB1, JUN, IL1R1, or HSP90AA1;
xxix) Fc epsilon RI signaling wherein, preferably, the gene is PRKCE, RAC1, PRKCZ, LYN, MAPK1, RAC2, PTPN11, AKT2, PIK3CA, SYK, PRKCI, PIK3CB, PIK3C3, MAPK8, PRKD1, MAPK3, MAPK10, KRAS, MAPK13, PRKCD, MAPK9, PIK3C2A, BTK, MAPK14, TNF, RAF1, FYN, MAP2K2, AKT1, PIK3R1, PDPK1, MAP2K1, AKT3, VAV3, or PRKCA;
xxx) G-protein coupled receptor signaling wherein, preferably, the gene is PRKCE, RAP1A, RGS16, MAPK1, GNAS, AKT2, IKBKB, PIK3CA, CREB1, GNAQ, NFKB2, CAMK2A, PIK3CB, PIK3C3, MAPK3, KRAS, RELA, SRC, PIK3C2A, RAF1, IKBKG, RELB, FYN, MAP2K2, AKT1, PIK3R1, CHUK, PDPK1, STAT3, MAP2K1, NFKB1, BRAF, ATF4, AKT3, or PRKCA;
xxxi) inositol phosphate metabolism wherein, preferably, the gene is PRKCE, IRAK1, PRKAA2, EIF2AK2, PTEN, GRK6, MAPK1, PLK1, AKT2, PIK3CA, CDK8, PIK3CB, PIK3C3, MAPK8, MAPK3, PRKCD, PRKAA1, MAPK9, CDK2, PIM1, PIK3C2A, DYRK1A, MAP2K2, PIP5K1A, PIK3R1, MAP2K1, PAK3, ATM, TTK, CSNK1A1, BRAF, or SGK;
xxxii) PDGF signaling wherein, preferably, the gene is EIF2AK2, ELK1, ABL2, MAPK1, PIK3CA, FOS, PIK3CB, PIK3C3, MAPK8, CAV1, ABL1, MAPK3, KRAS, SRC, PIK3C2A, PDGFRB, RAF1, MAP2K2, JAK1, JAK2, PIK3R1, PDGFRA, STAT3, SPHK1, MAP2K1, MYC, JUN, CRKL, PRKCA, SRF, STAT1, or SPHK2;
xxxiii) VEGF signaling wherein, preferably, the gene is ACTN4, ROCK1, KDR, FLT1, ROCK2, MAPK1, PGF, AKT2, PIK3CA, ARNT, PTK2, BCL2, PIK3CB, PIK3C3, BCL2L1, MAPK3, KRAS, HIF1A, NOS3, PIK3C2A, PXN, RAF1, MAP2K2, ELAVL1, AKT1, PIK3R1, MAP2K1, SFN, VEGFA, AKT3, FOXO1, or PRKCA;
xxxiv) natural killer cell signaling wherein, preferably, the gene is PRKCE, RAC1, PRKCZ, MAPK1, RAC2, PTPN11, KIR2DL3, AKT2, PIK3CA, SYK, PRKCI, PIK3CB, PIK3C3, PRKD1, MAPK3, KRAS, PRKCD, PTPN6, PIK3C2A, LCK, RAF1, FYN, MAP2K2, PAK4, AKT1, PIK3R1, MAP2K1, PAK3, AKT3, VAV3, or PRKCA;
xxxv) cell cycle G1/S checkpoint regulation wherein, preferably, the gene is HDAC4, SMAD3, SUV39H1, HDAC5, CDKN1B, BTRC, ATR, ABL1, E2F1, HDAC2, HDAC7A, RB1, HDAC11, HDAC9, CDK2, E2F2, HDAC3, TP53, CDKN1A, CCND1, E2F4, ATM, RBL2, SMAD4, CDKN2A, MYC, NRG1, GSK3B, RBL1, or HDAC6;
xxxvi) T cell receptor signaling wherein, preferably, the gene is RAC1, ELK1, MAPK1, IKBKB, CBL, PIK3CA, FOS, NFKB2, PIK3CB, PIK3C3, MAPK8, MAPK3, KRAS, RELA, PIK3C2A, BTK, LCK, RAF1, IKBKG, RELB, FYN, MAP2K2, PIK3R1, CHUK, MAP2K1, NFKB1, ITK, BCL10, JUN, or VAV3;
xxxvii) death receptor signaling wherein, preferably, the gene is CRADD, HSPB1, BID, BIRC4, TBK1, IKBKB, FADD, FAS, NFKB2, BCL2, MAP3K14, MAPK8, RIPK1, CASP8, DAXX, TNFRSF10B, RELA, TRAF2, TNF, IKBKG, RELB, CASP9, CHUK, APAF1, NFKB1, CASP2, BIRC2, CASP3, or BIRC3;
xxxviii) FGF signaling wherein, preferably, the gene is RAC1, FGFR1, MET, MAPKAPK2, MAPK1, PTPN11, AKT2, PIK3CA, CREB1, PIK3CB, PIK3C3, MAPK8, MAPK3, MAPK13, PTPN6, PIK3C2A, MAPK14, RAF1, AKT1, PIK3R1, STAT3, MAP2K1, FGFR4, CRKL, ATF4, AKT3, PRKCA, or HGF;
xxxix) GM-CSF signaling wherein, preferably, the gene is LYN, ELK1, MAPK1, PTPN11, AKT2, PIK3CA, CAMK2A, STAT5B, PIK3CB, PIK3C3, GNB2L1, BCL2L1, MAPK3, ETS1, KRAS, RUNX1, PIM1, PIK3C2A, RAF1, MAP2K2, AKT1, JAK2, PIK3R1, STAT3, MAP2K1, CCND1, AKT3, or STAT1;
xl) amyotrophic lateral sclerosis signaling wherein, preferably, the gene is BID, IGF1, RAC1, BIRC4, PGF, CAPNS1, CAPN2, PIK3CA, BCL2, PIK3CB, PIK3C3, BCL2L1, CAPN1, PIK3C2A, TP53, CASP9, PIK3R1, RAB5A, CASP1, APAF1, VEGFA, BIRC2, BAX, AKT3, CASP3, or BIRC3;
xli) JAK-Stat signaling wherein, preferably, the gene is PTPN1, MAPK1, PTPN11, AKT2, PIK3CA, STAT5B, PIK3CB, PIK3C3, MAPK3, KRAS, SOCS1, STAT5A, PTPN6, PIK3C2A, RAF1, CDKN1A, MAP2K2, JAK1, AKT1, JAK2, PIK3R1, STAT3, MAP2K1, FRAP1, AKT3, STAT1;
xlii) nicotinate or nicotinamide metabolism wherein, preferably, the gene is PRKCE, IRAK1, PRKAA2, EIF2AK2, GRK6, MAPK1, PLK1, AKT2, CDK8, MAPK8, MAPK3, PRKCD, PRKAA1, PBEF1, MAPK9, CDK2, PIM1, DYRK1A, MAP2K2, MAP2K1, PAK3, NT5E, TTK, CSNK1A1, BRAF, or SGK;
xliii) chemokine signaling wherein, preferably, the gene is CXCR4, ROCK2, MAPK1, PTK2, FOS, CFL1, GNAQ, CAMK2A, CXCL12, MAPK8, MAPK3, KRAS, MAPK13, RHOA, CCR3, SRC, PPP1CC, MAPK14, NOX1, RAF1, MAP2K2, MAP2K1, JUN, CCL2, or PRKCA;
xliv) IL-2 signaling wherein, preferably, the gene is ELK1, MAPK1, PTPN11, AKT2, PIK3CA, SYK, FOS, STAT5B, PIK3CB, PIK3C3, MAPK8, MAPK3, KRAS, SOCS1, STAT5A, PIK3C2A: LCK, RAF1, MAP2K2, JAK1, AKT1, PIK3R1, MAP2K1, JUN, or AKT3;
xlv) synaptic long term depression wherein, preferably, the gene is PRKCE, IGF1, PRKCZ, PRDX6, LYN, MAPK1, GNAS, PRKC1, GNAQ, PPP2R1A, IGF1R, PRKID1, MAPK3, KRAS, GRN, PRKCD, NOS3, NOS2A, PPP2CA, YWHAZ, RAF1, MAP2K2, PPP2R5C, MAP2K1, or PRKCA;
xlvi) estrogen receptor signaling wherein, preferably, the gene is TAF4B, EP300, CARM1, PCAF, MAPK1, NCOR2, SMARCA4, MAPK3, NRIP1, KRAS, SRC, NR3C1, HDAC3, PPARGC1A, RBM9, NCOA3, RAF1, CREBBP, MAP2K2, NCOA2, MAP2K1, PRKDC, ESR1, or ESR2;
xlvii) protein ubiquitination pathway wherein, preferably, the gene is TRAF6, SMURF1, BIRC4, BRCA1, UCHL1, NEDD4, CBL, UBE2I, BTRC, HSPA5, USP7, USP10, FBXW7, USP9X, STUB1, USP22, B2M, BIRC2, PARK2, USP8, USP1, VHL, HSP90AA1, or BIRC3;
xlviii) IL-10 signaling wherein, preferably, the gene is TRAF6, CCR1, ELK1, IKBKB, SP1, FOS, NFKB2, MAP3K14, MAPK8, MAPK13, RELA, MAPK14, TNF, IKBKG, RELB, MAP3K7, JAK1, CHUK, STAT3, NFKB1, JUN, IL1R1, or IL6;
xlix) VDR or RXR activation wherein, preferably, the gene is PRKCE, EP300, PRKCZ, RXRA, GADD45A, HES1, NCOR2, SP1, PRKC1, CDKN1B, PRKD1, PRKCD, RUNX2, KLF4, YY1, NCOA3, CDKN1A, NCOA2, SPP1, LRP5, CEBPB, FOXO1, or PRKCA;
l) TGF-beta signaling wherein, preferably, the gene is EP300, SMAD2, SMURF1, MAPK1, SMAD3, SMAD1, FOS, MAPK8, MAPK3, KRAS, MAPK9, RUNX2, SERPINE1, RAF1, MAP3K7, CREBBP, MAP2K2, MAP2K1, TGFBR1, SMAD4, JUN, or SMAD5;
li) toll-like receptor signaling wherein, preferably, the gene is IRAK1, EIF2AK2, MYD88, TRAF6, PPARA, ELK1, IKBKB, FOS, NFKB2, MAP3K14, MAPK8, MAPK13, RELA, TLR4, MAPK14, IKBKG, RELB, MAP3K7, CHUK, NFKB1, TLR2, or JUN;
lii) p38 MAPK signaling wherein, preferably, the gene is HSPB1, IRAK1, TRAF6, MAPKAPK2, ELK1, FADD, FAS, CREB1, DDIT3, RPS6KA4, DAXX, MAPK13, TRAF2, MAPK14, TNF, MAP3K7, TGFBR1, MYC, ATF4, IL1R1, SRF, or STAT1;
liii) neurotrophin or TRK Signaling wherein, preferably, the gene is NTRK2, MAPK1, PTPN11, PIK3CA, CREB1, FOS, PIK3CB, PIK3C3, MAPK8, MAPK3, KRAS, PIK3C2A, RAF1, MAP2K2, AKT1, PIK3R1, PDPK1, MAP2K1, CDC42, JUN, or ATF4;
liv) FXR or RXR activation wherein, preferably, the gene is INS, PPARA, FASN, RXRA, AKT2, SDC1, MAPK8, APOB, MAPK10, PPARG, MTTP, MAPK9, PPARGC1A, TNF, CREBBP, AKT1, SREBF1, FGFR4, AKT3, or FOXO1;
lv) synaptic long term potentiation wherein, preferably, the gene is PRKCE, RAP1A, EP300, PRKCZ, MAPK1, CREB1, PRKC1, GNAQ, CAMK2A, PRKD1, MAPK3, KRAS, PRKCD, PPP1CC, RAF1, CREBBP, MAP2K2, MAP2K1, ATF4, or PRKCA;
lvi) calcium signaling wherein, preferably, the gene is RAP1A, EP300, HDAC4, MAPK1, HDAC5, CREB1, CAMK2A, MYH9, MAPK3, HDAC2, HDAC7A, HDAC11, HDAC9, HDAC3, CREBBP, CALR, CAMKK2, ATF4, or HDAC6;
lvii) EGF signaling wherein, preferably, the gene is ELK1, MAPK1, EGFR, PIK3CA, FOS, PIK3CB, PIK3C3, MAPK8, MAPK3, PIK3C2A, RAF1, JAK1, PIK3R1, STAT3, MAP2K1, JUN, PRKCA, SRF, or STAT1;
lviii) hypoxia signaling in the cardiovascular system wherein, preferably, the gene is EDN1, PTEN, EP300, NQO1, UBE2I, CREB1, ARNT, HIF1A, SLC2A4, NOS3, TP53, LDHA, AKT1, ATM, VEGFA, JUN, ATF4, VHL, or HSP90AA1;
lix) LPS or IL-1 mediated inhibition of RXR function wherein, preferably, the gene is IRAK1, MYD88, TRAF6, PPARA, RXRA, ABCA1, MAPK8, ALDH1A1, GSTP1, MAPK9, ABCB1, TRAF2, TLR4, TNF, MAP3K7, NR1H2, SREBF1, JUN, or IL1R1;
lx) LXR or RXR activation wherein, preferably, the gene is FASN, RXRA, NCOR2, ABCA1, NFKB2, IRF3, RELA, NOS2A, TLR4, TNF, RELB, LDLR, NR1H2, NFKB1, SREBF1, IL1R1, CCL2, IL6, or MMP9;
lxi) amyloid processing wherein, preferably, the gene is PRKCE, CSNK1E, MAPK1, CAPNS1, AKT2, CAPN2, CAPN1, MAPK3, MAPK13, MAPT, MAPK14, AKT1, PSEN1, CSNK1A1, GSK3B, AKT3, or APP;
lxii) IL-4 signaling wherein, preferably, the gene is AKT2, PIK3CA, PIK3CB, PIK3C3, IRS1, KRAS, SOCS1, PTPN6, NR3C1, PIK3C2A, JAK1, AKT1, JAK2, PIK3R1, FRAP1, AKT3, or RPS6KB1;
lxiii) cell cycle: G2/M DNA damage checkpoint regulation wherein, preferably, the gene is EP300, PCAF, BRCA1, GADD45A, PLK1, BTRC, CHEK1, ATR, CHEK2, YWHAZ, TP53, CDKN1A, PRKDC, ATM, SFN, or CDKN2A;
lxiv) nitric oxide signaling in the cardiovascular system wherein, preferably, the gene is KDR, FLT1, PGF, AKT2, PIK3CA, PIK3CB, PIK3C3, CAV1, PRKCD, NOS3, PIK3C2A, AKT1, PIK3R1, VEGFA, AKT3, or HSP90AA1;
lxv) purine metabolism wherein, preferably, the gene is NME2, SMARCA4, MYH9, RRM2, ADAR, EIF2AK4, PKM2, ENTPD1, RAD51, RRM2B, TJP2, RAD51C, NT5E, POLD1, or NME1;
lxvi) cAMP-mediated Signaling wherein, preferably, the gene is RAP1A, MAPK1, GNAS, CREB1, CAMK2A, MAPK3, SRC, RAF1, MAP2K2, STAT3, MAP2K1, BRAF, or ATF4;
lxvii) mitochondrial dysfunction wherein, preferably, the gene is SOD2, MAPK8, CASP8, MAPK10, MAPK9, CASP9, PARK7, PSEN1, PARK2, APP, or CASP3;
lxviii) notch signaling wherein, preferably, the gene is HES1, JAG1, NUMB, NOTCH4, ADAM17, NOTCH2, PSEN1, NOTCH3, NOTCH1, or DLL4;
lxix) endoplasmic reticulum stress pathway wherein, preferably, the gene is HSPA5, MAPK8, XBP1, TRAF2, ATF6, CASP9, ATF4, EIF2AK3, or CASP3;
lxx) pyrimidine metabolism wherein, preferably, the gene is NME2, AICDA, RRM2, EIF2AK4, ENTPD1, RRM2B, NT5E, POLD1, or NME1;
lxxi) Parkinson's signaling wherein, preferably, the gene is UCHL1, MAPK8, MAPK13, MAPK14, CASP9, PARK7, PARK2, or CASP3;
lxxii) cardiac or beta adrenergic signaling wherein, preferably, the gene is GNAS, GNAQ, PPP2R1A, GNB2L1, PPP2CA, PPP1CC, or PPP2R5C;
lxxiii) glycolysis or gluconeogenesis wherein, preferably, the gene is HK2, GCK, GPI, ALDH1A1, PKM2, LDHA, or HK1;
lxxiv) interferon signaling wherein, preferably, the gene is IRF1, SOCS1, JAK1, JAK2, IFITM1, STAT1, or IFIT3;
lxxv) Sonic Hedgehog signaling wherein, preferably, the gene is ARRB2, SMO, GLI2, DYRK1A, GLI1, GSK3B, or DYRKIB;
lxxvi) glycerophospholipid metabolism wherein, preferably, the gene is PLD1, GRN, GPAM, YWHAZ, SPHK1, or SPHK2;
lxxvii) phospholipid degradation wherein, preferably, the gene is PRDX6, PLD1, GRN, YWHAZ, SPHK1, or SPHK2;
lxxviii) tryptophan metabolism wherein, preferably, the gene is SIAH2, PRMT5, NEDD4, ALDH1A1, CYP1B1, or SIAH1;
lxxix) lysine degradation wherein, preferably, the gene is SUV39H1, EHMT2, NSD1, SETD7, or PPP2R5C;
lxxx) nucleotide excision repair pathway wherein, preferably, the gene is ERCC5, ERCC4, XPA, XPC, or ERCC1;
lxxxi) starch or sucrose metabolism wherein, preferably, the gene is UCHL1, HK2, GCK, GPI, or HK1;
lxxxii) amino sugars metabolism wherein, preferably, the gene is NQO1, HK2, GCK, or HK1;
lxxxiii) arachidonic acid metabolism wherein, preferably, the gene is PRDX6, GRN, YWHAZ, or CYP1B1;
lxxxiv) circadian rhythm signaling wherein, preferably, the gene is CSNK1E, CREB1, ATF4, or NR1 D1;
lxxxv) coagulation system wherein, preferably, the gene is BDKRB1, F2R, SERPINE1, or F3;
lxxxvi) dopamine receptor signaling wherein, preferably, the gene is PPP2R1A, PPP2CA, PPP1CC, or PPP2R5C;
lxxxvii) glutathione metabolism wherein, preferably, the gene is IDH2, GSTP1, ANPEP, or IDH1;
lxxxviii) glycerolipid metabolism wherein, preferably, the gene is ALDH1A1, GPAM, SPHK1, or SPHK2;
lxxxix) linoleic acid metabolism wherein, preferably, the gene is PRDX6, GRN, YWHAZ, or CYP1B1;
xc) methionine metabolism wherein, preferably, the gene is DNMT1, DNMT3B, AHCY, or DNMT3A;
xci) pyruvate metabolism wherein, preferably, the gene is GLO1, ALDH1A1, PKM2, or LDHA;
xcii) arginine and proline metabolism wherein, preferably, the gene is ALDH1A1, NOS3, or NOS2A;
xciii) eicosanoid signaling wherein, preferably, the gene is PRDX6, GRN, or YWHAZ;
xciv) fructose and mannose metabolism wherein, preferably, the gene is HK2, GCK, or HK1;
xcv) galactose metabolism wherein, preferably, the gene is HK2, GCK, or HK1;
xcvi) stilbene, coumarine, or lignin biosynthesis wherein, preferably, the gene is PRDX6, PRDX1, or TYR;
xcvii) antigen presentation pathway wherein, preferably, the gene is CALR or B2M;
xcviii) biosynthesis of steroids wherein, preferably, the gene is NQO1 or DHCR7;
xcix) butanoate metabolism wherein, preferably, the gene is ALDH1A1 or NLGN1;
c) citrate cycle wherein, preferably, the gene is IDH2 or IDH1;
ci) fatty acid metabolism wherein, preferably, the gene is ALDH1A1 or CYP1B1;
cii) histidine metabolism wherein, preferably, the gene is PRMT5 or ALDH1A1;
ciii) inositol metabolism wherein, preferably, the gene is ERO1L or APEX1;
civ) metabolism of xenobiotics by Cytochrome p450 wherein, preferably, the gene is GSTP1 or CYP1B1;
cv) methane metabolism wherein, preferably, the gene is PRDX6 or PRDX1;
cvi) phenylalanine metabolism wherein, preferably, the gene is PRDX6 or PRDX1;
cvii) propanoate metabolism wherein, preferably, the gene is ALDH1A1 or LDHA;
ciii) selenoamino acid metabolism wherein, preferably, the gene is PRMT5 or AHCY;
cix) sphingolipid metabolism wherein, preferably, the gene is SPHK1 or SPHK2;
cx) aminophosphonate metabolism wherein, preferably, the gene is PRMT5;
cxi) androgen or estrogen metabolism wherein, preferably, the gene is PRMT5;
cxii) ascorbate and aldarate metabolism wherein, preferably, the gene is ALDH1A1;
cxiii) bile acid biosynthesis wherein, preferably, the gene is ALDH1A1;
cxiv) cysteine metabolism wherein, preferably, the gene is LDHA;
cxv) fatty acid biosynthesis wherein, preferably, the gene is FASN;
cxvi) glutamate receptor signaling wherein, preferably, the gene is GNB2L1;
cxvii) NRF2-mediated oxidative stress response wherein, preferably, the gene is PRDX1;
cxiii) pentose phosphate pathway wherein, preferably, the gene is GPI;
cxix) pentose and glucuronate interconversions wherein, preferably, the gene is UCHL1;
cxx) retinol metabolism wherein, preferably, the gene is ALDH1A1;
cxxi) riboflavin metabolism wherein, preferably, the gene is TYR;
cxxii) tyrosine metabolism wherein, preferably, the gene is PRMT5 or TYR;
cxxiii) ubiquinone biosynthesis wherein, preferably, the gene is PRMT5;
cxxiv) valine, leucine and isoleucine degradation wherein, preferably, the gene is ALDH1A1;
cxxv) glycine, serine and threonine metabolism wherein, preferably, the gene is CHKA;
cxxvi) lysine degradation wherein, preferably, the gene is ALDH1A1;
cxxvii) pain or taste wherein, preferably, the gene is TRPM5 or TRPA1;
cxxiii) pain wherein, preferably, the gene is TRPM7, TRPC5, TRPC6, TRPC1, CNR1, CNR2, GRK2, TRPA1, POMC, CGRP, CRF, PKA, ERA, NR2b, TRPM5, PRKACa, PRKACb, PRKAR1a, or PRKAR2a;
cxxix) mitochondrial function wherein, preferably, the gene is AIF, CYTC, SMAC (Diablo), AIFM-1, or AIFM-2;
cxxx) developmental neurology wherein, preferably, the gene is BMP-4, chordin (CHRD), noggin (Nog), WNT, WNT2, WNT2b, WNT3a, WNT4, WNT5a, WNT6, WNT7b, WNT8b, WNT9a, WNT9b, WNT10a, WNT10b, WNT16, beta-catenin, DKK-1, frizzled related proteins, OTX-2, GBX2, FGF-8, Reelin, DAB1, UNC-86, POU4f1, BRN3a, NUMB, or RELN.

108. The pharmaceutical composition according to claim 78, for use in treating a disease or disorder.

109. The pharmaceutical composition for use according to claim 108, wherein the disease or disorder is selected from a group consisting of age-related macular degeneration; a blood or coagulation disease or disorder; a cell dysregulation or oncology disease or disorder; a developmental disorder; drug addiction; an inflammation or immune related disease or disorder; a metabolic, liver, kidney, or protein disease or disorder; a muscular or skeletal disease or disorder; a neurological or neuronal disease or disorder; a neoplasia; an ocular disease or disorder; schizophrenia; epilepsy; Duchenne muscular dystrophy; a viral disease or disorder, such as AIDS (acquired immunodeficiency syndrome); an autoimmune disorder; and an Alpha 1-antitrypsin deficiency.

110. The pharmaceutical composition of claim 109, wherein the blood or coagulation disease or disorder is:
a) anemia wherein, preferable, the gene is CDAN1, CDA1, RPS19, DBA, PKLR, PK1, NT5C3, UMPH1, PSN1, RHAG, RH50A, NRAMP2, SPTB, ALAS2, ANH1, ASB, ABCB7, ABC7, and/or ASAT;
b) bare lymphocyte syndrome wherein, preferably, the gene is TAPBP, TPSN, TAP2, ABCB3, PSF2, RING11, MHC2TA, C2TA, RFX5, RFXAP, or RFX5;
c) a bleeding disorder, wherein, preferably, the gene is TBXA2R, P2RX1, or P2X1;
d) a hemolytic anemia, such as a complement Factor H deficiency disease, e.g., a typical hemolytic anemia syndrome (aHUS), wherein, preferably, the gene is HF1, CFH, or HUS;
e) a factor V or factor VIII deficiency disease, wherein, preferably, the gene is MCFD2;
f) a factor VII deficiency disease, wherein, preferably, the gene is F7;
g) a factor X deficiency disease, wherein, preferably, the gene is F10;
h) a factor XI deficiency disease, wherein, preferably, the gene is F11;
i) a factor XII deficiency disease, wherein, preferably, the gene is F12 or HAF;
j) a factor XIIIA deficiency disease, wherein, preferably, the gene is F13A1 or F13A;
k) a factor XIIIB deficiency disease, wherein, preferably, the gene is F13B;
l) Fanconi anemia, wherein, preferably, the gene is FANCA, FACA, FA1, FA, FAA, FAAP95, FAAP90, FLJ34064, FANCB, FANCC, FACC, BRCA2, FANCD1, FANCD2, FANCD, FACD, FAD, FANCE, FACE, FANCF, XRCC9, FANCG, BRIP1, BACH1, FANCJ, PHF9, FANCL, FANCM, or KIAA1596;
m) a hemophagocytic or lymphohistiocytosis disorder, wherein, preferably, the gene is PRF1, HPLH2, UNC13D, MUNC13-4, HPLH3, HLH3, or FHL3;
n) hemophilia A, wherein, preferably, the gene is F8, F8C, or HEMA;
o) hemophilia B, wherein, preferably, the gene is F9 or HEMB;
p) a hemorrhagic disorder, wherein, preferably, the gene is PI, ATT, F5;
q) a leukocyte deficiency or disorder, wherein, preferably, the gene is ITGB2, CD18, LCAMB, LAD, EIF2B1, EIF2BA, EIF2B2, EIF2B3, EIF2B5, LVWM, CACH, CLE, or EIF2B4;
r) sickle cell anemia, wherein, preferably, the gene is HBB; or
s) thalassemia, wherein, preferably, the gene is HBA2, HBB, HBD, LCRB, or HBA1.

111. The pharmaceutical composition of claim 109, wherein the cell dysregulation or oncology disease is:
a) B-cell non-Hodgkin lymphoma, wherein, preferably, the gene is BCL7A or BCL7; or
b) a leukemia, wherein, preferably, the gene is TAL1 TCL5, SCL, TAL2, FLT3, NBS1, NBS, ZNFN1A1, IK1, LYF1, HOXD4, HOX4B, BCR, CML, PHL, ALL, ARNT, KRAS2, RASK2, GMPS, AF10, ARHGEF12, LARG, KIAA0382, CALM, CLTH, CEBPA, CEBP, CHIC2, BTL, FLT3, KIT, PBT, LPP, NPM1, NUP214, D9S46E, CAN, CAIN, RUNX1, CBFA2, AML1, WHSC1L1, NSD3, FLT3, AF1Q, NPM1, NUMA1, ZNF145, PLZF, PML, MYL, STAT5B, AF10, CALM, CLTH, ARL11, ARLTS1, P2RX7, P2X7, BCR, CML, PHL, ALL, GRAF, NF1, VRNF, WSS, NFNS, PTPN11, PTP2C, SHP2, NS1, BCL2, CCND1, PRAD1, BCL1, TCRA, GATA1, GF1, ERYF1, NFE1, ABL1, NQO1, DIA4, NMOR1, NUP214, D9S46E, CAN, or CAIN.

112. The pharmaceutical composition of claim 109, wherein the developmental disease is:
a) Angelman syndrome, wherein, preferably, the gene is UBE3A or a 15q11-13 deletion;
b) Canavan disease, wherein, preferably, the gene is ASPA;
c) Cri-du-chat syndrome, wherein, preferably, the gene is 5P− (5p minus) or CTNND2;
d) Down syndrome, wherein, preferably, the gene is Trisomy 21;
e) Klinefelter syndrome, wherein, preferably, the gene is XXY or two or more X chromosomes in males;
f) Prader-Willi syndrome, wherein, preferably, the gene is deletion of chromosome 15 segment or a duplication of maternal chromosome 15; or
g) Turner syndrome where the gene is monosomy X or SHOX.

113. The pharmaceutical composition of claim 109, wherein the disease or disorder is a drug addiction disease wherein, preferably, the gene is PRKCE, DRD2, DRD4, ABAT (alcohol), GRIA2, GRM5, GRIN1, HTR1B, GRIN2A, DRD3, PDYN, GRIA1 (alcohol).

114. The pharmaceutical composition of claim 109, wherein the inflammation or immune related disease is:
a) autoimmune lymphoproliferative syndrome, wherein, preferably, the gene TNFRSF6, APT1, FAS, CD95, or ALPS1A;
b) combined immuno-deficiency, wherein, preferably, the gene is IL2RG, SCIDX1, SCIDX, or IMD4;
c) an immuno-deficiency, wherein, preferably, the gene is CD3E, CD3G, AICDA, AID, HIGM2, TNFRSF5, CD40, UNG, DGU, HIGM4, TNFSF5, CD40LG, HIGM1, IGM, FOXP3, IPEX, AIID, XPID, PIDX, TNFRSF14B, or TACI;
d) inflammation wherein, preferably, the gene is IL-10, IL-1 (IL-1a, IL-1 b), IL-13, IL-17 (IL-17a (CTLA8), IL-17b, IL-17c, IL-17d, IL-17f), Il-23, CX3CR1, PTPN22, TNFa, NOD2/CARD15 for IBD, IL-6, IL-12 (IL-12a, IL-12b), CTLA4, or CX3CL1; or
e) severe combined immunodeficiency disease, wherein, preferably, the gene is (SCIDs) (JAK3, JAKL, DCLRE1C, ARTEMIS, SCIDA, RAG1, RAG2, ADA, PTPRC, CD45, LCA, IL7R, CD3D, T3D, IL2RG, SCIDX1, SCIDX, or IMD4.

115. The pharmaceutical composition of claim 109, wherein the metabolic, liver, kidney, or protein disease is:
a) amyloid neuropathy, wherein, preferably, the gene is TTR or PALB;
b) amyloidosis, wherein, preferably, the gene is APOA1, APP, AAA, CVAP, AD1, GSN, FGA, LYZ, TTR, or PALB;
c) cirrhosis, wherein, preferably, the gene is KRT18, KRT8, CIRH1A, NAIC, TEX292, or KIAA1988;
d) cystic fibrosis, wherein, preferably, the gene is CFTR, ABCC7, CF, or MRP7;
e) a glycogen storage disease, wherein, preferably, the gene is SLC2A2, GLUT2, G6PC, G6PT, G6PT1, GAA, LAMP2, LAMPB, AGL, GDE, GBE1, GYS2, PYGL, or PFKM;
f) a hepatic adenoma, wherein, preferably, the gene is TCF1, HNF1A, or MODY3;
g) an early onset neurologic disorder, wherein, preferably, the gene is SCOD1 or SCO1;
h) a hepatic lipase deficiency, wherein, preferably, the gene is LIPC;
i) hepato-blastoma cancer, wherein, preferably, the gene is CTNNB1, PDGFRL, PDGRL, PRLTS, AXIN1, AXIN, CTNNB1, TP53, P53, LFS1, IGF2R, MPRI, MET, CASP8, or MCH5;
j) medullary cystic kidney disease, wherein, preferably, the gene is UMOD, HNFJ, FJHN, MCKD2, or ADMCKD2;
k) phenylketonuria, wherein, preferably, the gene is PAH, PKU1, QDPR, DHPR, or PTS; or
l) polycystic kidney or hepatic disease, wherein, preferably, the gene is FCYT, PKHD1, ARPKD, PKD1, PKD2, PKD4, PKDTS, PRKCSH, G19P1, PCLD, or SEC63.

116. The pharmaceutical composition of claim 109, wherein the muscular or skeletal disease is:
a) Becker muscular dystrophy, wherein, preferably, the gene is DMD, BMD, or MYF6;
b) Duchenne muscular dystrophy, wherein, preferably, the gene is DMD or BMD;
c) Emery-Dreifuss muscular dystrophy, wherein, preferably, the gene is LMNA, LMN1, EMD2, FPLD, CMD1A, HGPS, LGMD1B, LMNA, LMN1, EMD2, FPLD, or CMD1A;
d) Facio-scapulohumeral muscular dystrophy, wherein, preferably, the gene is FSHMD1A or FSHD1A;
e) muscular dystrophy, wherein, preferably, the gene is FKRP, MDC1C, LGMD2I, LAMA2, LAMM, LARGE, KIAA0609, MDC1D, FCMD, TTID, MYOT, CAPN3, CANP3, DYSF, LGMD2B, SGCG, LGMD2C, DMDA1, SCG3, SGCA, ADL, DAG2, LGMD2D, DMDA2, SGCB, LGMD2E, SGCD, SGD, LGMD2F, CMD1L, TCAP, LGMD2G, CMD1N, TRIM32, HT2A, LGMD2H, FKRP, MDC1C, LGMD2I, TTN, CMD1G, TMD, LGMD2J, POMT1, CAV3, LGMD1C, SEPN1, SELN, RSMD1, PLEC1, PLTN, or EBS1;
f) osteopetrosis, wherein, preferably, the gene is LRP5, BMND1, LRP7, LR3, OPPG, VBCH2, CLCN7, CLC7, OPTA2, OSTM1, GL, TCIRG1, TIRC7, OC116, or OPTB1;
g) muscular atrophy, wherein, preferably, the gene is VAPB, VAPC, ALS8, SMN1, SMA1, SMA2, SMA3, SMA4, BSCL2, SPG17, GARS, SMAD1, CMT2D, HEXB, IGHMBP2, SMUBP2, CATF1, or SMARD1; or
h) Tay-Sachs disease wherein, preferably, the gene is HEXA.

117. The pharmaceutical composition of claim 109, wherein the neurological and neuronal disease is:
a) amyotrophic lateral sclerosis (ALS), wherein, preferably, the gene is SOD1, ALS2, STEX, FUS, TARDBP, or VEGF (VEGF-a, VEGF-b, VEGF-c);
b) Alzheimer's disease, wherein, preferably, the gene is APP, AAA, CVAP, AD1, APOE, AD2, PSEN2, AD4, STM2, APBB2, FE65L1, NOS3, PLAU, URK, ACE, DCP1, ACE1, MPO, PACIP1, PAXIP1L, PTIP, A2M, BLMH, BMH, PSEN1, orAD3;
c) autism, wherein, preferably, the gene is Mecp2, BZRAP1, MDGA2, Sema5A, Neurexin 1, GLO1, MECP2, RTT, PPMX, MRX16, MRX79, NLGN3, NLGN4, KIAA1260, or AUTSX2;
d) Fragile X Syndrome, wherein, preferably, the gene is FMR2, FXR1, FXR2, or mGLUR5;
e) Huntington's disease or a Huntington's disease like disorder, wherein, preferably, the gene is HD, IT15, PRNP, PRIP, JPH3, JP3, HDL2, TBP, or SCA17;
f) Parkinson's disease, wherein, preferably, the gene is NR4A2, NURR1, NOT, TINUR, SNCAIP, TBP, SCA17, SNCA, NACP, PARK1, PARK4, DJ1, PARK7, LRRK2, PARK8, PINK1, PARK6, UCHL1, PARK5, SNCA, NACP, PARK1, PARK4, PRKN, PARK2, PDJ, DBH, or NDUFV2;
g) Rett syndrome, wherein, preferably, the gene is MECP2, RTT, PPMX, MRX16, MRX79, CDKL5, STK9, MECP2, RTT, PPMX, MRX16, MRX79, α-Synuclein, or DJ-1;
h) schizophrenia, wherein, preferably, the gene is NRG1, ERB4, CPLX1), TPH1, TPH2, Neurexin 1, GSK3, GSK3a, GSK3b, 5-HTT (SLC6A4), COMT, DRD (DRD1a), SLC6A3, DAOA, DTNBP1, or DAO (DAO1);
i) secretase related disorders, wherein, preferably, the gene is APH-1 (alpha and beta), presenilin (Psen1), nicastrin (Ncstn), PEN-2, Nos1, Parp1, Nat1, or Nat2; or
j) trinucleotide repeat disorders, wherein, preferably, the gene is HTT (Huntington's Dx), SBMA/SMAX1/AR (Kennedy's Dx), FXN/X25 (Friedrich's Ataxia), ATX3 (Machado-Joseph's Dx), ATXN1 and ATXN2 (spinocerebellar ataxias), DMPK (myotonic dystrophy), Atrophin-1 and Atn1 (DRPLA Dx), CBP (Creb-BP—global instability), VLDLR (Alzheimer's), Atxn7, or Atxn10.

118. The pharmaceutical composition of claim 109, wherein the disease or disorder is neoplasia, wherein, preferably, the gene is PTEN, ATM, ATR, EGFR, ERBB2, ERBB3, ERBB4, Notch1, Notch2, Notch3, Notch4, AKT, AKT2, AKT3, HIF, HIF1a, HIF3a, MET, HRG, Bcl2, PPAR alpha, PPAR gamma, WT1 (Wilms Tumor), FGF1, FGF2, FGF3, FGF4, FGF5, CDKN2a, APC, RB (retinoblastoma), MEN1, VHL, BRCA1, BRCA2, AR (androgen receptor), TSG101, IGF, IGF receptor, IGF1 (4 variants), IGF2 (3 variants), IGF 1 receptor, IGF 2 receptor, BAX, BCL2, caspase 1, 2, 3, 4, 6, 7, 8, 9, 12, KRAS, or APC.

119. The pharmaceutical composition of claim 109, wherein the ocular disease is:
a) age-related macular degeneration, wherein, preferably, the gene is Aber, CCL2, CC2, CP (ceruloplasmin), TIMP3, cathepsinD, VLDLR, or CCR2;
b) cataract, wherein, preferably, the gene is CRYAA, CRYA1, CRYBB2, CRYB2, PITX3, BFSP2, CP49, CP47, CRYAA, CRYA1, PAX6, AN2, MGDA, CRYBA1, CRYB1, CRYGC, CRYG3, CCL, LIM2, MP19, CRYGD, CRYG4, BFSP2, CP49, CP47, HSF4, CTM, HSF4, CTM, MIP, AQPO, CRYAB, CRYA2, CTPP2, CRYBB1, CRYGD, CRYG4, CRYBB2, CRYB2, CRYGC, CRYG3, CCL, CRYAA, CRYA1, GJA8, CX50, CAE1, GJA3, CX46, CZP3, CAE3, CCM1, CAM, or KRIT1;
c) corneal clouding or corneal dystrophy, wherein, preferably, the gene is APOA1, TGFBI, CSD2, CDGG1, CSD, BIGH3, CDG2, TACSTD2, TROP2, M1S1, VSX1, RINX, PPCD, PPD, KTCN, COL8A2, FECD, PPCD2, PIP5K3, or CFD;
d) cornea plana (congenital), wherein, preferably, the gene is KERA or CNA2;
e) glaucoma, wherein, preferably, the gene is MYOC, TIGR, GLC1A, JOAG, GPOA, OPTN, GLC1E, FIP2, HYPL, NRP, CYP1B1, GLC3A, OPA1, NTG, NPG, CYP1B1, or GLC3A;
f) Leber congenital amaurosis, wherein, preferably, the gene is CRB1, RP12, CRX, CORD2, CRD, RPGRIP1, LCA6, CORD9, RPE65, RP20, AIPL1, LCA4, GUCY2D, GUC2D, LCA1, CORD6, RDH12, or LCA3; or
g) macular dystrophy, wherein, preferably, the gene is ELOVL4, ADMD, STGD2, STGD3, RDS, RP7, PRPH2, PRPH, AVMD, AOFMD, or VMD2.

120. The pharmaceutical composition of claim 109, wherein the disease or disorder is schizophrenia, wherein, preferably, the gene is neuregulin1 (NRG1), ERB4, Complexin1 (CPLX1), TPH1, TPH2, NRXN1, GSK3, GSK3a, or GSK3b.

121. The pharmaceutical composition of claim 109, wherein the disease or disorder is epilepsy, wherein, preferably, the gene is EPM2A, MELF, EPM2, NHLRC1, EPM2A, or EPM2B.

122. The pharmaceutical composition of claim 109, wherein the disease is Duchenne muscular dystrophy, wherein, preferably, the gene is DMD or BMD.

123. The pharmaceutical composition of claim 109, wherein the viral disease or disorder is:
a) AIDS, wherein, preferably, the gene is KIR3DL1, NKAT3, NKB1, AMB11, KIR3DS1, IFNG, CXCL12, or SDF1
b) HIV, wherein, preferably, the gene is CCL5, SCYA5, D17S136E, or TCP228;
c) HIV susceptibility or infection, wherein, preferably, the gene is IL10, CSIF, CMKBR2, CCR2, CMKBR5, or CCCKR5 (CCR5).

124. The pharmaceutical composition of claim 109, wherein the disease or disorder is alpha 1-Antitrypsin deficiency, wherein, preferably, the gene is SERPINA1 [serpin peptidase inhibitor, clade A (alpha-1 antiproteinase, antitrypsin), member 1], SERPINA2, SERPINA3, SERPINA5, SERPINA6, or SERPINA7.

125. A method of homology directed repair, wherein the method comprises:
a) delivering to a target cell a gene editing system comprising:
i) a first guide ribonucleic acid (RNA) directed to a first genomic site of an endogenous DNA molecule of the target cell,
ii) a second guide RNA directed to a second genomic site of the endogenous DNA molecule of the target cell,
iii) a plurality of fusion proteins comprising a first domain comprising an active RNA programmable nuclease and a second domain comprising an exonuclease, and, optionally, and
iv) a donor DNA molecule,
wherein the first guide RNA forms a first complex with a first said fusion protein at the first genomic site and the second guide RNA forms a second complex with a second said fusion protein at the second genomic site, and wherein the first and second complexes promote the homology directed repair by creating a lesion between the first and second genomic sites and, optionally, wherein the homology directed repair comprises insertion of the donor DNA molecule at the lesion between the first and second genomic sites.

126. The method of claim 125, wherein the first and second guide RNAs specifically hybridize to the first and second genomic sites, respectively.

127. The method of claim 125, wherein the first genomic site and the second genomic site are between 10-100000 nucleotide base pairs apart.

128. The method of claim 125, wherein said first genomic site comprises a protospacer adjacent motif (PAM) recognition sequence positioned:
a) downstream from said first genomic site, and said second genomic site comprises a PAM recognition sequence downstream of said second genomic site;
b) downstream from said first genomic site, and said second genomic site comprises a PAM recognition sequence upstream of said second genomic site;
c) upstream from said first genomic site, and said second genomic site comprises a PAM recognition sequence upstream of said second genomic site; or
d) upstream from said first genomic site, and said second genomic site comprises a PAM recognition sequence downstream of said second genomic site.

129. The method of claim 125, wherein said first and second guide RNAs are two single guide RNAs, wherein said first guide RNA targets a first strand of the endogenous DNA molecule, and said second guide RNA targets a complementary strand of the endogenous DNA molecule, and said first domain of the fusion protein cleaves each strand of the endogenous DNA molecule, thereby creating a double-stranded break, and said second domain of the fusion protein cleaves the terminal nucleic acids of each strand of the endogenous DNA molecule, thereby creating elongated single stranded nucleic acid overhangs.

130. The method of claim 125, wherein a region between the first and second genomic sites is associated with a disease.

131. The method of claim 125, wherein the gene editing system further comprises a third and fourth guide RNA.

132. The method claim 125, wherein the donor DNA molecule further comprises flanking regions modified to allow for specificity of targeting of one or more guide RNAs.

133. The method of claim 132, wherein the one or more guide RNAs are the third and fourth guide RNAs.

134. The method of claim 133, wherein the third guide RNA forms a complex with a first said fusion protein at a first said flanking region on the donor DNA molecule and the fourth guide RNA forms a complex with a second said fusion protein at a second said flanking region on the donor DNA molecule, and wherein said complexes cleave the donor DNA molecule at the flanking regions thereby releasing the donor DNA molecule.

135. The method of claim 125, wherein the first domain is a Cas RNA programmable nuclease.

136. The method of claim 135, wherein the Cas RNA programmable nuclease is a Cas9 RNA programmable nuclease.

137. The method of claim 125, wherein the second domain comprises an exonuclease selected from the group consisting of Lambda exonuclease, RecJf exonuclease, exonuclease III, exonuclease I, thermolabile exonuclease I, exonuclease T, exonuclease V (RecBCD), exonuclease VIII (truncated), exonuclease VII, nuclease BAL-31, T5 exonuclease, and T7 exonuclease.

138. The method of claim 137, wherein the exonuclease is Lambda exonuclease.

139. The method of claim 125, wherein the method further comprises delivering an RNA programmable nuclease inhibitor to the target cell.

140. The method of claim 139, wherein the RNA programmable nuclease inhibitor is delivered as a nucleic acid comprising a sequence encoding the RNA programmable nuclease inhibitor.

141. The method of claim 139, wherein the donor DNA molecule comprises a polynucleotide sequence encoding the RNA programmable nuclease inhibitor.

142. The method of claim 139, wherein insertion of the donor DNA molecule at the lesion between the first and second genomic sites promotes expression of the RNA programmable nuclease inhibitor in the target cell, thereby inhibiting activity of the RNA programmable nuclease.

143. The method of claim 139, wherein the RNA programmable nuclease inhibitor is delivered as a polypeptide.

144. The method of claim 139, wherein the RNA programmable nuclease inhibitor is selected from the group consisting of AcrIIA1, AcrIIA2, AcrIIA3, AcrIIA5, AcrIIA5, AcrIIC1, AcrIIC2, or AcrIIC3.

145. The method of claim 144, wherein the RNA programmable nuclease is AcrIIA4.

146. The method of claim 125, wherein the first or second genomic site comprises a nucleotide polymorphism.

147. The method of claim 125, wherein the donor DNA molecule comprises a nucleic acid sequence encoding a gene not associated with a disease or disorder, wherein the homology directed repair comprises insertion of the donor DNA molecule at the lesion between the first and second genomic site, thereby correcting a nucleic acid sequence associated with a disease or disorder.

148. A nucleic acid comprising a polynucleotide comprising a nucleic acid sequence encoding a fusion protein comprising an RNA programmable nuclease and an exonuclease.

149. The nucleic acid of claim 148, further comprising a polynucleotide comprising a nucleic acid sequence encoding a first guide RNA and a second guide RNA.

150. The nucleic acid of claim 149, wherein the first and second guide RNA are directed to first and second genomic sites, respectively, of an endogenous DNA molecule of a cell.

151. The nucleic acid of claim 148, further comprising a polynucleotide comprising a nucleic acid sequence encoding a donor DNA molecule.

152. The nucleic acid of claim 148, further comprising a polynucleotide comprising a nucleic acid sequence encoding a third guide RNA and a fourth guide RNA.

153. The nucleic acid of claim 151, where the polynucleotide comprising a nucleic acid sequence encoding a donor DNA molecule further comprises flanking regions of said donor DNA molecule and wherein said flanking regions are modified to allow for specificity of targeting of one or more guide RNAs.

154. The nucleic acid of claim 151, wherein the donor DNA molecule comprises a nucleic acid sequence encoding a region of a gene, wherein preferably the region lacks a mutation or polymorphism associated with a disease or disorder.

155. The nucleic acid of claim 148, further comprising a promoter.

156. The nucleic acid of claim 148, wherein the RNA programmable nuclease is a Cas RNA programmable nuclease.

157. The nucleic acid of claim 156, wherein the Cas RNA programmable nuclease is a Cas9 RNA programmable nuclease.

158. The nucleic acid of claim 148, wherein the exonuclease is selected from the group consisting of Lambda exonuclease, RecJf exonuclease, exonuclease III, exonuclease I, thermolabile exonuclease I, exonuclease T, exonuclease V (RecBCD), exonuclease VIII (truncated), exonuclease VII, nuclease BAL-31, T5 exonuclease, and T7 exonuclease.

159. The nucleic acid of claim 158, wherein the exonuclease is Lambda exonuclease.

160. The nucleic acid of claim 148, wherein the nucleic acid comprises a nucleic acid encoding a fusion protein comprising an RNA programmable nuclease and an exonuclease, wherein the RNA programmable nuclease and the exonuclease are joined directly or through a linker.

161. The nucleic acid of claim 151, wherein the donor DNA molecule comprises a polynucleotide sequence encoding the RNA programmable nuclease inhibitor.

162. The nucleic acid of claim 161, wherein the RNA programmable nuclease is selected from the group consisting of AcrIIA1, AcrIIA2, AcrIIA3, AcrIIA5, AcrIIA5, AcrIIC1, AcrIIC2, or AcrIIC3.

163. The nucleic acid of claim 162, wherein the RNA programmable nuclease is AcrIIA4.

164. A vector comprising a polynucleotide comprising a nucleic acid sequence encoding a fusion protein comprising an RNA programmable nuclease and an exonuclease.

165. The vector of claim 164, wherein the vector further comprises a polynucleotide comprising a nucleic acid sequence encoding a first and second guide RNA directed to first and second genomic sites, respectively, of an endogenous DNA molecule of a cell.

166. The vector of claim 164, wherein the vector further comprises a polynucleotide comprising a nucleic acid sequence encoding a third guide RNA and a fourth guide RNA.

167. The vector of claim 164, wherein the vector further comprises a polynucleotide comprising a nucleic acid sequence encoding a donor DNA molecule.

168. The vector of claim 167, wherein flanking regions of said donor DNA molecule are modified to allow for specificity of targeting of one or more guide RNAs.

169. The vector of claim 167, wherein the donor DNA molecule further comprises a polynucleotide comprising a nucleic acid sequence encoding an RNA programmable nuclease inhibitor.

170. The vector of claim 164, wherein the RNA programmable nuclease is a Cas RNA programmable nuclease.

171. The vector of claim 170, wherein the Cas RNA programmable nuclease is a Cas9 RNA programmable nuclease.

172. The vector of claim 164, wherein the exonuclease is selected from the group consisting of Lambda exonuclease, RecJf exonuclease, exonuclease III, exonuclease I, thermolabile exonuclease I, exonuclease T, exonuclease V (RecBCD), exonuclease VIII (truncated), exonuclease VII, nuclease BAL-31, T5 exonuclease, and T7 exonuclease.

173. The vector of claim 172, wherein the exonuclease is Lambda exonuclease.

174. The vector of claim 169, wherein the RNA programmable nuclease inhibitor is selected from the group consisting of AcrIIA1, AcrIIA2, AcrIIA3, AcrIIA5, AcrIIA5, AcrIIC1, AcrIIC2, or AcrIIC3.

175. The vector of claim 174, wherein the RNA programmable nuclease is AcrIIA4.

176. The vector of claim 167, wherein the donor DNA molecule comprises a polynucleotide comprising a nucleic acid sequence encoding a region of a gene, wherein preferably the region lacks a mutation or polymorphism associated with a disease or disorder.

177. The vector of claim 164, wherein the RNA programmable nuclease and the exonuclease are joined directly or through a linker.

178. A vector comprising the nucleic acid of claim 148.

179. The vector of claim 178, wherein the vector is an expression vector or a viral vector.

180. The vector of claim 179, wherein the viral vector is a lentiviral vector.

181. A composition comprising:
a) a first guide ribonucleic acid (RNA) directed to a first genomic site of an endogenous DNA molecule of a target cell,
b) a second guide RNA directed to a second genomic site of the endogenous DNA molecule of the target cell,
c) a plurality of fusion proteins, wherein each fusion protein comprises a first domain comprising an active RNA programmable nuclease and a second domain comprising an exonuclease, and, optionally,
d) a donor DNA molecule.

182. The composition of claim 181, wherein the first guide RNA is in a first complex with a first said fusion protein and the second guide RNA is in a second complex with a second said fusion protein, wherein the first and second complexes are configured to promote homology directed repair of the endogenous DNA molecule, optionally, upon insertion of the donor DNA molecule between the first and second genomic sites.

183. The composition of claim 181, wherein the donor DNA molecule comprises a nucleic acid sequence encoding a region of a gene, wherein preferably the region lacks a mutation or polymorphism associated with a disease or disorder.

184. The composition of claim 181, further comprising an RNA programmable nuclease inhibitor.

185. The composition of claim 184, wherein the RNA programmable nuclease inhibitor is selected from the group consisting of AcrIIA1, AcrIIA2, AcrIIA3, AcrIIA5, AcrIIA5, AcrIIC1, AcrIIC2, or AcrIIC3.

186. The composition of claim 185, wherein the RNA programmable nuclease is AcrIIA4.

187. A composition comprising:
a) a first polynucleotide comprising a nucleic acid sequence encoding a first guide ribonucleic acid (RNA) directed to a first genomic site of an endogenous DNA molecule of a target cell;
b) a second polynucleotide comprising a nucleic acid sequence encoding a second guide RNA directed to a second genomic site of the endogenous DNA molecule of the target cell;
c) a third polynucleotide comprising a nucleic acid sequence encoding a fusion protein comprising a first domain comprising an active RNA programmable nuclease and a second domain comprising an exonuclease; and, optionally,
d) a fourth polynucleotide comprising a nucleic acid sequence encoding a donor DNA molecule.

188. The composition of claim 187, wherein the first guide RNA is configured to form a first complex with a first said fusion protein and the second guide RNA is configured to form a second complex with a second said fusion protein, and wherein the first and second complexes are configured to promote homology directed repair of the endogenous DNA molecule, optionally, upon insertion of the donor DNA molecule between the first and second genomic sites.

189. The composition of claim 187, wherein the active RNA programmable nuclease and the exonuclease are joined directly or through a linker.

190. The composition of claim 187, further comprising a fifth polynucleotide comprising a nucleic acid sequence encoding an RNA programmable nuclease inhibitor or wherein the nucleic acid sequence of the fourth polynucleotide further encodes an RNA programmable nuclease inhibitor

191. The composition of claim 187, further comprising:
i) a sixth polynucleotide comprising a nucleic acid sequence encoding a third guide RNA, and
ii) a seventh polynucleotide comprising a nucleic acid sequence encoding a fourth guide RNA.

192. The composition of claim 187, wherein the polynucleotide comprising a nucleic acid sequence encoding the donor DNA molecule further comprises flanking regions modified to allow for specificity of targeting of one or more guide RNAs.

193. The composition of claim 192, wherein the one or more guide RNAs are the third and fourth guide RNAs.

194. The composition of claim 193, wherein the third guide RNA is configured to form a complex with a first said fusion protein at a first said flanking region on the donor DNA molecule and the fourth guide RNA is configured to form a complex with a second said fusion protein at a second said flanking region on the donor DNA molecule, and wherein said complexes cut the donor DNA molecule at the flanking regions, thereby releasing the donor DNA molecule.

195. The composition of claim 187, wherein the donor DNA molecule comprises a nucleic acid sequence encoding a region of a gene, wherein preferably the region lacks a mutation or polymorphism associated with a disease or disorder.

196. The composition of claim 187, wherein the RNA programmable nuclease is a Cas RNA programmable nuclease.

197. The composition of claim 196, wherein the Cas RNA programmable nuclease is a Cas9 RNA programmable nuclease.

198. The composition of claim 187, wherein the exonuclease is selected from the group consisting of Lambda exonuclease, RecJf exonuclease, exonuclease III, exonuclease I, thermolabile exonuclease I, exonuclease T, exonuclease V (RecBCD), exonuclease VIII (truncated), exonuclease VII, nuclease BAL-31, T5 exonuclease, and T7 exonuclease.

199. The composition of claim 198, wherein the exonuclease is Lambda exonuclease.

200. The composition of claim 190, wherein the RNA programmable nuclease inhibitor is selected from the group consisting of AcrIIA1, AcrIIA2, AcrIIA3, AcrIIA5, AcrIIA5, AcrIIC1, AcrIIC2, or AcrIIC3.

201. The composition of claim 200, wherein the RNA programmable nuclease is AcrIIA4.

202. A pharmaceutical composition comprising the nucleic acid of claim 148, the vector of claim 164, or the composition of claim 181, and a pharmaceutically acceptable carrier, excipient, or diluent.

203. A kit comprising the nucleic acid of claim 148, the vector of claim 164, the composition of claim 181, or the pharmaceutical composition of claim 202.

204. The kit of claim 203, wherein the kit comprises the first and second guide RNAs, wherein the first and second guide RNAs are targeted to a genomic site of an endogenous DNA molecule of a target cell causing a disease.

205. The kit of claim 204, wherein the first and second guide RNAs target a nucleotide polymorphism at the genomic site of the endogenous DNA molecule of the target cell.

206. A fusion protein comprising a first domain comprising an active RNA programmable nuclease and a second domain comprising an exonuclease.

207. The fusion protein of claim 206, wherein the first domain is a Cas RNA programmable nuclease.

208. The fusion protein of claim 207, wherein the Cas RNA programmable nuclease is a Cas9 RNA programmable nuclease.

209. The fusion protein of claim 206, wherein the second domain comprises an exonuclease selected from the group consisting of Lambda exonuclease, RecJf exonuclease, exonuclease Ill, exonuclease I, thermolabile exonuclease I, exonuclease T, exonuclease V (RecBCD), exonuclease VIII (truncated), exonuclease VII, nuclease BAL-31, T5 exonuclease, and T7 exonuclease.

210. The fusion protein of claim 209, wherein the exonuclease is Lambda exonuclease.

211. The fusion protein of claim 206, wherein the two domains are joined directly or through a linker.

212. The method of claim 125, wherein the homology directed repair treats a disease or disorder.

213. The method of claim 212, wherein the disease or disorder is selected from a group consisting of age-related macular degeneration; a blood or coagulation disease or disorder; a cell dysregulation or oncology disease or disorder; a developmental disorder; drug addiction; an inflammation or immune related disease or disorder; a metabolic, liver, kidney, or protein disease or disorder; a muscular or skeletal disease or disorder; a neurological or neuronal disease or disorder; a neoplasia; an ocular disease or disorder; schizophrenia; epilepsy; Duchenne muscular dystrophy; a viral disease or disorder, such as AIDS (acquired immunodeficiency syndrome); an autoimmune disorder; and an alpha 1-antitrypsin deficiency.

214. The method of claim 212, wherein the blood or coagulation disease or disorder is:
a) anemia wherein, preferable, the gene is CDAN1, CDA1, RPS19, DBA, PKLR, PK1, NT5C3, UMPH1, PSN1, RHAG, RH50A, NRAMP2, SPTB, ALAS2, ANH1, ASB, ABCB7, ABC7, and/or ASAT;
b) bare lymphocyte syndrome, wherein, preferably, the gene is TAPBP, TPSN, TAP2, ABCB3, PSF2, RING11, MHC2TA, C2TA, RFX5, RFXAP, or RFX5;
c) a bleeding disorder, wherein, preferably, the gene is TBXA2R, P2RX1, or P2X1:
d) a hemolytic anemia, such as a complement Factor H deficiency disease, e.g., a typical hemolytic anemia syndrome (aHUS), wherein, preferably, the gene is HF1, CFH, or HUS;
e) a factor V or factor VIII deficiency disease, wherein, preferably, the gene is MCFD2;
f) a factor VII deficiency disease, wherein, preferably, the gene is F7;
g) a factor X deficiency disease, wherein, preferably, the gene is F10;
h) a factor XI deficiency disease, wherein, preferably, the gene is F11;
i) a factor XII deficiency disease, wherein, preferably, the gene is F12 or HAF;
j) a factor XIIIA deficiency disease, wherein, preferably, the gene is F13A1 or F13A;
k) a factor XIIIB deficiency disease, wherein, preferably, the gene is F13B;
l) Fanconi anemia, wherein, preferably, the gene is FANCA, FACA, FA1, FA, FAA, FAAP95, FAAP90, FLJ34064, FANCB, FANCC, FACC, BRCA2, FANCD1, FANCD2, FANCD, FACD, FAD, FANCE, FACE, FANCF, XRCC9, FANCG, BRIP1, BACH1, FANCJ, PHF9, FANCL, FANCM, or KIAA1596;
m) a hemophagocytic or lymphohistiocytosis disorder, wherein, preferably, the gene is PRF1, HPLH2, UNC13D, MUNC13-4, HPLH3, HLH3, or FHL3;
n) hemophilia A, wherein, preferably, the gene is F8, F8C, or HEMA;
o) hemophilia B, wherein, preferably, the gene is F9 or HEMB;
p) a hemorrhagic disorder, wherein, preferably, the gene is PI, ATT, F5;
q) a leukocyte deficiency or disorder, wherein, preferably, the gene is ITGB2, CD18, LCAMB, LAD, EIF2B1, EIF2BA, EIF2B2, EIF2B3, EIF2B5, LVWM, CACH, CLE, or EIF2B4;
r) sickle cell anemia, wherein, preferably, the gene is HBB; or
s) thalassemia, wherein, preferably, the gene is HBA2, HBB, HBD, LCRB, or HBA1.

215. The method of claim 213, wherein the cell dysregulation or oncology disease is:
a) B-cell non-Hodgkin lymphoma, wherein, preferably, the gene is BCL7A or BCL7; or
b) a leukemia, wherein, preferably, the gene is TAL1 TCL5, SCL, TAL2, FLT3, NBS1, NBS, ZNFN1A1, IK1, LYF1, HOXD4, HOX4B, BCR, CML, PHL, ALL, ARNT, KRAS2, RASK2, GMPS, AF10, ARHGEF12, LARG, KIAA0382, CALM, CLTH, CEBPA, CEBP, CHIC2, BTL, FLT3, KIT, PBT, LPP, NPM1, NUP214, D9S46E, CAN, CAIN, RUNX1, CBFA2, AML1, WHSC1L1, NSD3, FLT3, AF1Q, NPM1, NUMA1, ZNF145, PLZF, PML, MYL, STAT5B, AF10, CALM, CLTH, ARL11, ARLTS1, P2RX7, P2X7, BCR, CML, PHL, ALL, GRAF, NF1, VRNF, WSS, NFNS, PTPN11, PTP2C, SHP2, NS1, BCL2, CCND1, PRAD1, BCL1, TCRA, GATA1, GF1, ERYF1, NFE1, ABL1, NQO1, DIA4, NMOR1, NUP214, D9S46E, CAN, or CAIN.

216. The method of claim 213, wherein the developmental disease is:
a) Angelman syndrome, wherein, preferably, the gene is UBE3A or a 15q11-13 deletion;
b) Canavan disease, wherein, preferably, the gene is ASPA;
c) Cri-du-chat syndrome, wherein, preferably, the gene is 5P− (5p minus) or CTNND2;
d) Down syndrome, wherein, preferably, the gene is Trisomy 21;
e) Klinefelter syndrome, wherein, preferably, the gene is XXY or two or more X chromosomes in males;
f) Prader-Willi syndrome, wherein, preferably, the gene is deletion of chromosome 15 segment or a duplication of maternal chromosome 15; or
g) Turner syndrome where the gene is monosomy X or SHOX.

217. The method of claim 213, wherein the disease or disorder is a drug addiction, wherein, preferably, the gene is PRKCE, DRD2, DRD4, ABAT (alcohol), GRIA2, GRM5, GRIN1, HTR1B, GRIN2A, DRD3, PDYN, GRIA1 (alcohol).

218. The method of claim 213, wherein the inflammation or immune related disease is:
a) autoimmune lymphoproliferative syndrome, wherein, preferably, the gene TNFRSF6, APT1, FAS, CD95, or ALPS1A;
b) combined immuno-deficiency, wherein, preferably, the gene is IL2RG, SCIDX1, SCIDX, or IMD4;
c) an immuno-deficiency, wherein, preferably, the gene is CD3E, CD3G, AICDA, AID, HIGM2, TNFRSF5, CD40, UNG, DGU, HIGM4, TNFSF5, CD40LG, HIGM1, IGM, FOXP3, IPEX, AIID, XPID, PIDX, TNFRSF14B, or TACI;
d) inflammation wherein, preferably, the gene is IL-10, IL-1 (IL-1a, IL-1 b), IL-13, IL-17 (IL-17a (CTLA8), IL-17b, IL-17c, IL-17d, IL-17f), Il-23, CX3CR1, PTPN22, TNFa, NOD2/CARD15 for IBD, IL-6, IL-12 (IL-12a, IL-12b), CTLA4, or CX3CL1; or
e) severe combined immunodeficiency disease, wherein, preferably, the gene is JAK3, JAKL, DCLRE1C, ARTEMIS, SCIDA, RAG1, RAG2, ADA, PTPRC, CD45, LCA, IL7R, CD3D, T3D, IL2RG, SCIDX1, SCIDX, or IMD4.

219. The method of claim 213, wherein the metabolic, liver, kidney, or protein disease is:
a) amyloid neuropathy, wherein, preferably, the gene is TTR or PALB;
b) amyloidosis, wherein, preferably, the gene is APOA1, APP, AAA, CVAP, AD1, GSN, FGA, LYZ, TTR, or PALB;
c) cirrhosis, wherein, preferably, the gene is KRT18, KRT8, CIRH1A, NAIC, TEX292, or KIAA1988;
d) cystic fibrosis, wherein, preferably, the gene is CFTR, ABCC7, CF, or MRP7;
e) a glycogen storage disease, wherein, preferably, the gene is SLC2A2, GLUT2, G6PC, G6PT, G6PT1, GAA, LAMP2, LAMPB, AGL, GDE, GBE1, GYS2, PYGL, or PFKM;
f) hepatic adenoma, wherein, preferably, the gene is TCF1, HNF1A, or MODY3;
g) an early onset neurologic disorder, wherein, preferably, the gene is SCOD1 or SCO1;
h) hepatic lipase deficiency, wherein, preferably, the gene is LIPC;
i) hepato-blastoma cancer, wherein, preferably, the gene is CTNNB1, PDGFRL, PDGRL, PRLTS, AXIN1, AXIN, CTNNB1, TP53, P53, LFS1, IGF2R, MPRI, MET, CASP8, or MCH5;
j) medullary cystic kidney disease, wherein, preferably, the gene is UMOD, HNFJ, FJHN, MCKD2, or ADMCKD2;
k) phenylketonuria, wherein, preferably, the gene is PAH, PKU1, QDPR, DHPR, or PTS; or
l) polycystic kidney or hepatic disease, wherein, preferably, the gene is FCYT, PKHD1, ARPKD, PKD1, PKD2, PKD4, PKDTS, PRKCSH, G19P1, PCLD, or SEC63.

220. The method of claim 213, wherein the muscular or skeletal disease is:
a) Becker muscular dystrophy, wherein, preferably, the gene is DMD, BMD, or MYF6;
b) Duchenne muscular dystrophy, wherein, preferably, the gene is DMD or BMD;
c) Emery-Dreifuss muscular dystrophy, wherein, preferably, the gene is LMNA, LMN1, EMD2, FPLD, CMD1A, HGPS, LGMD1B, LMNA, LMN1, EMD2, FPLD, or CMD1A;
d) Facio-scapulohumeral muscular dystrophy, wherein, preferably, the gene is FSHMD1A or FSHD1A;
e) muscular dystrophy, wherein, preferably, the gene is FKRP, MDC1C, LGMD2I, LAMA2, LAMM, LARGE, KIAA0609, MDC1D, FCMD, TTID, MYOT, CAPN3, CANP3, DYSF, LGMD2B, SGCG, LGMD2C, DMDA1, SCG3, SGCA, ADL, DAG2, LGMD2D, DMDA2, SGCB, LGMD2E, SGCD, SGD, LGMD2F, CMD1L, TCAP, LGMD2G, CMD1N, TRIM32, HT2A, LGMD2H, FKRP, MDC1C, LGMD2I, TTN, CMD1G, TMD, LGMD2J, POMT1, CAV3, LGMD1C, SEPN1, SELN, RSMD1, PLEC1, PLTN, or EBS1;
f) osteopetrosis, wherein, preferably, the gene is LRP5, BMND1, LRP7, LR3, OPPG, VBCH2, CLCN7, CLC7, OPTA2, OSTM1, GL, TCIRG1, TIRC7, OC116, or OPTB1;
g) muscular atrophy, wherein, preferably, the gene is VAPB, VAPC, ALS8, SMN1, SMA1, SMA2, SMA3, SMA4, BSCL2, SPG17, GARS, SMAD1, CMT2D, HEXB, IGHMBP2, SMUBP2, CATF1, or SMARD1; or
h) Tay-Sachs disease, wherein, preferably, the gene is HEXA.

221. The method of claim 213, wherein the neurological and neuronal disease is:
a) amyotrophic lateral sclerosis (ALS), wherein, preferably, the gene is SOD1, ALS2, STEX, FUS, TARDBP, or VEGF (VEGF-a, VEGF-b, VEGF-c);
b) Alzheimer's disease, wherein, preferably, the gene is APP, AAA, CVAP, AD1, APOE, AD2, PSEN2, AD4, STM2, APBB2, FE65L1, NOS3, PLAU, URK, ACE, DCP1, ACE1, MPO, PACIP1, PAXIP1L, PTIP, A2M, BLMH, BMH, PSEN1, orAD3;
c) autism, wherein, preferably, the gene is Mecp2, BZRAP1, MDGA2, Sema5A, Neurexin 1, GLO1, MECP2, RTT, PPMX, MRX16, MRX79, NLGN3, NLGN4, KIAA1260, or AUTSX2;
d) Fragile X Syndrome, wherein, preferably, the gene is FMR2, FXR1, FXR2, or mGLUR5;
e) Huntington's disease or a Huntington's disease like disorder, wherein, preferably, the gene is HD, IT15, PRNP, PRIP, JPH3, JP3, HDL2, TBP, or SCA17;
f) Parkinson's disease, wherein, preferably, the gene is NR4A2, NURR1, NOT, TINUR, SNCAIP, TBP, SCA17, SNCA, NACP, PARK1, PARK4, DJ1, PARK7, LRRK2, PARK8, PINK1, PARK6, UCHL1, PARK5, SNCA, NACP, PARK1, PARK4, PRKN, PARK2, PDJ, DBH, or NDUFV2;
g) Rett syndrome, wherein, preferably, the gene is MECP2, RTT, PPMX, MRX16, MRX79, CDKL5, STK9, MECP2, RTT, PPMX, MRX16, MRX79, α-Synuclein, or DJ-1;
h) schizophrenia, wherein, preferably, the gene is NRG1, ERB4, CPLX1), TPH1, TPH2, Neurexin 1, GSK3, GSK3a, GSK3b, 5-HTT (SLC6A4), COMT, DRD (DRD1a), SLC6A3, DAOA, DTNBP1, or DAO (DAO1);
i) secretase related disorders, wherein, preferably, the gene is APH-1 (alpha and beta), presenilin (PSEN1), nicastrin (NCSTN), PEN-2, NOS1, PARP1, NAT1, or NAT2; or
j) trinucleotide repeat disorders, wherein, preferably, the gene is HTT, SBMA/SMAX1/AR, FXN/X25, ATX3, ATXN1, ATXN2, DMPK, Atrophin-1, Atn1, CBP, VLDLR, ATXN7, or ATXN10.

222. The method of claim 213, wherein the disease or disorder is neoplasia, wherein, preferably, the gene is PTEN, ATM, ATR, EGFR, ERBB2, ERBB3, ERBB4, Notch1, Notch2, Notch3, Notch4, AKT, AKT2, AKT3, HIF, HIF1a, HIF3a, MET, HRG, Bcl2, PPAR alpha, PPAR gamma, WT1 (Wilms Tumor), FGF1, FGF2, FGF3, FGF4, FGF5, CDKN2a, APC, RB (retinoblastoma), MEN1, VHL, BRCA1, BRCA2, AR (androgen receptor), TSG101, IGF, IGF receptor, IGF1 (4 variants), IGF2 (3 variants), IGF 1 receptor, IGF 2 receptor, BAX, BCL2, caspase 1, 2, 3, 4, 6, 7, 8, 9, 12, KRAS, or APC.

223. The method of claim 213, wherein the ocular disease is:
a) age-related macular degeneration, wherein, preferably, the gene is Aber, CCL2, CC2, CP (ceruloplasmin), TIMP3, cathepsin D, VLDLR, or CCR2;
b) cataract, wherein, preferably, the gene is CRYAA, CRYA1, CRYBB2, CRYB2, PITX3, BFSP2, CP49, CP47, CRYAA, CRYA1, PAX6, AN2, MGDA, CRYBA1, CRYB1, CRYGC, CRYG3, CCL, LIM2, MP19, CRYGD, CRYG4, BFSP2, CP49, CP47, HSF4, CTM, HSF4, CTM, MIP, AQPO, CRYAB, CRYA2, CTPP2, CRYBB1, CRYGD, CRYG4, CRYBB2, CRYB2, CRYGC, CRYG3, CCL, CRYAA, CRYA1, GJA8, CX50, CAE1, GJA3, CX46, CZP3, CAE3, CCM1, CAM, or KRIT1;
c) corneal clouding or corneal dystrophy, wherein, preferably, the gene is APOA1, TGFBI, CSD2, CDGG1, CSD, BIGH3, CDG2, TACSTD2, TROP2, M1S1, VSX1, RINX, PPCD, PPD, KTCN, COL8A2, FECD, PPCD2, PIP5K3, or CFD;
d) cornea plana (congenital), wherein, preferably, the gene is KERA or CNA2;
e) glaucoma, wherein, preferably, the gene is MYOC, TIGR, GLC1A, JOAG, GPOA, OPTN, GLC1E, FIP2, HYPL, NRP, CYP1B1, GLC3A, OPA1, NTG, NPG, CYP1B1, or GLC3A;
f) Leber congenital amaurosis, wherein, preferably, the gene is CRB1, RP12, CRX, CORD2, CRD, RPGRIP1, LCA6, CORD9, RPE65, RP20, AIPL1, LCA4, GUCY2D, GUC2D, LCA1, CORD6, RDH12, or LCA3; or
g) macular dystrophy, wherein, preferably, the gene is ELOVL4, ADMD, STGD2, STGD3, RDS, RP7, PRPH2, PRPH, AVMD, AOFMD, or VMD2.

224. The method of claim 213, wherein the disease or disorder is schizophrenia, wherein, preferably, the gene is neuregulin1 (NRG1), ERB4, Complexin1 (CPLX1), TPH1, TPH2, NRXN1, GSK3, GSK3a, or GSK3b.

225. The method of claim 213, wherein the disease or disorder is epilepsy, wherein, preferably, the gene is EPM2A, MELF, EPM2, NHLRC1, EPM2A, or EPM2B.

226. The method of claim 213, wherein the disease is Duchenne muscular dystrophy, wherein, preferably, the gene is DMD or BMD.

227. The method of claim 213, wherein the viral disease or disorder is:
a) AIDS, wherein, preferably, the gene is KIR3DL1, NKAT3, NKB1, AMB11, KIR3DS1, IFNG, CXCL12, or SDF1
b) caused by human immunodeficiency virus (HIV), wherein, preferably, the gene is CCL5, SCYA5, D17S136E, or TCP228;
c) HIV susceptibility or infection, wherein, preferably, the gene is IL10, CSIF, CMKBR2, CCR2, CMKBR5, or CCCKR5 (CCR5).

228. The method of claim 213, wherein the disease or disorder is alpha 1-antitrypsin deficiency, wherein, preferably, the gene is SERPINA1 [serpin peptidase inhibitor, clade A (alpha-1 antiproteinase, antitrypsin), member 1], SERPINA2, SERPINA3, SERPINA5, SERPINA6, or SERPINA7.

229. The method of claim 125, wherein the homology directed repair treats a cellular dysfunction.

230. The method of claim 229, wherein the cellular dysfunction is associated with PI3K/AKT signaling, ERK/MAPK signaling, glucocorticoid receptor signaling, axonal guidance signaling, ephrin receptor signaling, actin cytoskeleton signaling, Huntington's disease signaling, apoptosis signaling, B cell receptor signaling, leukocyte extravasation signaling, integrin signaling, acute phase response signaling, PTEN signaling, p53 signaling, aryl hydrocarbon receptor signaling, xenobiotic metabolism signaling, SAPK/JNK signaling, PPAr/RXR signaling, NF-KB signaling, neuregulin signaling, Wnt or beta catenin signaling, insulin receptor signaling, IL-6 signaling, hepatic cholestasis, IGF-1 signaling, NRF2-mediated oxidative stress response, hepatic signaling, fibrosis or hepatic stellate cell activation, PPAR signaling, Fc Epsilon RI signaling, G-protein coupled receptor signaling, inositol phosphate metabolism, PDGF signaling, VEGF signaling, natural killer cell signaling, cell cycle G1/S checkpoint regulation, T cell receptor signaling, death receptor signaling, FGF signaling, GM-CSF signaling, amyotrophic lateral sclerosis signaling, JAK/Stat signaling, nicotinate or nicotinamide metabolism, chemokine signaling, IL-2 signaling, synaptic long term depression, estrogen receptor signaling, protein ubiquitination pathway, IL-10 signaling, VDR/RXR activation, TGF-beta signaling, toll-like receptor signaling, p38 MAPK signaling, neurotrophin/TRK signaling, FXR/RXR Activation, synaptic long term potentiation, calcium signaling, EGF signaling, hypoxia signaling in the cardiovascular system, LPS/IL-1 mediated inhibition of RXR function, LXR/RXR activation, amyloid processing, IL-4 signaling, cell cycle G2/M DNA damage checkpoint regulation, nitric oxide signaling in the cardiovascular system, purine metabolism, cAMP-mediated signaling, mitochondrial dysfunction notch signaling, endoplasmic reticulum stress pathway, pyrimidine metabolism, Parkinson's signaling, cardiac or beta adrenergic signaling, glycolysis or gluconeogenesis, interferon signaling, sonic hedgehog signaling, glycerophospholipid metabolism, phospholipid degradation, tryptophan metabolism, lysine degradation, nucleotide excision repair pathway, starch and sucrose metabolism, amino sugars metabolism, arachidonic acid metabolism, circadian rhythm signaling, coagulation system, dopamine receptor signaling, glutathione metabolism, glycerolipid metabolism, linoleic acid metabolism, methionine metabolism, pyruvate metabolism, arginine and proline metabolism, eicosanoid signaling, fructose and mannose metabolism, galactose metabolism, stilbene, coumarine and lignin biosynthesis, antigen presentation, pathway, biosynthesis of steroids, butanoate metabolism, citrate cycle, fatty acid metabolism, histidine metabolism, inositol metabolism, metabolism of xenobiotics by cytochrome p450, methane metabolism, phenylalanine metabolism, propanoate metabolism, selenoamino acid metabolism, sphingolipid metabolism, aminophosphonate metabolism, androgen or estrogen metabolism, ascorbate or aldarate metabolism, bile acid biosynthesis, cysteine metabolism, fatty acid biosynthesis, glutamate receptor signaling, NRF2-mediated oxidative stress response, pentose phosphate pathway, pentose and glucuronate interconversions, retinol metabolism, riboflavin metabolism, tyrosine metabolism, ubiquinone biosynthesis, valine, leucine and isoleucine degradation, glycine, serine and threonine metabolism, lysine degradation, pain/taste, pain, mitochondrial function, or developmental neurology.

231. The method of claim 229, wherein the cellular dysfunction is associated with:
i) PI3K/AKT signaling, wherein, preferably, the gene is PRKCE, ITGAM, ITGA5, IRAK1, PRKAA2, EIF2AK2, PTEN, EIF4E, PRKCZ, GRK6, MAPK1, TSC1, PLK1, AKT2, IKBKB, PIK3CA, CDK8, CDKN1B, NFKB2, BCL2, PIK3CB, PPP2R1A, MAPK8, BCL2L1, MAPK3, TSC2, ITGA1, KRAS, EIF4EBP1, RELA, PRKCD, NOS3, PRKAA1, MAPK9, CDK2, PPP2CA, PIM1, ITGB7, YWHAZ, ILK, TP53, RAF1., IKBKG, RELB, DYRK1A, CDKN1A, ITGB1, MAP2K2, JAK1, AKT1, JAK2, PIK3R1, CHUK, PDPK1, PPP2R5C, CTNNB1., MAP2K1, NFKB1, PAK3, ITGB3, CCND1, GSK3A, FRAP1, SFN, ITGA2, TTK, CSNK1A1, BRAF, GSK3B, AKT3, FOXO1, SGK, HSP90AA1, or RPS6KB1;
ii) ERK/MAPK signaling, wherein, preferably, the gene is PRKCE, ITGAM, ITGA5, HSPB1, IRAK1, PRKAA2, EIF2AK2, RAC1, RAP1A, TLN1, EIF4E, ELK1, GRK6, MAPK1, RAC2, PLK1, AKT2, PIK3CA, CDK8, CREB1, PRKCI, PTK2, FOS, RPS6KA4, PIK3CB, PPP2R1A, PIK3C3, MAPK8, MAPK3, ITGA1, ETS1, KRAS, MYCN, EIF4EBP1, PPARG, PRKCD, PRKAA1, MAPK9, SRC, CDK2, PPP2CA, PIM1, PIK3C2A, ITGB7, YWHAZ, PPP1CC, KSR1, PXN, RAF1, FYN, DYRK1A, ITGB1, MAP2K2, PAK4, PIK3R1, STAT3, PPP2R5C, MAP2K1, PAK3, ITGB3, ESR1, ITGA2, MYC, TTK, CSNK1A1, CRKL, BRAF, ATF4, PRKCA, SRF, STAT1, or SGK;
iii) glucocorticoid receptor signaling, wherein, preferably, the gene is RAC1, TAF4B, EP300, SMAD2, TRAF6, PCAF, ELK1, MAPK1, SMAD3, AKT2, IKBKB, NCOR2, UBE2I, PIK3CA, CREB1, FOS, HSPA5, NFKB2, BCL2, MAP3K14, STAT5B, PIK3CB, PIK3C3, MAPK8, BCL2L1, MAPK3, TSC22D3, MAPK10, NRIP1, KRAS, MAPK13, RELA, STAT5A, MAPK9, NOS2A, PBX1, NR3C1, PIK3C2A, CDKN1C, TRAF2, SERPINE1, NCOA3, MAPK14, TNF, RAF1, IKBKG, MAP3K7, CREBBP, CDKN1A, MAP2K2, JAK1, IL8, NCOA2, AKT1, JAK2, PIK3R1, CHUK, STAT3, MAP2K1, NFKB1, TGFBR1, ESR1, SMAD4, CEBPB, JUN, AR, AKT3, CCL2, MMP1, STAT1, 1L6, or HSP90AA1;
iv) axonal guidance signaling, wherein, preferably, the gene is PRKCE, ITGAM, ROCK1, ITGA5, CXCR4, ADAM12, IGF1, RAC1, RAP1A, E1F4E, PRKCZ, NRP1, NTRK2, ARHGEF7, SMO, ROCK2, MAPK1, PGF, RAC2, PTPN11, GNAS, AKT2, PIK3CA, ERBB2, PRKC1, PTK2, CFL1, GNAQ, PIK3CB, CXCL12, PIK3C3, WNT11, PRKD1, GNB2L1, ABL1, MAPK3, ITGA1, KRAS, RHOA, PRKCD, PIK3C2A, ITGB7, GLI2, PXN, VASP, RAF1, FYN, ITGB1, MAP2K2, PAK4, ADAM17, AKT1, PIK3R1, GLI1, WNT5A, ADAM10, MAP2K1, PAK3, ITGB3, CDC42, VEGFA, ITGA2, EPHA8, CRKL, RND1, GSK3B, AKT3, or PRKCA;
v) ephrin receptor signaling, wherein, preferably, the gene is PRKCE, ITGAM, ROCK1, ITGA5, CXCR4, IRAK1, PRKAA2, EIF2AK2, RAC1, RAP1A, GRK6, ROCK2, MAPK1, PGF, RAC2, PTPN11, GNAS, PLK1, AKT2, DOK1, CDK8, CREB1, PTK2, CFL1, GNAQ, MAP3K14, CXCL12, MAPK8, GNB2L1, ABL1, MAPK3, ITGA1, KRAS, RHOA, PRKCD, PRKAA1, MAPK9, SRC, CDK2, PIM1, ITGB7, PXN, RAF1, FYN, DYRK1A, ITGB1, MAP2K2, PAK4, AKT1, JAK2, STAT3, ADAM10, MAP2K1, PAK3, ITGB3, CDC42, VEGFA, ITGA2, EPHA8, TTK, CSNK1A1, CRKL, BRAF, PTPN13, ATF4, AKT3, or SGK;
vi) actin cytoskeleton signaling, wherein, preferably, the gene is ACTN4, PRKCE, ITGAM, ROCK1, ITGA5, IRAK1, PRKAA2, EIF2AK2, RAC1, INS, ARHGEF7, GRK6, ROCK2, MAPK1, RAC2, PLK1, AKT2, PIK3CA, CDK8, PTK2, CFL1, PIK3CB, MYH9, DIAPH1, PIK3C3, MAPK8, F2R, MAPK3, SLC9A1, ITGA1, KRAS, RHOA, PRKCD, PRKAA1, MAPK9, CDK2, PIM1, PIK3C2A, ITGB7, PPP1CC, PXN, VIL2, RAF1, GSN, DYRK1A, ITGB1, MAP2K2, PAK4, PIP5K1A, PIK3R1, MAP2K1, PAK3, ITGB3, CDC42, APC, ITGA2, TTK, CSNK1A1, CRKL, BRAF, VAV3, or SGK;
vii) Huntington's disease signaling, wherein, preferably, the gene is PRKCE, IGF1, EP300, RCOR1., PRKCZ, HDAC4, TGM2, MAPK1, CAPNS1, AKT2, EGFR, NCOR2, SP1, CAPN2, PIK3CA, HDAC5, CREB1, PRKC1, HSPA5, REST, GNAQ, PIK3CB, PIK3C3, MAPK8, IGF1R, PRKD1, GNB2L1, BCL2L1, CAPN1, MAPK3, CASP8, HDAC2, HDAC7A, PRKCD, HDAC11, MAPK9, HDAC9, PIK3C2A, HDAC3, TP53, CASP9, CREBBP, AKT1, PIK3R1, PDPK1, CASP1, APAF1, FRAP1, CASP2, JUN, BAX, ATF4, AKT3, PRKCA, CLTC, SGK, HDAC6, or CASP3;
viii) apoptosis signaling, wherein, preferably, the gene is PRKCE, ROCK1, BID, IRAK1, PRKAA2, EIF2AK2, BAK1, BIRC4, GRK6, MAPK1, CAPNS1, PLK1, AKT2, IKBKB, CAPN2, CDK8, FAS, NFKB2, BCL2, MAP3K14, MAPK8, BCL2L1, CAPN1, MAPK3, CASP8, KRAS, RELA, PRKCD, PRKAA1, MAPK9, CDK2, PIM1, TP53, TNF, RAF1, IKBKG, RELB, CASP9, DYRK1A, MAP2K2, CHUK, APAF1, MAP2K1, NFKB1, PAK3, LMNA, CASP2, BIRC2, TTK, CSNK1A1, BRAF, BAX, PRKCA, SGK, CASP3, BIRC3, or PARP1;
ix) B cell receptor signaling, wherein, preferably, the gene is RAC1, PTEN, LYN, ELK1, MAPK1, RAC2, PTPN11, AKT2, IKBKB, PIK3CA, CREB1, SYK, NFKB2, CAMK2A, MAP3K14, PIK3CB, PIK3C3, MAPK8, BCL2L1, ABL1, MAPK3, ETS1, KRAS, MAPK13, RELA, PTPN6, MAPK9, EGR1, PIK3C2A, BTK, MAPK14, RAF1, IKBKG, RELB, MAP3K7, MAP2K2, AKT1, PIK3R1, CHUK, MAP2K1, NFKB1, CDC42, GSK3A, FRAP1, BCL6, BCL10, JUN, GSK3B, ATF4, AKT3, VAV3, or RPS6KB1;
x) leukocyte extravasation signaling wherein, preferably, the gene is ACTN4, CD44, PRKCE, ITGAM, ROCK1, CXCR4, CYBA, RAC1, RAP1A, PRKCZ, ROCK2, RAC2, PTPN11, MMP14, PIK3CA, PRKCI, PTK2, PIK3CB, CXCL12, PIK3C3, MAPK8, PRKD1, ABL1, MAPK10, CYBB, MAPK13, RHOA, PRKCD, MAPK9, SRC, PIK3C2A, BTK, MAPK14, NOX1, PXN, VIL2, VASP, ITGB1, MAP2K2, CTNND1, PIK3R1, CTNNB1, CLDN1, CDC42, F11R, ITK, CRKL, VAV3, CTTN, PRKCA, MMP1, or MMP9;
xi) integrin signaling wherein, preferably, the gene is ACTN4, ITGAM, ROCK1, ITGA5, RAC1, PTEN, RAP1A, TLN1, ARHGEF7, MAPK1, RAC2, CAPNS1, AKT2, CAPN2, P1K3CA, PTK2, PIK3CB, PIK3C3, MAPK8, CAV1, CAPN1, ABL1, MAPK3, ITGA1, KRAS, RHOA, SRC, PIK3C2A, ITGB7, PPP1CC, ILK, PXN, VASP, RAF1, FYN, ITGB1, MAP2K2, PAK4, AKT1, PIK3R1, TNK2, MAP2K1, PAK3, ITGB3, CDC42, RND3, ITGA2, CRKL, BRAF, GSK3B, or AKT3;
xii) acute phase response signaling wherein, preferably, the gene is IRAK1, SOD2, MYD88, TRAF6, ELK1, MAPK1, PTPN11, AKT2, IKBKB, PIK3CA, FOS, NFKB2, MAP3K14, PIK3CB, MAPK8, RIPK1, MAPK3, IL6ST, KRAS, MAPK13, IL6R, RELA, SOCS1, MAPK9, FTL, NR3C1, TRAF2, SERPINE1, MAPK14, TNF, RAF1, PDK1, IKBKG, RELB, MAP3K7, MAP2K2, AKT1, JAK2, PIK3R1, CHUK, STAT3, MAP2K1, NFKB1, FRAP1, CEBPB, JUN, AKT3, IL1R1, or IL6;
xiii) PTEN signaling wherein, preferably, the gene is ITGAM, ITGA5, RAC1, PTEN, PRKCZ, BCL2L11, MAPK1, RAC2, AKT2, EGFR, IKBKB, CBL, PIK3CA, CDKN1B, PTK2, NFKB2, BCL2, PIK3CB, BCL2L1, MAPK3, ITGA1, KRAS, ITGB7, ILK, PDGFRB, INSR, RAF1, IKBKG, CASP9, CDKN1A, ITGB1, MAP2K2, AKT1, PIK3R1, CHUK, PDGFRA, PDPK1, MAP2K1, NFKB1, ITGB3, CDC42, CCND1, GSK3A, ITGA2, GSK3B, AKT3, FOXO1, CASP3, or RPS6KB1;
xiv) p53 signaling wherein, preferably, the gene is PTEN, EP300, BBC3, PCAF, FASN, BRCA1, GADD45A, BIRC5, AKT2, PIK3CA, CHEK1, TP53INP1, BCL2, PIK3CB, PIK3C3, MAPK8, THBS1, ATR, BCL2L1, E2F1, PMAIP1, CHEK2, TNFRSF10B, TP73, RB1, HDAC9, CDK2, PIK3C2A, MAPK14, TP53, LRDD, CDKN1A, HIPK2, AKT1, RIK3R1, RRM2B, APAF1, CTNNB1, SIRT1, CCND1, PRKDC, ATM, SFN, CDKN2A, JUN, SNAI2, GSK3B, BAX, or AKT3;
xv) aryl hydrocarbon receptor signaling wherein, preferably, the gene is HSPB1, EP300, FASN, TGM2, RXRA, MAPK1, NQO1, NCOR2, SP1, ARNT, CDKN1B, FOS, CHEK1, SMARCA4, NFKB2, MAPK8, ALDH1A1, ATR, E2F1, MAPK3, NRIP1, CHEK2, RELA, TP73, GSTP1, RB1, SRC, CDK2, AHR, NFE2L2, NCOA3, TP53, TNF, CDKN1A, NCOA2, APAF1, NFKB1, CCND1, ATM, ESR1, CDKN2A, MYC, JUN, ESR2, BAX, IL6, CYP1B1, or HSP90AA1;
xvi) xenobiotic metabolism signaling wherein, preferably, the gene is PRKCE, EP300, PRKCZ, RXRA, MAPK1, NQO1, NCOR2, PIK3CA, ARNT, PRKCI, NFKB2, CAMK2A, PIK3CB, PPP2R1A, PIK3C3, MAPK8, PRKD1, ALDH1A1, MAPK3, NRIP1, KRAS, MAPK13, PRKCD, GSTP1, MAPK9, NOS2A, ABCB1, AHR, PPP2CA, FTL, NFE2L2, PIK3C2A, PPARGC1A, MAPK14, TNF, RAF1, CREBBP, MAP2K2, PIK3R1, PPP2R5C, MAP2K1, NFKB1, KEAP1, PRKCA, EIF2AK3, 1L6, CYP1B1, or HSP90AA1;
xvii) SAPK or JNK signaling wherein, preferably, the gene is PRKCE, IRAK1, PRKAA2, EIF2AK2, RAC1, ELK1, GRK6, MAPK1, GADD45A, RAC2, PLK1, AKT2, PIK3CA, FADD, CDK8, PIK3CB, PIK3C3, MAPK8, RIPK1, GNB2L1, IRS1, MAPK3, MAPK10, DAXX, KRAS, PRKCD, PRKAA1, MAPK9, CDK2, PIM1, PIK3C2A, TRAF2, TP53, LCK, MAP3K7, DYRK1A, MAP2K2, PIK3R1, MAP2K1, PAK3, CDC42, JUN, TTK, CSNK1A1, CRKL, BRAF, or SGK;
xviii) PPAr or RXR signaling wherein, preferably, the gene is PRKAA2, EP300, INS, SMAD2, TRAF6, PPARA, FASN, RXRA, MAPK1, SMAD3, GNAS, IKBKB, NCOR2, ABCA1, GNAQ, NFKB2, MAP3K14, STAT5B, MAPK8, IRS1, MAPK3, KRAS, RELA, PRKAA1, PPARGC1A, NCOA3, MAPK14, INSR, RAF1, IKBKG, RELB, MAP3K7, CREBBP, MAP2K2, JAK2, CHUK, MAP2K1, NFKB1, TGFBR1, SMAD4, JUN, IL1R1, PRKCA, IL6, HSP90AA1, or ADIPOQ;
xix) NF-KB signaling wherein, preferably, the gene is IRAK1, EIF2AK2, EP300, INS, MYD88, PRKCZ: TRAF6, TBK1, AKT2, EGFR, IKBKB, PIK3CA, BTRC, NFKB2, MAP3K14, PIK3CB, PIK3C3, MAPK8, RIPK1, HDAC2, KRAS, RELA, PIK3C2A, TRAF2, TLR4: PDGFRB, TNF, INSR, LCK, IKBKG, RELB, MAP3K7, CREBBP, AKT1, PIK3R1, CHUK, PDGFRA, NFKB1, TLR2, BCL10, GSK3B, AKT3, TNFAIP3, or IL1R1;
xx) neuregulin signaling wherein, preferably, the gene is ERBB4, PRKCE, ITGAM, ITGA5: PTEN, PRKCZ, ELK1, MAPK1, PTPN11, AKT2, EGFR, ERBB2, PRKCI, CDKN1B, STAT5B, PRKD1, MAPK3, ITGA1, KRAS, PRKCD, STAT5A, SRC, ITGB7, RAF1, ITGB1, MAP2K2, ADAM17, AKT1, PIK3R1, PDPK1, MAP2K1, ITGB3, EREG, FRAP1, PSEN1, ITGA2, MYC, NRG1, CRKL, AKT3, PRKCA, HSP90AA1, or RPS6KB1;
xxi) Wnt or beta catenin signaling wherein, preferably, the gene is CD44, EP300, LRP6, DVL3, CSNK1E, GJA1, SMO, AKT2, PIN1, CDH1, BTRC, GNAQ, MARK2, PPP2R1A, WNT11, SRC, DKK1, PPP2CA, SOX6, SFRP2: ILK, LEF1, SOX9, TP53, MAP3K7, CREBBP, TCF7L2, AKT1, PPP2R5C, WNT5A, LRP5, CTNNB1, TGFBR1, CCND1, GSK3A, DVL1, APC, CDKN2A, MYC, CSNK1A1, GSK3B, AKT3, or SOX2;
xxii) insulin receptor signaling wherein, preferably, the gene is PTEN, INS, EIF4E, PTPN1, PRKCZ, MAPK1, TSC1, PTPN11, AKT2, CBL, PIK3CA, PRKCI, PIK3CB, PIK3C3, MAPK8, IRS1, MAPK3, TSC2, KRAS, EIF4EBP1, SLC2A4, PIK3C2A, PPP1CC, INSR, RAF1, FYN, MAP2K2, JAK1, AKT1, JAK2, PIK3R1, PDPK1, MAP2K1, GSK3A, FRAP1, CRKL, GSK3B, AKT3, FOXO1, SGK, or RPS6KB1;
xxiii) IL-6 signaling wherein, preferably, the gene is HSPB1, TRAF6, MAPKAPK2, ELK1, MAPK1, PTPN11, IKBKB, FOS, NFKB2: MAP3K14, MAPK8, MAPK3, MAPK10, IL6ST, KRAS, MAPK13, IL6R, RELA, SOCS1, MAPK9, ABCB1, TRAF2, MAPK14, TNF, RAF1, IKBKG, RELB, MAP3K7, MAP2K2, IL8, JAK2, CHUK, STAT3, MAP2K1, NFKB1, CEBPB, JUN, IL1R1, SRF, or IL6;
xxiv) hepatic cholestasis wherein, preferably, the gene is PRKCE, IRAK1, INS, MYD88, PRKCZ, TRAF6, PPARA, RXRA, IKBKB, PRKCI, NFKB2, MAP3K14, MAPK8, PRKD1, MAPK10, RELA, PRKCD, MAPK9, ABCB1, TRAF2, TLR4, TNF, INSR, IKBKG, RELB, MAP3K7, IL8, CHUK, NR11H2, TJP2, NFKB1, ESR1, SREBF1, FGFR4, JUN, IL1R1, PRKCA, or IL6;
xxv) IGF-1 signaling wherein, preferably, the gene is IGF1, PRKCZ, ELK1, MAPK1, PTPN11, NEDD4, AKT2, PIK3CA, PRKC1, PTK2, FOS, PIK3CB, PIK3C3, MAPK8, 1GF1R, IRS1, MAPK3, IGFBP7, KRAS, PIK3C2A, YWHAZ, PXN, RAF1, CASP9, MAP2K2, AKT1, PIK3R1, PDPK1, MAP2K1, IGFBP2, SFN, JUN, CYR61, AKT3, FOXO1, SRF, CTGF, or RPS6KB1;
xxvi) NRF2-mediated oxidative stress response wherein, preferably, the gene is PRKCE, EP300, SOD2, PRKCZ, MAPK1, SQSTM1, NQO1, PIK3CA, PRKC1, FOS, PIK3CB, P1K3C3, MAPK8, PRKD1, MAPK3, KRAS, PRKCD, GSTP1, MAPK9, FTL, NFE2L2, PIK3C2A, MAPK14, RAF1, MAP3K7, CREBBP, MAP2K2, AKT1, PIK3R1, MAP2K1, PPIB, JUN, KEAP1, GSK3B, ATF4, PRKCA, EIF2AK3, or HSP90AA1;
xxvii) hepatic fibrosis or hepatic stellate cell activation wherein, preferably, the gene is EDN1, IGF1, KDR, FLT1, SMAD2, FGFR1, MET, PGF, SMAD3, EGFR, FAS, CSF1, NFKB2, BCL2, MYH9, IGF1R, IL6R, RELA, TLR4, PDGFRB, TNF, RELB, IL8, PDGFRA, NFKB1, TGFBR1, SMAD4, VEGFA, BAX, IL1R1, CCL2, HGF, MMP1, STAT1, IL6, CTGF, or MMP9;
xxviii) PPAR signaling wherein, preferably, the gene is EP300, INS, TRAF6, PPARA, RXRA, MAPK1, IKBKB, NCOR2, FOS, NFKB2, MAP3K14, STAT5B, MAPK3, NRIP1, KRAS, PPARG, RELA, STAT5A, TRAF2, PPARGC1A, PDGFRB, TNF, INSR, RAF1, IKBKG, RELB, MAP3K7, CREBBP, MAP2K2, CHUK, PDGFRA, MAP2K1, NFKB1, JUN, IL1R1, or HSP90AA1;
xxix) Fc epsilon RI signaling wherein, preferably, the gene is PRKCE, RAC1, PRKCZ, LYN, MAPK1, RAC2, PTPN11, AKT2, PIK3CA, SYK, PRKCI, PIK3CB, PIK3C3, MAPK8, PRKD1, MAPK3, MAPK10, KRAS, MAPK13, PRKCD, MAPK9, PIK3C2A, BTK, MAPK14, TNF, RAF1, FYN, MAP2K2, AKT1, PIK3R1, PDPK1, MAP2K1, AKT3, VAV3, or PRKCA;
xxx) G-protein coupled receptor signaling wherein, preferably, the gene is PRKCE, RAP1A, RGS16, MAPK1, GNAS, AKT2, IKBKB, PIK3CA, CREB1, GNAQ, NFKB2, CAMK2A, PIK3CB, PIK3C3, MAPK3, KRAS, RELA, SRC, PIK3C2A, RAF1, IKBKG, RELB, FYN, MAP2K2, AKT1, PIK3R1, CHUK, PDPK1, STAT3, MAP2K1, NFKB1, BRAF, ATF4, AKT3, or PRKCA;
xxxi) inositol phosphate metabolism wherein, preferably, the gene is PRKCE, IRAK1, PRKAA2, EIF2AK2, PTEN, GRK6, MAPK1, PLK1, AKT2, PIK3CA, CDK8, PIK3CB, PIK3C3, MAPK8, MAPK3, PRKCD, PRKAA1, MAPK9, CDK2, PIM1, PIK3C2A, DYRK1A, MAP2K2, PIP5K1A, PIK3R1, MAP2K1, PAK3, ATM, TTK, CSNK1A1, BRAF, or SGK;
xxxii) PDGF signaling wherein, preferably, the gene is EIF2AK2, ELK1, ABL2, MAPK1, PIK3CA, FOS, PIK3CB, PIK3C3, MAPK8, CAV1, ABL1, MAPK3, KRAS, SRC, PIK3C2A, PDGFRB, RAF1, MAP2K2, JAK1, JAK2, PIK3R1, PDGFRA, STAT3, SPHK1, MAP2K1, MYC, JUN, CRKL, PRKCA, SRF, STAT1, or SPHK2;
xxxiii) VEGF signaling wherein, preferably, the gene is ACTN4, ROCK1, KDR, FLT1, ROCK2, MAPK1, PGF, AKT2, PIK3CA, ARNT, PTK2, BCL2, PIK3CB, PIK3C3, BCL2L1, MAPK3, KRAS, HIF1A, NOS3, PIK3C2A, PXN, RAF1, MAP2K2, ELAVL1, AKT1, PIK3R1, MAP2K1, SFN, VEGFA, AKT3, FOXO1, or PRKCA;
xxxiv) natural killer cell signaling wherein, preferably, the gene is PRKCE, RAC1, PRKCZ, MAPK1, RAC2, PTPN11, KIR2DL3, AKT2, PIK3CA, SYK, PRKCI, PIK3CB, PIK3C3, PRKD1, MAPK3, KRAS, PRKCD, PTPN6, PIK3C2A, LCK, RAF1, FYN, MAP2K2, PAK4, AKT1, PIK3R1, MAP2K1, PAK3, AKT3, VAV3, or PRKCA;
xxxv) cell cycle G1/S checkpoint regulation wherein, preferably, the gene is HDAC4, SMAD3, SUV39H1, HDAC5, CDKN1B, BTRC, ATR, ABL1, E2F1, HDAC2, HDAC7A, RB1, HDAC11, HDAC9, CDK2, E2F2, HDAC3, TP53, CDKN1A, CCND1, E2F4, ATM, RBL2, SMAD4, CDKN2A, MYC, NRG1, GSK3B, RBL1, or HDAC6;
xxxvi) T cell receptor signaling wherein, preferably, the gene is RAC1, ELK1, MAPK1, IKBKB, CBL, PIK3CA, FOS, NFKB2, PIK3CB, PIK3C3, MAPK8, MAPK3, KRAS, RELA, PIK3C2A, BTK, LCK, RAF1, IKBKG, RELB, FYN, MAP2K2, PIK3R1, CHUK, MAP2K1, NFKB1, ITK, BCL10, JUN, or VAV3;
xxxvii) death receptor signaling wherein, preferably, the gene is CRADD, HSPB1, BID, BIRC4, TBK1, IKBKB, FADD, FAS, NFKB2, BCL2, MAP3K14, MAPK8, RIPK1, CASP8, DAXX, TNFRSF10B, RELA, TRAF2, TNF, IKBKG, RELB, CASP9, CHUK, APAF1, NFKB1, CASP2, BIRC2, CASP3, or BIRC3;
xxxviii) FGF signaling wherein, preferably, the gene is RAC1, FGFR1, MET, MAPKAPK2, MAPK1, PTPN11, AKT2, PIK3CA, CREB1, PIK3CB, PIK3C3, MAPK8, MAPK3, MAPK13, PTPN6, PIK3C2A, MAPK14, RAF1, AKT1, PIK3R1, STAT3, MAP2K1, FGFR4, CRKL, ATF4, AKT3, PRKCA, or HGF;
xxxix) GM-CSF signaling wherein, preferably, the gene is LYN, ELK1, MAPK1, PTPN11, AKT2, PIK3CA, CAMK2A, STAT5B, PIK3CB, PIK3C3, GNB2L1, BCL2L1, MAPK3, ETS1, KRAS, RUNX1, PIM1, PIK3C2A, RAF1, MAP2K2, AKT1, JAK2, PIK3R1, STAT3, MAP2K1, CCND1, AKT3, or STAT1;
xl) amyotrophic lateral sclerosis signaling wherein, preferably, the gene is BID, IGF1, RAC1, BIRC4, PGF, CAPNS1, CAPN2, PIK3CA, BCL2, PIK3CB, PIK3C3, BCL2L1, CAPN1, PIK3C2A, TP53, CASP9, PIK3R1, RAB5A, CASP1, APAF1, VEGFA, BIRC2, BAX, AKT3, CASP3, or BIRC3;
xli) JAK-Stat signaling wherein, preferably, the gene is PTPN1, MAPK1, PTPN11, AKT2, PIK3CA, STAT5B, PIK3CB, PIK3C3, MAPK3, KRAS, SOCS1, STAT5A, PTPN6, PIK3C2A, RAF1, CDKN1A, MAP2K2, JAK1, AKT1, JAK2, PIK3R1, STAT3, MAP2K1, FRAP1, AKT3, STAT1;
xlii) nicotinate or nicotinamide metabolism wherein, preferably, the gene is PRKCE, IRAK1, PRKAA2, EIF2AK2, GRK6, MAPK1, PLK1, AKT2, CDK8, MAPK8, MAPK3, PRKCD, PRKAA1, PBEF1, MAPK9, CDK2, PIM1, DYRK1A, MAP2K2, MAP2K1, PAK3, NT5E, TTK, CSNK1A1, BRAF, or SGK;
xliii) chemokine signaling wherein, preferably, the gene is CXCR4, ROCK2, MAPK1, PTK2, FOS, CFL1, GNAQ, CAMK2A, CXCL12, MAPK8, MAPK3, KRAS, MAPK13, RHOA, CCR3, SRC, PPP1CC, MAPK14, NOX1, RAF1, MAP2K2, MAP2K1, JUN, CCL2, or PRKCA;
xliv) IL-2 signaling wherein, preferably, the gene is ELK1, MAPK1, PTPN11, AKT2, PIK3CA, SYK, FOS, STAT5B, PIK3CB, PIK3C3, MAPK8, MAPK3, KRAS, SOCS1, STAT5A, PIK3C2A: LCK, RAF1, MAP2K2, JAK1, AKT1, PIK3R1, MAP2K1, JUN, or AKT3;
xlv) synaptic long term depression wherein, preferably, the gene is PRKCE, IGF1, PRKCZ, PRDX6, LYN, MAPK1, GNAS, PRKC1, GNAQ, PPP2R1A, IGF1R, PRKID1, MAPK3, KRAS, GRN, PRKCD, NOS3, NOS2A, PPP2CA, YWHAZ, RAF1, MAP2K2, PPP2R5C, MAP2K1, or PRKCA;
xlvi) estrogen receptor signaling wherein, preferably, the gene is TAF4B, EP300, CARM1, PCAF, MAPK1, NCOR2, SMARCA4, MAPK3, NRIP1, KRAS, SRC, NR3C1, HDAC3, PPARGC1A, RBM9, NCOA3, RAF1, CREBBP, MAP2K2, NCOA2, MAP2K1, PRKDC, ESR1, or ESR2;
xlvii) protein ubiquitination pathway wherein, preferably, the gene is TRAF6, SMURF1, BIRC4, BRCA1, UCHL1, NEDD4, CBL, UBE2I, BTRC, HSPA5, USP7, USP10, FBXW7, USP9X, STUB1, USP22, B2M, BIRC2, PARK2, USP8, USP1, VHL, HSP90AA1, or BIRC3;
xlviii) IL-10 signaling wherein, preferably, the gene is TRAF6, CCR1, ELK1, IKBKB, SP1, FOS, NFKB2, MAP3K14, MAPK8, MAPK13, RELA, MAPK14, TNF, IKBKG, RELB, MAP3K7, JAK1, CHUK, STAT3, NFKB1, JUN, IL1R1, or IL6;
xlix) VDR or RXR activation wherein, preferably, the gene is PRKCE, EP300, PRKCZ, RXRA, GADD45A, HES1, NCOR2, SP1, PRKC1, CDKN1B, PRKD1, PRKCD, RUNX2, KLF4, YY1, NCOA3, CDKN1A, NCOA2, SPP1, LRP5, CEBPB, FOXO1, or PRKCA;
l) TGF-beta signaling wherein, preferably, the gene is EP300, SMAD2, SMURF1, MAPK1, SMAD3, SMAD1, FOS, MAPK8, MAPK3, KRAS, MAPK9, RUNX2, SERPINE1, RAF1, MAP3K7, CREBBP, MAP2K2, MAP2K1, TGFBR1, SMAD4, JUN, or SMAD5;
li) toll-like receptor signaling wherein, preferably, the gene is IRAK1, EIF2AK2, MYD88, TRAF6, PPARA, ELK1, IKBKB, FOS, NFKB2, MAP3K14, MAPK8, MAPK13, RELA, TLR4, MAPK14, IKBKG, RELB, MAP3K7, CHUK, NFKB1, TLR2, or JUN;
lii) p38 MAPK signaling wherein, preferably, the gene is HSPB1, IRAK1, TRAF6, MAPKAPK2, ELK1, FADD, FAS, CREB1, DDIT3, RPS6KA4, DAXX, MAPK13, TRAF2, MAPK14, TNF, MAP3K7, TGFBR1, MYC, ATF4, IL1R1, SRF, or STAT1;
liii) neurotrophin or TRK Signaling wherein, preferably, the gene is NTRK2, MAPK1, PTPN11, PIK3CA, CREB1, FOS, PIK3CB, PIK3C3, MAPK8, MAPK3, KRAS, PIK3C2A, RAF1, MAP2K2, AKT1, PIK3R1, PDPK1, MAP2K1, CDC42, JUN, or ATF4;
liv) FXR or RXR activation wherein, preferably, the gene is INS, PPARA, FASN, RXRA, AKT2, SDC1, MAPK8, APOB, MAPK10, PPARG, MTTP, MAPK9, PPARGC1A, TNF, CREBBP, AKT1, SREBF1, FGFR4, AKT3, or FOXO1;
lv) synaptic long term potentiation wherein, preferably, the gene is PRKCE, RAP1A, EP300, PRKCZ, MAPK1, CREB1, PRKC1, GNAQ, CAMK2A, PRKD1, MAPK3, KRAS, PRKCD, PPP1CC, RAF1, CREBBP, MAP2K2, MAP2K1, ATF4, or PRKCA;
lvi) calcium signaling wherein, preferably, the gene is RAP1A, EP300, HDAC4, MAPK1, HDAC5, CREB1, CAMK2A, MYH9, MAPK3, HDAC2, HDAC7A, HDAC11, HDAC9, HDAC3, CREBBP, CALR, CAMKK2, ATF4, or HDAC6;
lvii) EGF signaling wherein, preferably, the gene is ELK1, MAPK1, EGFR, PIK3CA, FOS, PIK3CB, PIK3C3, MAPK8, MAPK3, PIK3C2A, RAF1, JAK1, PIK3R1, STAT3, MAP2K1, JUN, PRKCA, SRF, or STAT1;
lviii) hypoxia signaling in the cardiovascular system wherein, preferably, the gene is EDN1, PTEN, EP300, NQO1, UBE2I, CREB1, ARNT, HIF1A, SLC2A4, NOS3, TP53, LDHA, AKT1, ATM, VEGFA, JUN, ATF4, VHL, or HSP90AA1;
lix) LPS or IL-1 mediated inhibition of RXR function wherein, preferably, the gene is IRAK1, MYD88, TRAF6, PPARA, RXRA, ABCA1, MAPK8, ALDH1A1, GSTP1, MAPK9, ABCB1, TRAF2, TLR4, TNF, MAP3K7, NR1H2, SREBF1, JUN, or IL1R1;
lx) LXR or RXR activation wherein, preferably, the gene is FASN, RXRA, NCOR2, ABCA1, NFKB2, IRF3, RELA, NOS2A, TLR4, TNF, RELB, LDLR, NR1H2, NFKB1, SREBF1, IL1R1, CCL2, 1L6, or MMP9;
lxi) amyloid processing wherein, preferably, the gene is PRKCE, CSNK1E, MAPK1, CAPNS1, AKT2, CAPN2, CAPN1, MAPK3, MAPK13, MAPT, MAPK14, AKT1, PSEN1, CSNK1A1, GSK3B, AKT3, or APP;
lxii) IL-4 signaling wherein, preferably, the gene is AKT2, PIK3CA, PIK3CB, PIK3C3, IRS1, KRAS, SOCS1, PTPN6, NR3C1, PIK3C2A, JAK1, AKT1, JAK2, PIK3R1, FRAP1, AKT3, or RPS6KB1;
lxiii) cell cycle: G2/M DNA damage checkpoint regulation wherein, preferably, the gene is EP300, PCAF, BRCA1, GADD45A, PLK1, BTRC, CHEK1, ATR, CHEK2, YWHAZ, TP53, CDKN1A, PRKDC, ATM, SFN, or CDKN2A;
lxiv) nitric oxide signaling in the cardiovascular system wherein, preferably, the gene is KDR, FLT1, PGF, AKT2, PIK3CA, PIK3CB, PIK3C3, CAV1, PRKCD, NOS3, PIK3C2A, AKT1, PIK3R1, VEGFA, AKT3, or HSP90AA1;
lxv) purine metabolism wherein, preferably, the gene is NME2, SMARCA4, MYH9, RRM2, ADAR, EIF2AK4, PKM2, ENTPD1, RAD51, RRM2B, TJP2, RAD51C, NT5E, POLD1, or NME1;
lxvi) cAMP-mediated Signaling wherein, preferably, the gene is RAP1A, MAPK1, GNAS, CREB1, CAMK2A, MAPK3, SRC, RAF1, MAP2K2, STAT3, MAP2K1, BRAF, or ATF4;
lxvii) mitochondrial dysfunction wherein, preferably, the gene is SOD2, MAPK8, CASP8, MAPK10, MAPK9, CASP9, PARK7, PSEN1, PARK2, APP, or CASP3;
lxviii) notch signaling wherein, preferably, the gene is HES1, JAG1, NUMB, NOTCH4, ADAM17, NOTCH2, PSEN1, NOTCH3, NOTCH1, or DLL4;
lxix) endoplasmic reticulum stress pathway wherein, preferably, the gene is HSPA5, MAPK8, XBP1, TRAF2, ATF6, CASP9, ATF4, EIF2AK3, or CASP3;
lxx) pyrimidine metabolism wherein, preferably, the gene is NME2, AICDA, RRM2, EIF2AK4, ENTPD1, RRM2B, NT5E, POLD1, or NME1;
lxxi) Parkinson's signaling wherein, preferably, the gene is UCHL1, MAPK8, MAPK13, MAPK14, CASP9, PARK7, PARK2, or CASP3;
lxxii) cardiac or beta adrenergic signaling wherein, preferably, the gene is GNAS, GNAQ, PPP2R1A, GNB2L1, PPP2CA, PPP1CC, or PPP2R5C;
lxxiii) glycolysis or gluconeogenesis wherein, preferably, the gene is HK2, GCK, GPI, ALDH1A1, PKM2, LDHA, or HK1;
lxxiv) interferon signaling wherein, preferably, the gene is IRF1, SOCS1, JAK1, JAK2, IFITM1, STAT1, or IFIT3;
lxxv) Sonic Hedgehog signaling wherein, preferably, the gene is ARRB2, SMO, GLI2, DYRK1A, GLI1, GSK3B, or DYRKIB;
lxxvi) glycerophospholipid metabolism wherein, preferably, the gene is PLD1, GRN, GPAM, YWHAZ, SPHK1, or SPHK2;
lxxvii) phospholipid degradation wherein, preferably, the gene is PRDX6, PLD1, GRN, YWHAZ, SPHK1, or SPHK2;
lxxviii) tryptophan metabolism wherein, preferably, the gene is SIAH2, PRMT5, NEDD4, ALDH1A1, CYP1B1, or SIAH1;
lxxix) lysine degradation wherein, preferably, the gene is SUV39H1, EHMT2, NSD1, SETD7, or PPP2R5C;
lxxx) nucleotide excision repair pathway wherein, preferably, the gene is ERCC5, ERCC4, XPA, XPC, or ERCC1;
lxxxi) starch or sucrose metabolism wherein, preferably, the gene is UCHL1, HK2, GCK, GPI, or HK1;
lxxxii) amino sugars metabolism wherein, preferably, the gene is NQO1, HK2, GCK, or HK1;
lxxxiii) arachidonic acid metabolism wherein, preferably, the gene is PRDX6, GRN, YWHAZ, or CYP1B1;
lxxxiv) circadian rhythm signaling wherein, preferably, the gene is CSNK1E, CREB1, ATF4, or NR1 D1;
lxxxv) coagulation system wherein, preferably, the gene is BDKRB1, F2R, SERPINE1, or F3;
lxxxvi) dopamine receptor signaling wherein, preferably, the gene is PPP2R1A, PPP2CA, PPP1CC, or PPP2R5C;
lxxxvii) glutathione metabolism wherein, preferably, the gene is IDH2, GSTP1, ANPEP, or IDH1;
lxxxviii) glycerolipid metabolism wherein, preferably, the gene is ALDH1A1, GPAM, SPHK1, or SPHK2;
lxxxix) linoleic acid metabolism wherein, preferably, the gene is PRDX6, GRN, YWHAZ, or CYP1B1;
xc) methionine metabolism wherein, preferably, the gene is DNMT1, DNMT3B, AHCY, or DNMT3A;
xci) pyruvate metabolism wherein, preferably, the gene is GLO1, ALDH1A1, PKM2, or LDHA;
xcii) arginine and proline metabolism wherein, preferably, the gene is ALDH1A1, NOS3, or NOS2A;
xciii) eicosanoid signaling wherein, preferably, the gene is PRDX6, GRN, or YWHAZ;
xciv) fructose and mannose metabolism wherein, preferably, the gene is HK2, GCK, or HK1;
xcv) galactose metabolism wherein, preferably, the gene is HK2, GCK, or HK1;
xcvi) stilbene, coumarine, or lignin biosynthesis wherein, preferably, the gene is PRDX6, PRDX1, or TYR;
xcvii) antigen presentation pathway wherein, preferably, the gene is CALR or B2M;
xcviii) biosynthesis of steroids wherein, preferably, the gene is NQO1 or DHCR7;
xcix) butanoate metabolism wherein, preferably, the gene is ALDH1A1 or NLGN1;
c) citrate cycle wherein, preferably, the gene is IDH2 or IDH1;
ci) fatty acid metabolism wherein, preferably, the gene is ALDH1A1 or CYP1B1;
cii) histidine metabolism wherein, preferably, the gene is PRMT5 or ALDH1A1;
ciii) inositol metabolism wherein, preferably, the gene is ERO1L or APEX1;
civ) metabolism of xenobiotics by Cytochrome p450 wherein, preferably, the gene is GSTP1 or CYP1B1;
cv) methane metabolism wherein, preferably, the gene is PRDX6 or PRDX1;
cvi) phenylalanine metabolism wherein, preferably, the gene is PRDX6 or PRDX1;
cvii) propanoate metabolism wherein, preferably, the gene is ALDH1A1 or LDHA;
ciii) selenoamino acid metabolism wherein, preferably, the gene is PRMT5 or AHCY;
cix) sphingolipid metabolism wherein, preferably, the gene is SPHK1 or SPHK2;
cx) aminophosphonate metabolism wherein, preferably, the gene is PRMT5;
cxi) androgen or estrogen metabolism wherein, preferably, the gene is PRMT5;
cxii) ascorbate and aldarate metabolism wherein, preferably, the gene is ALDH1A1;
cxiii) bile acid biosynthesis wherein, preferably, the gene is ALDH1A1;
cxiv) cysteine metabolism wherein, preferably, the gene is LDHA;
cxv) fatty acid biosynthesis wherein, preferably, the gene is FASN;
cxvi) glutamate receptor signaling wherein, preferably, the gene is GNB2L1;
cxvii) NRF2-mediated oxidative stress response wherein, preferably, the gene is PRDX1;
cxiii) pentose phosphate pathway wherein, preferably, the gene is GPI;
cxix) pentose and glucuronate interconversions wherein, preferably, the gene is UCHL1;
cxx) retinol metabolism wherein, preferably, the gene is ALDH1A1;
cxxi) riboflavin metabolism wherein, preferably, the gene is TYR;
cxxii) tyrosine metabolism wherein, preferably, the gene is PRMT5 or TYR;
cxxiii) ubiquinone biosynthesis wherein, preferably, the gene is PRMT5;
cxxiv) valine, leucine and isoleucine degradation wherein, preferably, the gene is ALDH1A1;
cxxv) glycine, serine and threonine metabolism wherein, preferably, the gene is CHKA;
cxxvi) lysine degradation wherein, preferably, the gene is ALDH1A1;
cxxvii) pain or taste wherein, preferably, the gene is TRPM5 or TRPA1;
cxxiii) pain wherein, preferably, the gene is TRPM7, TRPC5, TRPC6, TRPC1, CNR1, CNR2, GRK2, TRPA1, POMC, CGRP, CRF, PKA, ERA, NR2b, TRPM5, PRKACa, PRKACb, PRKAR1a, or PRKAR2a;
cxxix) mitochondrial function wherein, preferably, the gene is AIF, CYTC, SMAC (Diablo), AIFM-1, or AIFM-2;
cxxx) developmental neurology wherein, preferably, the gene is BMP-4, chordin (CHRD), noggin (Nog), WNT, WNT2, WNT2b, WNT3a, WNT4, WNT5a, WNT6, WNT7b, WNT8b, WNT9a, WNT9b, WNT10a, WNT10b, WNT16, beta-catenin, DKK-1, frizzled related proteins, OTX-2, GBX2, FGF-8, Reelin, DAB1, UNC-86, POU4f1, BRN3a, NUMB, or RELN.

232. The pharmaceutical composition according to claim 202, for use in treating a disease or disorder.

233. The pharmaceutical composition for use according to claim 232, wherein the disease or disorder is selected from a group consisting of age-related macular degeneration; a blood or coagulation disease or disorder; a cell dysregulation or oncology disease or disorder; a developmental disorder; drug addiction; an inflammation or immune related disease or disorder; a metabolic, liver, kidney, or protein disease or disorder; a muscular or skeletal disease or disorder; a neurological or neuronal disease or disorder; a neoplasia; an ocular disease or disorder; schizophrenia; epilepsy; Duchenne muscular dystrophy; a viral disease or disorder, such as AIDS (acquired immunodeficiency syndrome); an autoimmune disorder; and an Alpha 1-antitrypsin deficiency.

234. The pharmaceutical composition of claim 233, wherein the blood or coagulation disease or disorder is:
a) anemia wherein, preferable, the gene is CDAN1, CDA1, RPS19, DBA, PKLR, PK1, NT5C3, UMPH1, PSN1, RHAG, RH50A, NRAMP2, SPTB, ALAS2, ANH1, ASB, ABCB7, ABC7, and/or ASAT;
b) bare lymphocyte syndrome wherein, preferably, the gene is TAPBP, TPSN, TAP2, ABCB3, PSF2, RING11, MHC2TA, C2TA, RFX5, RFXAP, or RFX5;
c) a bleeding disorder, wherein, preferably, the gene is TBXA2R, P2RX1, or P2X1;
d) a hemolytic anemia, such as a complement Factor H deficiency disease, e.g., a typical hemolytic anemia syndrome (aHUS), wherein, preferably, the gene is HF1, CFH, or HUS;
e) a factor V or factor VIII deficiency disease, wherein, preferably, the gene is MCFD2;
f) a factor VII deficiency disease, wherein, preferably, the gene is F7;
g) a factor X deficiency disease, wherein, preferably, the gene is F10;
h) a factor XI deficiency disease, wherein, preferably, the gene is F11;
i) a factor XII deficiency disease, wherein, preferably, the gene is F12 or HAF;
j) a factor XIIIA deficiency disease, wherein, preferably, the gene is F13A1 or F13A;
k) a factor XIIIB deficiency disease, wherein, preferably, the gene is F13B;
l) Fanconi anemia, wherein, preferably, the gene is FANCA, FACA, FA1, FA, FAA, FAAP95, FAAP90, FLJ34064, FANCB, FANCC, FACC, BRCA2, FANCD1, FANCD2, FANCD, FACD, FAD, FANCE, FACE, FANCF, XRCC9, FANCG, BRIP1, BACH1, FANCJ, PHF9, FANCL, FANCM, or KIAA1596;
m) a hemophagocytic or lymphohistiocytosis disorder, wherein, preferably, the gene is PRF1, HPLH2, UNC13D, MUNC13-4, HPLH3, HLH3, or FHL3;
n) hemophilia A, wherein, preferably, the gene is F8, F8C, or HEMA;
o) hemophilia B, wherein, preferably, the gene is F9 or HEMB;
p) a hemorrhagic disorder, wherein, preferably, the gene is PI, ATT, F5;
q) a leukocyte deficiency or disorder, wherein, preferably, the gene is ITGB2, CD18, LCAMB, LAD, EIF2B1, EIF2BA, EIF2B2, EIF2B3, EIF2B5, LVWM, CACH, CLE, or EIF2B4;
r) sickle cell anemia, wherein, preferably, the gene is HBB; or
s) thalassemia, wherein, preferably, the gene is HBA2, HBB, HBD, LCRB, or HBA1.

235. The pharmaceutical composition of claim 233, wherein the cell dysregulation or oncology disease is:
a) B-cell non-Hodgkin lymphoma, wherein, preferably, the gene is BCL7A or BCL7; or
b) a leukemia, wherein, preferably, the gene is TAL1 TCL5, SCL, TAL2, FLT3, NBS1, NBS, ZNFN1A1, IK1, LYF1, HOXD4, HOX4B, BCR, CML, PHL, ALL, ARNT, KRAS2, RASK2, GMPS, AF10, ARHGEF12, LARG, KIAA0382, CALM, CLTH, CEBPA, CEBP, CHIC2, BTL, FLT3, KIT, PBT, LPP, NPM1, NUP214, D9S46E, CAN, CAIN, RUNX1, CBFA2, AML1, WHSC1L1, NSD3, FLT3, AF1Q, NPM1, NUMA1, ZNF145, PLZF, PML, MYL, STAT5B, AF10, CALM, CLTH, ARL11, ARLTS1, P2RX7, P2X7, BCR, CML, PHL, ALL, GRAF, NF1, VRNF, WSS, NFNS, PTPN11, PTP2C, SHP2, NS1, BCL2, CCND1, PRAD1, BCL1, TCRA, GATA1, GF1, ERYF1, NFE1, ABL1, NQO1, DIA4, NMOR1, NUP214, D9S46E, CAN, or CAIN.

236. The pharmaceutical composition of claim 233, wherein the developmental disease is:
a) Angelman syndrome, wherein, preferably, the gene is UBE3A or a 15q11-13 deletion;
b) Canavan disease, wherein, preferably, the gene is ASPA;
c) Cri-du-chat syndrome, wherein, preferably, the gene is 5P− (5p minus) or CTNND2;
d) Down syndrome, wherein, preferably, the gene is Trisomy 21;
e) Klinefelter syndrome, wherein, preferably, the gene is XXY or two or more X chromosomes in males;
f) Prader-Willi syndrome, wherein, preferably, the gene is deletion of chromosome 15 segment or a duplication of maternal chromosome 15; or
g) Turner syndrome where the gene is monosomy X or SHOX.

237. The pharmaceutical composition of claim 233, wherein the disease or disorder is a drug addiction disease wherein, preferably, the gene is PRKCE, DRD2, DRD4, ABAT (alcohol), GRIA2, GRM5, GRIN1, HTR1B, GRIN2A, DRD3, PDYN, GRIA1 (alcohol).

238. The pharmaceutical composition of claim 233, wherein the inflammation or immune related disease is:
a) autoimmune lymphoproliferative syndrome, wherein, preferably, the gene TNFRSF6, APT1, FAS, CD95, or ALPS1A;
b) combined immuno-deficiency, wherein, preferably, the gene is IL2RG, SCIDX1, SCIDX, or IMD4;
c) an immunodeficiency, wherein, preferably, the gene is CD3E, CD3G, AICDA, AID, HIGM2, TNFRSF5, CD40, UNG, DGU, HIGM4, TNFSF5, CD40LG, HIGM1, IGM, FOXP3, IPEX, AIID, XPID, PIDX, TNFRSF14B, or TACI;
d) inflammation wherein, preferably, the gene is IL-10, IL-1 (IL-1a, IL-1b), IL-13, IL-17 (IL-17a (CTLA8), IL-17b, IL-17c, IL-17d, IL-17f), Il-23, CX3CR1, PTPN22, TNFa, NOD2/CARD15 for IBD, IL-6, IL-12 (IL-12a, IL-12b), CTLA4, or CX3CL1; or
e) severe combined immunodeficiency disease, wherein, preferably, the gene is (SCIDs) (JAK3, JAKL, DCLRE1C, ARTEMIS, SCIDA, RAG1, RAG2, ADA, PTPRC, CD45, LCA, IL7R, CD3D, T3D, IL2RG, SCIDX1, SCIDX, or IMD4.

239. The pharmaceutical composition of claim 233, wherein the metabolic, liver, kidney, or protein disease is:
a) amyloid neuropathy, wherein, preferably, the gene is TTR or PALB;
b) amyloidosis, wherein, preferably, the gene is APOA1, APP, AAA, CVAP, AD1, GSN, FGA, LYZ, TTR, or PALB;
c) cirrhosis, wherein, preferably, the gene is KRT18, KRT8, CIRH1A, NAIC, TEX292, or KIAA1988;
d) cystic fibrosis, wherein, preferably, the gene is CFTR, ABCC7, CF, or MRP7;
e) a glycogen storage disease, wherein, preferably, the gene is SLC2A2, GLUT2, G6PC, G6PT, G6PT1, GAA, LAMP2, LAMPB, AGL, GDE, GBE1, GYS2, PYGL, or PFKM;
f) a hepatic adenoma, wherein, preferably, the gene is TCF1, HNF1A, or MODY3;
g) an early onset neurologic disorder, wherein, preferably, the gene is SCOD1 or SCO1;
h) a hepatic lipase deficiency, wherein, preferably, the gene is LIPC;
i) hepato-blastoma cancer, wherein, preferably, the gene is CTNNB1, PDGFRL, PDGRL, PRLTS, AXIN1, AXIN, CTNNB1, TP53, P53, LFS1, IGF2R, MPRI, MET, CASP8, or MCH5;
j) medullary cystic kidney disease, wherein, preferably, the gene is UMOD, HNFJ, FJHN, MCKD2, or ADMCKD2;
k) phenylketonuria, wherein, preferably, the gene is PAH, PKU1, QDPR, DHPR, or PTS; or
l) polycystic kidney or hepatic disease, wherein, preferably, the gene is FCYT, PKHD1, ARPKD, PKD1, PKD2, PKD4, PKDTS, PRKCSH, G19P1, PCLD, or SEC63.

240. The pharmaceutical composition of claim 233, wherein the muscular or skeletal disease is:
a) Becker muscular dystrophy, wherein, preferably, the gene is DMD, BMD, or MYF6;
b) Duchenne muscular dystrophy, wherein, preferably, the gene is DMD or BMD;
c) Emery-Dreifuss muscular dystrophy, wherein, preferably, the gene is LMNA, LMN1, EMD2, FPLD, CMD1A, HGPS, LGMD1B, LMNA, LMN1, EMD2, FPLD, or CMD1A;
d) Facio-scapulohumeral muscular dystrophy, wherein, preferably, the gene is FSHMD1A or FSHD1A;
e) muscular dystrophy, wherein, preferably, the gene is FKRP, MDC1C, LGMD2I, LAMA2, LAMM, LARGE, KIAA0609, MDC1D, FCMD, TTID, MYOT, CAPN3, CANP3, DYSF, LGMD2B, SGCG, LGMD2C, DMDA1, SCG3, SGCA, ADL, DAG2, LGMD2D, DMDA2, SGCB, LGMD2E, SGCD, SGD, LGMD2F, CMD1L, TCAP, LGMD2G, CMD1N, TRIM32, HT2A, LGMD2H, FKRP, MDC1C, LGMD2I, TTN, CMD1G, TMD, LGMD2J, POMT1, CAV3, LGMD1C, SEPN1, SELN, RSMD1, PLEC1, PLTN, or EBS1;
f) osteopetrosis, wherein, preferably, the gene is LRP5, BMND1, LRP7, LR3, OPPG, VBCH2, CLCN7, CLC7, OPTA2, OSTM1, GL, TCIRG1, TIRC7, OC116, or OPTB1;
g) muscular atrophy, wherein, preferably, the gene is VAPB, VAPC, ALS8, SMN1, SMA1, SMA2, SMA3, SMA4, BSCL2, SPG17, GARS, SMAD1, CMT2D, HEXB, IGHMBP2, SMUBP2, CATF1, or SMARD1; or
h) Tay-Sachs disease wherein, preferably, the gene is HEXA.

241. The pharmaceutical composition of claim 233, wherein the neurological and neuronal disease is:
a) amyotrophic lateral sclerosis (ALS), wherein, preferably, the gene is SOD1, ALS2, STEX, FUS, TARDBP, or VEGF (VEGF-a, VEGF-b, VEGF-c);
b) Alzheimer's disease, wherein, preferably, the gene is APP, AAA, CVAP, AD1, APOE, AD2, PSEN2, AD4, STM2, APBB2, FE65L1, NOS3, PLAU, URK, ACE, DCP1, ACE1, MPO, PACIP1, PAXIP1L, PTIP, A2M, BLMH, BMH, PSEN1, orAD3;
c) autism, wherein, preferably, the gene is Mecp2, BZRAP1, MDGA2, Sema5A, Neurexin 1, GLO1, MECP2, RTT, PPMX, MRX16, MRX79, NLGN3, NLGN4, KIAA1260, or AUTSX2;
d) Fragile X Syndrome, wherein, preferably, the gene is FMR2, FXR1, FXR2, or mGLUR5;
e) Huntington's disease or a Huntington's disease like disorder, wherein, preferably, the gene is HD, IT15, PRNP, PRIP, JPH3, JP3, HDL2, TBP, or SCA17;
f) Parkinson's disease, wherein, preferably, the gene is NR4A2, NURR1, NOT, TINUR, SNCAIP, TBP, SCA17, SNCA, NACP, PARK1, PARK4, DJ1, PARK7, LRRK2, PARK8, PINK1, PARK6, UCHL1, PARK5, SNCA, NACP, PARK1, PARK4, PRKN, PARK2, PDJ, DBH, or NDUFV2;
g) Rett syndrome, wherein, preferably, the gene is MECP2, RTT, PPMX, MRX16, MRX79, CDKL5, STK9, MECP2, RTT, PPMX, MRX16, MRX79, α-Synuclein, or DJ-1;
h) schizophrenia, wherein, preferably, the gene is NRG1, ERB4, CPLX1), TPH1, TPH2, Neurexin 1, GSK3, GSK3a, GSK3b, 5-HTT (SLC6A4), COMT, DRD (DRD1a), SLC6A3, DAOA, DTNBP1, or DAO (DAO1);
i) secretase related disorders, wherein, preferably, the gene is APH-1 (alpha and beta), presenilin (Psen1), nicastrin (Ncstn), PEN-2, Nos1, Parp1, Nat1, or Nat2; or
j) trinucleotide repeat disorders, wherein, preferably, the gene is HTT (Huntington's Dx), SBMA/SMAX1/AR (Kennedy's Dx), FXN/X25 (Friedrich's Ataxia), ATX3 (Machado-Joseph's Dx), ATXN1 and ATXN2 (spinocerebellar ataxias), DMPK (myotonic dystrophy), Atrophin-1 and Atn1 (DRPLA Dx), CBP (Creb-BP—global instability), VLDLR (Alzheimer's), Atxn7, or Atxn10.

242. The pharmaceutical composition of claim 233, wherein the disease or disorder is neoplasia, wherein, preferably, the gene is PTEN, ATM, ATR, EGFR, ERBB2, ERBB3, ERBB4, Notch1, Notch2, Notch3, Notch4, AKT, AKT2, AKT3, HIF, HIF1a, HIF3a, MET, HRG, Bcl2, PPAR alpha, PPAR gamma, WT1 (Wilms Tumor), FGF1, FGF2, FGF3, FGF4, FGF5, CDKN2a, APC, RB (retinoblastoma), MEN1, VHL, BRCA1, BRCA2, AR (androgen receptor), TSG101, IGF, IGF receptor, IGF1 (4 variants), IGF2 (3 variants), IGF 1 receptor, IGF 2 receptor, BAX, BCL2, caspase 1, 2, 3, 4, 6, 7, 8, 9, 12, KRAS, or APC.

243. The pharmaceutical composition of claim 233, wherein the ocular disease is:
a) age-related macular degeneration, wherein, preferably, the gene is Aber, CCL2, CC2, CP (ceruloplasmin), TIMP3, cathepsin D, VLDLR, or CCR2;
b) cataract, wherein, preferably, the gene is CRYAA, CRYA1, CRYBB2, CRYB2, PITX3, BFSP2, CP49, CP47, CRYAA, CRYA1, PAX6, AN2, MGDA, CRYBA1, CRYB1, CRYGC, CRYG3, CCL, LIM2, MP19, CRYGD, CRYG4, BFSP2, CP49, CP47, HSF4, CTM, HSF4, CTM, MIP, AQPO, CRYAB, CRYA2, CTPP2, CRYBB1, CRYGD, CRYG4, CRYBB2, CRYB2, CRYGC, CRYG3, CCL, CRYAA, CRYA1, GJA8, CX50, CAE1, GJA3, CX46, CZP3, CAE3, CCM1, CAM, or KRIT1;
c) corneal clouding or corneal dystrophy, wherein, preferably, the gene is APOA1, TGFBI, CSD2, CDGG1, CSD, BIGH3, CDG2, TACSTD2, TROP2, M1S1, VSX1, RINX, PPCD, PPD, KTCN, COL8A2, FECD, PPCD2, PIP5K3, or CFD;
d) cornea plana (congenital), wherein, preferably, the gene is KERA or CNA2;
e) glaucoma, wherein, preferably, the gene is MYOC, TIGR, GLC1A, JOAG, GPOA, OPTN, GLC1E, FIP2, HYPL, NRP, CYP1B1, GLC3A, OPA1, NTG, NPG, CYP1B1, or GLC3A;
f) Leber congenital amaurosis, wherein, preferably, the gene is CRB1, RP12, CRX, CORD2, CRD, RPGRIP1, LCA6, CORD9, RPE65, RP20, AIPL1, LCA4, GUCY2D, GUC2D, LCA1, CORD6, RDH12, or LCA3; or
g) macular dystrophy, wherein, preferably, the gene is ELOVL4, ADMD, STGD2, STGD3, RDS, RP7, PRPH2, PRPH, AVMD, AOFMD, or VMD2.

244. The pharmaceutical composition of claim 233, wherein the disease or disorder is schizophrenia, wherein, preferably, the gene is neuregulin1 (NRG1), ERB4, Complexin1 (CPLX1), TPH1, TPH2, NRXN1, GSK3, GSK3a, or GSK3b.

245. The pharmaceutical composition of claim 233, wherein the disease or disorder is epilepsy, wherein, preferably, the gene is EPM2A, MELF, EPM2, NHLRC1, EPM2A, or EPM2B.

246. The pharmaceutical composition of claim 233, wherein the disease is Duchenne muscular dystrophy, wherein, preferably, the gene is DMD or BMD.

247. The pharmaceutical composition of claim 233, wherein the viral disease or disorder is:
a) AIDS, wherein, preferably, the gene is KIR3DL1, NKAT3, NKB1, AMB11, KIR3DS1, IFNG, CXCL12, or SDF1
b) HIV, wherein, preferably, the gene is CCL5, SCYA5, D17S136E, or TCP228;
c) HIV susceptibility or infection, wherein, preferably, the gene is IL10, CSIF, CMKBR2, CCR2, CMKBR5, or CCCKR5 (CCR5).

248. The pharmaceutical composition of claim 233, wherein the disease or disorder is alpha 1-Antitrypsin deficiency, wherein, preferably, the gene is SERPINA1 [serpin peptidase inhibitor, clade A (alpha-1 antiproteinase, antitrypsin), member 1], SERPINA2, SERPINA3, SERPINA5, SERPINA6, or SERPINA7.
